(19) United States
(12) Patent Application Publication
(10) Pub. No.: US 2022/0081691 A1
(43) Pub. Date: Mar. 17, 2022

(54) MODULATING PTPN2 TO INCREASE IMMUNE RESPONSES AND PERTURBING GENE EXPRESSION IN HEMATOPOIETIC STEM CELL LINEAGES

(71) Applicants: Dana-Farber Cancer Institute, Inc, Boston, MA (US); President and Fellows of Harvard College, Cambridge, MA (US)

(72) Inventors: William N. Haining, Newton, MA (US); Arlene H. Sharpe, Brookline, MA (US); Martin Lafleur, Cambridge, MA (US); Thao Nguyen, Cambridge, MA (US)

(21) Appl. No.: 17/265,934

(22) PCT Filed: Aug. 5, 2019
(86) PCT No.: PCT/US19/45067

§ 371 Date: Feb. 4, 2021

Related U.S. Application Data
(60) Provisional application No. 62/805,557, filed on Feb. 14, 2019.
(60) Provisional application No. 62/715,605, filed on Aug. 7, 2018.

(51) Int. Cl. C12N 15/113; A61P 37/04; A61K 31/7088; C12N 15/11; C07K 16/40; A61K 35/28; A61K 9/00; A61P 35/00; A61K 47/50; A61K 39/395; C12Q 1/6886; C12N 15/86; C12N 5/0789; A01K 67/027

(52) U.S. Cl. CPC C12N 15/1137; A61K 48/00; A61K 31/7088; C12N 15/11; C07K 16/40; A61K 35/28; A61K 9/0019; A61P 35/00; A61K 47/50; A61K 39/3955; C12Q 1/6886; C12N 15/86; C12N 5/0647; A01K 67/0271; C12N 2310/14; C12N 2310/141; C12N 2310/20; C12N 2310/122; C12N 2310/531; C07K 2317/77; C12N 2320/31; C12N 2320/32; C12N 2740/15043; A61P 37/04

(57) ABSTRACT

The present invention relates, in part, to methods of treating a subject with a condition that would benefit from an increased immune response comprising administering to the subject a therapeutically effective amount of an agent that inhibits PTPN2. The present invention also provides methods and compositions for perturbing gene expression in hematopoietic cell lineages.

### CROSS-REFERENCE TO RELATED APPLICATIONS

This application claims the benefit of U.S. Provisional Application No. 62/715,605, filed on 7 Aug. 2018, and U.S. Provisional Application No. 62/805,557, filed on 14 Feb. 2019; the entire contents of each of said applications are incorporated herein in their entirety by this reference.

### STATEMENT OF RIGHTS

This invention was made with government support under grant number T32CA207021, P50CA101942, and U19AI133524 awarded by the National Institutes of Health. The U.S. government has certain rights in the invention.

### BACKGROUND OF THE INVENTION

Understanding the mechanisms that regulate innate and adaptive immunity has accelerated the development of immunotherapies for autoimmune and allergic diseases, transplant rejection and cancer (Rainsford et al. (2007) Subcell. Biochem. 42:3-27; Li et al. (2017) Front. Pharmacol. 8:460). The dramatic clinical success of immune checkpoint blockade in a broad range of cancers illustrates how fundamental knowledge of immunoregulation can translate to therapy (LaFleur et al. (2018). J. Immunol. 200:375-383). However, a major rate-limiting step in the development of new immunotherapies is the relative paucity of new targets expressed by immune cells that can be exploited for therapeutic benefit. In particular, limitations in the tools available for perturbing genes of interest in immune populations has hindered the discovery and validation of new therapeutic targets for immune-mediated diseases.

The use of functional genomics and genetic perturbation strategies has provided effective tools for the rapid discovery of new targets in cancer (Moody et al. (2010) Curr. Opin. Mol. Ther. 12:284-293). In particular, shRNA-based screening enabled the classification of tumor suppressors and essential genes in cancer (Westbrook et al. (2005) Cell 121:837-848; Luo et al. (2008) Proc. Natl. Acad. Sci. U.S.A. 105:20380-20385). However, shRNA approaches are limited by the issues of incomplete knockdown and a high degree of off-target effects (Boettcher & McManus (2015) Mol. Cell 58:575-585). Targeted nucleases, such as TALENs and zinc finger nucleases, have enabled the complete knockout of gene targets with improved specificity, but require the custom design of proteins for each target gene (Carbery et al. (2010) Genetics 186:451-459; Sung et al. (2013) Nat. Biotechnol. 31:23-24), which makes screening difficult. CRISPR-Cas9 genome editing methods to knockout genes in mammalian cells have the advantages of targeted nuclease editing with improved modularity (Cong et al. (2013) Science 339:819-823; Jinek et al. (2013) Elife 2:e00471; Mali et al. (2013) Science 339:823-826). Furthermore, CRISPR-Cas9 screening provides several advantages over shRNA-based approaches, such as improved consistency across distinct sgRNAs and higher validation rates for scoring genes (Shalem et al. (2014) Science 343:84-87).

Genetic perturbation approaches in immune cells have the potential to accelerate the discovery and validation of new therapies (Wucherpfennig et al. (2016) Curr. Opin. Immunol. 41:55-61). One current approach is to stimulate T cells to allow transduction with a shRNA/sgRNA-expressing lentiviral vector followed by in vivo transfer of edited T cells (Zhou et al. (2014) Nature 506:52-57; Singer et al. (2017) Cell 171:1221-1223; Milner et al. (2017) Nature 552:253-257). Although this method is rapid, in vitro stimulation of T cells perturbs their long-term differentiation, does not allow for the study of genes expressed during T cell priming, and is only applicable to immune cell populations that are easily transferred intravenously (Godec et al. (2015) Proc. Natl. Acad. Sci. U.S.A. 112:512-517). To circumvent some of these issues, lentiviral transduction of bone marrow precursors has been used and bone marrow chimeras for shRNA-based perturbation of naive T cells were increased without disrupting their differentiation or homeostasis (Godec et al. (2015)Proc. Natl. Acad. Sci. U.S.A. 112:512-517). CRISPR-Cas9 transduction of bone marrow precursors has enabled editing of genes involved in oncogenesis to model hematologic malignancies and in the development of hematopoietic precursors (Heckl et al. (2014) Nat. Biotechnol. 32:941-946; Aubrey et al. (2015) Cell Rep. 10:1422-1432; Giladi et al. (2018) Nat. Cell Biol. doi:10.1038/s41556-018-0121-4). However, these approaches have not been adapted or used for analyzing immune responses in different disease models.

T cell exhaustion is a state of dysfunction observed in CD8⁺ T cells during chronic viral infection and cancer (Zajac et a. (1998) J. Exp. Med. 188: 2205-2213; Wherry (2011) Nature Immunogloy 12:492-499: Baitsch et al. (2011) J. Clin. Invest. 121:2350-2360; Miller et al. (2019) Nat. Immunol.). During T cell exhaustion, CD8⁺ T cells progressively lose functional capabilities such as cytokine production and cytotoxicity, as well as proliferative capacity in response to chronic viral infections (Wherry et al. (2003) J. Virol. 77:4911-4927; Day et al. (2006) Nature 443:350-354). This program is initiated during chronic antigen stimulation (Shin et al. (2007) J. Exp. Med. 204:941-949: Utzschneider et al. (2016), J. Exp. Med 213:1819-1834) and likely evolved as a mechanism to prevent excessive immunopathology during chronic antigenic insults (Cornberg et al. (2013) Front Immunol. 4:475). Exhausted CD8⁺ T cells have distinct transcriptional and epigenetic profiles, indicating that exhaustion is a result of a unique differentiation program (Doering et al. (2012) Immunity 37:1130-1144; Sen et al. (2016) Science 354:1165-1169). PD-1 pathway blockade induces an increase in the functionality of exhausted CD8⁺ T cells, but does not change the epigenetic landscape associated with this state, and as a result, the enhanced functionality is only transient (Pauken et al. (2016) Science 354:1160-1165).

There are at least two subpopulations of exhausted CD8⁺ T cells, each with distinct functional properties and roles during responses to chronic infections. The progenitor population of exhausted cells, defined as PD-1^{int} (Paley et al. (2012) Science 338:1220-1225), CXCR5⁺ (Im et al. (2016) Nature 537:417-421; He et al. (2016) Nature 537:412-428), or Slamf6⁺ (Miller et al. (2019) Nat. Immunol.), possesses enhanced proliferative capacity, polyfunctional cytokine production, and serves as a reservoir of cells for the terminally exhausted population. The terminally exhausted population is defined as PD-1^{hi} (Paley et al. (2012) Science 338:1220-1225) or Tim-3⁺ (Im et al. (2016) Nature 537:417-421; He et al. (2016) Nature 537:412-428) and is the major cytotoxic population, albeit having reduced proliferative capacity and longevity. During responses to PD-1 checkpoint blockade, the progenitor population specifically expands and converts into the terminally exhausted subset (Im et al. (2016) Nature 537:417-421). These subsets have been found in murine and human tumors (Wu et al. (2016) Sci. Immunol. 1:eaai8593; Philip et al. (2017) Nature 545:452-456; Brummelman et al. (2018) J. Exp. Med. 215:2520-2535; Sade-Feldman et al. (2018) Cell 175:998-1013; Thommen et al. (2018) Nat. Med. 24:994-1004; Miller et al. (2019) Nat. Immunol.; Siddiqui et al. (2019) Immunity 50:195-211; Kurtulus et al. (2019) Immunity 50:181-94), and an increased progenitor exhausted to terminally exhausted cell ratio is correlated with responsiveness to checkpoint blockade in melanoma patients (Sade-Feldman et al. (2018) Cell 175:998-1013).

Given the relationship between the exhausted subsets and response to checkpoint blockade, there is great interest in finding ways to regulate their formation and longevity. The transcription factors Eomes, Id2, and Runx3 (Paley et al. (2012) Science 338:1220-1225; He et al. (2016) Nature 537:412-428; Shan et al. (2017) Nat. Immunol. 18:931-939) promote the formation of the terminally exhausted subpopulation, while Tbet, Tcf7, and Bcl6 enhance the formation of the progenitor exhausted subpopulation (Paley et al. (2012) Science 338:1220-1225; Im et al. (2016) Nature 537:417-421; Wu et al. (2016) Sci. Immunol. 1:eaai8593). Moreover, terminally exhausted cells derive from progenitor exhausted cells and there is no evidence to indicate that terminally exhausted cells can revert back to the progenitor subset (Im et al. (2016) Nature 537:417-421; He et al. (2016) Nature 537:412-428). TCR stimulation and the cytokines IL-2, IL-21, IL-12, and type 1 interferon also promote formation of the terminally exhausted subset in LCMV infection (Wu et al. (2016) Sci. Immunol. 1:eaai8593; Snell et al. (2018) Immunity 49:678-94; Danilo et al. (2018) Cell Reports 22:2107-2117).

Thus, there remains a great need in the art for generating systems for in vivo deletion of genes in the hematopoietic system and using such systems to identify oncology targets whose perturbation can effectively increase immune responses to treat conditions of interest, such as cancer and infections. There is also an urgent need to identify therapeutic targets that regulate the balance and functionality of these exhausted subpopulations in chronic viral infection and cancer.

### SUMMARY OF THE INVENTION

The present invention is based, at least in part, on the establishment of Cas9-sgRNA delivery systems that enables rapid in vivo deletion of immunologic genes of interest in hematopoietic stem cells, including the major immune cell lineages thereof, without altering differentiation of mature immune cells and having the ability to form bone marrow chimera in animal hosts. In addition, the system was used to identify new oncology targets whose perturbation can effectively increase immune responses to treat conditions of interest, such as cancer and infections. For example, Ptpn2 was identified as a new negative regulator of CD8⁺ T cell-mediated responses, especially in the context of cancer and infections. It was demonstrated that Ptpn2 is a new regulator of the differentiation of the Tim-3⁺ subpopulation through its action on type 1 interferon signaling early during the response to chronic LCMV viral infection. Deletion of Ptpn2 in CD8⁺ T cells increased differentiation of Tim-3⁺ cells without altering the numbers of Slamf6⁺ cells during LCMV Clone 13 infection. It was found that deletion of Ptpn2 in CD8⁺ T cells promotes their differentiation into cytotoxic CD8⁺ effector T cells and that deletion of Ptpn2 in the hematopoietic system enables clearance of both primary tumors and secondary tumors upon re-challenge. Specifically, Ptpn2 deletion in CD8⁺ T cells promoted the formation of the cytotoxic Tim-3⁺ subset and enhanced cytotoxic CD8⁺ T responses to MC38 colorectal tumors. Deletion of Ptpn2 throughout the immune system resulted in complete clearance of immunogenic MC38 tumors and augmented responses of less immunogenic B16 melanoma tumors to PD-1 checkpoint blockade. These results indicate that increasing the number of cytotoxic Tim-3⁺ CD8⁺ T cells early in the response to tumors can promote effective anti-tumor immunity and implicate Ptpn2 in immune cells as an attractive cancer immunotherapy target, as Ptpn2 inhibition may enhance CD8⁺ T cell-mediated tumor immunity and improve tumor control.

For example, in one aspect, a method of treating a subject having a condition that would benefit from an increased immune response, comprising administering to the subject a therapeutically effective amount of an agent that decreases the copy number, the expression level, and/or the activity of tyrosine-protein phosphatase non-receptor type 2 (Ptpn2) or a fragment thereof, is provided.

Numerous embodiments are further provided that can be applied to any aspect of the present invention and/or combined with any other embodiment described herein. For example, in one embodiment, the agent selectively decreases the phosphatase activity and/or the substrate binding activity of Ptpn2. In another embodiment, the agent is a small molecule inhibitor, CRISPR single-guide RNA (sgRNA), RNA interfering agent, antisense oligonucleotide, peptide or peptidomimetic inhibitor, aptamer, or intrabody. The RNA interfering agent may be, for example, a small interfering RNA (siRNA), CRISPR RNA (crRNA), a small hairpin RNA (shRNA), a microRNA (miRNA), or a piwi-interacting RNA (piRNA). In still another embodiment, the agent is a CRISPR single-guide RNA (sgRNA). In yet another embodiment, the sgRNA comprises a nucleic acid sequence selected from the group consisting of nucleic acid sequence listed in Table 2. In another embodiment, the agent comprises an intrabody, or an antigen binding fragment thereof, which specifically binds to Ptpn2 and/or a substrate of Ptpn2. In still another embodiment, the intrabody, or antigen binding fragment thereof, is murine, chimeric, humanized, composite, or human. In yet another embodiment, the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabodies fragments. In another embodiment, the agent decreases the copy number, the expression level, and/or the activity of Ptpn2 or a fragment thereof in hematopoietic stem cells (HSCs) and/or cells derived therefrom. In still another embodiment, the agent targets HSCs and/or cells derived therefrom, optionally wherein the cells are chimeric antigen receptor (CAR)-T cells. In yet another embodiment, the agent is cell-based. In another embodiment, the agent comprises engineered HSCs and/or cells derived therefrom which have a decreased copy number, expression level, and/or activity of Ptpn2 or a fragment thereof. In still another embodiment, the engineered HSCs and/or cells derived therefrom are administered focally or systemically. In yet another embodiment, the systemic administration is intravenous, intramuscular, intraperitoneal, or intra-articular. In another embodiment, the engineered HSCs and/or cells derived therefrom administered to the subject are autologous, syngeneic, allogeneic, or xenogeneic to the subject. In still another embodiment, the engineered HSCs and/or cells derived therefrom maintain at least 5% decreased copy number, expression level, and/or activity of Ptpn2 or a fragment thereof after administration to the subject. In yet another embodiment, HSCs and/or cells derived therefrom give rise to T cells which maintain a decreased copy number, expression level, and/or activity of Ptpn2 or a fragment thereof, optionally wherein the T cells are CD4⁺ T cells, CD8⁺ T cells, and/or CAR-T cells. In another embodiment, the HSCs and/or cells derived therefrom are CD4⁺ T cells, CD8⁺ T cells, and/or CAR-T cells. In still another embodiment, the agent increases CD4⁺ T cell responses and/or CD8⁺ T cell responses. In yet another embodiment, the agent increases expression of genes specific to CD4⁺ T cells and/or CD8⁺ T cells.

In another embodiment, the condition is a cancer. In still another embodiment, the cancer is selected from the group consisting of a solid tumor, a hematologic cancer, bladder cancer, brain cancer, breast cancer, colon cancer, gastric cancer, glioma, head cancer, leukemia, liver cancer, lung cancer, lymphoma, myeloma, neck cancer, ovarian cancer, melanoma, pancreatic cancer, renal cancer, salivary cancer, stomach cancer, thymic epithelial cancer, and thyroid cancer. In yet another embodiment, the agent reduces the number of proliferating cells in the cancer and/or reduces the volume or size of a tumor comprising the cancer cells. In another embodiment, the agent increases the number of CD4⁺ T cells and/or CD8⁺ T cells in a tumor comprising the cancer cells. In still another embodiment, the agent increases activity of the CD4⁺ T cells and/or CD8⁺ T cells. In yet another embodiment, the agent increases the percentage of CD4⁺ T cells and/or CD8⁺ T cells in tumor, spleen, draining lymph node, and/or blood, optionally wherein the CD8⁺ T cells are Granzyme B+. In another embodiment, the agent leads to an increase in CD25 and a decrease in CD127 expression in CD8⁺ T cells in the tumor-draining lymph node. In still another embodiment, the agent increases TIL Tim3+ signature, mTORC1 signaling, and/or effector-related signatures in CD8⁺ T cells in the tumor. In yet another embodiment, the agent increases TIL Tim3+ signature, mTORC1 signaling, and/or effector-related signatures in CD8⁺ T cells in the tumor. In yet another embodiment, the agent increases the percentage of CD4⁺ T cells, Slamf6-Tim3+ CD8⁺ T cells, Granzyme B+ CD8⁺ T cells, and/or CD44+CD62L− effector CD8⁺ T cells in blood. In still another embodiment, the agent decreases the percentage of CD4⁺ T cells, Slamf6+ Tim3− CD8⁺ T cells, and/or CD127+ CD8⁺ T cells in blood. In yet another embodiment, the methods described herein further comprise administering to the subject at least one additional cancer therapy or regimen, optionally wherein the at least one additional cancer therapy or regimen is administered before, after, or concurrently with the agent and/or the immunotherapy. In another embodiment, the cancer therapy is not an immunotherapy.

In another embodiment, the condition is an infection. In still another embodiment, the infection is a viral infection, bacterial infection, protozoan infection, or helminth infection. In yet another embodiment, the viral infection is a chronic viral infection. In another embodiment,

the viral infection is LCMV Clone 13 viral infection. In still another embodiment, the agent increases the number of CD4⁺ T cells and/or CD8⁺ T cells in spleen, lung, and/or liver. In yet another embodiment, the agent increases CD4⁺ T cells and/or CD8⁺ T cells, optionally wherein the CD8⁺ T cells are Granzyme B+. In another embodiment, the agent increases the ratio of Tim-3+ to Slamf6+ cells. In still another embodiment, the agent increases the percentage of Tim-3+ cells and/or decreases the percentage of CXCR5+ cells. In yet another embodiment, the agent increases the number of Tim-3+ cells. In another embodiment, the agent decreases CD127 expression and/or TCF7 expression in CD8⁺ T cells. In still another embodiment, the agent promotes the formation of terminally exhausted CD4⁺ T cells and/or terminally exhausted CD8⁺ T cells. In yet another embodiment, the terminally exhausted T cells express Gzma, Cd7, Cd244, and/or Cd160. In another embodiment, the terminally exhausted T cells comprise Tim3+CXCR5−, Tim3+Slamf6−, and/or Tim3+Granzyme B+ T cells. In still another embodiment, the agent decreases the formation of stem-like exhausted CD4⁺ T cells and/or stem-like exhausted CD8⁺ T cells. In yet another embodiment, the stem-like exhausted T cells comprise Tim3− CXCR5+, Tim3-Slamf6+, and/or CXCR5+ TCF7+ T cells. In another embodiment, the agent decreases the formation of progenitor exhausted CD8⁺ T cells. In still another embodiment, the progenitor exhausted CD8⁺ T cells express Slamf6, Id3, and/or Tcf7. In yet another embodiment, the agent increases expression of Gzma, Cd160, Stat1, Cd7, Ccl4, and Ccl5 in the terminally exhausted CD8⁺ T cells. In another embodiment, the agent increases expression of Gzma, Gzmk, Cd160, Stat1, Cd7, Ccl4, Ccl5, Pdcd1, Lag3, and Id2 in the progenior exhausted CD8⁺ T cells. In still another embodiment, the agent increases expression of effector-related genes or gene signatures in the terminally exhausted CD8⁺ T cells and/or the progenitor exhausted CD8⁺ T cells. In yet another embodiment, the effector-related gene signature is selected from the group consisting of mTORC1 signaling and effector versus memory profiles. In another embodiment, the agent increases expression of the effector-related genes both early and late post LCMV infection. In still another embodiment, the agent increases Tim3+ CD8⁺ T cell differentiation. In yet another embodiment, the agent increases Tim3+ CD8⁺ T cell differentiation through enhanced IFN-α signaling.

In another aspect, provided herein is a method for monitoring the progression of a condition that would benefit from an increased immune response in a subject, wherein the subject is administered a therapeutically effective amount of an agent that inhibits the copy number, amount, and/or activity of Ptpn2, the method comprising: a) detecting in a subject sample at a first point in time the copy number, amount, and/or activity of Ptpn2 in HSCs and/or cells derived therefrom; b) repeating step a) at a subsequent point in time; and c) comparing the amount or activity of Ptpn2 detected in steps a) and b) to monitor the progression of the cancer in the subject.

In still another aspect, provided herein is a method of assessing the efficacy of an agent that inhibits the copy number, amount, and/or activity of Ptpn2 for treating a condition that would benefit from an increased immune response in a subject, comprising: a) detecting in a subject sample at a first point in time the copy number, amount, and/or or activity of Ptpn2 in HSCs and/or cells derived therefrom; b) repeating step a) during at least one subsequent point in time after administration of the agent; and c) comparing the copy number, amount, and/or activity detected in steps a) and b), wherein the absence of, or a significant decrease in, the copy number, amount, and/or activity of, Ptpn2, in the subsequent sample as compared to the copy number, amount, and/or activity in the sample at the first point in time, indicates that the agent treats the condition in the subject.

As described above, numerous embodiments are further provided that can be applied to any aspect of the present invention and/or combined with any other embodiment described herein. For example, in one embodiment, the first and/or at least one subsequent sample is selected from the group consisting of ex vivo and in vivo samples. In another embodiment, the first and/or at least one subsequent sample is a portion of a single sample or pooled samples obtained from the subject. In still another embodiment, the condition is a cancer or infection. In yet another embodiment, the cancer is selected from the group consisting of a solid tumor, a hematologic cancer, bladder cancer, brain cancer, breast cancer, colon cancer, gastric cancer, glioma, head cancer, leukemia, liver cancer, lung cancer, lymphoma, myeloma, neck cancer, ovarian cancer, melanoma, pancreatic cancer, renal cancer, salivary cancer, stomach cancer, thymic epithelial cancer, and thyroid cancer. In another embodiment, between the first point in time and the subsequent point in time, the subject has undergone treatment, completed treatment, and/or is in remission for the cancer. In still another embodiment, the cancer treatment is selected from the group consisting of immunotherapy, targeted therapy, chemotherapy, radiation therapy, hormonal therapy, an anti-cancer vaccine, an anti-cancer virus, and a checkpoint inhibitor. In yet another embodiment, the sample comprises cells, serum, peritumoral tissue, and/or intratumoral tissue obtained from the subject. In another embodiment, the agent is administered in a pharmaceutically acceptable formulation. In still another embodiment, Ptpn2 comprises a nucleic acid sequence having at least 95% identity to a nucleic acid sequence listed in Table 1 and/or encodes an amino acid sequence having at least 95% identity to an amino acid sequence listed in Table 1. In yet another embodiment, Ptpn2 is human, mouse, chimeric, or a fusion. In another embodiment, the subject is an animal model of the condition that would benefit from an increased immune response. In still another embodiment, the animal model is a mouse model. In yet another embodiment, the subject is a mammal. In another embodiment, the mammal is a mouse or a human. In still another embodiment, the mammal is a human.

In yet another aspect, provided herein is a method of generating transduced hematopoietic stem cells (HSCs) and/or cells derived therefrom that are differentiated in vivo, comprising transducing the cells with at least one viral vector, wherein each viral vector integrates an exogenous nucleic acid into the genome of the cell.

As described above, numerous embodiments are further provided that can be applied to any aspect of the present invention and/or combined with any other embodiment described herein. For example, in one embodiment, the methods described herein further comprise obtaining HSCs and/or cells derived therefrom prior to transducing the cells. In another embodiment, the methods described herein further comprise transplanting the transduced cells to an immunocompromised incubator animal, wherein the transplanted transduced cells reconstitute the immunocompromised incubator animal immune system. In still another embodiment, the methods described herein further comprise selecting populations of reconstituted immune cells of interest from the incubator animal. In yet another embodiment, the cells are transduced with a single viral vector. In another embodiment, the viral vector is a lentiviral vector. In still another embodiment, the viral vector inducibly expresses an RNA encoded by the exogenous nucleic acid. In yet another embodiment, the inducible expression is regulated using lactose operon operator (LacO) and lactose operon repressor (LacI) sequences. In another embodiment, the exogenous nucleic acid is selected from the group consisting of mRNA, antisense RNA, shRNA, siRNA, microRNA, PiwiRNA, and combinations thereof. In still another embodiment, the exogenous nucleic acid is an shRNA. In yet another embodiment, the exogenous nucleic acid comprises a) an engineered, non-naturally occurring Clustered Regularly Interspaced Short Palindromic Repeats (CRISPR) guide RNA that hybridizes with a target nucleic acid sequence of interest and/or b) a nucleotide sequence encoding a Type-II Cas9 protein, optionally wherein the cells are transgenic for Cas9. In another embodiment, the viral vector further comprises a nucleic acid encoding a reporter. In still another embodiment, the reporter is a fluorescent protein. In yet another embodiment, the incubator animal is immunocompromised using lethal irradiation or chemotherapy. In another embodiment, the incubator animal is immunodeficient. In still another embodiment, the immunocompromised incubator animal and the animal from which the HSCs and/or cells derived therefrom were obtained are congenic. In yet another embodiment, transplantation of the transduced cells to the immunocompromised incubator animal is autologous, syngeneic, allogeneic, or xenogeneic. In another embodiment, the reconstituted immune cells of interest are selected from the group consisting of terminally differentiated cells, post-mitotic cells, and/or unactivated cells. In still another embodiment, the reconstituted immune cells of interest have not been exogenously stimulated to divide. In yet another embodiment, the reconstituted immune cells of interest are T cells, dendritic cells, macrophages, or B cells. In another embodiment, the reconstituted immune cells of interest are isolated. In still another embodiment, the methods described herein further comprise culturing the selected cells in vitro and monitoring the selected cells in response to exogenous perturbation. In yet another embodiment, the methods described herein further comprise transplanting the transduced HSCs and/or cells derived therefrom into an experimental animal for differentiation in vivo. In another embodiment, the methods described herein further comprise monitoring the transplanted cells in response to exogenous perturbation. In still another embodiment, the exogenous perturbation is the application of an assay for testing autoimmune, allergic, vaccination, immunotolerance, cancer immunotherapy, immune exhaustion, immunological memory, immunological epitope, stem cell, hematopoietic stem cell, viral infection, or immune disease responses.

In another aspect, transduced HSCs and/or cells derived therefrom that are differentiated in vivo produced according to any one of methods described herein, are provided.

In still another aspect, non-human animals comprising transduced HSCs and/or cells derived therefrom that are produced according to any one of methods described herein, are provided.

As described above, numerous embodiments are further provided that can be applied to any aspect of the present invention and/or combined with any other embodiment described herein. For example, in one embodiment, the HSCs and/or cells derived therefrom are murine or human. In another embodiment, the HSCs and/or cells derived therefrom are selected from the group consisting of hematopoietic stem cells (HSC), common myeloid progenitor cells (CMP), common lymphoid progenitor cells (CLP), committed lymphoid progenitor cells, granulocyte/macrophage progenitor cells (GMP), megakaryocyte/erythroid progenitor cells (MEP), granulocyte progenitor cells, macrophage progenitor cells, erythroid progenitor cells, megakaryocyte progenitor cells (MKP), NK cell progenitor cells (NKP), B cell progenitor cells (BCP), and T cell progenitor cells (TCP). In still another embodiment, the HSCs and/or cells derived therefrom comprise or are T cells, such as CD8⁺ T cells. In yet another embodiment, the T cells are CAR-T cells. In another embodiment, the HSCs and/or cells derived therefrom are not terminally differentiated or post-mitotic. In still another embodiment, the HSCs and/or cells derived therefrom are not thymocytes or are not derived from the thymus. In yet another embodiment, the HSCs and/or cells derived are obtained from a biological source selected from the group consisting of bone marrow, umbilical cord blood, amniotic fluid, peripheral blood, and fetal liver.

### BRIEF DESCRIPTION OF THE DRAWINGS

FIG. 1A-FIG. 1J show that a chimeric CRISPR system enables efficient deletion of genes in the hematopoietic system without affecting immune homeostasis or function. FIG. 1A shows a schematic of chimeric CRISPR-Cas9 system. FIG. 1B shows flow cytometry plots of PD-1 expression in CD4⁺ T cells (top panel) and CD8⁺ T cells (bottom panel) from representative non-targeting control sgRNA or Pdcd1 sgRNA chimeras following αCD3/CD28 stimulation. FIG. 1C shows quantification of PD-1 expression for two control and three Pdcd1 sgRNAs from FIG. 1B. FIG. 1D shows TIDE assay results of naïve CD4⁺ and CD8⁺ T cells for three Pdcd1 targeting sgRNAs. FIG. 1E shows results of the TIDE assay on naïve CD8⁺ T cells designed to detect the top three predicted off-target sites (1st, 2nd, and 3rd) for three Pdcd1 targeting sgRNAs. The dashed line represents the aberrant sequence (%) when comparing two non-targeting control sgRNAs (background aberrant sequence). FIG. 1F shows the expression of CD20 (left), CD64 (middle), or DEC205 (right) on B cells, macrophages, or dendritic cells, respectively, from chimeric animals following transduction with a non-targeting control sgRNA or targeting sgRNAs to Ms4a1, Fcgr1, or Ly75. FIG. 1G shows quantification of CD20, CD64, and DEC205 expression on relevant lineages from FIG. 1F. FIG. 1H shows a comparison of frequencies of major immune lineages (of CD45) in chimera mice (WT: WT stem cells mock transduced, Cas9+sgRNA: Cas9 stem cells transduced with the Vex sgRNA expression vector) at homeostasis. FIGS. 1I and 1J show the results of chimeric mice infected with 4×10⁶ plaque-forming units (PFU) LCMV Clone 13 and their resulting weight loss (FIG. 1I) and serum viral titer (FIG. 1J). All experiments had at least four biological replicate animals per group and are representative of two independent experiments. Bar graphs represent mean and error bars represent standard deviation. Statistical significance was assessed by one-way ANOVA (FIG. 1C, FIG. 1G, FIG. 1H), or two-way ANOVA (FIG. 1I, FIG. 1J) (*p<0.05, **p<0.01, ***p<0.001, ****p<0.0001).

FIG. 2A-FIG. 2G further show that a chimeric CRISPR system enables efficient deletion of genes in the hematopoietic system without affecting immune homeostasis or function. FIG. 2A shows representative flow cytometry plots for gating of LSK cells. FIG. 2B shows representative flow cytometry plots for gating of Vex⁺ naive CD4⁺ and CD8⁺ T cells as in FIG. 1D. FIG. 2C and FIG. 2D show the results of next-generation sequencing of projected Pdcd1 sgRNA cut site in Vex⁺ naive CD4⁺ and CD8⁺ T cells, indel % (FIG. 2C) and frameshift % (FIG. 2D). FIG. 2E shows the results of a TIDE assay on naive CD4⁺ T cells designed to detect the top three predicted off-target sites (1st, 2nd, and 3rd) for three Pdcd1 targeting sgRNAs. The dashed line represents the aberrant sequence % when comparing two non-targeting control sgRNAs (background aberrant sequence). FIG. 2F shows representative flow cytometry plots for gating of Vex⁺ CD19⁺ B cells in FIG. 1F. FIG. 2G shows representative flow cytometry plots for gating of Vex⁺ red-pulp macrophages in FIG. 1F. All experiments had at least four biological replicate animals per group and are representative of two independent experiments. Bar graphs represent mean and error bars represent standard deviation.

FIG. 3A-FIG. 3I further show that a chimeric CRISPR system enables efficient deletion of genes in the hematopoietic system without affecting immune homeostasis or function. FIG. 3A shows representative flow cytometry plots for gating of Vex⁺ cross-presenting dendritic cells in FIG. 1F. FIG. 3B shows representative flow cytometry plots of Vex expression on major immune lineages in mice transduced with a non-targeting control sgRNA. FIG. 3C shows quantification of thymic subsets (CD4⁻ CD8⁻, CD4⁻ CD8⁺, CD4⁺ CD8⁻, CD4⁺ CD8⁺) from wild-type (WT) or Cas9+sgRNA chimeric mice. FIG. 3D shows representative flow cytometry plots of CD44 and CD62L from splenic CD8⁺ T cells. FIG. 3E shows representative flow cytometry plots of CD69 from splenic CD8⁺ T cells. FIG. 3F shows the quantification of naïve status of CD8⁺ T cells in FIGS. 3D-3E. FIG. 3G shows the day 30 kidney viral titer following LCMV Clone 13 infection of WT or Cas9+sgRNA chimeras. FIG. 3H shows representative flow cytometry plots of Granzyme B, Ki67, PD-1, GP₃₃₋₄₁ tetramer, and Tim-3 expression on CD8⁺ T cells at day 30 post LCMV Clone 13 infection as in FIG. 3G. FIG. 3I shows quantification of data presented in FIG. 3H. All experiments had at least four biological replicate animals per group and are representative of two independent experiments. Bar graphs represent mean and error bars represent standard deviation. Statistical significance was assessed by one-way ANOVA (FIGS. 3C, 3F, and 3I), or two-sided unpaired t-test (FIG. 3G) (ns p>0.05, *p<0.05, **p<0.01, ***p<0.001, ****p<0.0001).

FIG. 4A-FIG. 4F show that a chimeric CRISPR system recovers known negative and positive regulators of effector CD8⁺ T cell responses. FIG. 4A shows a schematic of competitive assays. FIG. 4B shows the representative input and output flow cytometry plots of P14 T cells containing control sgRNA versus control, Batf or Pdcd1 sgRNAs in the spleen 8 days post-LCMV Clone 13 viral infection. FIG. 4C shows quantification of P14 T cells containing control, Batf or Pdcd1 sgRNAs from the spleen (Batf-left), liver (Batf-middle), and spleen (Pdcd1-right) in FIG. 4B with normalization of the output flow cytometry plots to the input ratios at day 0 and log₂ transformation of the data. FIG. 4D shows representative output flow cytometry plots of OT-1 T cells containing control sgRNA vs. control, Pdcd1, or Batf sgRNAs in MC38-OVA tumors on day 7 post injection. FIG. 4E shows quantification of OT-1 T cells containing control or Batf sgRNAs of data presented in FIG. 4D with normalization of the output flow cytometry plots to the input ratios at day 0 and log₂ transformation of the data. FIG. 4F shows quantification of OT-1 T cells containing control or Pdcd1 sgRNAs of data presented in FIG. 4D with normalization of the output flow cytometry plots to the input ratios at day 0 and log ₂ transformation of the data. All experiments had five biological replicate animals per group and are representative of two independent experiments. Bar graphs represent mean and error bars represent standard deviation. Statistical significance was assessed by one-way ANOVA (FIGS. 4C, 4E, and 4F) (*p<0.05, **p<0.01, ***p<0.001, ****p<0.0001).

FIG. 5A-FIG. 5E further shows that a chimeric CRISPR system recovers known negative and positive regulators of effector CD8⁺ T cell responses. FIG. 5A shows representative flow cytometry plots for gating of Vex⁺ naive CD8⁺ T cells for transfer experiments described in FIG. 4A. FIG. 5B shows the results of a TIDE assay on naïve CD8⁺ T cells containing Batf sgRNAs prior to transfer into the competitive assay. FIG. 5C shows the gating strategy for analyzing transferred CD8⁺ T cells from the spleen following LCMV Clone 13 infection as in FIG. 4B. FIG. 5D shows representative output flow cytometry plots of P14 T cells containing control sgRNA vs. control or Pdcd1 sgRNAs in the liver 8 days post-LCMV Clone 13 infection. FIG. 5E shows quantification of data shown in FIG. 5D normalizing the output flow cytometry plots to the input ratios at day 0 and log₂ transforming the data. All experiments had at least five biological replicate animals per group and are representative of two independent experiments. Bar graphs represent mean and error bars represent standard deviation. Statistical significance was assessed by one-way ANOVA (FIG. 5E) (ns p>0.05, *p<0.05, **p<0.01, ***p<0.001, ****p<0001).

FIG. 6A-FIG. 6N show the results of deletion of Pdcd1 or Ptpn2 in different disease models. FIG. 6A shows the gating strategy for analyzing transferred CD8⁺ T cells from the tumor, lymph node, and spleen following tumor challenge as in FIG. 4D and FIG. 8A. FIG. 6B shows the results of flow cytometry profiling of PD-1 expression in control sgRNA or Pdcd1 sgRNA-containing OT-1 T cells responding to MC38-OVA tumors day 7 post-challenge. FIG. 6C shows quantification of data shown in FIG. 6B. FIG. 6D shows quantification of PTPN2 protein intensity normalized by β-ACTIN from Western blot data shown in FIG. 7B described below. FIG. 6E shows the uncropped Western blot from FIG. 7B described below. FIG. 6F shows representative output flow cytometry plots of P14 T cells containing control sgRNA vs. control or Ptpn2 sgRNAs in the liver 8 days post-LCMV Clone 13 infection. FIG. 6G shows quantification of FIG. 6F resulting from normalizing the output flow cytometry plots to the input ratios at day 0 and log ₂ transforming the data. FIG. 6H shows quantification of Vex expression in CD45⁺ blood cells from chimeric animals with control or Ptpn2 sgRNAs. FIG. 6I shows representative flow cytometry plots of CD44 and CD62L expression from peripheral blood of chimeric animals with control or Ptpn2 sgRNAs at day 14 after tumor implantation, as pre-gated on CD8β⁺ Vex⁺ cells. FIG. 6J shows quantification of CD44⁻ CD62L⁺, CD44⁺ CD62L⁺, and CD44⁺ CD62L⁻ populations in chimeric animals with control or Ptpn2 sgRNAs at day 14 after tumor implantation, as pre-gated on CD8β⁺ Vex⁺ cells. FIG. 6K shows TIDE assay results of naïve CD8⁺ T cells for three Batf targeting sgRNAs. FIG. 6L shows quantification of IFNγ and TNFα cytokine expression in co-transferred OT-1 CD8⁺ T cells day 7 post MC38-OVA implantation in the tumor for control vs. P1pn2-2 sgRNA co-transferred mix as in FIG. 8A. FIG. 6M shows quantification of immune infiltrate per gram of tumor in MC38 tumors 9 days post implantation, in control or P1pn2 sgRNA-containing bone marrow chimeras. FIG. 6N shows quantification of CD8⁺ T cells in the blood of chimeric animals with control or Ptpn2 sgRNAs treated with isotype or CD8-depleting antibody day 10 post MC38 tumor challenge. All experiments (except Western blot experiments) had at least five biological replicate animals per group and are representative of two independent experiments. Western blot experiments had two pooled mice per group and are representative of three independent experiments. Bar graphs represent mean and error bars represent standard deviation. Statistical significance was assessed by one-way ANOVA (FIGS. 6C and 6G), or two-way ANOVA (FIG. 6J) (ns p>0.05, *p<0.05, **p<0.01, ***P<0.001, ****p<0.0001).

FIG. 7A-FIG. 7M show that loss of Ptpn2 enhances CD8⁺ T cell responses to LCMV Cl.13. FIG. 7A shows the results of a TIDE assay on naive CD8⁺ T cells containing control sgRNA or Ptpn2 sgRNAs. FIG. 7B shows a Western blot of splenic CD8⁺ T cells from control sgRNA or Ptpn2 sgRNA-containing chimeras (cropped image; see FIG. 6E for the full-length blot). FIG. 7C shows representative output flow cytometry plots of P14 T cells containing control sgRNA vs. control or Ptpn2 sgRNAs in the spleen day 8 post-LCMV Clone 13 viral infection. FIG. 7D shows quantification of FIG. 7C with normalization of output flow cytometry plots to the input ratios at day 0 and log ₂ transformation of the data. FIG. 7E shows quantification of P14 T cells containing control sgRNA vs. control or Ptpn2 sgRNAs in the lung day 8 post-LCMV Clone 13 viral infection with normalization of the output flow cytometry plots to the input ratios at day 0 and log ₂ transformation of the data. FIG. 7F shows representative flow cytometry plots of splenic CD8⁺ T cells for control vs. Ptpn2 sgRNA mixes as in FIG. 7C, as analyzed for Granzyme B expression day 8 post-LCMV Clone 13 viral infection. FIG. 7G shows quantification of FIG. 7F for the two competitive mixes (control sgRNA vs. Ptpn2-1/Ptpn2-2 sgRNAs). FIG. 7H shows quantification of CD127 expression for mixes as in FIG. 7F. FIG. 7I shows quantification of TCF7 expression for mixes as in FIG. 7F. FIG. 7J shows representative flow cytometry plots of CXCR5 and Tim-3 expression on P14 T cells for control sgRNA vs. Ptpn2 sgRNA in the spleen 8 days post LCMV Clone 13 viral infection. FIG. 7K shows quantification of subpopulations of Tim-3⁺ CXCR5⁻ and Tim-3⁻ CXCR5⁺ shown in FIG. 7J for the two competitive mixes. FIG. 7L shows representative flow cytometry plots of Slamf6 and Tim-3 expression on P14 T cells containing control sgRNA vs. Ptpn2 sgRNA in the spleen 8 days post-LCMV Clone 13 viral infection. FIG. 7M shows quantification of subpopulations of Tim-3⁺ Slamf6- and Tim-3⁻ Slamf6⁺ shown in FIG. 7L for the two competitive mixes. All experiments (except Western blot experiments) had five biological replicate animals per group and are representative of two independent experiments. Western blot experiments had two pooled mice per group and are representative of three independent experiments. Bar graphs represent mean and error bars represent standard deviation. Statistical significance was assessed by one-way ANOVA (FIGS. 7D and 7E) and two-way ANOVA (FIGS. 7G-7I, 7K, and 7M) (*p<0.05, **p<0.01, ***p<0.001, ****p<0.0001).

FIG. 8A-FIG. 8O show that loss of Ptpn2 enhances the CD8⁺ T cell response to MC38 tumors. FIG. 8A shows quantification of OT-1 T cells containing control sgRNA vs. control or P1pn2 sgRNAs in MC38-OVA tumors with normalization of the output ratios on day 7 post-tumor implantation to the input ratios at day 0 and log ₂ transformation of the data. FIG. 8B shows quantification of Granzyme B expression in co-transferred OT-1 CD8⁺ T cells day 7 post-MC38-OVA implantation in the tumor, draining lymph node, and spleen, for control vs. Ptpn2-2 sgRNA co-transferred mix as in FIG. 8A. FIG. 8C shows quantification of the percent change in number of viable tumor cells when tumor cells were cultured alone or co-cultured with pre-activated control or Ptpn2 sgRNA-containing CD8⁺ T cells. FIG. 8D shows overlaid GSEA plots for co-transferred OT-1 T cells containing control sgRNA vs. Ptpn2 sgRNA in MC38-OVA tumors 7 days post-injection. FIG. 8E shows a schematic for generation of chimeric mice in which Ptpn2 is targeted in about 50% of immune cells. FIG. 8F shows tumor growth curves for chimeric mice containing a non-targeting control sgRNA or one of two Ptpn2 sgRNAs following 1×10⁶ cell MC38-WT challenge. FIG. 8G shows survival curves of tumor-bearing mice from FIG. 8F. FIGS. 8H and 8I show quantification of Granzyme B (FIG. 8H) and CD127 (FIG. 8I) from peripheral blood of mice as in FIG. 8F on day 14 post-tumor implantation, as pre-gated on CD80-Vex⁺ cells. FIG. 8J shows tumor growth curves for mice as in FIG. 8F that were rechallenged with 5×10⁶ MC38-WT tumor cells following a 60-day rest post primary tumor clearance. FIGS. 8K and 8L show quantification of (FIG. 8K) CD25 and (FIG. 8L) CD127 expression in co-transferred OT-1 CD8⁺ T cells day 7 post MC38-OVA implantation in the tumor-draining lymph node for control vs. Ptpn2-2 sgRNA co-transferred mix as in (FIG. 8A). FIG. 8M shows GSEA TIL Slamf6⁺ vs. Tim-3⁺ UP and DOWN signature enrichment for co-transferred OT-1 T cells containing control sgRNA vs. Ptpn2 sgRNA in MC38-OVA tumors 7 days post injection. FIG. 8N shows GSEA curves for significantly enriched signatures for RNA-seq of control and Ptpn2 sgRNA mixes day 7 post MC38-OVA injection. FIG. 8O shows quantification of Slamf6⁺ and Tim-3⁺ Vex⁺ CD8⁺ T cells from the blood of mice in (FIG. 8E) day 12 post tumor implantation. The data shown in FIGS. 8A, 8B, 8F-8J, 8K-8L, and 8O result from at least five biological replicate animals per group and are representative of two independent experiments. The data shown in FIG. 8C result from two pooled biological replicate animals per group with at least three technical replicates and are representative of two independent experiments. The data shown in FIG. 8D results from three pooled mice per group with at least two technical replicates and are representative of one experiment. Experiments in FIG. 8M and FIG. 8N had three pooled mice per group with at least two technical replicates and is representative of one experiment. Bar graphs represent mean and error bars represent standard deviation (except for FIGS. 8F and 8J where error bars represent standard error). Statistical significance was assessed by one-way ANOVA (FIGS. 8A-8C, 8H, and 8I), paired t-test (FIGS. 8K and 8L), Kolmogorov-Smirnov test (FIGS. 8D, 8M and 8N), two-way ANOVA (FIGS. 8F, 8O, and 8J), or log-rank Mantel-Cox test (FIG. 8G) (ns p>0.05, *p<0.05, **p<0.01, ***p<0.001, ****p<0.0001).

FIG. 9A-FIG. 9H show that loss of Ptpn2 promotes the early expansion of CD8⁺ T cells during LCMV Clone 13 infection. FIG. 9A shows the schematic of CHIME system. FIG. 9B shows the TIDE assay on naïve CD8⁺ T cells for a Ptpn2 targeting sgRNA. FIG. 9C shows the representative flow cytometry plot of P14 T cells containing control sgRNA vs. Ptpn2 sgRNA in the spleen 8 days post LCMV Clone 13 viral infection. FIGS. 9D-9E show the frequency (FIG. 9D) and number (FIG. 9E) of control or Ptpn2 sgRNA-containing P14 T cells in the spleen 8, 15, 22, and 30 days post LCMV Clone 13 viral infection. FIG. 9F shows the quantification of BrdU incorporation for control vs. Ptpn2 P14 T cells days 8, 15, and 30 days post LCMV Clone 13 viral infection. FIG. 9G shows the representative flow cytometry plots of splenic CD8⁺ T cells for control vs. Ptpn2 sgRNA mixes as in (FIG. 9C) analyzed for Granzyme B expression day 8 post LCMV Clone 13 infection. FIG. 9H shows the quantification of (FIG. 9G) days 8, 15, 22, and 30 post LCMV Clone 13 viral infection. All experiments had at least four biological replicate animals per group and are representative of two independent experiments. Bar graphs represent mean and error bars represent standard deviation. Statistical significance was assessed by paired t-test (d, e, f, h) (*p<0.05, **p<0.01, ***p<0.001, ****p<0.0001). See also FIG. 10.

FIG. 10A-FIG. 10B show that loss of Ptpn2 promotes the early expansion of CD8⁺ T cells during LCMV Clone 13 infection. FIG. 10A shows the gating strategy for analyzing transferred CD8⁺ T cells from the spleen following LCMV Clone 13 infection as in FIG. 9C. FIG. 10B shows the quantification of IFNγ⁺ TNFα⁺ P14 T cells for control vs. Ptpn2 sgRNA mixes days 8, 15, 22, and 30 days post LCMV Clone 13 viral infection. All experiments had at least four biological replicate animals per group and are representative of two independent experiments. Bar graphs represent mean and error bars represent standard deviation. Statistical significance was assessed by paired t-test (b) (*p<0.05, **p<0.01, ***p<0.001, ****p<0.0001).

FIG. 11A-FIG. 11E show that deletion of Ptpn2 enhances formation of the Tim-3⁺ subpopulation during LCMV Clone 13 infection. FIG. 11A shows the ratio of Tim-3⁺/Slamf6⁺ P14 T cells containing a control or Ptpn2 sgRNA in the spleen 8, 15, 22, and 30 days post LCMV Clone 13 viral infection. FIG. 11B shows the number of Tim-3⁺ control or Ptpn2 sgRNA-containing P14 T cells in the spleen 8, 15, 22, and 30 days post LCMV Clone 13 viral infection. FIG. 11C shows the number of Slamf6⁺ control or Ptpn2 sgRNA-containing P14 T cells in the spleen 8, 15, 22, and 30 days post LCMV Clone 13 viral infection. FIG. 11D shows the quantification of Granzyme B expression for Tim-3⁺ P14 T cells containing a control or Ptpn2 sgRNA in the spleen 8, 15, 22, and 30 days post LCMV Clone 13 viral infection. FIG. 11E shows the quantification of BrdU incorporation for Tim-3⁺ P14 T cells containing a control or Ppn2 sgRNA in the spleen 8, 15, 22, and 30 days post LCMV Clone 13 viral infection. All experiments had at least four biological replicate animals per group and are representative of two independent experiments. Bar graphs represent mean and error bars represent standard deviation. Statistical significance was assessed by paired t-test (b-f) (*p<0.05, **p<0.01, ***p<0.001, ****p<0.0001). See also FIG. 12.

FIG. 12A-FIG. 12E show that deletion of Ptpn2 enhances formation of the Tim-3⁺ subpopulation during LCMV Clone 13 infection. FIG. 12A-12B show the frequency of (FIG. 12A) Tim-3⁺ or (FIG. 12B) Slamf6⁺ control or Ptpn2 sgRNA-containing P14 T cells in the spleen 8, 15, 22, and 30 days post LCMV Clone 13 viral infection. FIG. 12C shows the frequency of Tim-3⁺ CXCR5⁻ and Tim-3⁻ CXCR5⁺ P14 T cells containing a control or Ptpn2 sgRNA in the spleen 8 days post LCMV Clone 13 viral infection. FIG. 12D shows TCF7 and CD127 expression on control or Ptpn2 sgRNA-containing P14 T cells in the spleen 8 days post LCMV Clone 13 viral infection. FIG. 12E shows the number of Tim-3⁺ CXCR5⁻ and Tim-3⁻ CXCR5⁺ control or Ptpn2 sgRNA-containing P14 T cells in the spleen 8 days post LCMV Clone 13 viral infection. All experiments had at least five biological replicate animals per group and are representative of two independent experiments. Bar graphs represent mean and error bars represent standard deviation. Statistical significance was assessed by paired t-test (FIGS. 12A-12B, FIGS. 12C-12E) (ns p>0.05, *p<0.05, **p<0.01, ***p<0.001, ****p<0.0001).

FIG. 13A-FIG. 13I show that Ppn2 deletion promotes effector-skewed Slamf6⁺ and Tim-3⁺ subpopulations during LCMV infection. FIG. 13A shows the t-SNE projection of single-cell RNA-seq profiles from 7,027 control or Ptpn2-deleted P14⁺ CD8⁺ T cells responding to day 30 LCMV Clone 13 infection. Clusters are distinct colors. FIG. 13B shows the enrichment of gene signatures in the clusters. FIG. 13C shows plots depicting the inter-cluster density for control or Ptpn2-deleted cells. FIG. 13D shows the quantification of the proportion of control or Ptpn2-deleted cells in each cluster. Error bars represent the 95% confidence interval. FIGS. 13E-13F show the signature enrichments of control vs Ptpn2-deleted cells from the (FIG. 13E) progenitor or (FIG. 13F) terminally exhausted clusters. FIG. 13G shows representative GSEA curves for RNA-seq of control and Ptpn2 sgRNA mixes day 8 post LCMV Clone 13 infection. LCMV Slamf6 vs. Tim-3 Up and Down signatures are depicted. FIGS. 13H-13I show GSEA curves for significantly enriched signatures in (FIG. 13H) Slamf6⁺ cells and (FIG. 13I) Tim-3⁺ cells for RNA-seq of control and Pqpn2 sgRNA mixes day 8 post LCMV Clone 13 infection. All experiments had at least two biological replicate animals per group and are representative of one experiment. Statistical significance was assessed by the Wilcoxon rank sum test (FIGS. 13B, 13E, and 13F), binomial test (FIG. 13D), and Kolmogorov-Smirnov test (FIGS. 13G-13I) (*p<0.05, **p<0.01, ***p<0.001, ****p<0.0001). See also FIG. 14.

FIG. 14A-FIG. 14H show that Ptpn2 deletion promotes effector-skewed Slamf6⁺ and Tim-3⁺ subpopulations during LCMV infection. FIG. 14A shows the heat map of the t-SNE clusters in FIG. 13E with representative genes highlighted. FIG. 14B shows expression of indicated genes in individual cells overlaid on the defined clusters. FIG. 14C shows the quantification of Slamf6 and Tim-3 expression assessed by flow cytometry on control and Ptpn2-deleted cells analyzed by single cell RNA-seq. FIG. 14D shows plots depicting the intra-cluster density for control or Ppn2-deleted cells. FIG. 14E shows principal components analysis of transcriptional profiles of control and Ptpn2 sgRNA mixes day 8 post LCMV Clone 13 infection. FIG. 14F shows venn diagrams of ATAC-seq peak overlaps of Tim-3⁺ and Slamf6⁺ control and Ptpn2 sgRNA mixes day 8 post LCMV Clone 13 infection. FIGS. 14G-14H show representative ATAC-seq tracks for (FIG. 14G) Tcf7 and (FIG. 14H) Tox for Tim-3⁺ and Slamf6⁺ populations in control and Ptpn2 sgRNA mixes day 8 post LCMV Clone 13 infection. All experiments had at least two biological replicate animals per group and are representative of one experiment. Statistical significance was assessed by paired t-test (FIG. 14C) or hypergeometric test (FIG. 14F) (*p<0.05, **p<0.01, ***p<0.001, ****p<0.0001).

FIG. 15A-FIG. 15L show that Ptpn2 deletion increases Tim-3⁺ cell differentiation through enhanced IFN-α signaling. FIG. 15A shows quantification of co-transferred control or Ptpn2-deleted CD8⁺ T cells day 4 post LCMV Clone 13 infection. Frequencies at day 4 were normalized to input frequencies at day 0. FIG. 15B shows representative flow cytometry plots of splenic CD8⁺ T cells day 4 post LCMV Clone 13 infection for control vs. P1pn2 sgRNA mixes. FIG. 15C shows quantification of Tim-3⁺ and Slamf6⁺ populations in FIG. 15B. FIG. 15D shows quantification of Granzyme B expression of cells as in FIG. 15A. FIGS. 15E-15G show quantification of (FIG. 15E) Slamf6⁺ Tim-3⁻, (FIG. 15F) Slamf6⁺ Tim-3⁺, and (FIG. 15G) Slamf6⁻ Tim-3⁻ subsets following in vitro stimulation (αCD3/CD28) of control or Ptpn2-deleted CD8⁺ T cells in the presence of indicated cytokines or blocking antibodies. FIG. 15H shows quantification of pSTAT1 expression of splenic CD8⁺ T cells day 8 post LCMV Clone 13 infection for control vs. Ptpn2 sgRNA mixes following restimulation with IFN-α. FIGS. 15I-15J show quantification of pSTAT1 in (FIG. 15I) Slamf6⁺ or (FIG. 15J) Tim-3⁺ cells following IFN-α restimulation of control and Ptpn2 sgRNA mixes as in (FIG. 15H). FIG. 15K shows quantification of co-transferred control or Ptpn2-deleted CD8⁺ T cells day 4 post LCMV Clone 13 infection following treatment with isotype (left graph) or IFNAR blocking antibody (right graph). Frequencies at day 4 were normalized to input frequencies at day 0. FIG. 15L shows quantification of Slamf6⁺ Tim-3⁻, Slamf6⁺ Tim-3⁺, and Slamf6⁻ Tim-3⁺ subsets day 4 post LCMV Clone 13 infection in mice that received control and Ptpn2 sgRNA mixes and were treated with isotype or IFNAR blocking antibody. All experiments had at least three biological replicate animals per group and are representative of two independent experiments. Bar graphs represent mean and error bars represent standard deviation. Statistical significance was assessed by paired t-test (a, c-d, h-k) or one-way ANOVA (e-g, 1) (*p<0.05, **p<0.01, ***p<0.001, ****p<0.0001). See also FIG. 16.

FIG. 16A-FIG. 16F show that Ppn2 deletion increases Tim-3⁺ cell differentiation through enhanced IFN-α signaling. FIG. 16A shows quantification of CD25 MFI on CD8⁺ CD25⁺ control or Ptpn2-deleted CD8⁺ T cells following in vitro stimulation (αCD3/CD28) in the presence of indicated cytokines or blocking antibodies. FIG. 16B shows a representative flow cytometry plot of Slamf6 and Tim-3 expression on control or Ptpn2-deleted CD8⁺ T cells following in vitro stimulation (αCD3/CD28) in the presence of IL-2 and IFN-α. FIG. 16C shows quantification of Slamf6⁺ Tim-3⁻, Slamf6⁺ Tim-3⁺, Slamf6⁻ Tim-3⁺ subsets in control or Ptpn2-deleted CD8⁺ T cells following in vitro stimulation (αCD3) in the presence of IL-2 and IFN-α. FIG. 16D shows quantification of Slamf6⁺ Tim-3⁻, Slamf6+ Tim-3⁺, Slamf6⁻ Tim-3⁺ subsets in control cells following in vitro stimulation (αCD3/CD28) in the presence of indicated cytokines or blocking antibodies and addition of pre-conditioned supernatant from stimulated control (left) or Ppn2-deleted CD8⁺ T cells (right). FIG. 16E shows representative histograms of IFNAR1 expression on naive control or Ptpn2-deleted CD8⁺ T cells. FIG. 16F shows quantification of Slamf6⁺ Tim-3⁻, Slamf6⁺ Tim-3⁺, and Slamf6⁻ Tim-3⁺ subsets day 4 post LCMV Clone 13 infection in mice that received control and Ptpn2 sgRNA mixes and were treated with isotype or IFNAR blocking antibody. All experiments had at least two technical replicates and are representative of two independent experiments. Bar graphs represent mean and error bars represent standard deviation. Statistical significance was assessed by one-way ANOVA (FIGS. 16A, 16C, 16D, and 16F) (ns p>0.05, *p<0.05, **p<0.01, ***p<0.001, ****p<0.0001).

FIG. 17A-FIG. 17F show loss of Ptpn2 increases cytotoxic CD8⁺ T cells and improves checkpoint blockade responses. FIG. 17A shows quantification of frequency of immune infiltrate (pre-gated on CD45) in MC38 tumors 9 days post implantation, in control or Ptpn2 sgRNA-containing bone marrow chimeras. FIG. 17B shows quantification of Granzyme B expression in CD8⁺ T cells infiltrating MC38 tumors implanted in control or Ptpn2 sgRNA-containing bone marrow chimeras. FIG. 17C shows tumor growth curves for mice as in (FIG. 17A) challenged with 1×10⁶ MC38-WT tumor cells following treatment with CD8-depleting antibody or isotype control. FIG. 17D shows tumor growth curves for mice as in (FIG. 17A) rechallenged with 5×10⁶ MC38-WT tumor cells following a 60-day rest post primary tumor clearance and treatment with CD8-depleting antibody or isotype control. FIG. 17E shows tumor growth curves for control or Ptpn2 sgRNA-containing bone marrow chimeric mice challenged with 1×10⁶ B16 tumor cells treated with GVAX (green triangles) on days 1, 4 and αPD-1 (black triangles) on days 12, 14, 16, 18, 20, 22, 24, and 26. FIG. 17F shows quantification of Granzyme B from peripheral blood of chimeras in (FIG. 17E) day 14 post B16 tumor implantation, pregated on CD8β⁺ Vex⁺ cells. Experiments FIGS. 17A-17C and 17E had at least seven biological replicate animals per group and are representative of two independent experiments. Experiment in FIG. 17D had at least four biological replicate animals per group and is representative of two independent experiments. Bar graphs represent mean and error bars represent standard deviation (except for FIGS. 17C-17E where error bars represent standard error). Statistical significance was assessed by one-way ANOVA (FIGS. 17A-17B and 17F), or two-way ANOVA (FIGS. 17C-17E) (ns p>0.05, *p<0.05, **p<0.01, ***p<0.001, ****p<0.0001). See also FIG. 6.

For any figure showing a bar histogram, curve, or other data associated with a legend, the bars, curve, or other data presented from left to right for each indication correspond directly and in order to the boxes from top to bottom, or from left to right, of the legend.

### DETAILED DESCRIPTION OF THE INVENTION

The present invention is based, at least in part, on the discovery that loss of function of Ptpn2 enhances CD8⁺ T cell responses to chronic pathogens and cancer. The phosphatase Ptpn2 was identified as a regulator of the generation of the Tim-3⁺ subpopulation in response to LCMV Clone 13 viral infection and tumors. In models of both chronic viral infection and transplantable tumors, Ptpn2 null CD8⁺ T cells expand more than wild-type cells and show increased expression of effector genes. Deletion of Ptpn2 in the immune system induces complete clearance of tumors, accompanied by increased cytotoxic effector CD8⁺ T cell responses. Specifically, loss of Ptpn2 is associated with enhanced IFN-I cytokine signaling, and increases the number of Tim-3⁺ cytotoxic CD8⁺ T cells at an early time point during LCMV Clone 13 infection without altering the numbers of the Slamf6⁺ subpopulation. In addition, Ptpn2 deletion promotes the differentiation of the Tim-3⁺ cytotoxic subset and increases cytotoxic CD8⁺ T cell responses in both the MC38 colorectal and B16 melanoma cancer models. This increase in cytotoxicity is accompanied by complete clearance of immunogenic MC38 tumors and improved PD-1 checkpoint blockade responses to less immunogenic B16 tumors. This discovery was made using a bone marrow chimeric CRISPR-Cas9 delivery system that can rapidly evaluate gene function in immune cells in vivo without prior ex vivo manipulation. This approach enables efficient deletion of genes of interest in any of the major immune lineages without altering their homeostatic frequencies or function. These data demonstrate that increasing the number of Tim-3⁺ cytotoxic CD8 T cells can promote effective tumor immunity, and provide rationale for Ptpn2 as a cancer immunotherapy target that may enhance CD8⁺ T cell-mediated anti-tumor immunity and improve tumor control. These findings also demonstrate that this genetic platform can enable rapid target discovery by allowing deletion of genes in immune cell lineages in vivo while maintaining normal immune development and function. Accordingly, methods of treating, diagnosing, prognosing disorders that would benefit from increased immune responses using agent that inhibits the copy number, the expression level, and/or the activity of PTPN2, are provided. In addition, methods and compositions for perturbing gene expression in hematopoietic cell lineages in vivo are also provided.

### I. Definitions

The articles “a” and “an” are used herein to refer to one or to more than one (i.e. to at least one) of the grammatical object of the article. By way of example, “an element” means one element or more than one element.

The term “administering” is intended to include routes of administration which allow an agent to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo.

The term “activating receptor” includes immune cell receptors that bind antigen, complexed antigen (e.g., in the context of MHC polypeptides), or bind to antibodies. Such activating receptors include T cell receptors (TCR), B cell receptors (BCR), cytokine receptors, LPS receptors, complement receptors, and Fc receptors.

T cell receptors are present on T cells and are associated with CD3 polypeptides. T cell receptors are stimulated by antigen in the context of MHC polypeptides (as well as by polyclonal T cell activating reagents). T cell activation via the TCR results in numerous changes, e.g., protein phosphorylation, membrane lipid changes, ion fluxes, cyclic nucleotide alterations, RNA transcription changes, protein synthesis changes, and cell volume changes.

The term “chimeric antigen receptor” or “CAR” refers to engineered T cell receptors (TCR) having a desired antigen specificity. T lymphocytes recognize specific antigens through interaction of the T cell receptor (TCR) with short peptides presented by major histocompatibility complex (MHC) class I or II molecules. For initial activation and clonal expansion, naive T cells are dependent on professional antigen-presenting cells (APCs) that provide additional co-stimulatory signals. TCR activation in the absence of costimulation can result in unresponsiveness and clonal anergy. To bypass immunization, different approaches for the derivation of cytotoxic effector cells with grafted recognition specificity have been developed. CARs have been constructed that consist of binding domains derived from natural ligands or antibodies specific for cell-surface components of the TCR-associated CD3 complex. Upon antigen binding, such chimeric antigen receptors link to endogenous signaling pathways in the effector cell and generate activating signals similar to those initiated by the TCR complex. Since the first reports on chimeric antigen receptors, this concept has steadily been refined and the molecular design of chimeric receptors has been optimized and routinely use any number of well-known binding domains, such as scFV, Fav, and another protein binding fragments described herein.

In some embodiments, the CAR includes an antigen binding domain, a transmembrane domain and an intracellular domain. The antigen binding domain binds to an antigen on a target cell. Examples of cell surface markers that can act as an antigen that binds to the antigen binding domain of the CAR include those associated with viral, bacterial, parasitic infections, autoimmune disease and cancer cells (e.g., tumor antigens).

In some embodiments, the antigen binding domain binds to a tumor antigen, such as an antigen that is specific for a tumor or cancer of interest. Non-limiting examples of tumor associated antigens include BCMA, CD19, CD24, CD33, CD38; CD44v6, CD123, CD22, CD30, CD117, CD171, CEA, CS-1, CLL-1, EGFR, ERBB2, EGFRvIII, FLT3, GD2, NY-BR-1, NY-ESO-1, p53, PRSS21, PSMA, ROR1, TAG72, Tn Ag, VEGFR2.

In some embodiments, the transmembrane domain is naturally associated with one or more of the domains in the CAR. The transmembrane domain can be derived either from a natural or from a synthetic source. Transmembrane regions of particular use in this invention can be derived from (i.e. comprise at least the transmembrane region(s) of) the alpha, beta or zeta chain of the T-cell receptor, CD28, CD3 epsilon, CD45, CD4, CD5, CD8, CD9, CD 16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and TLR9. In some instances, a variety of human hinges can be employed as well including the human Ig (immunoglobulin) hinge.

In some embodiments, the intracellular domain of the CAR includes a domain responsible for signal activation and/or transduction. Examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP 12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD 1 id, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

In some embodiments, engineered T cells encompassed by the present invention can be used to re-engineer monocytes and macrophages to increase their ability to present antigens to other immune effector cells, for example, T cells. Engineered monocytes and macrophages as antigen presenting cells (APCs) will process tumor antigens and present antigenic epitopes to T cells to stimulate adaptive immune responses to attack tumor cells.

Generally, CARs are one type of “cell therapy” (e.g., “T cell therapy) contemplated for use according to the present invention. In some embodiments, such CAR-T cells can be engineered using any one or more of numerous representative embodiments of agents and methods for modulating immune cell activity to modulate the PTPN2 pathway therein, such as modulating the copy number, the expression level, and/or activity of Ptpn2. Such modified T cells and uses thereof, such as in immune cell-based therapies and other methods described herein, are also encompassed. For example, T cells, such as T cells engineered to express a T cell receptor having a desired antigen specificity, engineered to have a knockout, knockdown, or decreased expression of Ptpn2 are contemplated. Similarly, immune cells or other cells engineered to have a decreased phosphatase activity and/or substrate binding activity of Ptpn2, are also contemplated.

B cell receptors are present on B cells. B cell antigen receptors are a complex between membrane Ig (mIg) and other transmembrane polypeptides (e.g., Igαand Igβ). The signal transduction function of mIg is triggered by crosslinking of receptor polypeptides by oligomeric or multimeric antigens. B cells can also be activated by anti-immunoglobulin antibodies. Upon BCR activation, numerous changes occur in B cells, including tyrosine phosphorylation.

Fc receptors are found on many cells which participate in immune responses. Fc receptors (FcRs) are cell surface receptors for the Fc portion of immunoglobulin polypeptides (Igs). Among the human FcRs that have been identified so far are those which recognize IgG (designated Fcγ R), IgE (Fcε R1), IgA (Fcα), and polymerized IgM/A (Fcμα R). FcRs are found in the following cell types: Fcε R I (mast cells), Fcε R.II (many leukocytes), Fcα R (neutrophils), and Fcμα R (glandular epithelium, hepatocytes) (Hogg, N. (1988) Immunol. Today 9:185-86). The widely studied FcγRs are central in cellular immune defenses, and are responsible for stimulating the release of mediators of inflammation and hydrolytic enzymes involved in the pathogenesis of autoimmune disease (Unkeless, J. C. et al. (1988) Anim. Rev. Immunol. 6:251-81). The FcγRs provide a crucial link between effector cells and the lymphocytes that secrete Ig, since the macrophage/monocyte, polymorphonuclear leukocyte, and natural killer (NK) cell FcγRs confer an element of specific recognition mediated by IgG. Human leukocytes have at least three different receptors for IgG: h Fcγ RI (found on monocytes/macrophages), hFcγ RI (on monocytes, neutrophils, eosinophils, platelets, possibly B cells, and the K562 cell line), and Fcγ III (on NK cells, neutrophils, eosinophils, and macrophages).

With respect to T cells, transmission of a costimulatory signal to a T cell involves a signaling pathway that is not inhibited by cyclosporin A. In addition, a costimulatory signal can induce cytokine secretion (e.g., IL-2 and/or IL-10) in a T cell and/or can prevent the induction of unresponsiveness to antigen, the induction of anergy, or the induction of cell death (deletion) in the T cell.

The term “activity,” when used with respect to a polypeptide, e.g., PTPN2, includes activities that are inherent in the structure of the protein. For example, with regard to PTPN2, the term “activity” includes the phosphatase activity and/or the substrate binding activity.

The term “altered amount” or “altered level” refers to increased or decreased copy number (e.g., germline and/or somatic) of a biomarker nucleic acid, e.g., increased or decreased expression level in a disease sample, as compared to the expression level or copy number of the biomarker nucleic acid in a control sample. The term “altered amount” of a biomarker also includes an increased or decreased protein level of a biomarker protein in a disease sample, e.g., a cancer sample, as compared to the corresponding protein level in a normal, control sample. Furthermore, an altered amount of a biomarker protein may be determined by detecting posttranslational modification such as methylation status of the marker, which may affect the expression or activity of the biomarker protein.

The amount of a biomarker in a subject is “significantly” higher or lower than the normal amount of the biomarker, if the amount of the biomarker is greater or less, respectively, than the normal or control level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternatively, the amount of the biomarker in the subject can be considered “significantly” higher or lower than the normal and/or control amount if the amount is at least about two, and preferably at least about 5%, 10%, 15%, 200%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, two times, three times, four times, five times, or more, or any range in between, such as 5%-100%, higher or lower, respectively, than the normal and/or control amount of the biomarker. Such significant modulation values can be applied to any metric described herein, such as altered level of expression, altered activity, changes in cancer cell hyperproliferative growth, changes in cancer cell death, changes in biomarker inhibition, changes in test agent binding, and the like.

The term “altered level of expression” of a marker refers to an expression level or copy number of a marker in a test sample e.g., a sample derived from a subject suffering from a condition that would benefit from an increased immune response (e.g., cancer or viral infection), that is greater or less than the standard error of the assay employed to assess expression or copy number, and is preferably at least twice, and more preferably three, four, five or ten or more times the expression level or copy number of the marker or chromosomal region in a control sample (e.g., sample from a healthy subject not having the associated disease) and preferably, the average expression level or copy number of the marker or chromosomal region in several control samples. The altered level of expression is greater or less than the standard error of the assay employed to assess expression or copy number, and is preferably at least twice, and more preferably three, four, five or ten or more times the expression level or copy number of the marker in a control sample (e.g., sample from a healthy subject not having the associated disease) and preferably, the average expression level or copy number of the marker in several control samples.

The term “altered activity” of a marker refers to an activity of a marker which is increased or decreased in a disease state, e.g., in a cancer sample, as compared to the activity of the marker in a normal, control sample. Altered activity of a marker may be the result of, for example, altered expression of the marker, altered protein level of the marker, altered structure of the marker, or, e.g., an altered interaction with other proteins involved in the same or different pathway as the marker, or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

The “amount” of a marker, e.g., expression or copy number of a marker or MCR, or protein level of a marker, in a subject is “significantly” higher or lower than the normal amount of a marker, if the amount of the marker is greater or less, respectively, than the normal level by an amount greater than the standard error of the assay employed to assess amount, and preferably at least twice, and more preferably three, four, five, ten or more times that amount. Alternately, the amount of the marker in the subject can be considered “significantly” higher or lower than the normal amount if the amount is at least about two, and preferably at least about three, four, or five times, higher or lower, respectively, than the normal amount of the marker.

Unless otherwise specified herein, the terms “antibody” and “antibodies” broadly encompass naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

In addition, intrabodies are well-known antigen-binding molecules having the characteristic of antibodies, but that are capable of being expressed within cells in order to bind and/or inhibit intracellular targets of interest (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Pubis. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P. et al. (1993) Proc. Natl. Acad. Sci. USA 90:6444-6448; Poljak, R. J. et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M. et al. (1995) Human Antibodies and Hybridomas 6:93-101) and use of a cysteine residue, a marker peptide and a C-terminal polyhistidine tag to make bivalent and biotinylated scFv polypeptides (Kipriyanov, S. M. et al. (1994)Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g., humanized, chimeric, etc.). Antibodies may also be fully human. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts. In addition, antibodies can be “humanized,” which includes antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies encompassed by the present invention may include amino acid residues not encoded by human germline immunoglobulin sequences (e.g., mutations introduced by random or site-specific mutagenesis in vitro or by somatic mutation in vivo), for example in the CDRs. The term “humanized antibody,” as used herein, also includes antibodies in which CDR sequences derived from the germline of another mammalian species, such as a mouse, have been grafted onto human framework sequences.

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “antisense” nucleic acid polypeptide comprises a nucleotide sequence which is complementary to a “sense” nucleic acid encoding a protein, e.g., complementary to the coding strand of a double-stranded cDNA polypeptide, complementary to an mRNA sequence or complementary to the coding strand of a gene. Accordingly, an antisense nucleic acid polypeptide can hydrogen bond to a sense nucleic acid polypeptide.

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, peritoneal fluid, pus, saliva, sebum, semen, serum, sweat, synovial fluid, tears, urine, vaginal lubrication, vitreous humor, and vomit).

The term “a condition that would benefit from an increase immune response” refers to conditions in which upregulation of an immune response is desired. Such conditions are well-known in the art and include, without limitation, disorders requiring increased CD8+ effector T cell production or function such as combating cancer, infections (e.g., parasitic, bacterial, helminthic, or viral infections), and the like.

The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer whose phenotype is determined by the method encompassed by the present invention is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostrate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, brenner, or undifferentiated. In some embodiments, the present invention is used in the treatment, diagnosis, and/or prognosis of lymphoma or its subtypes, including, but not limited to, mantle cell lymphoma.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The terms “conjoint therapy” and “combination therapy,” as used herein, refer to the administration of two or more therapeutic substances. The different agents comprising the combination therapy may be administered concomitant with, prior to, or following the administration of one or more therapeutic agents.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control patient (can be stored sample or previous sample measurement) with a known outcome; normal tissue or cells isolated from a subject, such as a normal patient or the patient having a condition of interest (cancer is used below as a representative condition), cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods encompassed by the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods encompassed by the present invention are not limited to use of a specific cut-point in comparing the level of expression product in the test sample to the control.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The “normal” copy number (e.g., germline and/or somatic) of a biomarker nucleic acid or “normal” level of expression of a biomarker nucleic acid, or protein is the activity/level of expression or copy number in a biological sample, e.g., a sample containing tissue, whole blood, serum, plasma, buccal scrape, saliva, cerebrospinal fluid, urine, stool, and bone marrow, from a subject, e.g., a human, not afflicted with a condition that would benefit from an increased immune response, or from a corresponding non-affected tissue in the same subject who has a condition that would benefit from an increased immune response.

The term “determining a suitable treatment regimen for the subject” is taken to mean the determination of a treatment regimen (i.e., a single therapy or a combination of different therapies that are used for the prevention and/or treatment of a condition that would benefit from an increased immune response (e.g., cancer or viral infection) in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “expression signature” or “signature” refers to a group of two or more coordinately expressed biomarkers. For example, the genes, proteins, and the like making up this signature may be expressed in a specific cell lineage, stage of differentiation, or during a particular biological response. The biomarkers can reflect biological aspects of the tumors in which they are expressed, such as the cell of origin of the cancer, the nature of the non-malignant cells in the biopsy, and the oncogenic mechanisms responsible for the cancer. Expression data and gene expression levels can be stored on computer readable media, e.g., the computer readable medium used in conjunction with a microarray or chip reading device. Such expression data can be manipulated to generate expression signatures.

As used herein, the term “composite antibody” refers to an antibody which has variable regions comprising germline or non-germline immunoglobulin sequences from two or more unrelated variable regions. Additionally, the term “composite, human antibody” refers to an antibody which has constant regions derived from human germline or non-germline immunoglobulin sequences and variable regions comprising human germline or non-germline sequences from two or more unrelated human variable regions. A composite, human antibody is useful as an effective component in a therapeutic agent according to the present invention since the antigenicity of the composite, human antibody in the human body is lowered.

As used herein, the term “Fc region” is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Suitable native-sequence Fc regions for use in the antibodies encompassed by the present invention include human IgG1, IgG2 (IgG2A, IgG2B), IgG3 and IgG4.

As used herein, “Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

A molecule is “fixed” or “affixed” to a substrate if it is covalently or non-covalently associated with the substrate such the substrate can be rinsed with a fluid (e.g. standard saline citrate, pH 7.4) without a substantial fraction of the molecule dissociating from the substrate.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

As used herein, the term “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The terms “high,” “low,” “intermediate,” and “negative” in connection with cellular biomarker expression refers to the amount of the biomarker expressed relative to the cellular expression of the biomarker by one or more reference cells. Biomarker expression can be determined according to any method described herein including, without limitation, an analysis of the cellular level, activity, structure, and the like, of one or more biomarker genomic nucleic acids, ribonucleic acids, and/or polypeptides. In one embodiment, the terms refer to a defined percentage of a population of cells expressing the biomarker at the highest, intermediate, or lowest levels, respectively. Such percentages can be defined as the top 0.1%, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10%, 11%, 12%, 13%, 14%, 15% or more, or any range in between, inclusive, of a population of cells that either highly express or weakly express the biomarker. The term “low” excludes cells that do not detectably express the biomarker, since such cells are “negative” for biomarker expression. The term “intermediate” includes cells that express the biomarker, but at levels lower than the population expressing it at the “high” level. In another embodiment, the terms can also refer to, or in the alternative refer to, cell populations of biomarker expression identified by qualitative or statistical plot regions. For example, cell populations sorted using flow cytometry can be discriminated on the basis of biomarker expression level by identifying distinct plots based on detectable moiety analysis, such as based on mean fluorescence intensities and the like, according to well-known methods in the art. Such plot regions can be refined according to number, shape, overlap, and the like based on well-known methods in the art for the biomarker of interest. In still another embodiment, the terms can also be determined according to the presence or absence of expression for additional biomarkers.

The term “homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “host cell” is intended to refer to a cell into which a nucleic acid encompassed by the present invention, such as a recombinant expression vector encompassed by the present invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “humanized antibody”, as used herein, is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies encompassed by the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

As used herein, the term “hypervariable region,” “HVR,” or “HV,” refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al. (2000) Immunity 13, 37-45; Johnson and Wu in Methods in Molecular Biology 248, 1-25 (Lo, ed., Human Press, Totowa, N.J., 2003)). Indeed, naturally occurring camelid antibodies consisting of a heavy chain only are functional and stable in the absence of light chain (see, e.g., Hamers-Casterman et al. (1993) Nature 363:446-448 (1993) and Sheriff et al. (1996) Nature Struct. Biol. 3, 733-736).

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

Immune cells can be obtained from a single source or a plurality of sources (e.g., a single subject or a plurality of subjects). A plurality refers to at least two (e.g., more than one). In still another embodiment, the non-human mammal is a mouse. The animals from which cell types of interest are obtained may be adult, newborn (e.g., less than 48 hours old), immature, or in utero. Cell types of interest may be primary cells, stem cells, established cancer cell lines, immortalized primary cells, and the like.

The term “T cell” includes CD4⁺ T cells and CD8⁺ T cells. The term T cell also includes both T helper 1 type T cells and T helper 2 type T cells. The term “antigen presenting cell” includes professional antigen presenting cells (e.g., B lymphocytes, monocytes, dendritic cells, and Langerhans cells), as well as other antigen presenting cells (e.g., keratinocytes, endothelial cells, astrocytes, fibroblasts, and oligodendrocytes).

Conventional T cells, also known as Tconv or Teffs, have effector functions (e.g., cytokine secretion, cytotoxic activity, anti-self-recognization, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4⁺ Teffs or CD8+ Teffs, such as CD4⁺ helper T lymphocytes (e.g., Th0, Th1, Tfh, or Th17) and CD8+ cytotoxic T lymphocytes. As described further herein, cytotoxic T cells are CD8⁺ T lymphocytes. “Naïve Tcons” are CD4⁺ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637). In tumors, exhausted cells can present hallmarks of anergy.

The term “immunotherapy” refers to a form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in immunomodulatory therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer. As described above, immunotherapy against immune checkpoint targets, such as PD-1, PD-L1, PD-L2, CTLA-4, and the like are useful.

The term “immune checkpoint” refers to a group of molecules on the cell surface of CD4⁺ and/or CD8⁺ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein.

Immune checkpoints and their sequences are well-known in the art and representative embodiments are described below. For example, the term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

“Anti-immune checkpoint” or “immune checkpoint inhibitor or “immune checkpoint blockade” therapy refers to the use of agents that inhibit immune checkpoint nucleic acids and/or proteins. Immune checkpoints share the common function of providing inhibitory signals that suppress immune response and inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can bind to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. These embodiments are also applicable to specific therapy against particular immune checkpoints, such as the PD-1 pathway (e.g., anti-PD-1 pathway therapy, otherwise known as PD-1 pathway inhibitor therapy). Numerous immune checkpoint inhibitors are known and publicly available including, for example, Keytruda® (pembrolizumab; anti-PD-1 antibody), Opdivo® (nivolumab; anti-PD-1 antibody), Tecentriq® (atezolizumab; anti-PD-L1 antibody), durvalumab (anti-PD-L 1 antibody), and the like.

The term “immune response” includes T cell mediated and/or B cell mediated immune responses that are influenced by modulation of T cell costimulation. Exemplary immune responses include T cell responses, e.g., cytokine production, and cellular cytotoxicity. In addition, the term immune response includes immune responses that are indirectly effected by T cell activation, e.g., antibody production (humoral responses) and activation of cytokine responsive cells, e.g., macrophages.

As used herein, the term “immunotherapeutic agent” can include any molecule, peptide, antibody or other agent which can stimulate a host immune system to promote immunomodulation in the subject. Various immunotherapeutic agents are useful in the compositions and methods described herein.

The term “inhibit” or “downregulate” includes the decrease, limitation, or blockage, of, for example a particular action, function, or interaction. In some embodiments, a condition that would benefit from an increased immune response is “inhibited” if at least one symptom of the condition is alleviated, terminated, slowed, or prevented. As used herein, the condition is also “inhibited” if recurrence or spread of the condition is reduced, slowed, delayed, or prevented. Similarly, a biological function, such as the function of a protein, is inhibited if it is decreased as compared to a reference state, such as a control like a wild-type state. Such inhibition or deficiency can be induced, such as by application of agent at a particular time and/or place, or can be constitutive, such as by a heritable mutation. Such inhibition or deficiency can also be partial or complete (e.g., essentially no measurable activity in comparison to a reference state, such as a control like a wild-type state). Essentially complete inhibition or deficiency is referred to as blocked. The term “promote” or “upregulate” has the opposite meaning.

The term “interaction,” when referring to an interaction between two molecules, refers to the physical contact (e.g., binding) of the molecules with one another. Generally, such an interaction results in an activity (which produces a biological effect) of one or both of said molecules. The activity may be a direct activity of one or both of the molecules, (e.g., signal transduction). Alternatively, one or both molecules in the interaction may be prevented from binding their ligand, and thus be held inactive with respect to ligand binding activity (e.g., binding its ligand and triggering or inhibiting costimulation). To inhibit such an interaction results in the disruption of the activity of one or more molecules involved in the interaction. To enhance such an interaction is to prolong or increase the likelihood of said physical contact, and prolong or increase the likelihood of said activity.

The term “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of polypeptide, in which the protein is separated from cellular components of the cells from which it is isolated or recombinantly produced. In one embodiment, the language “substantially free of cellular material” includes preparations of protein, having less than about 30% (by dry weight) of non-desired protein (also referred to herein as a “contaminating protein”), more preferably less than about 20% of non-desired protein, still more preferably less than about 10% of non-desired protein, and most preferably less than about 5% non-desired protein. When antibody, polypeptide, peptide or fusion protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The term “isotype” refers to the antibody class (e.g., IgM or IgG1) that is encoded by heavy chain constant region genes.

The term “K_{D}” is intended to refer to the dissociation equilibrium constant of a particular antibody-antigen interaction. The binding affinity of antibodies of the disclosed invention may be measured or determined by standard antibody-antigen assays, for example, competitive assays, saturation assays, or standard immunoassays such as ELISA or RIA.

The term “modulate” includes up-regulation and down-regulation, e.g., enhancing or inhibiting a response.

The term “naturally-occurring” nucleic acid polypeptide refers to an RNA or DNA polypeptide having a nucleotide sequence that occurs in nature (e.g., encodes a natural protein).

A “kit” is any manufacture (e.g. a package or container) comprising at least one reagent, e.g. a probe or small molecule, for specifically detecting and/or affecting the expression of a marker encompassed by the present invention. The kit may be promoted, distributed, or sold as a unit for performing the methods encompassed by the present invention. The kit may comprise one or more reagents necessary to express a composition useful in the methods encompassed by the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit can be included.

The term “neoadjuvant therapy” refers to a treatment given before the primary treatment. Examples of neoadjuvant therapy can include chemotherapy, radiation therapy, and hormone therapy.

The “normal” level of expression of a marker is the level of expression of the marker in cells of a subject, e.g., a human patient, not afflicted with a condition that would benefit from an increased immune response. An “over-expression” or “significantly higher level of expression” of a marker refers to an expression level in a test sample that is greater than the standard error of the assay employed to assess expression, and is preferably at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the marker in a control sample (e.g., sample from a healthy subject not having the marker associated disease) and preferably, the average expression level of the marker in several control samples. A “significantly lower level of expression” of a marker refers to an expression level in a test sample that is at least twice, and more preferably 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the marker in a control sample (e.g., sample from a healthy subject not having the marker associated disease) and preferably, the average expression level of the marker in several control samples. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples. An “underexpression” or “significantly lower level of expression or copy number” of a marker (e.g., PTPN2) refers to an expression level or copy number in a test sample that is greater than the standard error of the assay employed to assess expression or copy number, but is preferably at least twice, and more preferably three, four, five or ten or more times less than the expression level or copy number of the marker in a control sample (e.g., sample from a healthy subject not afflicted with a condition that would benefit from an increased immune response) and preferably, the average expression level or copy number of the marker in several control samples.

Such “significance” levels can also be applied to any other measured parameter described herein, such as for expression, inhibition, cytotoxicity, cell growth, and the like.

The term “pre-determined” biomarker amount and/or activity measurement(s) may be a biomarker amount and/or activity measurement(s) used to, by way of example only, evaluate a subject that may be selected for a particular treatment, evaluate a response to a treatment such as one or more inhibitors of PTPN2 pathway, either alone or in combination with one or more therapies, and/or evaluate the disease state. A pre-determined biomarker amount and/or activity measurement(s) may be determined in populations of patients with or without a condition that would benefit from an increased immune response. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., cell ratios or serum biomarker normalized to the expression of housekeeping or otherwise generally constant biomarker). The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

The term “predictive” includes the use of a biomarker nucleic acid and/or protein status, e.g., over- or under-activity, emergence, expression, growth, remission, recurrence or resistance of tumors before, during or after therapy, for determining the likelihood of response of a condition that would benefit from an increased immune response (e.g., cancer or viral infection) to immunomodulatory therapy, such as PTPN2 pathway inhibitor therapy (e.g., inhibitor of the copy number, the expression level, and/or the activity of PTPN2, either alone or in combination with additional treatments). Such predictive use of the biomarker may be confirmed by, e.g., (1) decreased copy number (e.g., by FISH, FISH plus SKY, single-molecule sequencing, e.g., as described in the art at least at J. Biotechnol., 86:289-301, or qPCR), underexpression of a biomarker nucleic acid (e.g., by ISH, Northern Blot, or qPCR), decreased biomarker protein (e.g., by IHC) and/or biomarker target, or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with a condition that would benefit from an increased immune response; (3) its absolute or relatively modulated presence or absence in clinical subset of patients with a condition that would benefit from an increased immune response (e.g., those responding to a particular immunomodulatory therapy (e.g., PTPN2 pathway modulator therapy (e.g., inhibitor of the copy number, the expression level, and/or the activity of PTPN2, either alone or in combination with additional treatments) or those developing resistance thereto).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “probe” refers to any molecule which is capable of selectively binding to a specifically intended target molecule, for example, a nucleotide transcript or protein encoded by or corresponding to a biomarker nucleic acid. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

The term “prognosis” includes a prediction of the probable course and outcome of a condition that would benefit from an increased immune response or the likelihood of recovery from the disease. In some embodiments, the use of statistical algorithms provides a prognosis of the condition that would benefit from an increased immune response in an individual. For example, the prognosis can be surgery, development of a clinical subtype of the condition that would benefit from an increased immune response (e.g., cancer or chronic viral infection), development of one or more clinical factors, or recovery from the disease.

The term “response to therapy” relates to any response of a condition that would benefit from an increased immune response to therapy (e.g., PTPN2 pathway modulator therapy (e.g., inhibitor of the copy number, the expression level, and/or the acticity of PTPN2, either alone or in combination with additional treatments), preferably to a change in symptoms, such as reduced infection or viral load, tumor mass and/or volume after initiation of neoadjuvant or adjuvant chemotherapy, and the like. T cell function, such as CD4⁺ and/or CD8⁺ effector function, as well as antigen-specific function thereof, can be assessed according to numerous assays well-known in the art and/or described herein. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any immunomodulatory therapy. The outcome measurement may be pathologic response to therapy given in the neoadjuvant setting. Alternatively, outcome measures, such as overall survival and disease-free survival can be monitored over a period of time for subjects following immunomodulatory therapy for whom biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months.

The term “resistance” refers to an acquired or natural resistance of a sample or a mammal with a condition that would benefit from an increased immune response (e.g., cancer or viral infection) to an immunomodulatory therapy (i.e., being nonresponsive to or having reduced or limited response to the therapeutic treatment), such as having a reduced response to a therapeutic treatment by 5% or more, for example, 10%, 15%, 20%, 25%, 30%, 35%, 40%, 45%, 50%, 55%, 60%, 65%, 70%, 75%, 80%, 85%, 90%, 95%, 100%, or more, to 2-fold, 3-fold, 4-fold, 5-fold, 10-fold, 15-fold, 20-fold or more. The reduction in response can be measured by comparing with the same disease sample or mammal before the resistance is acquired, or by comparing with a different disease sample or a mammal who is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to response to therapy. For example, an anti-cancer response includes reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

The term “tolerance” or “unresponsiveness” includes refractivity of cells, such as immune cells, to stimulation, e.g., stimulation via an activating receptor or a cytokine. Unresponsiveness can occur, e.g., because of exposure to immunosuppressants or exposure to high doses of antigen. Several independent methods can induce tolerance. One mechanism is referred to as “anergy,” which is defined as a state where cells persist in vivo as unresponsive cells rather than differentiating into cells having effector functions. Such refractivity is generally antigen-specific and persists after exposure to the tolerizing antigen has ceased. For example, anergy in T cells is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134). Another mechanism is referred to as “exhaustion.” T cell exhaustion is a state of T cell dysfunction that arises during many chronic infections and cancer. It is defined by poor effector function, sustained expression of inhibitory receptors and a transcriptional state distinct from that of functional effector or memory T cells.

The term “peripheral blood cell subtypes” refers to cell types normally found in the peripheral blood including, but is not limited to, eosinophils, neutrophils, T cells, monocytes, NK cells, granulocytes, and B cells.

The term “recombinant human antibody” includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline and/or non-germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline VH and VL sequences, may not naturally exist within the human antibody germline repertoire in vivo.

The term “sample” used for detecting or determining the presence or level of at least one biomarker is typically whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method encompassed by the present invention further comprises obtaining the sample from the individual prior to detecting or determining the presence or level of at least one marker in the sample.

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene encompassed by the present invention, or a fragment thereof, short interfering RNA (siRNA), and small molecules which interfere with or inhibit expression of a target biomarker nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs, shRNAs, or other RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 0.95% or 99% or more as compared to the expression of a target biomarker nucleic acid or the activity or level of the protein encoded by a target biomarker nucleic acid which has not been targeted by an RNA interfering agent.

In addition to RNAi, genome editing can be used to modulate the copy number or genetic sequence of a biomarker of interest, such as constitutive or induced knockout or mutation of a biomarker of interest, such as PTPN2. For example, the CRISPR-Cas system can be used for precise editing of genomic nucleic acids (e.g., for creating non-functional or null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

“Piwi-interacting RNA (piRNA)” is the largest class of small non-coding RNA molecules. piRNAs form RNA-protein complexes through interactions with piwi proteins. These piRNA complexes have been linked to both epigenetic and post-transcriptional gene silencing of retrotransposons and other genetic elements in germ line cells, particularly those in spermatogenesis. They are distinct from microRNA (miRNA) in size (26-31 nt rather than 21-24 nt), lack of sequence conservation, and increased complexity. However, like other small RNAs, piRNAs are thought to be involved in gene silencing, specifically the silencing of transposons. The majority of piRNAs are antisense to transposon sequences, indicating that transposons are the piRNA target. In mammals it appears that the activity of piRNAs in transposon silencing is most important during the development of the embryo, and in both C. elegans and humans, piRNAs are necessary for spermatogenesis. piRNA has a role in RNA silencing via the formation of an RNA-induced silencing complex (RISC).

“Aptamers” are oligonucleotide or peptide molecules that bind to a specific target molecule. “Nucleic acid aptamers” are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. “Peptide aptamers” are artificial proteins selected or engineered to bind specific target molecules. These proteins consist of one or more peptide loops of variable sequence displayed by a protein scaffold. They are typically isolated from combinatorial libraries and often subsequently improved by directed mutation or rounds of variable region mutagenesis and selection. The “Affimer protein”, an evolution of peptide aptamers, is a small, highly stable protein engineered to display peptide loops which provides a high affinity binding surface for a specific target protein. It is a protein of low molecular weight, 12-14 kDa, derived from the cysteine protease inhibitor family of cystatins. Aptamers are useful in biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart et al. (2003) RNA April; 9(4):493-501 incorporated by reference herein).

RNA interfering agents, e.g., siRNA molecules, may be administered to a patient having or at risk for having a condition that would benefit from an increased immune response, to inhibit expression of a biomarker gene which is overexpressed in the condition and thereby treat, prevent, or inhibit the condition in the subject.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. 1998. Science 282:63), and natural product extract libraries. In another embodiment, the compounds are small, organic non-peptidic compounds. In a further embodiment, a small molecule is not biosynthetic.

The term “selective” refers to a preferential action or function. The term “selective” can be quantified in terms of the preferential effect in a particular target of interest relative to other targets. For example, a measured variable (e.g., the copy number, the expression level, and/or the activity of PTPN2) can be 10%, 20%, 25%, 30%, 35%, 40%, 45%, 50%, 55%, 60%, 65%, 70%, 75%, 80%, 85%, 90%, 95%, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 70-fold, 80-fold, 90-fold, 100-fold, or greater or any range in between inclusive (e.g., 50% to 16-fold), different in a target of interest versus unintended or undesired targets. The same fold analysis can be used to confirm the magnitude of an effect in a given tissue, cell population, measured variable, measured effect, and the like, such as cellular ratios, hyperproliferative cell growth rate or volume, T cell function or rate of proliferation, and the like.

By contrast, the term “specific” refers to an exclusionary action or function. For example, specific inhibition of the copy number, the expression level, and/or the activity of PTPN2 refers to the exclusive inhibition of the copy number, the expression level, and/or the activity of PTPN2 and not inhibition of another biomarker. In another example, specific binding of an antibody to a predetermined antigen refers to the ability of the antibody to bind to the antigen of interest without binding to other antigens. Typically, the antibody binds with an affinity (K_{D}) of approximately less than 1×10⁻⁷ M, such as approximately less than 10⁻⁸ M, 10⁻⁹ M, 10⁻¹⁰ M, 10⁻¹¹ M, or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. In addition. K_{D} is the inverse of K_{A}. The phrases “an antibody recognizing an antigen” and “an antibody specific for an antigen” are used interchangeably herein with the term “an antibody which binds specifically to an antigen.”

The term “sensitize” means to alter disease cells, such as infected or cancer cells, in a way that allows for more effective treatment of the associated condition with a therapy (e.g., PTPN2 pathway modulator therapy (e.g., inhibitor of the copy number, the expression level, and/or the activity of PTPN2), either alone or in combination with additional treatments). In some embodiments, normal cells are not affected to an extent that causes the normal cells to be unduly injured by the therapy (e.g., PTPN2 pathway modulator therapy (e.g., inhibitor of the copy number, the expression level, and/or the activity of PTPN2), either alone or in combination with additional treatments). An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42: 2159-2164), cell death assays (Weisenthal L M, Shoemaker R H, Marsden J A, Dill P L, Baker J A, Moran E M, Cancer Res 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet 1994; 19: 82-90). The sensitivity or resistance may also be measured in animal by measuring the tumor size reduction over a period of time, for example, 6 months for human and 4-6 weeks for mouse. A composition or a method sensitizes response to a therapeutic treatment if the increase in treatment sensitivity or the reduction in resistance is 5% or more, for example, 10%, 15%, 20%, 25%, 30%, 35%, 40%, 45%, 50%, 55%, 60%, 65%, 70%, 75%, 80%, 85%, 90%, 95%, 100%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of an immunomodulatory can be equally applied to methods for sensitizing hyperproliferative or otherwise cancerous cells (e.g., resistant cells) to the therapy.

The term “subject” refers to any healthy animal, mammal or human, or any animal, mammal or human afflicted with a condition of interest (e.g., a condition that would benefit from an increased immune response (e.g., cancer or viral infection)). The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include a condition that would benefit from an increased immune response (e.g., cancer or viral infection) and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to therapy, probability of survival, probability of recurrence within a given time period, and the like.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods encompassed by the present invention may be administered in a sufficient amount to produce a reasonable benefit/risk ratio applicable to such treatment.

The terms “therapeutically-effective amount” and “effective amount” as used herein means that amount of a compound, material, or composition comprising a compound encompassed by the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 400%, 50%, 60%, 70%, 80%, 90%, 100%, 200%, 300%, 400%, 500%, 600%, 700%, 800%, 900%, 1000% or more increased for the agent relative to no administration of the agent. Also, similarly, the IC₅₀ (i.e., the concentration which achieves a half-maximal effect, such as cytotoxic or cytostatic effect on cancer cells or inhibition of viral replication or load) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, an effect in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a malignancy or viral load can be achieved.

The term “substantially free of chemical precursors or other chemicals” includes preparations of antibody, polypeptide, peptide or fusion protein in which the protein is separated from chemical precursors or other chemicals which are involved in the synthesis of the protein. In one embodiment, the language “substantially free of chemical precursors or other chemicals” includes preparations of antibody, polypeptide, peptide or fusion protein having less than about 30% (by dry weight) of chemical precursors or non-antibody, polypeptide, peptide or fusion protein chemicals, more preferably less than about 20% chemical precursors or non-antibody, polypeptide, peptide or fusion protein chemicals, still more preferably less than about 10% chemical precursors or non-antibody, polypeptide, peptide or fusion protein chemicals, and most preferably less than about 5% chemical precursors or non-antibody, polypeptide, peptide or fusion protein chemicals.

A “transcribed polynucleotide” or “nucleotide transcript” is a polynucleotide (e.g. an mRNA, hnRNA, cDNA, mature miRNA, pre-miRNA, pri-miRNA, miRNA*, anti-miRNA, or a miRNA binding site, or a variant thereof or an analog of such RNA or cDNA) which is complementary to or homologous with all or a portion of a mature mRNA made by transcription of a marker encompassed by the present invention and normal post-transcriptional processing (e.g. splicing), if any, of the RNA transcript, and reverse transcription of the RNA transcript.

The term “vector” refers to a nucleic acid capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

Protein tyrosine phosphatases (PTPs or PTPases) are a group of enzymes that remove phosphate groups from phosphorylated tyrosine residues on proteins (He et al. (2014) Acta Pharmacol. Sin. 35:1227-1246; Barr et al. (2009) Cell 136:352-363). Protein tyrosine (pTyr) phosphorylation is a common post-translational modification that can create novel recognition motifs for protein interactions and cellular localization, affect protein stability, and regulate enzyme activity. As a consequence, maintaining an appropriate level of protein tyrosine phosphorylation is essential for many cellular functions. Tyrosine-specific protein phosphatases (PTPase: EC 3.1.3.48) catalyze the removal of a phosphate group attached to a tyrosine residue, using a cysteinyl-phosphate enzyme intermediate. These enzymes are key regulatory components in signal transduction pathways (such as the MAP kinase pathway) and cell cycle control, and are important in the control of cell growth, proliferation, differentiation, transformation, and synaptic plasticity (Denu and Dixon (1998) Curr. Opin. Chem. Biol. 2:633-641; Lombroso (2003) Cell. Mol. Life Sci. 60:2465-2482). Together with tyrosine kinases, PTPs regulate the phosphorylation state of many important signaling molecules, such as the MAP kinase family. PTPs are increasingly viewed as integral components of signal transduction cascades. PTPs have been implicated in regulation of many cellular processes, including, but not limited to: cell growth, cellular differentiation, mitotic cycles, oncogenic transformation, receptor endocytosis, etc. The classification of PTPs can be achieved by mechanism or location. By mechanism, PTP activity can be found in four protein families, including: 1) class I PTPs, which is the largest group of PTPs comprising at least 99 members, such as at least 38 classical PTPs (21 receptor tyrosine phosphatase and 17 non-receptor-type PTPs) and 61 VH-1-like or dual-specific (dTyr and dSer/dThr) phosphatases (DSPs) (e.g., 11 MAPK phosphatases (MPKs), 3 Slingshots, 3 PRLs, 4 CDC14s, 19 atypical DSPs, 5 Phosphatase and tensin homologs (PTENs), and 16 Myotubularins); 2) class II PTP, comprising only one member low-molecular-weight phosphotyrosine phosphatase (LMPTP); 3) class III PTPs, comprising at least CDCl₂₅ A, B, and C proteins; and 4) Class IV PTPs, comprising at least Eya 1-4 proteins, which are pTyr-specific phosphatases and believed to have evolved separately from the other three classes. By cellular location, PTPs can be classified as receptor-like PTPs and non-receptor (intracellular) PTPs. The former are transmembrane receptors that contain PTPase domains. In terms of structure, all known receptor PTPases are made up of a variable-length extracellular domain, followed by a transmembrane region and a C-terminal catalytic cytoplasmic domain. Some of the receptor PTPases contain fibronectin type III (FN-III) repeats, immunoglobulin-like domains, MAM domains, or carbonic anhydrase-like domains in their extracellular region. In general, the cytoplasmic region contains two copies of the PTPase domain. The first has enzymatic activity, whereas the second is inactive (Sun et al. (2003) Curr Top Med Chem. 3:739-748; Alonso et al. (2004) Cell 117:699-711). All class I, II, and III PTPs carry a highly conserved active site motif C(X)₅R (PTP signature motif), employ a common catalytic mechanism, and possess a similar core structure made of a central parallel beta-sheet with flanking alpha-helices containing a beta-loop-alpha-loop that encompasses the PTP signature motif (Barford et al. (1998) Ann. Rev. Biophys. Biomol. Struct. 27:133-164). Functional diversity between PTPases is endowed by regulatory domains and subunits. For most PTPs, the consensus sequence (I/V)HCXAGXXR(S/T)G (i.e., the C(X)₅R PTP signature motif) contains the catalytically essential Cys and Arg residues. Intracellular PTPs are often modular molecules containing structural motifs such as Src homology 2 (SH2) domains, PEST sequences, and band 4.1 domains on either the N- or C-terminal side of their catalytic domains.

Among non-receptor PTPs, tyrosine-protein phosphatase non-receptor type 2 (PTPN2) is an enzyme that in humans is encoded by PTPN2 gene (Brown-Shimer et al. (1990) Proc. Natl. Acad Sci. USA 87:5148-5152). Epidermal growth factor receptor and the adaptor protein Shc were reported to be substrates of this PTP, which indicates a role in growth factor-mediated cell signaling. Three alternatively spliced variants of this gene, which encode isoforms differing at their extreme C-termini, have been described. The different C-termini are thought to determine the substrate specificity, as well as the cellular localization of the isoforms. Two highly related but distinctly processed pseudogenes that localize to distinct human chromosomes have been reported. The human PTPN2 gene localizes to chromosome 18p11.2-p11.3, whereas pseudogenes (gene symbol PTPN2P1 and PTPN2P2) are mapped to chromosomes 1q22-q24 and 13q12-q13, respectively. A direct comparison of the specificity of genomic and cDNA probes demonstrated that under identical conditions the genomic probes (containing both exon and intron sequences) readily identified a single specific site of hybridization, whereas the cDNA identified sites of both the gene and its pseudogenes (Johnson et al. (1993) Genomics 16:619-629). Human PTPN2 exists as two forms generated by alternative splicing: a 48-kDa endoplasmic reticulum (ER)-associated form (TC48, 415 amino acid) and a 45-kDa nuclear form (TC45). The three-dimensional PDB structure of PTPN2 is also well-known and described in at least the OCA database (protein ID: 1L8K) at the Weizmann Institute of Science (Rehovot, Israel) available on the World Wide Web at oca.weizmann.ac.il/oca-bin/ocashort?id=1L8K. PTPN2 has a protein tryrosine phosphatase catalytic (PTPc) domain, for example, from amino acid residues 5 to 275 of SEQ ID NO: 2. The PTPc domain comprises different motifs for various functions, such as substrate binding (amino acid residues 216-222 of SEQ ID NO: 2), endoplasmic reticulum (ER) location (amino acid residues 346-415 of SEQ ID NO: 2), and STX17 interaction (amino acid residues 376-415 of SEQ ID NO: 2, also see Muppirala et al. (2012) Biochim. Biophys. Acta 1823:2109-2119).

The nucleic acid and amino acid sequences of a representative human PTPN2 is available to the public at the GenBank database (Gene ID 5771) and is shown in Table 1. Human PTPN2 isoforms include the longest isoform 1 (GenBank database numbers NM_002828.3 and NP_002819.2), and the shorter isoforms 2 (NM_080422.2 and NP_536347.1, which contains an alternate 3′ region including a part of the C-terminal coding region, resulting in a shorter and distinct C-terminus, as compared to isoform 1), 3 (NM_080423.2 and NP_536348.1; which contains an alternate 3′ region including a part of the C-terminal coding region, resulting in a shorter and distinct C-terminus, as compared to isoform 1), 4 (NM_001207013.1 and NP_001193942.1; which contains an additional in-frame exon in the middle coding region and an alternate 3′ region including a part of the C-terminal coding region, resulting in an additional internal segment and a shorter and distinct C-terminus, as compared to isoform 1), and 5 (NM_001308287.1 and NP_001295216.1; which differs in the 5′ UTR by lacking a portion of the 5′ coding region and using an alternative start codon to initiates translation, resulting in a shorter and distinct N-terminus, as compared to isoform 1).

Nucleic acid and polypeptide sequences of PTPN2 orthologs in organisms other than humans are well-known and include, for example, chimpanzee (Pan troglodytes) PTPN2 (XM_009433614.2 and XP_009431889.2; XM_009433613.2 and XP_009431888.2; XM_009433615.2 and XP_009431890.2; XM_003953237.2 and XP_003953286.2; XM_001171536.4 and XP_001171536.2; XM_009433617.2 and XP 009431892.1; XM 016933257.1 and XP 016788746.1; XM 009433619.2 and XP_009431894.2; XM_009433618.2 and XP_009431893.2; XM_016933256.1 and XP_016788745.1; XM_016933258.1 and XP_016788747.1; and XM_009433620.2 and XP_009431895.2), dog PTPN2 (XM_014115598.1 and XP_013971073.1; XM 005623101.2 and XP 005623158.1; XM 005623100.2 and XP 005623157.1; and XM_005623099.2 and XP_005623156.1), mouse PTPN2 (NM_001127177.1 and NP_001120649.1, which represent the longer transcript, and NM_008977.3 and NP_033003.1, which differs in the 3′ UTR and has multiple coding region differences, resulting in a distinct C-terminus and is shorter than the isoform encoded by the longer transcript), cattle PTPN2 (NM_001035431.2 and NP_001030508.1), Norway rat (Rattus norvegicus) PTPN2 (NM_053990.1 and NP_446442.1), chicken PTPN2 (NM_001199387.1 and NP_001186316.1), tropical clawed frog (Xenopus tropicalis) PTPN2 (XM_004915252.3 and XP_004915309.2; and XM_002936076.4 and XP_002936122.1); zebrafish (Danio rerio) PTPN2 (NM_200466.2 and NP_956760.2; and NM_212654.1 and NP_997819.1); and fruit fly (Drosophila melanogaster) PTPN2 (NM_167874.2 and NP_728600.1; NM_057340.4 and NP_476688.1; NM_001274324.2 and NP_001261253.1; NM 167875.2 and NP 728601.1; and NM 057339.5 and NP_476687.1).

The term “PTPN2 activity,” includes the ability of a PTPN2 polypeptide (and its fragments, domains, and/or motifs thereof, discussed herein) to bind and catalyze the removal of one or more phosphate groups from one or more its substrates in a cell (e.g., a cancer cell, and/or an immune cell), e.g., by engaging a natural PTPN2 substrate (e.g., INSR, EGFR, CSF1R, PDGFR, JAK1, JAK2, JAK3, Src family kinases, STAT1, STAT3, STAT5A, STAT5B, STAT6, etc.) either in the nucleus or the cytoplasm of the cell (Shuai et al. (2003) Nat. Rev. Immunol. 3:900-911; Wiede et al. (2011) J. Clin. Invest. 121:4758-4774). Thus, the term “PTPN2 activity” includes the ability of a PTPN2 polypeptide to bind its natural substrate(s), the ability to modulate dephosphorylation of such substrate(s), and the ability to modulate the immune response through such substrate(s) in PTPN2-regulated signaling pathways.

The term “PTPN2 substrate(s)” refers to binding partners of a PTPN2 polypeptide (and its fragments, domains, and/or motifs thereof, discussed herein) from which one or more phosphate groups can be removed by PTPN2 polypeptide. Such binding partners are usually members in PTPN2-regulated signaling pathways, such as INSR, EGFR, CSF1R, PDGFR, JAK1, JAK2, JAK3, Src family kinases, STAT1, STAT3, STAT5A, STAT5B, STAT6, etc. The term “INSR” refers to a member of gene superfamily that functions as insulin receptors. INSR is also commonly known under the names CD220, HHF5, insulin receptor isoform Long preproprotein, insulin receptor isoform Short preproprotein, and IR. INSR, localizing on 19p13.2, encodes a long protein that may be cleaved into four parts: two alpha subunits and two beta subunits. These subunits work together as a functioning receptor. The alpha subunits stick out from the surface of the cell, while the beta subunits remain inside the cell. The alpha subunits attach (bind) to insulin, which causes the beta subunits to trigger signaling pathways within the cell that influence many cell functions. The INSR gene mutations have been associated with at least Donohue Syndrome, Rabson-Mendenhall Syndrome, and type A insulin resistance syndrome. The nucleic acid and amino acid sequences of a representative human INSR is available to the public at the GenBank database. Human INSR isoforms include the longer isoform 1 (GenBank database numbers NM_000208.3 and NP_000199.2; a.k.a. insulin receptor isoform Long preproprotein) and the shorter isoform 2 (NM_001079817.2 and NP_001073285.1; a.k.a. insulin receptor isoform Short preproprotein).

The term “PTPN2-regulated signaling pathway(s)” includes signaling pathways in which PTPN2 (and its fragments, domains, and/or motifs thereof, discussed herein) binds to at least one of its substrate, through which at least one cellular function and/or activity and/or cellular protein profiles is changed. In some embodiments, PTPN2 dephosphorylates at least one of its substrates which bind to it. PTPN2-regulated signaling pathways include at least NF-kappaB signaling pathway, MAP kinase signaling pathway, Jak-STAT signaling pathway, cytokine signaling pathway, interferon gamma (IFNγ) signaling pathway, etc. In some embodiments, PTPN2-regulated signaling pathway is a type I interferon signaling pathway and/or type II interferon signaling pathway, which are summarized in at least Platanias (2005) Nat. Rev. Immunol. 5:375-386. The most studied members of the Type I interferons (IFNs) are the multiple IFNα isotypes and IFNβ. Type I IFNs are responsible for inducing transcription of a large group of genes which play a role in host resistance to viral infections, as well as activating key components of the innate and adaptive immune systems, including antigen presentation and production of cytokines involved in activation of T cells, B cells, and natural killer cells. Type I IFNs are transcriptionally regulated, and are induced following recognition of pathogen components during infection by various host pattern recognition receptors. Virtually all humans cells are able to synthesize IFNα/β, however some cells have a more pronounced ability to produce these cytokines. At least three pathways (i.e., the RIG-I pathway, the TRIF pathway, and TLR7/8/9-IRF7 pathway) the are involved in producing Type I IFN. Following their production, Type I IFNs trigger antiviral responses by binding to a common receptor (IFNAR). IFNα/β binding to IFNAR stimulates the JAK1-STAT pathway leading to the assembly of the ISGF3 complex which is composed of STAT1-STAT2 dimers and IRF9. ISGF3 binds to IFN-stimulated response elements (ISRE) in the promoters of IFN-stimulated genes to regulate their expression. Among these genes is IRF7 which initiates the transcription of a second wave of Type I IFNs. This autocrine/paracrine feed-back allows Type I IFNs to create an antiviral state in surrounding cells. IFNγ is the only type II interferon. While it does not share structural homology or a common receptor with the type I IFNs, it too has antiviral and immunomodulatory properties. The biologically active form of IFNγ is a noncovalently-linked homodimer. This homodimer binds to the extracellular domain of two IFNγR1/CD119 chains, which interact with IFNγR2 to form the functional IFNγ receptor complex. The IFNγR1 subunits of the receptor complex are associated with Jak1, while the IFNγR2 subunits are associated with Jak2. Activation of Jak1 and Jak2 results in phosphorylation of the receptor and subsequent recruitment and phosphorylation of STAT1. STAT1 phosphorylation leads to its homodimerization and nuclear translocation. Once in the nucleus, STAT1 homodimers bind to IFNγ-activated sequence (GAS) elements in the promoters of target genes to regulate their transcription. Many of the target genes that are induced by IFNγ/STAT1 signaling are transcription factors that then drive the expression of secondary response genes. In addition, IFNγ signaling can activate MAPK, PI3K-Akt, and NF-kappa B signaling pathways to regulate the expression of a number of other genes. IFNγ signaling plays a key role in host defense by promoting macrophage activation, upregulating the expression of antigen processing and presentation molecules, driving the development and activation of Th1 cells, enhancing natural killer cell activity, regulating B cell functions, and inducing the production of chemokines that promote effector cell trafficking to sites of inflammation. While IFNγ has historically been known for its cytotoxic, cytostatic, and anti-tumor properties, multiple studies have also suggested that IFNγ may also have context-dependent proliferative and pro-tumorigenic effects. For a summary of the Type II interferon signaling pathway, see at least Raza et al. (2008) BMC Sys. Biol. 2:36). IFNγ signaling can at least promote NK cell activity, increase antigen presentation and lysosome activity of macrophages, activate inducible nitric oxide synthase (iNOS), and induce the production of IgG2a and IgG3 from activated plasma B cells. Many IFN-stimulated genes control viral, bacterial, and parasite infection by directly targeting pathways and functions required during pathogen life cycles. The detection methods for such activation or inhibitor of IFNγ responsive genes are also well known in the art. In some embodiments, the cancer cells described herein have functional IFNγ signaling pathway but inactivated or at least reduced activation of IFNγ-responsive target genes, probably due to the inhibition by PTPN2. Upon a treatment with the antagonizing agent for PTPN2, as described herein, such cancer cells restore active IFNγ signaling. Such restoration of IFNγ signaling can be detected and/or measured through the expression and/or function of IFN-responsive genes, as described herein, using any known method in the art.

The term “PTPN2 inhibitor(s)” includes any natural or non-natural agent prepared, synthesized, manufactured, and/or purified by human that is capable of reducing, inhibiting, blocking, preventing, and/or that inhibits the ability of a PTPN2 polypeptide (and its fragments, domains, and/or motifs thereof, discussed herein). In one embodiment, such inhibitors may reduce or inhibit the binding/interaction between PTPN2 and its substrates or other binding partners. In another embodiment, such inhibitors may reduce or inhibit the catalytic function of PTPN2 as a phosphatase. In still another embodiment, such inhibitors may increase or promote the turnover rate, reduce or inhibit the expression and/or the stability (e.g., the half-life), and/or change the cellular localization of PTPN2, resulting in at least a decrease in PTPN2 levels and/or activity. Such inhibitors may be any molecule, including but not limited to small molecule compounds, antibodies or intrabodies, RNA interfereing (RNAi) agents (including at least siRNAs, shRNAs, microRNAs (miRNAs), piwi, and other well-known agents). Smalle molecule inhibitors of PTPN2 are known in the art and include, for example, 4-(aminosulfonyl)-7-fluoro-2,1,3-benzoxadiazole (ABDF; see Hansen et al. (2005) Biochemistry 44(21):7704-7712), imatinib mesylate (STI571; see Shimizu et al. (2004) Erp Hematol. 32(11):1057-1063), PTP inhibitor V (PHPS1, see Kim and Cho (2013) Bull. Korean Chem. Soc. 34:3874-3876), ethyl-3,4-dephospatin or other PTPN2 inhibitors described in the PCT Publ. No. WO 2015/188228, inhibitors described in Romsicki et al. (2003) Arch Biochem Biophys. 414:40-50, Iversen et al. (2002) J. Biol. Chem. 277(22):19982-19990, and Asante-Appiah et al. (2001) J. Biol. Chem. 276(28):26036-26043; commercial available PTP and/or PTPN2 inhibitors (e.g., products from EMD Millipore, Billerica, Mass., such as anti-PTPN2 antibodies, ursolic acid, sodium orthovanadate, Dephostatin, Phenylarsine Oxide, PTP Inhibitor I, bpV(HOpic), bpV(phen), bpV(bipy), etc.), inhibitory nucleotide-related inhibitors (such as CRISPR products from OriGene (Rockville, Md.) (e.g., gRNA vectors KN202161G1 and KN202161G2), GenScript® (Piscataway, N.J.), or Santa Cruz Biotechnology (Dallas, Tex.) (e.g., TC-PTP CRISPR/Cas9 KO Plasmid (h); sc-403071), miRNA products from ViGene Biosciences, inhibitory RNA products from Origene (e.g., siRNA, shRNA, etc.) and ViGene Biosciences (e.g., ready-to-package AAV shRNA), etc. Methods for developoing PTPN2-specific inhibitors based on its structure can be found in, e.g., Iversen et al. (2001) Biochemistry 40(49):14812-14820. An exemplary method, without limitation, of analyzing the activity of PTPN2 inhibitors described herein is to analyze whether such inhibitors are capable of 1) inhibiting the phosphatase function of Ptpn2; and/or 2) restoring IFNγ signaling and/or cellular sensitivity to immunotherapy. Methods for detecting the phosphatase function of Ptpn2 are well-known in the art. For example, the phosphorylation of Ptpn2 targets, including receptor tyrosine kinases (e.g., INSR, EGFR, CSF1R, PDGFR, etc.), non-receptor tyrosine kinases (e.g., JAK1, JAK2, JAK3, etc.), Src family kinases (e.g., Fyn, Lck, etc.) and STAT family members (e.g., STAT1, STAT3, STAT5A, STAT5B, STAT6, etc.), can be measured and compared before and after PTPN2 inhibitor treatment.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for nucleic acid and polypeptide molecules useful in the present invention are well-known in the art and readily available on publicly available databases, such as the National Center for Biotechnology Information (NCBI). For example, exemplary nucleic acid and amino acid sequences derived from publicly available sequence databases are provided in Table 1 below.

TABLE 1
SEQ ID NO: 1 Human PTPN2 isoform 1 cDNA Sequence (NM 002828.3)

| | |
|---|---|
| 1 | gctcgggcgc cgagtctgcg cgctgacgtc cgacgctcca ggtactttcc ccacggccga |
| 61 | cagggcttgg cgtgggggcg gggcgcggcg cgcagcgcgc atgcgccgca gcgccagcgc |
| 121 | tctccccgga tcgtgcgggg cctgagcctc tccgccggcg caggctctgc tcgcgccagc |
| 181 | tcgctcccgc agccatgccc accaccatcg agcgggagtt cgaagagttg gatactcagc |
| 241 | gtcgctggca gccgctgtac ttggaaattc gaaatgagtc ccatgactat cctcatagag |
| 301 | tggccaagtt tccagaaaac agaaatcgaa acagatacag agatgtaagc ccatatgatc |
| 361 | acagtcgtgt taaactgcaa aatgctgaga atgattatat taatgccagt ttagttgaca |
| 421 | tagaagaggc acaaaggagt tacatcttaa cacagggtcc acttcctaac acatgctgcc |
| 481 | atttctggct tatggtttgg cagcagaaga ccaaagcagt tgtcatgctg aaccgcattg |
| 541 | tggagaaaga atcggttaaa tgtgcacagt actggccaac agatgaccaa gagatgctgt |
| 601 | ttaaagaaac aggattcagt gtgaagctct tgtcagaaga tgtgaagtcg tattatacag |
| 661 | tacatctact acaattagaa aatatcaata gtggtgaaac cagaacaata tctcactttc |
| 721 | attatactac ctggccagat tttggagtcc ctgaatcacc agcttcattt ctcaatttct |
| 781 | tgtttaaagt gagagaatct ggctccttga accctgacca tgggcctgdg gtgatccact |
| 841 | gtagtgcagg cattgggcgc tctggcacct tctctctggt agacacttgt cttgttttga |
| 901 | tggaaaaagg agatgatatt aacataaaac aagtgttact gaacatgaga aaataccgaa |
| 961 | tgggtattat tcagaccdca gatcaactga gattctcata catggctata atagaaggag |
| 1021 | caaaatgtat aaagggagat tctagtatac agaaacgatg gaaagaactt tctaaggaag |
| 1081 | acttatctcc tgcctttgat cattcaccaa acaaaataat gactgaaaaa tacaatggga |
| 1141 | acagaatagg tdtagaagaa gaaaaactga caggtgaccg atgtacagga ctttcctcta |
| 1201 | aaatgcaaga tacaatggag gagaacagtg agagtgctct acggaaacgt attcgagagg |
| 1261 | acagaaaggc caccacagdt cagaaggtgc agcagatgaa adagaggcta aatgagaatg |
| 1321 | aacgaaaaag aaaaaggtgg ttatattggc aacctattct cactaagatg gggtttatgt |
| 1381 | cagtcatttt ggttggcgct tttgttggct ggacactgtt ttttcagcaa aatgccctat |
| 1441 | aaacaattaa ttttgcccag caagettctg cactagtaac tgacagtgct acattaatca |
| 1501 | taggggtttg tctgcagcaa acgcctcata tcccaaaaac ggtgcagtag aatagacatc |
| 1561 | aaccagataa gtgatattta cagtcacaag cccaacatct caggactctt gactgcaggt |
| 1621 | tcctctgaac cccaaactgt aaatggctgt ctaaaataaa gacattcatg tttgttaaaa |
| 1681 | actggtaaat tttgcaactg tattcataca tgtcaaacac agtatttcac ctgaccaaca |
| 1741 | ttgagatatc ctttatcaca ggatttgttt ttggaggcta tdtggatttt aacctgcact |
| 1801 | tgatataagc aataaatatt gtggttttat ctacgttatt ggaaagaaaa tgacatttaa |
| 1861 | ataatgtgtg taatgtataa tgtactattg acatgggcat caacactttt attcttaagc |
| 1921 | atttcagggt aaatatattt tataagtatc tatttaatct tttgtagtta actgtacttt |
| 1981 | ttaagagctc aatttgaaaa atctgttact aaaaaaataa attgtatgtc gattgaattg |
| 2041 | tactggatac attttccatt tttctaaaga gaagtttgat atgagcagtt agaagttgga |
| 2101 | ataagcaatt tctactatat attgcatttc ttttatgttt tacagttttc cccattttaa |
| 2161 | aaagaaaagc aaacaaagaa acaaaagttt ttcctaaaaa tatctttgaa ggaaaattct |
| 2221 | ccttactggg atagtcaggt aaacagttgg tcaagacttt gtaaagaaat tggtttctgt |
| 2281 | aaatcccatt attgatatgt ttatttttca tgaaaatttc aatgtagttg gggtagatta |
| 2341 | tgatttagga agcaaaagta agaagcagca ttttatgatt cataatttca gtttactaga |
| 2401 | ctgaagtttt gaagtaaaca cttLtdagtt tctttctact tcaataaata gtatgattat |
| 2461 | atgdaaacct tacattgtca ttttaactta atgaatattL Lttaaagcaa actgtLtaat |
| 2521 | gaatttaact gctcatttga atgctagctt tcctcagatt tcaacattcc attcagtgtt |
| 2581 | taatttgtct tacttaaact tgaaattgtt gttacaaatt taattgctag gaggcatgga |
| 2641 | tagcatacat tattatggat agcatacctt atttcagtgg ttLtdaaact atgdtcattg |
| 2701 | gatgtccagg tgggtcaaga ggttactttc aaccacagca tctctgcctt gtctctttat |
| 2761 | atgccacata agatttctgc ataaggctta agtattttaa agggggcagt tatcatttaa |
| 2821 | aaacagtttg gtcgggcgcg gtggctcatg cctgtaatcc cagcactttg ggaggctgaa |
| 2881 | gtgggcagat cacctgaggt caggagttca agaccagcct ggccaacgtg gtgaaacacc |
| 2941 | atctctacta aaaatgcaaa aattagdtgg gcatggtgga gggcacctgt aatctcagct |
| 3001 | actcaggagg ctgaggtagg agaattgctt gaacccagga gatggaggtt gcagtgagct |
| 3061 | gagatcacgt cactgcactc cagccagggc gacagagcga gactccatct caaaagaaac |
| 3121 | aaacaaaaaa aacagtttgg gccgggtgtg gtggctcacg cttgtaatcc cagcacttcg |
| 3181 | gaaggccaag gcgggcggat cacgaggtca agagatggag actgtcdtgg ccaacatggt |
| 3241 | gaaatccctt ctttactaaa aatacaaaaa ttatctgggc gtggtggtgc atgdctgtag |
| 3301 | tcccagctcc ttgggaggct aaggcaggag aatcacttga acccgggagg cagaggttgc |
| 3361 | agtgagccga gattgcacca ctgcactcca gcctggcaac agagcaagac ttcgtctc |

SEQ ID NO: 2 Human PTPN2 isoform 1 Amino Acid Sequence (NP 002819.2)

| | |
|---|---|
| 1 | mpttierefe eldtgrrwqp lyleirnesh dyphrvakfp enrnrnryrd vspydhsrvk |
| 61 | lqnaendyin aslvdieeaq rsyiltggpl pntcchfwim vwqgktkavv mlnrivekes |
| 121 | vkcagywptd dgemifketg fsvkllsedv ksyytvhllq leninsgetr tishfhyttw |
| 181 | pdfgvpespa sfinfifkvr esgslnpdhg pavihcsagi grsgtfslvd tcivimekgd |
| 241 | dinikqvlln mrkyrmglig tpdglrfsym aiiegakcik gdssigkrwk eiskeddspa |
| 301 | fdhspnkimt ekyngnrigl eeekltgdrc tglsskmqdt meensesalr kriredrkat |
| 361 | taqkvqqmkq rinenerkrk rwlywqpilt kmgfmsvilv gafvgwtlff qqnal |

SEQ ID NO: 3 Human PTPN2 isoform 2 cDNA Sequence (NM 080422.2)

| | |
|---|---|
| 1 | gctcgggcgc cgagtctgcg cgctgacgtc cgacgctcca ggtactttcc ccacggccga |
| 61 | cagggcttgg cgtgggggcg gggcgcggcg cgcagcgdgc atgcgccgca gcgccagcgc |
| 121 | tctccccgga tcgtgcgggg cctgagcctc tccgccggcg caggctctgc tcgcgccagc |
| 181 | tcgctcccgc agccatgcdc accaccatcg agcgggagtt cgaagagttg gatactcagc |
| 241 | gtcgctggca gccgctgtac ttggaaattc gaaatgagtc ccatgactat cctcatagag |
| 301 | tggdcaagtt tccagaaaac agaaatcgaa acagatacag agatgtaagd ccatatgatc |
| 361 | acagtcgtgt taaactgcaa aatgctgaga atgattatat taatgccagt ttagttgaca |
| 421 | tagaagaggc acaaaggagt tacatcttaa cacagggtcc acttcctaac acatgctgcc |
| 481 | atttctggct tatggtttgg cagcagaaga ccaaagcagt tgtcatgctg aaccgcattg |
| 541 | tggagaaaga atcggttaaa tgtgcacagt actggccaac agatgaccaa gagatgctgt |
| 601 | ttaaagaaac aggattcagt gtgaagctct tgtcagaaga tgtgaagtcg tattatacag |
| 661 | tacatctact acaattagaa aatatcaata gtggtgaaac cagaacaata tctcactttc |
| 721 | attatactac ctggccagat tttggagtcc ctgaatcacc agcttcattt ctcaatttct |
| 781 | tgtttaaagt gagagaatct ggctccttga accctgacca tgggcctgcg gtgatccact |
| 841 | gtagtgcagg cattgggcgc tctggdacct tctctctggt agacacttgt cttgttttga |
| 901 | tggaaaaagg agatgatatt aacataaaac aagtgttact gaacatgaga aaataccgaa |
| 961 | tgggtcttat tcagacccca gatcaactga gattctcata catggctata atagaaggag |
| 1021 | caaaatgtat aaagggagat tctagtatac agaaacgatg gaaagaactt tctaaggaag |
| 1081 | acttatctcc tgcctttgat cattcaccaa acaaaataat gactgaaaaa tacaatggga |
| 1141 | acagaatagg tctagaagaa gaaaaactga caggtgaccg atgtacagga ctttcctcta |
| 1201 | aaatgcaaga tacaatggag gagaacagtg agagtgctct acggaaacgt attcgagagg |
| 1261 | acagaaaggc caccacagct cagaaggtgc agcagatgaa acagaggcta aatgagaatg |
| 1321 | aacgaaaaag aaaaaggcca agattgacag acacctaata ttcatgactt gagaatattc |
| 1381 | tgcagctata aattttgaac cattgatgtg caaagcaaga cctgaagccc actccggaaa |
| 1441 | ctaaagtgag gctcgctaac cctctagatt gcctcacagt tgtttgttta caaagtaaac |
| 1501 | tttacatcca ggggatgaag agcacccacc agcagaagac tttgcagaac ctttaattgg |
| 1561 | atgtgttaag tgtttttaat gagtgtatga aatgtagaaa gatgtacaag aaataaatta |
| 1621 | ggggagatta ctttgtattg tactgccatt cctactgtat ttttatactt tttggcagca |
| 1681 | ttaaatattt ttgttaaata gtcaaaaaaa aaaaaaaaaa a |

SEQ ID NO: 4 Human PTPN2 isoform 2 Amino Acid Sequence (NP 536347.1)

| | |
|---|---|
| 1 | mpttierefe eldtqrrwgp lyleirnesh dvphrvakfp enrnrnrvrd vspvdhsrvk |
| 61 | lgnaendyin aslvdieeaq rsviltggpl pntcchfwlm vwqqktkavv mlnrivekes |
| 121 | vkcagywptd dgemifketg fsvklisedv ksyytvhilq leninsgetr tishfhyttw |
| 181 | pdfgvpespa sflnflfkvr esgsinpdhq pavihcsagi grsgtfslvd tclvlmekqd |
| 241 | dinikgvlln mrkyrmgliq tpdglrfsvm aiiegakcik gdssigkrwk elskedispa |
| 301 | fdhspnkimt ekvngnrigl eeekltgdrc tglsskmgdt meensesalr kriredrkat |
| 361 | taqkvqqmkq rlnenerkrk rprltdt |

SEQ ID NO: 5 Human PTPN2 isoform 3 cDNA Sequence (NM 080423.2)

| | |
|---|---|
| 1 | gctcgggcgc cgagtctgcg cgctgacgtc cgacgctcca ggtactttcc ccacggccga |
| 61 | cagggcttgg cgtgggggcg gggcgcggcg cgcagcgcgc atgcgccgca gcgccagcgc |
| 121 | tctccccgga tcgtgcgggg cctgagcctc tccgccggcg caggctctgc tcgcgccagc |
| 181 | tcgctcccgc agccatgccc accaccatcg agcgggagtt cgaagagttg gatactcagc |
| 241 | qtcgctggca gccgctqtac ttggaaattc gaaatgagtc ccatgactat cctcatagag |
| 301 | tggccaagtt tccagaaaac agaaatcgaa acagatacag agatgtaagc ccatatgatc |
| 361 | acagtcgtgt taaactgcaa aatgctgaga atgattatat taatgccagt ttagttgaca |
| 421 | tagaagaggc acaaaggagt tacatcttaa cacagggtcc acttcctaac acatgctqcc |
| 481 | atttctggct tatggtttgg cagcagaaqa ccaaaqcagt tgtcatgctg aaccgcattg |
| 541 | tggagaaaga atcggttaaa tgtgcacaqt actggccaac agatgaccaa gagatgctqt |
| 601 | ttaaagaaac aggattcagt gtgaagctct tgtcagaaga tgtgaagtcg tattatacag |
| 661 | tacatctact acaattagaa aatatcaata gtggtgaaac cagaacaata tctcactttc |
| 721 | attatactac ctggccagat tttggagtcc ctgaatcacc agcttcattt ctcaatttct |
| 781 | tqtttaaagt gagagaatct ggctccttga accctgacca tgggcctgcg gtgatccact |
| 841 | qtagtgcagg cattgggcgc tctqgcacct tctctctggt agacacttgt cttgttttga |
| 901 | tggaaaaagg agatgatatt aacataaaac aagtgttact gaacatgaga aaataccgaa |
| 961 | tgggtcttat tcagacccca gatcaactga gattctcata catggctata atagaaggag |
| 1021 | caaaatgtat aaagggagat tctagtatac agaaacgatg gaaagaactt tctaaggaag |
| 1081 | acttatctcc tgcctttgat cattcaccaa acaaaataat gactgaaaaa tacaatggga |
| 1141 | acagaatagg tctagaagaa gaaaaactga caggtgaccg atgtacagga ctttcctcta |
| 1201 | aaatgcaaga tacaatggag gagaacagtg agaggccaag attgacagac acctaatatt |
| 1261 | catgacttga gaatattctg cagctataaa ttttgaacca ttgatgtgca aagcaagacc |
| 1321 | tgaagcccac tccggaaact aaagtgaggc tcgctaaccc tctagattgc ctcacagttg |
| 1381 | LLLqtttaca aagtaaactt tacatccagg ggatgaagaq cacccaccag cagaagactt |
| 1441 | tgcagaacct ttaattggat gtgttaagtg tttttaatga gtgtatgaaa tgtagaaaga |
| 1501 | tqtacaagaa ataaattagg ggagattact ttgtattgta ctgccattcc tactqtattt |
| 1561 | ttatactttt tggcagcatt aaatattttt gttaaatagt caaaaaaaaa aaaaaaaaa |

SEQ ID NO: 6 Human PTPN2 isoform 3 Amino Acid Sequence (NP 536348.1)

| | |
|---|---|
| 1 | mpttierefe eldtqrrwqp lyleirnesh dyphrvakfp enrnrnryrd vspvdhsrvk |
| 61 | lqnaendyin aslvdieeaq rsviltqgpl pntcchfwim vwqqktkavv mlnrivekes |
| 121 | vkcagywptd dgemlfketg fsvklisedv ksyytvhllq ieninsgetr tishfhyttw |
| 181 | pdfgvpespa sflnflfkvr esgsinpdhq pavihcsagi grsgtfsivd tclvimekqd |
| 241 | dinikqvlln mrkyrmgliq tpdqlrfsym aiiegakcik gdssiqkrwk elskedispa |
| 301 | fdhspnkimt ekvngnrigi eeekltgdrc tglsskmqdt meenserprl tdt |

SEQ ID NO: 7 Human PTPN2 isoform 4 cDNA Sequence (NM 001207013.1)

| | |
|---|---|
| 1 | gctcgggcgc cgagtctgcg cgctgacgtc cgacgctcca ggtactttcc ccacggccga |
| 61 | cagggcttgg cgtgggggcg gggcgcggcg cgcagcgcgc atgcgccgca gcgccagcgc |
| 121 | tctccccgga tcgtgcgggg cctgagcctc tccgccggcg caggctctgc tcgcgccagc |
| 181 | tcgctcccgc agccatgccc accaccatcg agcgggagtt cgaagagttg gatactcagc |
| 241 | gtcgctggca gccgctgtac ttggaaattc gaaatgagtc ccatgactat cctcatagag |
| 301 | tggccaagtt tccagaaaac agaaatcgaa acagatacag agatgtaagc ccatatgatc |
| 361 | acagtcgtgt taaactgcaa aatgctgaga atgattatat taatgccagt ttagttgaca |
| 421 | tagaagaggc acaaaggagt tacatcttaa cacagggtcc acttcctaac acatgctgcc |
| 481 | atttctggct tatggtttgg cagcagaaga ccaaagcagt tgtcatgctg aaccgcattg |
| 541 | tggagaaaga atcggttaaa tgtgcacagt actggccaac agatgaccaa gagatgctgt |
| 601 | ttaaagaaac aggattcagt gtgaagctct tgtcagaaga tgtg.agtcg tattatacag |
| 661 | tacatctact acaattagaa aatatcaatt atattgagaa cttgtggatc acactgtatt |
| 721 | tgaaattatt aatgctggat gttaaaaggt cactaaaaag tggtgaaacc agaacaatat |
| 781 | ctcactttca ttatactacc tggccagatt ttggagtccc tgaatcacca gcttcatttc |
| 841 | tcaatttctt gtttaaagtg agagaatctg gctccttgaa ccctgaccat gggcctgcgg |
| 901 | tgatccactg tagtgcaggc attcmgcgct ctggcacctt ctctctggta gacacttgtc |
| 961 | ttgttttgat ggaaaaagga gatgatatta acataaaaca agtgttactg aacatgagaa |
| 1021 | aataccgaat gggtcttatt cagaccccag atcaactgag attctcatac atggctataa |
| 1081 | tagaaggagc aaaatgtata aagggagatt ctagtataca gaaacgatgg aaagaacttt |
| 1141 | ctaaggaaga cttatctcct gcctttgatc attcaccaaa caaaataatg actgaaaaat |
| 1201 | acaatgggaa cagaataggt ctagaagaag aaaaactgac aggtgaccga tgtacaggac |
| 1261 | tttcctctaa aatgcaagat acaatggagg agaacagtga gagtgctcta cggaaacgta |
| 1321 | ttcgagagga cagaaaggcc accacagctc agaaggtgca gcagatgaaa cagaggctaa |
| 1381 | atgagaatga acgaaaaaga aaaaggccaa gaLtgacaga cacctaatat tcatgacttg |
| 1441 | agaatattct gcagctataa attttgaacc attgatgtgc aaagcaagac ctgaagccca |
| 1501 | ctccggaaac taaagtgagg ctcgctaacc ctctagattg cctcacagtt gtttgtttac |
| 1561 | aaagtaaact ttacatccag gggatgaaga gcacccacca gcagaagact ttgcagaacc |
| 1621 | tttaattgga tgtgttaagt gtttttaatg agtgtatgaa atgtagaaag atgtacaaga |
| 1681 | aataaattag gggagattac tttgtattgt actgccattc ctactgtatt tttatacttt |
| 1741 | ttggcagcat taaatatttt tgttaaatag tcaaaaaaaa aaaaaaaaaa |

SEQ ID NO: NO: 8 Human PTPN2 isoform 4 Amino Acid Sequence
(NP 001193942.1)

| | |
|---|---|
| 1 | mpttierefe eldtgrrwqp lyleirnesh dyphrvakfp enrnrnryrd vspydhsrvk |
| 61 | lgnaendyin asivdieeaq rsyiltqgpl pntcchfwlm vwqqktkavv mlnrivekes |
| 121 | vkcaqywptd dqemlfketg fsvklisedv ksyytvhllq leninyienl witlyikllm |
| 181 | ldvkrslksg etrtishfhv ttwpdfgvpe spasflnflf kvresgslnp dhgpavihcs |
| 241 | agigrsgtfs lvdtclvlme kgddinikqv llnmrkyrmg liqtpdqlrf symaiiegak |
| 301 | cikgdssiqk rwkelskedl spafdhspnk imtekyngnr igleeekltg drctqlsskm |
| 361 | qdtmeenses alrkriredr kattaqhvgq mkqr1nener krkrprltdt |

SEQ ID NO: 9 Human PTPN2 isoform 5 cDNA Sequence (NM 001308287.1)

| | |
|---|---|
| 1 | tattcaatgc agggaacaga ccagttcatc atggaggcat tccatcagag cgtctagtta |
| 61 | gaccagatat gtcatggact gcatcggcac agaagtgggg Uttatgtgag agaggagttg |
| 121 | gaagtcacac ctgagtggag agcaacgtga aaaggtgatg tcagcaagaa tttaggatgt |
| 181 | atggaaagga tggtaaaggc accaactgga tggatcaggg agacatggaa tgcagaatgc |
| 241 | aggaaataga tgatcacagt cgtgttaaac tgcaaaatgc tgagaatgat tatattaatg |
| 301 | ccagtttagt tgacatagaa gaggcacaaa ggagttacat cttaacacag ggtccacttc |
| 361 | ctaacacatg ctgccatttc tggcttatgg tttggcagca gaagaccaaa gcagttgtca |
| 421 | tgctgaaccg cattgtggag aaagaatcgg ttaaatgtgc acagtactgg ccaacagatg |
| 481 | accaagagat gctgtttaaa gaaacaggat tcagtgtgaa gctcttgtca gaagatgtga |
| 541 | agtcgtatta tacagtacat ctactacaat tagaaaatat caatagtggt gaaaccagaa |
| 601 | caatatctca ctttcattat actacctggc cagattttgg agtccctgaa tcaccagctt |
| 661 | catttctcaa tttcttgttt aaagtgagag aatctggctc cttgaaccct gaccatgggc |
| 721 | ctgcggtgat ccactgtagt gcaggcattg ggcgctctgg caccttctct ctggtagaca |
| 781 | cttgtcttgt tttgatggaa aaaggagatg atattaacat aaaacaagtg ttactgaaca |
| 841 | tgagaaaata ccgaatgggt dttatLcaga ccccagatca adtgagattc tcatacatgg |
| 901 | ctataataga aggagcaaaa tgtataaagg gagattctag tatacagaaa cgatggaaag |
| 961 | aactttctaa ggaagactta tctcctgcct ttgatcattc accaaacaaa ataatgactg |
| 1021 | aaaaatacaa tgggaacaga ataggtctag aagaagaaaa actgacaggt gaccgatgta |
| 1081 | caggactttc ctetaaaatg caagatacaa tggaggagaa cagtgagagt gctctacgga |
| 1141 | aacgtattcg agaggacaga aaggcdacca cagctcagaa ggtgdagcag atgaaacaga |
| 1201 | ggctaaatga gaatgaacga aaaagaaaaa ggtggttata ttggcaacct attctcacta |
| 1261 | agatggggtt tatgtcagtd attttggttg gcgcttttgt tggctggaca ctgttttttc |
| 1321 | agcaaaatgc cctataaaca attaattttg cccagcaagc ttctgcacta gtaactgaca |
| 1381 | gtgdtacatt aatcataggg gtttgtctgc agcaaacgcc tcatatccca aaaacggtgc |
| 1441 | agtagaatag acatcaacca gataagtgat atttacagtc acaagcccaa catctcagga |
| 1501 | ctcttgactg caggttcctc tgaaccccaa actgtaaatg gctgtctaaa ataaagacat |
| 1561 | tcatgtttgt taaaaactgg taaattttgc aactgtattc atacatgtca aacacagtat |
| 1621 | ttcacctgac caacattgag atatccttta tcacaggatt tgtttttgga ggdtatctgg |
| 1681 | attttaacct gcacttgata taagcaataa atattgtggt tttatctacg ttattggaaa |
| 1741 | gaaaatgaca tttaaataat gtgtgtaatg tataatgtac tattgacatg ggcatcaaca |
| 1801 | cttttattct taagcatttc agggtaaata tattttataa gtatctattt aatcttttgt |
| 1861 | agttaactgt actttttaag agctcaattt gaaaaatctg ttactaaaaa aataaattgt |
| 1921 | atgtcgattg aattgtactg gatacatttt ccatttttct aaagagaagt ttgatatgag |
| 1981 | cagttagaag ttggaataag caatttctac tatatattgc atttctttta tgttttacag |
| 2041 | ttttccccat tttaaaaaga aaagcaaaca aagaaacaaa agtttttcct aaaaatatct |
| 2101 | ttgaaggaaa attctcctta ctgggatagt caggtaaaca gttggtcaag actttgtaaa |
| 2161 | gaaattggtt tctgtaaatc ccattattga tatgtttatt tttcatgaaa atttcaatgt |
| 2221 | agttggggta gattatgatt taggaagcaa aagtaagaag cagcatttta tgattcataa |
| 2281 | tttcagttta ctagactgaa gttttgaagt aaacactttt cagtttdttt ctacttcaat |
| 2341 | aaatagtatg attatatgca aaccttacat tgtcatttta acttaatgaa tattttttaa |
| 2401 | agcaaactgt ttaatgaatt taactgctca tttgaatgct agctttcctd agatttcaac |
| 2461 | attccattca gtgtttaatt tgtctLactt aaacttgaaa ttgttgttac aaatttaatt |
| 2521 | gctaggaggc atggatagda tacattatta tggatagcat accttaLLLc agtggttttc |
| 2581 | aaactatgct cattggatgt ccaggtgggt caagaggtta ctttcaacca cagcatctct |
| 2641 | gccttgtctc tttatatgcc acataagatt tctgcataag gcttaagtat tttaaagggg |
| 2701 | gcagttatca tttaaaaaca gtttggtcgg gcgcggtggc tcatgcctgt aatcccagca |
| 2761 | ctttgggagg ctgaagtggg cagatcacct gaggtcagga gttcaagacc agdctggcca |
| 2821 | acgtggtgaa acaccatctc tactaaaaat gcaaaaatta gctgggcatg gtggagggca |
| 2881 | cctgtaatct cagctactca ggaggctgag gtaggagaat tgcttgaacc caggagatgg |
| 2941 | aggttgcagt gagctgagat cacgtcactg cactccagcc agggcgacag agcgagactc |
| 3001 | catctcaaaa gaaacaaaca aaaaaaacag tttgggccgg gtgtggtggc tcacgcttgt |
| 3061 | aatcccagca cttcggaagg ccaaggcggg cggatcacga ggtcaagaga tggagactgt |
| 3121 | cctggccaac atggtgaaat cccttcttta ctaaaaatac aaaaatLaLc tgggcgtggt |
| 3181 | ggtgcatgcc tgtagtccca gctccttggg aggctaaggc aggagaatca cttgaacccg |
| 3241 | ggaggcagag gttgcagtga gccgagattg caccactgca ctccagcctg gcaacagagc |
| 3301 | aagacttcgt ctc |

SEQ ID NO: 10 Human PITN2 isoform 5 Amino Acid Sequence
(NP 001295216A)

| | |
|---|---|
| 1 | mvgkdgkgtn wmdqgdmecr mqeiddhsry klqnaendvi naslvdieea qrsviltqgp |
| 61 | lpntcchfwl mvwqqktkav vminriveke svkcaqywpt ddqemifket gfsvklised |
| 121 | vksyytvhll gleninsget rtishfhytt wpdfgvpesp asfinfifkv resgsinpdh |
| 181 | gpavihcsag igrsgtfsiv dtcivimekg ddinikqvll nmrkyrmgli qtpdqlrfsy |
| 241 | maiiegakci kgdssiqkrw keiskedisp afdhspnkim tekyngnrig leeekltgdr |
| 301 | ctglsskmqd tmeensesal rkriredrka ttaqkvqqmk qrinenerkr krwlywqpil |
| 361 | tkmgfmsvil vgafvgwtlf fqqnal |

SEQ ID NO: 11 Mouse PTPN2 isoform 1 eDNA Sequence (NM 001127177.1)

| | |
|---|---|
| 1 | ggcggggcgg ggcgcggagc gcgcatgcgc cacagtgcca gcgctctccc cggatagagc |
| 61 | ggggcccgag cctgtccgct gtggtagttc cgctcgcgct gccccgccgc catgtcggca |
| 121 | accatcgagc gggagttcga ggaactagat gctcagtgtc gctggcagcc gttatacttg |
| 181 | gaaattcgaa atgaatccca tgactatcct catagagtgg ccaagtttcc agaaaacaga |
| 241 | aaccgaaaca gatacagaga tgtaagccca tatgatcaca gtcgtgttaa actgcaaagt |
| 301 | actgaaaatg attatattaa tgccagctta gttgacatag aagaggcaca aagaagttac |
| 361 | atattaacac agggcccact tccgaacaca tgctgccatt tctggctcat ggtgtggcag |
| 421 | caaaagacca aagcagttgt catgctaaac cgaactgtag aaaaagaatc ggttaaatgt |
| 481 | gcacagtact ggccaacgga tgacagagaa atggtgttta aggaaacggg attcagtgtg |
| 541 | aagctcttat ctgaagatgt aaaatcatat tatacagtac atctactaca gttagaaaat |
| 601 | atcaatactg gtgaaaccag aaccatatct cacttccatt ataccacctg gccagatttt |
| 661 | ggggttccag agtcaccagc ttcatttcta aacttcttgt ttaaagttag agaatctggt |
| 721 | tgtttgaccc ctgaccatgg acctgcagtg atccattgca gtgcgggcat cgggcgctct |
| 781 | ggcaccttct ctcttgtaga tacctgtctt gttctgatgg aaaaaggaga ggatgttaat |
| 841 | gtgaaacaat tattactgaa tatgagaaag tatcgaatgg gacttattca gacaccggac |
| 901 | caactcagat tctcctacat ggccataata gaaggagcaa agtacacaaa aggagattca |
| 961 | aatatacaga aacggtggaa agaactttct aaagaagatt tatctcctat ttgtgatcat |
| 1021 | tcacagaaca gagtgatggt tgagaagtac aatgggaaga gaataggttc agaagatgaa |
| 1081 | aagttaacag ggcttccttc taaggtgcag gatactgtgg aggagagcag tgagagcatt |
| 1141 | ctacggaaac gtattcgaga ggatagaaag gctacgacgg ctcagaaggt gcagcagatg |
| 1201 | aaacagaggc taaatgaaac tgaacgaaaa agaaaaaggt ggttatattg gcaacctatt |
| 1261 | ctcactaaga tggggtttgt gtcagtcatt ttggttggcg ctttggttgg ctggacactg |
| 1321 | ctttttcact aaatgttcta taaattaata gttttaccca gcacctttct gcactagtag |
| 1381 | ctgaccgtgg tgcattaatc tcaagggttt gttagcaatg cctcatacgc agaaacactg |
| 1441 | cgctagagta gacatcagcc agataaggga tattacagtc acaagcccag catctcagga |
| 1501 | ctcatcactg caggttcctc tgagacccag actgtcaatg gctcacaata aagacaagca |
| 1561 | tgcttgttgg atactgttac ttcttacagc tgcgttcaca ccagtgtatt gagaaatcct |
| 1621 | ttatcccaag gattggcttt tggaggcctt ctggatttta acctgcactt gatataagca |
| 1681 | ataaacattg tggttttttt ctacattatt aatggaaaga aaatatcctt taaacaatgt |
| 1741 | atgtaatatg taatgtactg ttgaaatggg cattacaact ttatataacc attttagggt |
| 1801 | aaatatattt tataagtacc tatttaatct tacttttgta gttaaatgta ctttttaaag |
| 1861 | gttcaatctg aaagtctgtt atcatagaaa aataaattgt atgttgactc agttgtatac |
| 1921 | tgaatacatt ttccctttcc taagcagacg tttgatagag gcagttgaaa ctataagcaa |
| 1981 | gctaagacta ctacacattc ttatttcctt tctatttatg ctttatctta ttttaaaaag |
| 2041 | aaaaacaaaa attttctaaa catgtcattg aaggaaattg tttttttctg cgatagttaa |
| 2101 | gaagtgacag ttggtcaaaa tatagttgaa aacaaacaaa aacttggttt ctgcaggatg |
| 2161 | tggtagcaca cacagtgctc aggaagctaa aacaagaggc tcaatggttt gaagccagcc |
| 2221 | aaaactacat agcaaggtcc tatctttaaa gataagagaa aaatagaggt ggtggaggag |
| 2281 | agatcagaca acaccaagaa taagaaatcg attcttagcc atatttaatg gacaaacctg |
| 2341 | tcatctcagc ttttgggaga tagaggcaga aggctcacaa gttcaaggcc agcttcaact |
| 2401 | acatagctag ccccagagtt tggggccagt caggactgca agaaacactg tatcagaaac |
| 2461 | tgaagtggtt taaaaacatt ttgatttctg taaagtaaag cccatgcatg actacactgt |
| 2521 | taattttttg tgaaaatgta aatgtaatta cccagacggg ataaattatg gttagtaagt |
| 2581 | taaaggaacc agtgttttat acttttgatt tcagttcact agactgaaat tttgaagtaa |
| 2641 | aaaaaaattt aatttcttta caagttcaat aaatagtaca atggtgtaca aacttacatt |
| 2701 | gtcccttacc tttgtaatga gtatttttaa agcataacca ctaattgggt tttggtggtt |
| 2761 | tcaaaccctg cttggtggaa aggttccaaa ccattaggac agcattgctg cttcatctct |
| 2821 | tttatatatc acgtaaaagt gcgtggtaaa tcttaattag tttaaatgag acagttaatt |
| 2881 | tcttaatgca gtttgaaccc cataggtgta gttagaaatt gtgaatggcc ttgaaaagca |
| 2941 | tctcacaaag cgtatgatgt atgtgtgtgt cctgactcag catagctgtc ctaaggcttt |
| 3001 | gaaatggaga gcaggtaaga aggatgtttc ctcttgtctg tttaatctct gtttaagcgg |
| 3061 | aggccttaga attagatggc tatgggtttt gagctttcta acacttactg gtttgttttt |
| 3121 | ccaaaatgta gtatgttatc ctactagacc ttattaaaac ttacagtcca agccaataag |
| 3181 | gtggcgtaca cctttaatct caacactaag aacaccaaga cagacagatc cctgtgagtt |
| 3241 | caaggctagt ctggtccaca taataagttc ccaggcagcc agaaatagac attgagatcc |
| 3301 | tgtcttgaaa gaaagcaaac caactgaaga tagcctgagc ttaaacaact tcccacaaga |
| 3361 | aaaactgata aggctgagac cagtccttcc ttggacgata tgctttctag agatagcatt |
| 3421 | gagcaccact ctttctgcct cttggtgtgt attttatgtt tgtgaggatt cctttggcat |
| 3481 | acggaaccct cagtgctcct ccccggagcc cgtctttctc ccctgaacac atctttaagg |
| 3541 | atgagtttta acaggagaac ctttaagtca cactgtcatg ttgcttacta aaggtacatg |
| 3601 | gcctgtggtg acagtgtcac tggcatcatc ctgagcctgt atgagatgtg ctgtgctgat |
| 3661 | gagagaaggg tgctgggcag agaagggata ctagcagttt ctgatgggtt cacggcttta |
| 3721 | aacacagtgt gcgtcagtct cggtagcagc ttattttaac tdatttagga ataatagttt |
| 3781 | gtcttggatc aaattctgtt ttttgtttgt ttgtttgttt tttgtttttg gtgtttggtt |
| 3841 | tttttttaat ttggggaaaa aataggcttt ttaaagggga ttattgttta ctggaaagaa |
| 3901 | tcctcacttc ctgtttcctc ccaccttgct gtaatgtcag tggtcacaag attcaccagg |
| 3961 | tactgtgtta tctcagcctc ctgatttcta tccatgctca aacctaaagt gtaaaagtac |
| 4021 | acattccttt ttaaaaatac gcatatgcat catttctacg ttcagcagaa tctacacatt |
| 4081 | tgtcaagttt tccacagttc tcagttcttt ttatccattc cgttatgtgt cacctcatgt |
| 4141 | atcaaacagt gaacataaaa agatatgaag acctgtatta attagttttt gtccaaacag |
| 4201 | ctgtgctctg aagctgcgtc agaggaaagg tcctaatttc tgagctcagc ttccatgcac |
| 4261 | tcggctcggc cctttgtctt aaagtaaagc tagtgctgtg agtttagaac tgtggcccac |
| 4321 | gtttcaagtt atgacacaga acagccctct ctggttgtca tttcatttcc ttgtttgctt |
| 4381 | ttagcaccag tcccagggtg ctggctccca ttttctgcca ggcacagaaa ggctacagct |
| 4441 | gactgcttta aaaatagctc tgcgtagatt ctgcagagaa gctggaacct aatggtagta |
| 4501 | aaagtacttt tttttggcca ttgtatacaa tctacttaac aagtttacat ttctgtcaag |
| 4561 | acattgcaga ctgaagatct acattgcctt aatttgttac ttactgatac aaatctttat |
| 4621 | ttgtagttgt tgttttggat aggtttgtat attctttttt tttttttttt ttttttttgt |
| 4681 | atgtgtgttg agatagtacc ttgccattgc ccaagcctgg ccttaaactc agctcaaacg |
| 4741 | actttcctac ctcagcctgt tgagtaacta acaccacagg tacacactgt gcacacagct |
| 4801 | ttcaagtata aatcttaaag agattatttt aaaactgtag ataagatttc aggcccttag |
| 4861 | tcaagcgtgg tgcatacctt ctctgagtag ggccatctct gggtcctggt gagtagtgtc |
| 4921 | tatgtctgtg ggaaggaagg gctgctcggg gccttcatct ggctgagctc gattcatctg |
| 4981 | ttcatagcat gggacaaaat accaacagaa atgtccattc tatttacatg ccaacaccta |
| 5041 | acaaagtctc ttatttttaa aactccttta tatggctttg ccatagddtc ttgtatatac |
| 5101 | tttttttttt ttttcaaaat agaaatgatt ttttttctca ttaaatttgt catcttatta |
| 5161 | cttgaaacgt gggcctttgt tattggcagt ggcttgctcc cgaggaggcc tgttctgtcc |
| 5221 | accctgtccc agaacgcact catttgagtc agatgccaca gttcttcctc acactggtct |
| 5281 | ttggtttata ccatgcagca ccatacctag agtcacagct gtctctaatt gtcccctgaa |
| 5341 | tatggdatga gagactcagg ctgtgccctc attcactgct gctctgcact ggagcctgtc |
| 5401 | cccaatcaga gaacttgcct cgtggccagc agtcttcctt cctgggtcct gagcagcttc |
| 5461 | aagccttctg cattagtgct ttctcttagc cgtggctgtt gggaagaaga cccactgttc |
| 5521 | tccacaggtt gggttgtttt tttttttttt cctggctgtc cttgtcccag cacagtgcca |
| 5581 | tcagccattg tgagcagtgc ttaaagtgga aagctacacc agcctaagag getttgtgta |
| 5641 | agctgacgtt taggatttaa agagcctgga ccatctgagt tctgactctg aagctctgct |
| 5701 | tggttgtaaa gttccagttg attctgagca gtgaggtgtg aggccactgt caccggtagg |
| 5761 | gtctgcttgg atgccgcctg ctttacttgg atctgttttg ttggggactg ctgcaaggag |
| 5821 | aattgcatgg gaattttctt ctttttcttt acagagactt ataagcatcg agttattctt |
| 5881 | tgtagtcact cattaggcat agtttttttt ttttaagacc catgatgctg ttgctattcc |
| 5941 | cccccccctt ttttttttgg ttttttgaga cagggtttct ctgtataact ctggctgtcc |
| 6001 | tggaactcac tttgtagacc tcaaactcag aaatccccct gcctttgcct cctgagtgct |
| 6061 | gggattaaag gcatgcgcca ccacggcccg gctgctgttg atattttaaa tgactatttt |
| 6121 | aaaaagtcgt tcagtgtgga aagttgagga gaggaagcct aggtaagttc ctttaaagca |
| 6181 | tgcttggctc acctcggtta gtcctgatca atctcagtcg gatgctaatg taaatgtcgt |
| 6241 | gtggcaaaac aacttttaat gcagtctgac tttccctcta acacgggcaa ggaagaagac |
| 6301 | accagcattt gcctctgcag cacagaggca gcccccagga tacccacgta gctcattgct |
| 6361 | tggtttgctc gcccatttta cttttgcctt attaaaaata aaatggtgaa gatccattca |
| 6421 | agtgaatata atagaattat ctcaaaagcc atttatctta atagtcttac aaataaagtc |
| 6481 | atttcttaga agctattcca ttgatttcct cttattttgc tacccctaaa cactatttga |
| 6541 | aaagaagtaa tgagtttcaa aaaccacagc gtgtctgtta aatggcaaat ttattattct |
| 6601 | tggtaaatgt gtatttaaca aacactagga aaggatatct cgtgtgtatg tgagagagaa |
| 6661 | agagagagtg cttcacaaca ctttaaataa tgccagccat attttcagat aagaaaccca |
| 6721 | gtggaggtgt gactcacgcc ttattttcca gcctgtgcag atagagctga gatgcagact |
| 6781 | ccaggctgtg gtttcagtcc ctccaaggct caggctcatt gtgctactcc actgtgtatt |
| 6841 | tacttaaacc agatgtttaa gcggggaaat agtagacacc ccactagtgg aggggtggaa |
| 6901 | tcccttttac aatgcttcac tgactatggc ggaccagaac gtttctgtgc caaagcccca |
| 6961 | cttcattcct ttctgttctg ttccacattc tgccagagtc agaaccagcc gtttggtccc |
| 7021 | aggtcctgcg acccattgct atctaaagag tatggttccc taatgagaac actgcagaga |
| 7081 | atcactgttg ggaaatcaaa caagactttg tagaccacca caggggcttg gtagatctgc |
| 7141 | ctgcctatgg agaaagaagc cagtagacag gaagaagctt cattctcatg gttggggagg |
| 7201 | agcctaagtg gtggagatct agtgtattgc ctgtttatac agtgataaag tcaagtattt |
| 7261 | tcatgggtag agagcgaggg tggaggaagg gaggggctgc gatcggtgca aaaatggaaa |
| 7321 | tagctttaat ctcccaaaag ctttgaccac tggcaaacaa ttgaaatatc agcaaagact |
| 7381 | actgctctta atggtcacac cctcttgttt aaatggcgtc cccctcccaa gcattaaatt |
| 7441 | gcgctgaact atcacagttt tacttagttc tagtagttat aatcattagc attctccttc |
| 7501 | aggagaaaat ctaaatgctg gaaatctaat tcagagataa caagccaact ttatgtgcaa |
| 7561 | actttatatt taaactgttt ctagcagtgt tacagtgatt gtccaaactg gattagactt |
| 7621 | ttgcgttgaa atcaaagtat gggtaagtct agcacatgta ataaaacctt gctgtttctt |
| 7681 | gtggctacat tttttttttt aacttgtctg tctcttagcc taccatgtag aggtcatttc |
| 7741 | ttgagttaag atgggatggc ctaaaagatt cagtgtgtag ttactgaaga agtaagtccc |
| 7801 | ggcgcctcag agcagtctgt ctcacagccc cgcttccatt tggaaacctg ccattctgga |
| 7861 | aggaagcact tggtgttctt ggaatgttca tgttggaatg atttttgttg ttgttgttgt |
| 7921 | tgttgacttt ttagttgagt cttagttctt ttgtgtttgt atctatctat gtacatctgt |
| 7981 | gtgtgtggtg gccatggatt gaatagatga cttcttattt tatgttttag gccaagattg |
| 6041 | acagacacct aaatgttcat gacttgagac tattctgcag ctataaaatt tgaacctttg |
| 6101 | atgtgcaaag caagacctga agcccactcc ggaaactaaa gtgaggcttg ctaaccctgt |
| 8161 | agattgcctc acaagttgtc tgtttacaaa gtaagctttc catccagggg atgaagaacg |
| 8221 | ccaccagcag aagacttgca aaccctttaa tttgatgtat tgttttttaa catgtgtatg |
| 8281 | aaatgtagaa agatgtaaag gaaataaatt aggagcgact actttgtatt gtactgccat |
| 8341 | tcctaatgta tttttatact ttttggcagc attaaatatt tttattaaat agactatgtt |
| 8401 | ggttaaaaaa aaaaaaaaaa aaa |

SEQ ID NO: 12 Mouse PTPN2 isoform 1 Amino Acid Sequence
(NP 001120649.1)

| | |
|---|---|
| 1 | msatierefe eldaqcrwqp lyleirnesh dyphrvakfp enrnrnrytd vspydhsrvk |
| 61 | lgstendyin asivdieeaq rsyiltqgpl pntcchfwim vwqqktkavv mlnrtvekes |
| 121 | vkcaqywptd dremvfketg fsvkllsedv ksyytvhilq ienintgetr tishfhyttw |
| 181 | pdfgvpespa sflnfifkvr esgcltpdhg pavihcsagi grsgtfslvd tclvlmekge |
| 241 | dvnvkqllln mrkyrmgliq tpdqlrfsym aiiegakytk gdsndqkrwk elskedlspi |
| 301 | cdhsqnrvmv ekyngkrigs edekitglps kvqdtveess esilrkrire drkattaqkv |
| 361 | qqmkqrlnet erkrkrwlyw qpiltkmgfv svilvgalvg wtllfh |

SEQ ID NO: 13 Mouse PTPN2 isoform 2 cDNA Sequence (NM 008977.3)

| | |
|---|---|
| 1 | ggcggggcgg ggcgcggagc gcgcatgcgc cacagtgcca gcgctctccc cggatagagc |
| 61 | ggggcccgag cctgtccgct gtggtagttc cgctcgcgct gccccgccgc catgtcggca |
| 121 | accatcgagc gggagttcga ggaactggat gctcagtgtc gctggcagcc gttatacttg |
| 181 | gaaattcgaa atgaatccca tgactatcct catagagtgg ccaagtttcc agaaaacaga |
| 241 | aaccgaaaca gatacagaga tgtaagccca tatgatcaca gtcgtgttaa actgcaaagt |
| 301 | actgaaaatg attatattaa tgccagctta gttgacatag aagaggcaca aagaagttac |
| 361 | atcttaacac agggcccact tccgaacaca tgctgccatt tctggctcat ggtgtggcag |
| 421 | caaaagacca aagcagttgt catgctaaac cgaactgLag aaaaagaatc ggttaaatgt |
| 481 | gcacagtact ggccaacgga tgacagagaa atggtgttta aggaaacggg attcagtgtg |
| 541 | aagctcttat ctgaagatgt aaaatcatat tatacagtac atctactaca gttagaaaat |
| 601 | atcaatactg gtgaaaccag aaccatatct cacttccatt ataccacctg gccagatttt |
| 661 | ggggttccag agtcaccagc ttcatttcta aacttcttgt ttaaagttag agaatctggt |
| 721 | tgtttgaccc ctgaccatgg acctgcagtg atccattgca gtgcgggcat cgggcgctct |
| 781 | ggcaccttct ctcttgtaga tacctgLett gtLctgatgg aaaaaggaga ggatgttaat |
| 841 | gtgaaacaat tattactgaa tatgagaaag tatcgaatgg gacttattca gacaccggac |
| 901 | caactcagat tctcctacat ggccataata gaaggagcaa agtacacaaa aggagattca |
| 961 | aatatacaga aacggtggaa agaactttct aaagaagatt tatctcctat ttgtgatcat |
| 1021 | tcacagaaca gagtgatggt tgagaagtac aatgggaaga gaataggttc agaagatgaa |
| 1081 | aagttaacag ggcttccttc taaggtgcag gatactgtgg aggagagcag tgagagcatt |
| 1141 | ctacggaaac gtattcgaga ggatagaaag gctacgacgg ctcagaaggt gcagcagatg |
| 1201 | aaacagaggc taaatgaaac tgaacgaaaa agaaaaaggc caagattgsc agacacctaa |
| 1261 | aLgttcatga cttgagacta ttctgcagct ataaaatttg aacctttgat gtgcaaagca |
| 1321 | agacctgaag cccactccgg aaactaaagt gaggcttgct aaccctgtag attgcctcac |
| 1381 | aagttgtctg tttacaaagt aagctttcca tccaggggat gaagaacgcc accagcagaa |
| 1441 | gacttgcaaa ccctttaatt tgatgtattg ttttttaaca tgtgtatgaa atgtagaaag |
| 1501 | atgtaaagga aataaattag gagcgactac tttgtattgt actgccattc ctaatgtatt |
| 1561 | tttatacftt ttagcagcat taaatatttt tattaaatag actatgttgg ttaaaaaaaa |
| 1621 | aaaaaaaaaa a |

SEQ ID NO: 14 Mouse PTPN2 isoform 2 Amino Acid Sequence (NP 033003.1)

| | |
|---|---|
| 1 | msatierefe eldegcrwqp lyleirnesh dyphrvakfp enrnrnryrd vspydhsrvk |
| 61 | lqstendyin aslvdieeaq rsyiltqgpl pntcchfwlm vwqqktkavv mlnrtvekes |
| 121 | vkcagywptd dremvfketg fsvklisedv ksyytvhllq lenintgetr tishfhyttw |
| 181 | pdfgvpespa sflnflfkvr esgcltpdhg pavihcsagi grsgtfslvd tclvlmekge |
| 241 | dvnvkqllln mrkyrmgliq tpdqlrfsym aiiegakytk gdsniqkrwk eiskedispi |
| 301 | cdhsgnrvmv ekyngkrigs edekltglps kvqdtveess esilrkrire drkattaqkv |
| 361 | qqmkgrlnet erkrkrprlt dt |

Included in Table 1 are RNA nucleic acid molecules (e.g., thymines replaced with uridines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 1, or a portion thereof. Such nucleic acid molecules can have a function of the full-length nucleic acid as described further herein.
Included in Table 1 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 1, or a portion thereof. Such polypeptides can have a function of the full-length polypeptide as described further herein.
Included in Table 1 are other known PTPN2 nucleic acid and amino acid sequences.

### II. Agents that Upregulate Immune Responses

It is demonstrated herein that PTPN2 is a negative regulator of immune responses, such that modulating the copy number, the expression, and/or the activity of PTPN2 can modulate immune responses. Thus, the agents encompassed by the present invention described herein are PTPN2 pathway inhibitors (e.g., inhibitor of the copy number, the expression, and/or the activity of PTPN2) that can upregulate the immune responses and, thereby treating a subject with a condition that would benefit from increased immune responses. Agents that inhibit the copy number, the expression, and/or the activity of PTPN2 can do so either directly or indirectly.

Agents useful in the methods encompassed by the present invention include antibodies, small molecules, peptides, peptidomimetics, natural ligands, and derivatives of natural ligands, that can bind and/or inhibit PTPN2, or fragments thereof; RNA interference, antisense, nucleic acid aptamers, etc. that can downregulate the expression and/or activity of PTPN2, or fragments thereof.

In one embodiment, isolated nucleic acid molecules that specifically hybridize with or encode PTPN2 or biologically active portions thereof. As used herein, the term “nucleic acid molecule” is intended to include DNA molecules (i.e., cDNA or genomic DNA) and RNA molecules (i.e., mRNA) and analogs of the DNA or RNA generated using nucleotide analogs. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecules corresponding to PTPN2 can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (i.e., a lymphoma cell). Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule encompassed by the present invention, e.g., a nucleic acid molecule having the nucleotide sequence of PTPN2 listed in Table 1 or a nucleotide sequence which is at least about 50%, preferably at least about 60%, more preferably at least about 70%, yet more preferably at least about 80%, still more preferably at least about 90%, and most preferably at least about 95% or more (e.g., about 98%) homologous to the nucleotide sequence of PTPN2 listed in Table 1 or a portion thereof (i.e., 100, 200, 300, 400, 450, 500, or more nucleotides), can be isolated using standard molecular biology techniques and the sequence information provided herein. For example, a human cDNA can be isolated from a human cell line (from Stratagene, LaJolla, Calif., or Clontech, Palo Alto, Calif.) using all or portion of the nucleic acid molecule, or fragment thereof, as a hybridization probe and standard hybridization techniques (i.e., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of the nucleotide sequence of PTPN2 listed in Table 1 or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence, or fragment thereof, can be isolated by the polymerase chain reaction using oligonucleotide primers designed based upon PTPN2 sequence listed in Table 1, or fragment thereof, or the homologous nucleotide sequence. For example, mRNA can be isolated from muscle cells (i.e., by the guanidinium-thiocyanate extraction procedure of Chirgwin et al. (1979) Biochemistry 18: 5294-5299) and cDNA can be prepared using reverse transcriptase (i.e., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for PCR amplification can be designed according to well-known methods in the art. A nucleic acid encompassed by the present invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to the nucleotide sequence of PTPN2 listed in Table 1 can be prepared by standard synthetic techniques, i.e., using an automated DNA synthesizer.

Probes based on the nucleotide sequences of PTPN2 listed in Table 1 can be used to detect or confirm the desired transcripts or genomic sequences encoding the same or homologous proteins. In preferred embodiments, the probe further comprises a label group attached thereto, i.e., the label group can be a radioisotope, a fluorescent compound, an enzyme, or an enzyme co-factor. Such probes can be used as a part of a diagnostic test kit for identifying cells or tissue which express PTPN2, such as by measuring a level of PTPN2 nucleic acid in a sample of cells from a subject, i.e., detecting mRNA levels of PTPN2.

Nucleic acid molecules encoding proteins corresponding to PTPN2 from different species are also contemplated. For example, rat or monkey cDNA can be identified based on the nucleotide sequence of a human and/or mouse sequence and such sequences are well-known in the art. In one embodiment, the nucleic acid molecule(s) encompassed by the present invention encodes a protein or portion thereof which includes an amino acid sequence which is sufficiently homologous to an amino acid sequence of PTPN2 listed in Table 1, such that the protein or portion thereof modulates (e.g., enhance), one or more of the following biological activities: a) binding to the biomarker; b) modulating the copy number of the biomarker; c) modulating the expression level of the biomarker; and d) modulating the activity level of the biomarker.

As used herein, the language “sufficiently homologous” refers to proteins or portions thereof which have amino acid sequences which include a minimum number of identical or equivalent (e.g., an amino acid residue which has a similar side chain as an amino acid residue in PTPN2 listed in Table 1, or fragment thereof) amino acid residues to an amino acid sequence of the biomarker, or fragment thereof, such that the protein or portion thereof modulates (e.g., enhance) one or more of the following biological activities: a) binding to the biomarker; b) modulating the copy number of the biomarker; c) modulating the expression level of the biomarker; and d) modulating the activity level of the biomarker.

In another embodiment, the protein is at least about 50%, preferably at least about 60%, more preferably at least about 70%, 75%, 80%, 85%, 90%, 91%, 92%, 93%, 94%, 95%, 96%, 97%, 98%, 99% or more homologous to the entire amino acid sequence of the biomarker, or a fragment thereof.

Portions of proteins encoded by nucleic acid molecules of PTPN2 listed in Table 1 are preferably biologically active portions of the protein. As used herein, the term “biologically active portion” of PTPN2 is intended to include a portion, e.g., a domain/motif, that has one or more of the biological activities of the full-length protein.

Standard binding assays, e.g., immunoprecipitations and yeast two-hybrid assays, as described herein, or functional assays, e.g., RNAi or overexpression experiments, can be performed to determine the ability of the protein or a biologically active fragment thereof to maintain a biological activity of the full-length protein.

The invention further encompasses nucleic acid molecules that differ from the nucleotide sequence of PTPN2 listed in Table 1, or fragment thereof due to degeneracy of the genetic code and thus encode the same protein as that encoded by the nucleotide sequence, or fragment thereof. In another embodiment, an isolated nucleic acid molecule encompassed by the present invention has a nucleotide sequence encoding a protein having an amino acid sequence of PTPN2 listed in Table 1, or fragment thereof, or a protein having an amino acid sequence which is at least about 70%, 75%, 80%, 85%, 90%, 91%, 92%, 93%, 94%, 95%, 96%, 97%, 98%, 99% or more homologous to the amino acid sequence of PTPN2 listed in Table 1, or fragment thereof. In another embodiment, a nucleic acid encoding a polypeptide consists of nucleic acid sequence encoding a portion of a full-length fragment of interest that is less than 195, 190, 185, 180, 175, 170, 165, 160, 155, 150, 145, 140, 135, 130, 125, 120, 115, 110, 105, 100, 95, 90, 85, 80, 75, or 70 amino acids in length.

It will be appreciated by those skilled in the art that DNA sequence polymorphisms that lead to changes in the amino acid sequences of PTPN2 listed in Table 1 may exist within a population (e.g., a mammalian and/or human population). Such genetic polymorphisms may exist among individuals within a population due to natural allelic variation. As used herein, the terms “gene” and “recombinant gene” refer to nucleic acid molecules comprising an open reading frame encoding PTPN2, preferably a mammalian, e.g., human, protein. Such natural allelic variations can typically result in 1-5% variance in the nucleotide sequence of PTPN2 listed in Table 1. Any and all such nucleotide variations and resulting amino acid polymorphisms in PTPN2 that are the result of natural allelic variation and that do not alter the functional activity of PTPN2 are intended to be within the scope encompassed by the present invention. Moreover, nucleic acid molecules encoding PTPN2 proteins from other species.

In addition to naturally-occurring allelic variants of PTPN2 sequence listed in Table 1 that may exist in the population, the skilled artisan will further appreciate that changes can be introduced by mutation into the nucleotide sequence, or fragment thereof, thereby leading to changes in the amino acid sequence of the encoded PTPN2, without altering the functional ability of PTPN2. For example, nucleotide substitutions leading to amino acid substitutions at “non-essential” amino acid residues can be made in the sequence, or fragment thereof. A “non-essential” amino acid residue is a residue that can be altered from the wild-type sequence of PTPN2 without altering the activity of PTPN2, whereas an “essential” amino acid residue is required for the activity of PTPN2. Other amino acid residues, however, (e.g., those that are not conserved or only semi-conserved between mouse and human) may not be essential for activity and thus are likely to be amenable to alteration without altering the activity of PTPN2.

The term “sequence identity or homology” refers to the sequence similarity between two polypeptide molecules or between two nucleic acid molecules. When a position in both of the two compared sequences is occupied by the same base or amino acid monomer subunit, e.g., if a position in each of two DNA molecules is occupied by adenine, then the molecules are homologous or sequence identical at that position. The percent of homology or sequence identity between two sequences is a function of the number of matching or homologous identical positions shared by the two sequences divided by the number of positions compared×100. For example, if 6 of 10, of the positions in two sequences are the same then the two sequences are 60% homologous or have 60% sequence identity. By way of example, the DNA sequences ATTGCC and TATGGC share 50% homology or sequence identity. Generally, a comparison is made when two sequences are aligned to give maximum homology. Unless otherwise specified “loop out regions”, e.g., those arising from, from deletions or insertions in one of the sequences are counted as mismatches.

The comparison of sequences and determination of percent homology between two sequences can be accomplished using a mathematical algorithm. Preferably, the alignment can be performed using the Clustal Method. Multiple alignment parameters include GAP Penalty=10, Gap Length Penalty=10. For DNA alignments, the pairwise alignment parameters can be Htuple=2, Gap penalty=5, Window=4, and Diagonal saved=4. For protein alignments, the pairwise alignment parameters can be Ktuple=1, Gap penalty=3, Window=5, and Diagonals Saved=5.

In a preferred embodiment, the percent identity between two amino acid sequences is determined using the Needleman and Wunsch (J. Mol. Biol. (48):444-453 (1970)) algorithm which has been incorporated into the GAP program in the GCG software package (available online), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available online), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0) (available online), using a PAM120 weight residue table, a gap length penalty of 12 and a gap penalty of 4.

An isolated nucleic acid molecule encoding a protein homologous to PTPN2, or fragment thereof, can be created by introducing one or more nucleotide substitutions, additions or deletions into the nucleotide sequence, or fragment thereof, or a homologous nucleotide sequence such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), branched side chains (e.g., threonine, valine, isoleucine) and aromatic side chains (e.g., tyrosine, phenylalanine, tryptophan, histidine). Thus, a predicted nonessential amino acid residue in PTPN2 is preferably replaced with another amino acid residue from the same side chain family. Alternatively, in another embodiment, mutations can be introduced randomly along all or part of the coding sequence of PTPN2, such as by saturation mutagenesis, and the resultant mutants can be screened for an activity described herein to identify mutants that retain desired activity. Following mutagenesis, the encoded protein can be expressed recombinantly according to well-known methods in the art and the activity of the protein can be determined using, for example, assays described herein.

The levels of PTPN2 levels may be assessed by any of a wide variety of well-known methods for detecting expression of a transcribed molecule or protein. Non-limiting examples of such methods include immunological methods for detection of proteins, protein purification methods, protein function or activity assays, nucleic acid hybridization methods, nucleic acid reverse transcription methods, and nucleic acid amplification methods.

In preferred embodiments, the levels of PTPN2 levels are ascertained by measuring gene transcript (e.g., mRNA), by a measure of the quantity of translated protein, or by a measure of gene product activity. Expression levels can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In a particular embodiment, the mRNA expression level can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Aolecular Biology, John Wiley & Sons, New York 1987-1999). Additionally, large numbers of tissue samples can readily be processed using techniques well-known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding PTPN2. Other suitable probes for use in the diagnostic assays encompassed by the present invention are described herein. Hybridization of an mRNA with the probe indicates that PTPN2 is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in a gene chip array, e.g., an Affymetrix™ gene chip array. A skilled artisan can readily adapt known mRNA detection methods for use in detecting the level of PTPN2 mRNA expression levels.

An alternative method for determining mRNA expression level in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well-known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to PTPN2 mRNA.

As an alternative to making determinations based on the absolute expression level, determinations may be based on the normalized expression level of PTPN2. Expression levels are normalized by correcting the absolute expression level by comparing its expression to the expression of a non-biomarker gene, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a normal sample, or between samples from different sources.

The level or activity of a protein corresponding to PTPN2 can also be detected and/or quantified by detecting or quantifying the expressed polypeptide. The polypeptide can be detected and quantified by any of a number of means well-known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like. A skilled artisan can readily adapt known protein/antibody detection methods for use in determining whether cells express the biomarker of interest.

The present invention further provides soluble, purified and/or isolated polypeptide forms of PTPN2, or fragments thereof. In addition, it is to be understood that any and all attributes of the polypeptides described herein, such as percentage identities, polypeptide lengths, polypeptide fragments, biological activities, antibodies, etc. can be combined in any order or combination with respect to PTPN2.

In one aspect, a polypeptide may comprise a full-length amino acid sequence corresponding to PTPN2 or a full-length amino acid sequence with 1 to about 20 conservative amino acid substitutions. An amino acid sequence of any described herein can also be at least 50, 55, 60, 65, 70, 75, 80, 85, 90, 91, 92, 93, 94, 95, 96, 97, 98, 99, or 99.5% identical to the full-length sequence of PTPN2, which is either described herein, well-known in the art, or a fragment thereof. In another aspect, the present invention contemplates a composition comprising an isolated polypeptide corresponding to PTPN2 polypeptide and less than about 25%, or alternatively 15%, or alternatively 5%, contaminating biological macromolecules or polypeptides.

The present invention further provides compositions related to producing, detecting, or characterizing such polypeptides, or fragment thereof, such as nucleic acids, vectors, host cells, and the like. Such compositions may serve as compounds that modulate the expression and/or activity of PTPN2.

An isolated polypeptide or a fragment thereof (or a nucleic acid encoding such a polypeptide) corresponding to PTPN2, including the ones listed in Table 1 or fragments thereof, can be used as an immunogen to generate antibodies that bind to said immunogen, using standard techniques for polyclonal and monoclonal antibody preparation according to well-known methods in the art. An antigenic peptide comprises at least 8 amino acid residues and encompasses an epitope present in the respective full length molecule such that an antibody raised against the peptide forms a specific immune complex with the respective full length molecule. Preferably, the antigenic peptide comprises at least 10 amino acid residues. In one embodiment such epitopes can be specific for a given polypeptide molecule from one species, such as mouse or human (i.e., an antigenic peptide that spans a region of the polypeptide molecule that is not conserved across species is used as immunogen; such non conserved residues can be determined using an alignment such as that provided herein).

In one embodiment, an antibody, especially an intrabody, binds substantially specifically to PTPN2 (e.g., one or more kinase signaling inhibitors, such as PTPN2) and inhibits or blocks its biological function, such as by interrupting its interaction with a substrate like STAT or JAK proteins. In another embodiment, an antibody, especially an intrabody, binds substantially specifically to a binding partner of PTPN2, such as PTPN2 substrates described herein, and inhibits or blocks its biological function, such as by interrupting its interaction to PTPN2.

Antibodies for use according to the present invention can be generated according to well-known methods in the art. For example, a polypeptide immunogen typically is used to prepare antibodies by immunizing a suitable subject (e.g., rabbit, goat, mouse or other mammal) with the immunogen. An appropriate immunogenic preparation can contain, for example, a recombinantly expressed or chemically synthesized molecule or fragment thereof to which the immune response is to be generated. The preparation can further include an adjuvant, such as Freund's complete or incomplete adjuvant, or similar immunostimulatory agent. Immunization of a suitable subject with an immunogenic preparation induces a polyclonal antibody response to the antigenic peptide contained therein.

Polyclonal antibodies can be prepared as described above by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography, to obtain the IgG fraction. At an appropriate time after immunization, e.g., when the antibody titers are highest, antibody-producing cells can be obtained from the subject and used to prepare monoclonal antibodies by standard techniques, such as the hybridoma technique (originally described by Kohler and Milstein (1975) Nature 256:495-497) (see also Brown et al. (1981) J. Immunol. 127:539-46; Brown et al. (1980)J. Biol. Chem. 255:4980-83; Yeh et al. (1976) Proc. Natl. Acad. Sci. 76:2927-31; Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well-known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

Any of the many well-known protocols used for fusing lymphocytes and immortalized cell lines can be applied for the purpose of generating a monoclonal antibody against PTPN2, or a fragment thereof (see, e.g., Galfre, G. et al. (1977) Nature 266:55052; Gefter et al. (1977) supra; Lerner (1981) supra; Kenneth (1980) supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation encompassed by the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O—Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody encompassed by the present invention are detected by screening the hybridoma culture supernatants for antibodies that bind a given polypeptide, e.g., using a standard ELISA assay.

As an alternative to preparing monoclonal antibody-secreting hybridomas, a monoclonal specific for one of the above described polypeptides can be identified and isolated by screening a recombinant combinatorial immunoglobulin library (e.g., an antibody phage display library) with the appropriate polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene Sur/ZAP™ Phage Display Kit, Catalog No. 240612). Additionally, examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Since it is well-known in the art that antibody heavy and light chain CDR3 domains play a particularly important role in the binding specificity/affinity of an antibody for an antigen, the recombinant monoclonal antibodies encompassed by the present invention prepared as set forth above preferably comprise the heavy and light chain CDR3s of variable regions of antibodies of interest. The antibodies further can comprise the CDR2s of variable regions encompassed by the present invention. The antibodies further can comprise the CDR1s of variable regions encompassed by the present invention. In other embodiments, the antibodies can comprise any combinations of the CDRs.

The CDR1, 2, and/or 3 regions of the engineered antibodies described above can comprise the exact amino acid sequence(s) as those of variable regions encompassed by the present invention. However, the ordinarily skilled artisan will appreciate that some deviation from the exact CDR sequences may be possible while still retaining the ability of the antibody to bind a target of interest, such as PTPN2 and/or one or more natural binding partners effectively (e.g., conservative sequence modifications). Accordingly, in another embodiment, the engineered antibody may be composed of one or more CDRs that are, for example, 50%, 60% 0, 70%, 80%, 85%, 90%, 91%, 92%, 93%, 94%, 95%, 96%, 97%, 98%, 99%, or 99.5% identical to one or more CDRs encompassed by the present invention.

For example, the structural features of non-human or human antibodies (e.g., a rat anti-mouse/anti-human antibody) can be used to create structurally related human antibodies, especially intrabodies, that retain at least one functional property of the antibodies encompassed by the present invention, such as binding to PTPN2, PTPN2 binding partners/substrates, and/or an immune checkpoint. Another functional property includes inhibiting binding of the original known, non-human or human antibodies in a competition ELISA assay.

A skilled artisan will note that such percentage homology is equivalent to and can be achieved by introducing 1, 2, 3, 4, 5, 6, 7, 8, 9, 10, or more conservative amino acid substitutions within a given CDR.

The monoclonal antibodies encompassed by the present invention can comprise a heavy chain, wherein the variable domain comprises at least a CDR having a sequence selected from the group consisting of the heavy chain variable domain CDRs described herein, and a light chain, wherein the variable domain comprises at least a CDR having a sequence selected from the group consisting of the light chain variable domain CDRs described herein.

Such monoclonal antibodies can comprise a light chain, wherein the variable domain comprises at least a CDR having a sequence selected from the group consisting of CDR-L1, CDR-L2, and CDR-L3, as described herein; and/or a heavy chain, wherein the variable domain comprises at least a CDR having a sequence selected from the group consisting of CDR-H1, CDR-H2, and CDR-H3, as described herein. In some embodiments, the monoclonal antibodies capable of binding PTPN2, comprises or consists of CDR-L1, CDR-L2, CDR-L3, CDR-H1, CDR-H2, and CDR-H3, as described herein.

The heavy chain variable domain of the monoclonal antibodies encompassed by the present invention can comprise or consist of the vH amino acid sequence set forth herein and/or the light chain variable domain of the monoclonal antibodies encompassed by the present invention can comprise or consist of the vK amino acid sequence set forth herein.

The present invention further provides fragments of said monoclonal antibodies which include, but are not limited to, Fv, Fab, F(ab′)2, Fab′, dsFv, scFv, sc(Fv)2 and diabodies; and multispecific antibodies formed from antibody fragments. For example, a number of immunoinhibitory molecules, such as PTPN2, PD-L1, PD-1, CTLA-4, and the like, can be bound in a bispecific or multispecific manner.

Other fragments of the monoclonal antibodies encompassed by the present invention are also contemplated. For example, individual immunoglobulin heavy and/or light chains are provided, wherein the variable domains thereof comprise at least a CDR described herein. In one embodiment, the immunoglobulin heavy chain comprises at least a CDR having a sequence that is at least 80%, 85%, 90%, 91%, 92%, 93%, 94%, 95%, 96%, 97%, 98%, 99%, 99.5% or 100% identical from the group of heavy chain or light chain variable domain CDRs described herein. In another embodiment, an immunoglobulin light chain comprises at least a CDR having a sequence that is at least 80%, 85%, 90%, 91%, 92%, 93%, 94%, 95%, 96%, 97%, 98%, 99%, 99.5% or 100% identical from the group of light chain or heavy chain variable domain CDRs described herein, are also provided.

In some embodiments, the immunoglobulin heavy and/or light chain comprises a variable domain comprising at least one of CDR-L1, CDR-L2, CDR-L3, CDR-H1, CDR-H2, or CDR-H3 described herein. Such immunoglobulin heavy chains can comprise or consist of at least one of CDR-H1, CDR-H2, and CDR-H3. Such immunoglobulin light chains can comprise or consist of at least one of CDR-L1, CDR-L2, and CDR-L3.

In other embodiments, an immunoglobulin heavy and/or light chain according to the present invention comprises or consists of a vH or vK variable domain sequence, respectively, described herein.

The present invention further provides polypeptides which have a sequence selected from the group consisting of vH variable domain, vκ variable domain, CDR-L1, CDR-L2, CDR-L3, CDR-H1, CDR-H2, and CDR-H3 sequences described herein.

Antibodies, immunoglobulins, and polypeptides encompassed by the present invention can be use in an isolated (e.g., purified) form or contained in a vector, such as a membrane or lipid vesicle (e.g. a liposome).

Amino acid sequence modification(s) of the antibodies described herein are contemplated. For example, it may be desirable to improve the binding affinity and/or other biological properties of the antibody. It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce binding activity and can be corrected by replacing the amino acids with amino acid residues of the original antibody derived from a non-human animal.

Modifications and changes may be made in the structure of the antibodies encompassed by the present invention, and in the DNA sequences encoding them, and still obtain a functional molecule that encodes an antibody and polypeptide with desirable characteristics. For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the antibodies sequences encompassed by the present invention, or corresponding DNA sequences which encode said polypeptides, without appreciable loss of their biological activity.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (45).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

Another type of amino acid modification of the antibody encompassed by the present invention may be useful for altering the original glycosylation pattern of the antibody to, for example, increase stability. By “altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagines-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. For example, such methods are described in WO87/05330.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr et al. (1987) and by Edge et al. (1981). Enzymatic cleavage of carbohydrate moieties on antibodies can be achieved by the use of a variety of endo- and exo-glycosidases as described by Thotakura et al. (1987).

Other modifications can involve the formation of immunoconjugates. For example, in one type of covalent modification, antibodies or proteins are covalently linked to one of a variety of non proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Conjugation of antibodies or other proteins encompassed by the present invention with heterologous agents can be made using a variety of bifunctional protein coupling agents including but not limited to N-succinimidyl (2-pyridyldithio) propionate (SPDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody (WO 94/11026).

In another aspect, the present invention features antibodies conjugated to a therapeutic moiety, such as a cytotoxin, a drug, and/or a radioisotope. When conjugated to a cytotoxin, these antibody conjugates are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). An antibody encompassed by the present invention can be conjugated to a radioisotope, e.g., radioactive iodine, to generate cytotoxic radiopharmaceuticals for treating a related disorder, such as a cancer.

Conjugated antibodies can be used diagnostically or prognostically to monitor polypeptide levels in tissue as part of a clinical testing procedure, e.g., to determine the efficacy of a given treatment regimen. Detection can be facilitated by coupling (i e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (PE); an example of a luminescent material includes luminol; examples of bioluminescent materials include luciferase, luciferin, and acquorin, and examples of suitable radioactive material include ¹²⁵I, ¹³¹I, ³⁵S, or ³H. [0134] As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody, as well as indirect labeling of the antibody by reactivity with a detectable substance.

The antibody conjugates encompassed by the present invention can be used to modify a given biological response. The therapeutic moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, Pseudomonas exotoxin, or diphtheria toxin; a protein such as tumor necrosis factor or interferon-.gamma.; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other cytokines or growth factors.

Techniques for conjugating such therapeutic moiety to antibodies are well-known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243 56 (Alan R. Liss, Inc. 1985); Hellstrom et al., “Antibodies For Drug Delivery”, in Controlled Drug Delivery (2nd Ed.), Robinson et al. (eds.), pp. 623 53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475 506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303 16 (Academic Press 1985), and Thorpe et al., “The Preparation And Cytotoxic Properties Of Antibody-Toxin Conjugates”, Immunol. Rev., 62:119 58 (1982).

In some embodiments, conjugations can be made using a “cleavable linker” facilitating release of the cytotoxic agent or growth inhibitory agent in a cell. For example, an acid-labile linker, peptidase-sensitive linker, photolabile linker, dimethyl linker or disulfide-containing linker (See e.g. U.S. Pat. No. 5,208,020) may be used. Alternatively, a fusion protein comprising the antibody and cytotoxic agent or growth inhibitory agent may be made, by recombinant techniques or peptide synthesis. The length of DNA may comprise respective regions encoding the two portions of the conjugate either adjacent one another or separated by a region encoding a linker peptide which does not destroy the desired properties of the conjugate.

Additionally, recombinant polypeptide antibodies, such as chimeric and humanized monoclonal antibodies, comprising both human and non-human portions, which can be made using standard recombinant DNA techniques, are within the scope encompassed by the present invention. Such chimeric and humanized monoclonal antibodies can be produced by recombinant DNA techniques known in the art, for example using methods described in Robinson et al. International Patent Publication PCT/US86/02269; Akira et al. European Patent Application 184,187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immnol. 139:3521-3526; Sun et al. (1987) Proc. Natl. Acad. Sci. 84:214-218; Nishimura et al. (1987) Cancer Res. 47:999-1005: Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. Nos. 5,565,332, 5,871,907, or 5,733,743. The use of intracellular antibodies to inhibit protein function in a cell is also known in the art (see e.g., Carlson, J. R. (1988) Mol. Cell. Biol. 8:2638-2646; Biocca, S. et al. (1990) EMBOJ. 9:101-108; Werge, T. M. et al. (1990) FEBS Lett. 274:193-198; Carlson, J. R. (1993) Proc. Natl. Acad. Sci. USA 90:7427-7428; Marasco, W. A. et al. (1993) Proc. Natl. Acad Sci. USA 90:7889-7893; Biocca, S. et al. (1994) Biotechnology (NY) 12:396-399; Chen, S-Y. et al. (1994) Hum. Gene Ther. 5:595-601; Duan, L et al. (1994) Proc. Natl. Acad Sci. USA 91:5075-5079; Chen, S-Y. et al. (1994) Proc. Natl. Acad. Sci. ISA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad Sci. USA 92:3137-3141; PCT Publication No. WO 94/02610 by Marasco et al.; and PCT Publication No. WO 95/03832 by Duan et al.).

Additionally, fully human antibodies could be made against PTPN2, or fragments thereof. Fully human antibodies can be made in mice that are transgenic for human immunoglobulin genes, e.g. according to Hogan et al., “Manipulating the Mouse Embryo: A Laboratory Manuel,” Cold Spring Harbor Laboratory. Briefly, transgenic mice are immunized with purified immunogen. Spleen cells are harvested and fused to myeloma cells to produce hybridomas. Hybridomas are selected based on their ability to produce antibodies which bind to the immunogen. Fully human antibodies would reduce the immunogenicity of such antibodies in a human.

In one embodiment, an antibody for use in the instant invention is a bispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893. Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. USA, 83:1453, and Staerz and Bevan (1986)Immunol. Today 7:241). Bispecific antibodies are also described in U.S. Pat. No. 5,959,084. Fragments of bispecific antibodies are described in U.S. Pat. No. 5,798,229.

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences. The antibody component can bind to a polypeptide or a fragment thereof of one or more biomarkers encompassed by the present invention, including PTPN2, or a fragment thereof. In one embodiment, the bispecific antibody could specifically bind to both a polypeptide or a fragment thereof and its natural binding partner(s) or a fragment(s) thereof.

In another aspect encompassed by the present invention, peptides or peptide mimetics can be used to antagonize or agonize the activity of one or more biomarkers encompassed by the present invention, including PTPN2, or a fragment(s) thereof. In one embodiment, variants of PTPN2 which function as a modulating agent for the respective full length protein, can be identified by screening combinatorial libraries of mutants, e.g., truncation mutants, for antagonist activity. In one embodiment, a variegated library of variants is generated by combinatorial mutagenesis at the nucleic acid level and is encoded by a variegated gene library. A variegated library of variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of interest (Arkin and Youvan (1992) Proc. Natl. Acad Sci. USA 89:7811-7815; Delagrave et al. (1993) Protein Eng. 6(3):327-331). In one embodiment, cell based assays can be exploited to analyze a variegated polypeptide library. For example, a library of expression vectors can be transfected into a cell line which ordinarily synthesizes one or more biomarkers encompassed by the present invention, including PTPN2, or a fragment thereof. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences disclosed herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well-known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969)J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisyntheic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments encompassed by the present invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides disclosed herein can be used therapeutically to treat disease, e.g., by altering costimulation in a patient.

Peptidomimetics (Fauchere, J. (1986) Adv. Drug Res. 15:29; Veber and Frei dinger (1985) TINS p. 392; and Evans et al. (1987) J. Med. Chem. 30:1229, which are incorporated herein by reference) are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH2NH—, —CH2S—, —CH2-CH2-, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982)(—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds to produce the therapeutic effect. Derivitization (e.g., labeling) of peptidomimetics should not substantially interfere with the desired biological or pharmacological activity of the peptidomimetic.

Also encompassed by the present invention are small molecules which can modulate (inhibit) interactions, e.g., between PTPN2 and their natural binding partners. The small molecules encompassed by the present invention can be obtained using any of the numerous approaches in combinatorial library methods known in the art, including: spatially addressable parallel solid phase or solution phase libraries; synthetic library methods requiring deconvolution; the ‘one-bead one-compound’ library method; and synthetic library methods using affinity chromatography selection. (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. ISA 91:11422; Zuckermann et al. (1994) J. Med. Chem. 37:2678; Cho et al. (1993) Science 261:1303; Carrell et al. (1994) Angew. Chem. Int. Ld. Engl. 33:2059; Carell et al. (1994) Angew. Chem. Int. Engl. 33:2061; and in Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.). Compounds can be screened in cell based or non-cell based assays. Compounds can be screened in pools (e.g. multiple compounds in each testing sample) or as individual compounds.

The invention also relates to chimeric or fusion proteins of the biomarkers encompassed by the present invention, including PTPN2, or fragments thereof. As used herein, a “chimeric protein” or “fusion protein” comprises one or more biomarkers encompassed by the present invention, including PTPN2, or a fragment thereof, operatively linked to another polypeptide having an amino acid sequence corresponding to a protein which is not substantially homologous to the respective biomarker. In a preferred embodiment, the fusion protein comprises at least one biologically active portion of one or more biomarkers encompassed by the present invention, including PTPN2, or fragments thereof. Within the fusion protein, the term “operatively linked” is intended to indicate that the biomarker sequences and the non-biomarker sequences are fused in-frame to each other in such a way as to preserve functions exhibited when expressed independently of the fusion. The “another” sequences can be fused to the N-terminus or C-terminus of the biomarker sequences, respectively.

Such a fusion protein can be produced by recombinant expression of a nucleotide sequence encoding the first peptide and a nucleotide sequence encoding the second peptide. The second peptide may optionally correspond to a moiety that alters the solubility, affinity, stability or valency of the first peptide, for example, an immunoglobulin constant region. In another preferred embodiment, the first peptide consists of a portion of a biologically active molecule (e.g. the extracellular portion of the polypeptide or the ligand binding portion). The second peptide can include an immunoglobulin constant region, for example, a human Cγ1 domain or Cy4 domain (e.g., the hinge, CH2 and CH3 regions of human IgCγ 1, or human IgCγ4, see e.g., Capon et al. U.S. Pat. Nos. 5,116,964; 5,580,756; 5,844,095 and the like, incorporated herein by reference). Such constant regions may retain regions which mediate effector function (e.g. Fc receptor binding) or may be altered to reduce effector function. A resulting fusion protein may have altered solubility, binding affinity, stability and/or valency (i.e., the number of binding sites available per polypeptide) as compared to the independently expressed first peptide, and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture typically includes host cells, media and other byproducts. Suitable media for cell culture are well-known in the art. Protein and peptides can be isolated from cell culture media, host cells, or both using techniques known in the art for purifying proteins and peptides. Techniques for transfecting host cells and purifying proteins and peptides are known in the art.

Preferably, a fusion protein encompassed by the present invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992).

In another embodiment, the fusion protein contains a heterologous signal sequence at its N-terminus. In certain host cells (e.g., mammalian host cells), expression and/or secretion of a polypeptide can be increased through use of a heterologous signal sequence.

The fusion proteins encompassed by the present invention can be used as immunogens to produce antibodies in a subject. Such antibodies may be used to purify the respective natural polypeptides from which the fusion proteins were generated, or in screening assays to identify polypeptides which inhibit the interactions between one or more biomarkers polypeptide or a fragment thereof and its natural binding partner(s) or a fragment(s) thereof.

Also provided herein are compositions comprising one or more nucleic acids comprising or capable of expressing at least 1, 2, 3, 4, 5, 10, 20 or more small nucleic acids or antisense oligonucleotides or derivatives thereof, wherein said small nucleic acids or antisense oligonucleotides or derivatives thereof in a cell specifically hybridize (e.g., bind) under cellular conditions, with cellular nucleic acids (e.g., small non-coding RNAS such as miRNAs, pre-miRNAs, pri-miRNAs, miRNA*, anti-miRNA, a miRNA binding site, a variant and/or functional variant thereof, cellular mRNAs or a fragments thereof). In one embodiment, expression of the small nucleic acids or antisense oligonucleotides or derivatives thereof in a cell can enhance or upregulate one or more biological activities associated with the corresponding wild-type, naturally occurring, or synthetic small nucleic acids. In another embodiment, expression of the small nucleic acids or antisense oligonucleotides or derivatives thereof in a cell can inhibit expression or biological activity of cellular nucleic acids and/or proteins, e.g., by inhibiting transcription, translation and/or small nucleic acid processing of, for example, one or more biomarkers encompassed by the present invention, including PTPN2, or fragment(s) thereof. In one embodiment, the small nucleic acids or antisense oligonucleotides or derivatives thereof are small RNAs (e.g., microRNAs) or complements of small RNAs. In another embodiment, the small nucleic acids or antisense oligonucleotides or derivatives thereof can be single or double stranded and are at least six nucleotides in length and are less than about 1000, 900, 800, 700, 600, 500, 400, 300, 200, 100, 50, 40, 30, 25, 24, 23, 22, 21, 20, 19, 18, 17, 16, 15, or 10 nucleotides in length. In another embodiment, a composition may comprise a library of nucleic acids comprising or capable of expressing small nucleic acids or antisense oligonucleotides or derivatives thereof, or pools of said small nucleic acids or antisense oligonucleotides or derivatives thereof. A pool of nucleic acids may comprise about 2-5, 5-10, 10-20, 10-30 or more nucleic acids comprising or capable of expressing small nucleic acids or antisense oligonucleotides or derivatives thereof.

In one embodiment, binding may be by conventional base pair complementarity, or, for example, in the case of binding to DNA duplexes, through specific interactions in the major groove of the double helix. In general, “antisense” refers to the range of techniques generally employed in the art, and includes any process that relies on specific binding to oligonucleotide sequences.

It is well-known in the art that modifications can be made to the sequence of a miRNA or a pre-miRNA without disrupting miRNA activity. As used herein, the term “functional variant” of a miRNA sequence refers to an oligonucleotide sequence that varies from the natural miRNA sequence, but retains one or more functional characteristics of the miRNA (e.g. cancer cell proliferation inhibition, induction of cancer cell apoptosis, enhancement of cancer cell susceptibility to chemotherapeutic agents, specific miRNA target inhibition). In some embodiments, a functional variant of a miRNA sequence retains all of the functional characteristics of the miRNA. In certain embodiments, a functional variant of a miRNA has a nucleobase sequence that is a least about 60%, 65%, 70%, 75%, 80%, 85%, 90%, 91%, 92%, 93%, 94%, 95%, 96%, 97%, 98% or 99% identical to the miRNA or precursor thereof over a region of about 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the functional variant hybridizes to the complement of the miRNA or precursor thereof under stringent hybridization conditions. Accordingly, in certain embodiments the nucleobase sequence of a functional variant is capable of hybridizing to one or more target sequences of the miRNA.

miRNAs and their corresponding stem-loop sequences described herein may be found in miRBase, an online searchable database of miRNA sequences and annotation, found on the world wide web at microrna.sanger.ac.uk. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence.

In some embodiments, miRNA sequences encompassed by the present invention may be associated with a second RNA sequence that may be located on the same RNA molecule or on a separate RNA molecule as the miRNA sequence. In such cases, the miRNA sequence may be referred to as the active strand, while the second RNA sequence, which is at least partially complementary to the miRNA sequence, may be referred to as the complementary strand. The active and complementary strands are hybridized to create a double-stranded RNA that is similar to a naturally occurring miRNA precursor. The activity of a miRNA may be optimized by maximizing uptake of the active strand and minimizing uptake of the complementary strand by the miRNA protein complex that regulates gene translation. This can be done through modification and/or design of the complementary strand.

In some embodiments, the complementary strand is modified so that a chemical group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules known in the art, including NH₂, NHCOCH₃, and biotin. In another embodiment, the uptake of the complementary strand by the miRNA pathway is reduced by incorporating nucleotides with sugar modifications in the first 2-6 nucleotides of the complementary strand. It should be noted that such sugar modifications can be combined with the 5′ terminal modifications described above to further enhance miRNA activities.

In some embodiments, the complementary strand is designed so that nucleotides in the 3′ end of the complementary strand are not complementary to the active strand. This results in double-strand hybrid RNAs that are stable at the 3′ end of the active strand but relatively unstable at the 5′ end of the active strand. This difference in stability enhances the uptake of the active strand by the miRNA pathway, while reducing uptake of the complementary strand, thereby enhancing miRNA activity.

Small nucleic acid and/or antisense constructs of the methods and compositions presented herein can be delivered, for example, as an expression plasmid which, when transcribed in the cell, produces RNA which is complementary to at least a unique portion of cellular nucleic acids (e.g., small RNAs, mRNA, and/or genomic DNA). Alternatively, the small nucleic acid molecules can produce RNA which encodes mRNA, miRNA, pre-miRNA, pri-miRNA, miRNA*, anti-miRNA, or a miRNA binding site, or a variant thereof. For example, selection of plasmids suitable for expressing the miRNAs, methods for inserting nucleic acid sequences into the plasmid, and methods of delivering the recombinant plasmid to the cells of interest are within the skill in the art. See, for example, Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee et al. (2002), Nat. Biotechnol. 20:500-505; and Paul et al. (2002), Nat. Biotechnol. 20:505-508, the entire disclosures of which are herein incorporated by reference.

Alternatively, small nucleic acids and/or antisense constructs are oligonucleotide probes that are generated ex vivo and which, when introduced into the cell, results in hybridization with cellular nucleic acids. Such oligonucleotide probes are preferably modified oligonucleotides that are resistant to endogenous nucleases, e.g., exonucleases and/or endonucleases, and are therefore stable in vivo. Exemplary nucleic acid molecules for use as small nucleic acids and/or antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

Antisense approaches may involve the design of oligonucleotides (either DNA or RNA) that are complementary to cellular nucleic acids (e.g., complementary to PTPN2 nucleic acids listed in Table 1). Absolute complementarity is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a nucleic acid (e.g., RNA) it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well (Wagner, R. (1994) Nature 372:333). Therefore, oligonucleotides complementary to either the 5′ or 3′ untranslated, non-coding regions of genes could be used in an antisense approach to inhibit translation of endogenous mRNAs. Oligonucleotides complementary to the 5′ untranslated region of the mRNA may include the complement of the AUG start codon. Antisense oligonucleotides complementary to mRNA coding regions are less efficient inhibitors of translation but could also be used in accordance with the methods and compositions presented herein. Whether designed to hybridize to the 5′, 3′ or coding region of cellular mRNAs, small nucleic acids and/or antisense nucleic acids should be at least six nucleotides in length, and can be less than about 1000, 900, 800, 700, 600, 500, 400, 300, 200, 100, 50, 40, 30, 25, 24, 23, 22, 21, 20, 19, 18, 17, 16, 15, or 10 nucleotides in length.

Regardless of the choice of target sequence, it is preferred that in vitro studies are first performed to quantitate the ability of the antisense oligonucleotide to inhibit gene expression. In one embodiment these studies utilize controls that distinguish between antisense gene inhibition and nonspecific biological effects of oligonucleotides. In another embodiment these studies compare levels of the target nucleic acid or protein with that of an internal control nucleic acid or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

Small nucleic acids and/or antisense oligonucleotides can be DNA or RNA or chimeric mixtures or derivatives or modified versions thereof, single-stranded or double-stranded. Small nucleic acids and/or antisense oligonucleotides can be modified at the base moiety, sugar moiety, or phosphate backbone, for example, to improve stability of the molecule, hybridization, etc., and may include other appended groups such as peptides (e.g., for targeting host cell receptors), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents. (See, e.g., Krol et al. (1988) BioTechniques 6:958-976) or intercalating agents. (See, e.g., Zon (1988), Pharm. Res. 5:539-549). To this end, small nucleic acids and/or antisense oligonucleotides may be conjugated to another molecule, e.g., a peptide, hybridization triggered cross-linking agent, transport agent, hybridization-triggered cleavage agent, etc.

Small nucleic acids and/or antisense oligonucleotides may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Small nucleic acids and/or antisense oligonucleotides may also comprise at least one modified sugar moiety selected from the group including but not limited to arabinose, 2-fluoroarabinose, xylulose, and hexose.

In certain embodiments, a compound comprises an oligonucleotide (e.g., a miRNA or miRNA encoding oligonucleotide) conjugated to one or more moieties which enhance the activity, cellular distribution or cellular uptake of the resulting oligonucleotide. In certain such embodiments, the moiety is a cholesterol moiety (e.g., antagomirs) or a lipid moiety or liposome conjugate. Additional moieties for conjugation include carbohydrates, phospholipids, biotin, phenazine, folate, phenanthridine, anthraquinone, acridine, fluoresceins, rhodamines, coumarins, and dyes. In certain embodiments, a conjugate group is attached directly to the oligonucleotide. In certain embodiments, a conjugate group is attached to the oligonucleotide by a linking moiety selected from amino, hydroxyl, carboxylic acid, thiol, unsaturations (e.g., double or triple bonds), 8-amino-3,6-dioxaoctanoic acid (ADO), succinimidyl 4-(N-maleimidomethyl) cyclohexane-1-carboxylate (SMCC), 6-aminohexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, carboxy, benzyl, phenyl, nitro, thiol, thioalkoxy, halogen, alkyl, aryl, alkenyl and alkynyl.

In certain such embodiments, the compound comprises the oligonucleotide having one or more stabilizing groups that are attached to one or both termini of the oligonucleotide to enhance properties such as, for example, nuclease stability. Included in stabilizing groups are cap structures. These terminal modifications protect the oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

Suitable cap structures include a 4,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threo-pentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-2′-inverted nucleotide moiety, a 3′-2′-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5′-5′-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

Small nucleic acids and/or antisense oligonucleotides can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, small nucleic acids and/or antisense oligonucleotides comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In a further embodiment, small nucleic acids and/or antisense oligonucleotides are α-anomeric oligonucleotides. An α-anomeric oligonucleotide forms specific double-stranded hybrids with complementary RNA in which, contrary to the usual b-units, the strands run parallel to each other (Gautier et al. (1987) Nucl. Acids Res. 15:6625-6641). The oligonucleotide is a 2′-O-methylribonucleotide (Inoue et al. (1987) Nucl. Acids Res. 15:6131-6148), or a chimeric RNA-DNA analogue (Inoue et al. (1987) FEBS Lett. 215:327-330).

Small nucleic acids and/or antisense oligonucleotides of the methods and compositions presented herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988) Nucl. Acids Res. 16:3209, methylphosphonate oligonucleotides can be prepared by use of controlled pore glass polymer supports (Sarin et al. (1988) Proc. Natl. Acad. Sci. U.S.A. 85:7448-7451), etc. For example, an isolated miRNA can be chemically synthesized or recombinantly produced using methods known in the art. In some instances, miRNA are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), Cruachem (Glasgow, UK), and Exiqon (Vedbaek, Denmark).

Small nucleic acids and/or antisense oligonucleotides can be delivered to cells in vivo. A number of methods have been developed for delivering small nucleic acids and/or antisense oligonucleotides DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

In one embodiment, small nucleic acids and/or antisense oligonucleotides may comprise or be generated from double stranded small interfering RNAs (siRNAs), in which sequences fully complementary to cellular nucleic acids (e.g. mRNAs) sequences mediate degradation or in which sequences incompletely complementary to cellular nucleic acids (e.g., mRNAs) mediate translational repression when expressed within cells. In another embodiment, double stranded siRNAs can be processed into single stranded antisense RNAs that bind single stranded cellular RNAs (e.g., microRNAs) and inhibit their expression. RNA interference (RNAi) is the process of sequence-specific, post-transcriptional gene silencing in animals and plants, initiated by double-stranded RNA (dsRNA) that is homologous in sequence to the silenced gene. in vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. (2001) Nature 411:494-498). Accordingly, translation of a gene in a cell can be inhibited by contacting the cell with short double stranded RNAs having a length of about 15 to 30 nucleotides or of about 18 to 21 nucleotides or of about 19 to 21 nucleotides. Alternatively, a vector encoding for such siRNAs or short hairpin RNAs (shRNAs) that are metabolized into siRNAs can be introduced into a target cell (see, e.g., McManus et al. (2002) RNA 8:842; Xia et al. (2002) Nature Biotechnology 20:1006; and Brummelkamp et al. (2002) Science 296:550). Vectors that can be used are commercially available, e.g., from OligoEngine under the name pSuper RNAi System™.

Ribozyme molecules designed to catalytically cleave cellular mRNA transcripts can also be used to prevent translation of cellular mRNAs and expression of cellular polypeptides, or both (See, e.g., PCT International Publication WO90/11364, published Oct. 4, 1990; Sarver et al. (1990) Science 247:1222-1225 and U.S. Pat. No. 5,093,246). While ribozymes that cleave mRNA at site specific recognition sequences can be used to destroy cellular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well-known in the art and is described more fully in Haseloff and Gerlach (1988) Nature 334:585-591. The ribozyme may be engineered so that the cleavage recognition site is located near the 5′ end of cellular mRNAs; i.e., to increase efficiency and minimize the intracellular accumulation of non-functional mRNA transcripts.

The ribozymes of the methods and compositions presented herein also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug et al. (1984) Science 224:574-578; Zaug et al. (1986) Science 231:470-475; Zaug et al. (1986) Nature 324:429-433; published International patent application No. WO88/04300 by University Patents Inc.; Been et al. (1986) Cell 47:207-216). The Cech-type ribozymes have an eight base pair active site which hybridizes to a target RNA sequence whereafter cleavage of the target RNA takes place. The methods and compositions presented herein encompasses those Cech-type ribozymes which target eight base-pair active site sequences that are present in cellular genes.

As in the antisense approach, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous cellular messages and inhibit translation. Because ribozymes unlike antisense molecules, are catalytic, a lower intracellular concentration is required for efficiency.

Nucleic acid molecules to be used in triple helix formation for the inhibition of transcription of cellular genes are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Small nucleic acids (e.g., miRNAs, pre-miRNAs, pri-miRNAs, miRNA*, anti-miRNA, or a miRNA binding site, or a variant thereof), antisense oligonucleotides, ribozymes, and triple helix molecules of the methods and compositions presented herein may be prepared by any method known in the art for the synthesis of DNA and RNA molecules. These include techniques for chemically synthesizing oligodeoxyribonucleotides and oligoribonucleotides well-known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. One of skill in the art will readily understand that polypeptides, small nucleic acids, and antisense oligonucleotides can be further linked to another peptide or polypeptide (e.g., a heterologous peptide), e.g., that serves as a means of protein detection. Non-limiting examples of label peptide or polypeptide moieties useful for detection in the invention include, without limitation, suitable enzymes such as horseradish peroxidase, alkaline phosphatase, beta-galactosidase, or acetylcholinesterase; epitope tags, such as FLAG, MYC, HA, or HIS tags; fluorophores such as green fluorescent protein; dyes; radioisotopes; digoxygenin; biotin; antibodies; polymers; as well as others known in the art, for example, in Principles of Fluorescence Spectroscopy, Joseph R. Lakowicz (Editor), Plenum Pub Corp, 2nd edition (July 1999).

The modulatory agents described herein (e.g., antibodies, small molecules, peptides, fusion proteins, or small nucleic acids) can be incorporated into pharmaceutical compositions and administered to a subject in vivo. The compositions may contain a single such molecule or agent or any combination of agents described herein. “Single active agents” described herein can be combined with other pharmacologically active compounds (“second active agents”) known in the art according to the methods and compositions provided herein. It is believed that certain combinations work synergistically in the treatment of conditions that would benefit from the modulation of immune responses. Second active agents can be large molecules (e.g., proteins) or small molecules (e.g., synthetic inorganic, organometallic, or organic molecules).

Biomarker (e.g., PTPN2) nucleic acids and/or biomarker polypeptides can be analyzed according to the methods described herein and techniques known to the skilled artisan to identify such genetic or expression alterations useful for the present invention including, but not limited to, 1) an alteration in the level of a biomarker transcript or polypeptide, 2) a deletion or addition of one or more nucleotides from a biomarker gene, 4) a substitution of one or more nucleotides of a biomarker gene, 5) aberrant modification of a biomarker gene, such as an expression regulatory region, and the like.

a. Methods for Detection of Copy Number

Methods of evaluating the copy number of a biomarker nucleic acid are well-known to those of skill in the art. The presence or absence of chromosomal gain or loss can be evaluated simply by a determination of copy number of the regions or markers identified herein.

In one embodiment, a biological sample is tested for the presence of copy number changes in genomic loci containing the genomic marker. A copy number of at least 3, 4, 5, 6, 7, 8, 9, or 10 is predictive of poorer outcome of inhibitors of PTPN2.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In one embodiment, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well-known in the art to detect RNA can be used, such that higher or lower expression relative to an appropriate control (e.g., a non-amplified portion of the same or related cell tissue, organ, etc.) provides an estimate of the relative copy number of the target nucleic acid.

An alternative means for determining genomic copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonucleotides (e.g., to detect single nucleotide polymorphisms) and the like. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well-known in the art (see, e.g., U.S. Pat. Nos. 6,335,167; 6,197,501; 5,830,645; and 5,665,549 and Albertson (1984) EMBO J. 3: 1227-1234; Pinkel (1988) Proc. Natl. Acad Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc.). In another embodiment, the hybridization protocol of Pinkel et al. (1998) Nature Genetics 20: 207-211, or of Kallioniemi (1992) Proc. Natl Acad Sci USA 89:5321-5325 (1992) is used.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well-known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). Measurement of DNA copy number at microsatellite loci using quantitative PCR analysis is described in Ginzonger et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods encompassed by the present invention. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4: 560, Landegren et al. (1988) Science 241:1077, and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

Loss of heterozygosity (LOH) and major copy proportion (MCP) mapping (Wang, Z. C. et al. (2004) Cancer Res 64(1):64-71; Seymour, A. B. et al. (1994) Cancer Res 54, 2761-4; Hahn, S. A. et al. (1995) Cancer Res 55, 4670-5; Kimura, M. et al. (1996) Genes Chromosomes Cancer 17, 88-93; Li et al., (2008)MBC Bioinform. 9, 204-219) may also be used to identify regions of amplification or deletion.

b. Methods for Detection of Biomarker Nucleic Acid Expression

Biomarker expression may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a biomarker and functionally similar homologs thereof, including a fragment or genetic alteration thereof (e.g., in regulatory or promoter regions thereof) comprises detecting or determining RNA levels for the marker of interest. In one embodiment, one or more cells from the subject to be tested are obtained and RNA is isolated from the cells. In a preferred embodiment, a sample of breast tissue cells is obtained from the subject.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also be possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAs in.

The RNA sample can then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) Proc. Natl. Acad. Sci. U.S.A. 86: 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope encompassed by the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall et al., PCR Methods and Applications 4: 80-84 (1994). Real time PCR may also be used.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315: target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well-known in the art (see, e.g., U.S. Pat. Nos. 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

In one embodiment, the biological sample contains polypeptide molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

c. Methods for Detection of Biomarker Protein Expression

The activity or level of a biomarker protein can be detected and/or quantified by detecting or quantifying the expressed polypeptide. The polypeptide can be detected and quantified by any of a number of means well-known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a condition that would benefit from an increased immune response to inhibitors of PTPN2. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired biomarker protein standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In one embodiment, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Anti-biomarker protein antibodies, such as intrabodies, may also be used for imaging purposes, for example, to detect the presence of biomarker protein in cells and tissues of a subject. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a K_{d} of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

Antibodies are commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies. Antibodies produced from a library, e.g., phage display library, may also be used.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

d. Methods for Detection of Biomarker Structural Alterations

The following illustrative methods can be used to identify the presence of a structural alteration in a biomarker nucleic acid and/or biomarker polypeptide molecule in order to, for example, identify PTPN2, or other biomarkers used in the immunotherapies described herein that are overexpressed, overfunctional, and the like.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a biomarker nucleic acid such as a biomarker gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

In an alternative embodiment, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in biomarker nucleic acid can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, biomarker genetic mutations can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Such biomarker genetic mutations can be identified in a variety of contexts, including, for example, germline and somatic mutations.

In yet another embodiment, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized when performing the diagnostic assays (Naeve (1995) Biolechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a biomarker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type biomarker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a biomarker sequence, e.g., a wild-type biomarker treated with a DNA mismatch repair enzyme, and the cleavage products, if any, can be detected from electrophoresis protocols or the like (e.g., U.S. Pat. No. 5,459,039.)

In other embodiments, alterations in electrophoretic mobility can be used to identify mutations in biomarker genes. For example, single strand conformation polymorphism (SSCP) may be used to detect differences in electrophoretic mobility between mutant and wild type nucleic acids (Orita et al. (1989) Proc Natl. Acad Sci USA 86:2766; see also Cotton (1993)Mutat. Res. 285:125-144 and Hayashi (1992) Genet. Anal. Tech. Appl. 9:73-79). Single-stranded DNA fragments of sample and control biomarker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992)Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

### III. Hematopoietic Stem Cell Lineages, T Cells, and Cell Sources for Transduction and/or Administration

In one aspect, the agent for administration in the present invention is cell-based. In some embodiments, the cell-based agent comprises engineered cells of the hematopoietic stem cell (HSC) lineage which have decreased coy number, expression level, and/or activity of PTPN2. In another aspect, the methods and compositions described herein use cells of the hematopoietic stem cell (HSC) lineage for transduction purposes.

Various cell types in the HSC lineage, as well as methods for selecting, purifying, and isolating such cell types, are well known in the art (see, for example, U.S. Pat. No. 8,481,315). Cell types of interest may be obtained from any animal having an immune system. In one embodiment, cell types of interest are obtained from a mammal, including humans. As used herein, the terms “mammal” and “mammalian” refer to any vertebrate animal, including monotremes, marsupials and placental, that suckle their young and either give birth to living young (eutharian or placental mammals) or are egg-laying (metatharian or nonplacental mammals). For example, cell types of interest having a defined genetic background or unknown genetic background may be obtained from a human for use in the methods encompassed by the present invention. In one embodiment, cells having a defined T Cell receptor are useful for analysis since all progeny will be specific for a limited range of specific peptides. For example, TCRalpha knockout/transgenic LCMV P14 TCR transgenic mice does not develop endogenous mature TCR alpha beta cells and whose peripheral T cells are almost all CD8⁺ and express transgenic TCR specific for a peptide (P14) from the lymphocytic choriomeningitis virus (LCMV) presented by the MHC class I molecule H-2Db (see, for example, Bettini et al. (2012) Immunol. 136:265-272). In another embodiment, cell types of interest may be obtained from non-human mammals. Representative, non-limiting examples of non-human mammals include non-human primates (e.g., monkeys and chimpanzees), rodents (e.g., rats, mice, and guinea pigs), canines, felines, birds, fish, and ruminants (e.g., cows, sheep, pigs, and horses). In still another embodiment, the non-human mammal is a mouse. The animals from which cell types of interest are obtained may be adult, newborn (e.g., less than 48 hours old), immature, or in utero. Cell types of interest may be primary cells, stem cells, and zygotes. In yet another embodiment, human progenitor cells are used to reconstitute human immune systems in host animals such as mice. Such systems are well known in the art and include, for example, SCID:Hu models in which human cells are reconstituted in SCID mice (see, for example, McCune et al. (1988) Science 241:1632-1639).

As used herein, “obtained” from a biological material source means any conventional method of harvesting or partitioning a source of biological material from a donor. For example, biological material may obtained from a blood sample, such as a peripheral or cord blood sample, or harvested from bone marrow or amniotic fluid. Methods for obtaining such samples are well known to the artisan. In the present invention, the samples may be fresh (i.e., obtained from a donor without freezing). Moreover, the samples may be further manipulated to remove extraneous or unwanted components prior to expansion. The samples may also be obtained from a preserved stock. For example, in the case of peripheral or cord blood, the samples may be withdrawn from a cryogenically or otherwise preserved bank of such blood. Such samples may be obtained from any suitable donor.

“Hematopoietic stem cells” or “HSC” are clonogenic, self-renewing pluripotent cells capable of ultimately differentiating into all cell types of the hematopoietic system, including B cells T cells, NK cells, lymphoid dendritic cells, myeloid dendritic cells, granulocytes, macrophages, megakaryocytes, and erythroid cells. HSC self-renewal refers to the ability of an HSC cell to divide and produce at least one daughter cell with the same self-renewal and differentiation potential of a HSC; that is, cell division gives rise to additional HSCs. Self-renewal provides a continual source of undifferentiated stem cells for replenishment of the hematopoietic system. Several sub-types of HSC are known. For example, “short term repopulating hematopoietic stem cells” or “ST-HSC” refers to HSC that have limited, short term self-renewing capacity, and are characterized by their capacity to differentiate into cells of the myeloid and lymphoid lineage. ST-HSC are distinguished from long-term repopulating (LT) HSC by their limited length of self-renewal activity in culture assays (e.g., approximately 8 weeks; see, for example, Christensen and Weissman (2001) Proc. Natl. Acad. Sci. U.S.A. 98:14541-14546).

“Self-renewal” refers to the ability of a cell to divide and generate at least one daughter cell with the identical (e.g., self-renewing) characteristics of the parent cell. The second daughter cell may commit to a particular differentiation pathway. For example, a self-renewing hematopoietic stem cell divides and forms one daughter stem cell and another daughter cell committed to differentiation in the myeloid or lymphoid pathway. A committed progenitor cell has typically lost the self-renewal capacity, and upon cell division produces two daughter cells that display a more differentiated (i.e., restricted) phenotype.

In some embodiments, “HSCs and/or cells derived therefrom” may refer to any cell type, stage of development, marker expression state, and the like of a cell that may be naturally obtained from an HSC. In other embodiments, HSCs and/or cells derived therefrom are limited to cells of the hematopoietic stem cell lineage that are not terminally differentiated, post-mitotic, thymocytes, derived from the thymus, and/or otherwise functional.

As with other cells of the hematopoietic system, HSCs are typically defined by the presence of a characteristic set of cell markers. “Enriched” when used in the context of HSC refers to a cell population selected based on the presence of a single cell marker, generally CD34+, while “purified” in the context of HSC refers to a cell population resulting from a selection on the basis of two or more markers, such as CD34+ and CD90+.

“Marker phenotyping” refers to identification of markers or antigens on cells for determining their phenotype (e.g., differentiation state and/or cell type). This may be done by immunophenotyping, which uses antibodies that recognize antigens present on a cell. The antibodies may be monoclonal or polyclonal, but are generally chosen to have minimal cross reactivity with other cell markers. It is to be understood that certain cell differentiation or cell surface markers are unique to the animal species from which the cells are derived, while other cell markers will be common between species. These markers defining equivalent cell types between species are given the same marker identification even though there are species differences in structure (e.g., amino acid sequence). Cell markers include cell surfaces molecules, also referred to in certain situations as cell differentiation (CD) markers, and gene expression markers. The gene expression markers are those sets of expressed genes indicative of the cell type or differentiation state. In part, the gene expression profile will reflect the cell surface markers, although they may include non-cell surface molecules.

As used herein, “enriched” means that the percentage of marker phenotyped cells relative to other cells in a population is increased. In one embodiment, “purified” means that the percentage of marker phenotyped cells is substantially pure and excludes cells that are not marker phenotyped. A “substantially pure cell population” refers to a population of cells having a specified cell marker characteristic and differentiation potential that is at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, or more, or any value or range in between, of the cells making up the total cell population. Thus, a “substantially pure cell population” refers to a population of cells that contain fewer than about 50%, preferably fewer than about 20-25%, more preferably fewer than about 10-15%, and most preferably fewer than about 5% of cells that do not display a specified marker characteristic and differentiation potential under designated assay conditions.

In one embodiment, “isolated” refers to a product, compound, or composition which is separated from at least one other product, compound, or composition with which it is associated in its naturally occurring state, whether in nature or as made synthetically. In other embodiments, “isolated” means that desired marker phenotyped cells are physically separated from other cell populations. Methods for the enrichment, purification, and/or isolation of marker phenotyped cells are disclosed herein and are also well known in the art, such as by using fluorescence-activated cell scanning (FACS), magnetic cell sorting, and centrifugation (see, for example, U.S. Pat. Nos. 5,474,687, 5,677,136, and 6,004,743; and U.S. Pat. Publ. 2001/0039052).

The marker phenotypes useful for identifying HSC are well known in the art. For human HSC, for example, the cell marker phenotypes preferably include CD34⁺ CD38⁻ CD90(Thy1)⁺ Lin⁻. For mouse HSCs, an exemplary cell marker phenotype is Sca-1⁺ CD90⁺ (see, e.g., Spangrude et al. (1988) Science 1:661-673) or c-kit⁺ Thy^{lo} Lin⁻ Sca-1⁺ (see, Uchida et al (1990). J. Clin. Invest. 101:961-966). Alternative HSC markers such as aldehyde dehydrogenase and AC133 may also be used (see, for example, Storms et al. (1999) Proc. Natl. Acad. Sci. U.S.A. 96:9118-9123 and Yin et al. (1997) Blood 90:5002-5012).

As stated above, HSC are clonogenic cells, which possess the properties of both self-renewal (expansion) and multilineage potential giving rise to all types of mature blood cells. HSC are responsible for hematopoiesis and undergo proliferation and differentiation to produce mature blood cells of various lineages while still maintaining their capacity for self-renewal. The ability to self-renew maintains the HSC population for the lifespan of an animal and also allows HSC to repopulate the bone marrow of lethally irradiated hosts. Early HSC development displays a hierarchical arrangement, starting from long-term (LT-) HSCs, which have extensive self-renewal capability, followed by the expansion state, which corresponds to short-term (ST-) HSCs (having limited self-renewal ability) and proliferative multipotent progenitors (MPP) (having multipotent potential but no self-renewal capability). MPP is also a stage of priming or preparation for differentiation. An MPP differentiates and, during this process, the more primitive population gives rise to a less primitive population of cells, which is unable to give rise to a more primitive population of cells. Genetic programs control these processes, including the multipotential, self-renewal, and activation (or transient amplification) of HSCs, and lineage commitment from MPP to lymphoid and myeloid progenitor cells.

Thus, HSCs give rise to committed lymphoid or myeloid progenitor cells. “Committed myeloid progenitor cells” refer to cell populations capable of differentiating into any of the terminally differentiated cells of the myeloid lineage. Encompassed within the myeloid progenitor cells are the “common myeloid progenitor cells (CMP)”, a cell population characterized by limited or non-self-renewal capacity but which is capable of cell division to form granulocyte/macrophage progenitor cells (GMP) and megakaryocyte/erythroid progenitor cells (MEP). Such cell populations may then give rise to myeloid dendritic, myeloid erythroid, erythroid, megakaryocytes, granulocyte/macrophage, granulocyte, and macrophage cells. Non-self-renewing cells refers to cells that undergo cell division to produce daughter cells, neither of which have the differentiation potential of the parent cell type, but instead generates differentiated daughter cells. Committed progenitor cells of the myeloid lineage include oligopotent CMP, GMP, and MEP as defined herein, but also encompass unipotent erythroid progenitor, megakaryocyte progenitor, granulocyte progenitor, and macrophage progenitor cells. Different cell populations of myeloid progenitor cells are distinguishable from other cells by their differentiation potential, and the presence of a characteristic set of cell markers. The marker phenotypes useful for identifying CMPs include those well known in the art. For CMP cells of murine origin, for example, the cell population is characterized by the marker phenotype c-Kit^{high} (CD117) CD16^{low} CD34^{low} Sca-1^{neg} Lin^{neg} and further characterized by the marker phenotypes FcγR^{lo} IL-7Rα^{neg} (CD127). The murine CMP cell population is also characterized by the absence of expression of markers that include B220, CD4, CD8, CD3, Ter 19, Gr-1 and Mac-1. For CMP cells of human origin, the cell population is characterized by CD34⁺ CD38⁺ and further characterized by the marker phenotype, CD123⁺ (IL-3Rα) CD45RA^{neg}. The human CMP cell population is also characterized by the absence of cell markers CD3, CD4, CD7, CD8, CD10, CD11b, CD14, CD19, CD20, CD56, and CD234a. Descriptions of marker phenotypes for various myeloid progenitor cells are described in, for example, U.S. Pat. Nos. 6,465,247 and 6,761,883 and Akashi (2000) Nature 404:193-197.

A committed progenitor cell of the myeloid lineage is the “granulocyte/macrophage progenitor cell (GMP)”. GMP are cells derived from common myeloid progenitor cells, and characterized by a capacity to give rise to granulocyte (e.g., basophils, eosinophils, and neutrophils) and macrophage cells, but which do not typically give rise to erythroid cells or megakaryocytes of the myeloid lineage. Similar to other committed progenitor cells, GMPs lack self-renewal capacity. Murine GMPs may be characterized by the marker phenotype c-Kit^{hi} (CD 117) Sca-1^{neg}FcγR^{hi} (CD16) IL-7Rγ^{neg}CD34^{pos}. Murine GMPs also lack expression of markers B220, CD4, CD8, CD3, Gr-1, Mac-1, and CD90. Human GMPs may be characterized by the marker phenotype CD34⁺ CD38⁺ CD123+CD45RA⁺. Human GMP cell populations are also characterized by the absence of markers CD3, CD4, CD7, CD8, CD10, CD11b, CD14, CD19, CD20, CD56, and CD235a.

“Megakaryocyte/erythroid progenitor cells (MEP)” are derived from the CMPs and are characterized by their capability of differentiating into committed megakaryocyte progenitor and erythroid progenitor cells. MEP give rise to erythroid cells and megakaryocytes, but do not typically give rise to granulocytes, macrophages, or myeloid dendritic cells. Mature megakaryocytes are polyploid cells that are precursors for formation of platelets, a developmental process regulated by thrombopoietin. Erythroid cells are formed from the committed erythroid progenitor cells through a process regulated by erythropoietin, and ultimately differentiate into mature red blood cells. Murine MEPs may be characterized by cell marker phenotype c-Kit^{hi} and IL-7Rα^{neg} and further characterized by marker phenotypes FcγRₗₒ and CD34^{low}. Murine MEP cell populations may also be characterized by the absence of markers B220, CD4, CD8, CD3, Gr-1, and CD90. Another exemplary marker phenotype for mouse MEPs is c-kitu^{high} Sca-1^{neg} Lin^{neg/low} CD16^{low} CD16^{low} CD34^{low}. Human MEPs may be characterized by marker phenotypes CD34⁺ CD38⁺ CD123^{neg} CD45RA^{neg}. Human MEP cell populations may also be characterized by the absence of markers CD3, CD4, CD7, CD8, CD10, CD11b, CD14, CD19, CD20, CD56, and CD235a.

Further restricted progenitor cells in the myeloid lineage are the granulocyte progenitor, macrophage progenitor, megakaryocyte progenitor, and erythroid progenitor cell types. “Granulocyte progenitor (GP)” cells are characterized by their capability to differentiate into terminally differentiated granulocytes, including eosinophils, basophils, neutrophils. The GP typically do not differentiate into other cells of the myeloid lineage. “Megakaryocyte progenitor cell (MKP)” cells are characterized by their capability to differentiate into terminally differentiated megakaryocytes but generally not other cells of the myeloid lineage (see, e.g., WO 2004/024875).

For the lymphoid lineage, a “committed lymphoid progenitor cell” refers to an oligopotent or unipotent progenitor cell capable of differentiating into any of the terminally differentiated cells of the lymphoid lineage, such as T cell, B cell, NK cell, or lymphoid dendritic cells, but which do not typically differentiate into cells of the myeloid lineage. As with cells of the myeloid lineage, different cell populations of lymphoid progenitors are distinguishable from other cells by their differentiation potential, and the presence of a characteristic set of cell markers. Encompassed within the lymphoid progenitor cells are the “common lymphoid progenitor cells (CLP)”, which are oligopotent cells characterized by a capacity to give rise to B-cell progenitors (BCP), T-cell progenitors (TCP), NK cells, and dendritic cells. These progenitor cells have little or no self-renewing capacity, but are capable of giving rise to T lymphocytes, B lymphocytes, NK cells, and lymphoid dendritic cells. The marker phenotypes useful for identifying CLPs are commonly known in the art. For CLP cells of mouse, the cell population may be characterized by the presence of markers as described in, for example, Kondo et. al., (1997) Cell 91:661-672, while for human CLPs, a marker phenotype of CD34⁺ CD38⁺ CD10⁺ IL7R+ may be used (Galy et al. (1995) Immunity 3:459-473 and Akashi et al. (1999) Int. J. Hematol. 69:217-226).

Numerous other suitable cell surface markers are presently known to the skilled artisan and such markers will find advantageous use in the methods and compositions described herein. For instance, several additional potential murine markers have recently been identified for the various myeloid progenitor cell populations based on array analysis of mRNA expression. See, e.g., Iwasaki-Arai et al. (2003) J. Exp. Med. 197:1311-1322; Akashi et al. (2000) Nature 404:193-197; Miyamoto et al. (2002) Dev. Cell 3:137-147; Traver et al. (2001) Blood 98:627-635; Akashi et al. (2003) Blood 101:383-390; and Terskikh et al. (2003) Blood 102:102:94-101. Based on this same type of mRNA expression analysis, additional cell surface markers such as CD110, CD 114, CD 116, CD 117, CD127, and CD135 may also find use for isolating one or more of the identified myeloid progenitor subpopulations in humans, as described in Manz et al. (2002) Proc. Natl. Acad. Sci. U.S.A. 99:11872-11877.

Useful cells of the HSC lineage to be transduced may be capable of differentiating into cells of the myeloid lineage, i.e., granulocytes, macrophages, megakaryocytes, erythroid cells, and/or myeloid dendritic cells. These include, among others, HSCs, and committed myeloid progenitor cells CMPs, GMPs, and MEPs. These cells will have the relevant characteristics, particularly differentiation potential and cell marker characteristics described above. Such cells may be obtained from a variety of sources, including bone marrow, peripheral blood, cord blood, amniotic fluid, and other sources known to harbor HSCs and/or cells derived therefrom, including liver, particularly fetal liver. Peripheral and cord blood is a rich source of HSC and related lineage cells.

Cells may be obtained using methods well known in the art. For example, methods for preparing bone marrow cells are described in Sutherland et al. (1991) Bone Marrow Processing and Purging. A Practical Guide (Gee, A. P. ed.), CRC Press Inc. Umbilical cord blood or placental cord blood is typically obtained by puncture of the umbilical vein, in both term or preterm, before or after placental detachment (see, e.g., Turner (1992) Bone Marrow Transplant. 10:89 and Bertolini et a. (1995) J. Hematother. 4:29). HSCs and myeloid progenitor cells may also be obtained from peripheral blood by leukapheresis, a procedure in which blood drawn from a suitable subject is processed by continuous flow centrifugation (e.g., Cobe BCT Spectra blood cell separators) to remove white blood cells while the other blood components are returned to the donor. Another type of isolation procedure is centrifugation through a medium of varying density, such as Ficoll-Hypaque (Amersham Pharmacia Biotech, Piscataway, N.J.).

Cells may be derived from any animal species with a hematopoietic system, as generally described herein. Preferably, suitable animals will be mammals, including, by way of example and without limitation, rodents, rabbits, canines, felines, pigs, horses, cows, primates (e.g., human), and the like. The cells may be obtained from a single subject or a plurality of subjects. A plurality refers to at least two (e.g., more than one) donors. When cells obtained are from a plurality of donors, their relationships may be syngeneic, allogeneic, or xenogeneic, as defined herein.

Where applicable, HSC and related lineage cells may be mobilized from the bone marrow into the peripheral blood by prior administration of cytokines or drugs to the subject (see, e.g., Lapidot et al. (2002) Exp. Hematol. 30:973-981). The term “cytokine” refers to compounds or compositions that in the natural state are made by cells and affect physiological states of the cells that produce the cytokine (i.e., autocrine factors) or other cells. Cytokine also encompasses any compounds or compositions made by recombinant or synthetic processes, where the products of those processes have identical or similar structure and biological activity as the naturally occurring forms. Lymphokines refer to natural, synthetic, or recombinant forms of cytokines naturally produced by lymphocytes, including, but not limited to, IL-1, IL-3, IL-4, IL-6, IL-11, and the like. Cytokines and chemokines capable of inducing mobilization include, by way of example and not limitation, granulocyte colony stimulating factor (G-CSF), granulocyte macrophage colony stimulating factor (GM-CSF), erythropoietin (Kiessinger et al. (1995) Exp. Hematol. 23:609-612), stem cell factor (SCF), AMD3100 (AnorMed, Vancouver, Canada), interleukin-8 (IL-8), and variants of these factors (e.g., pegfilgastrim and darbopoietin). Combinations of cytokines and/or chemokines, such as G-CSF and SCF or GM-CSF and G-CSF, may act synergistically to promote mobilization and may be used to increase the number of HSC and progenitor cells in the peripheral blood, particularly for subjects who do not show efficient mobilization with a single cytokine or chemokine (see, for example, Morris et al. (2003) J. Haematol. 120:413-423).

Cytoablative agents may be used at inducing doses (i.e., cytoreductive doses) to also mobilize HSCs and progenitor cells, and are useful either alone or in combination with cytokines. This mode of mobilization is applicable when the subject is to undergo myeloablative treatment, and is carried out prior to the higher dose chemotherapy. Cytoreductive drugs for mobilization, include, among others, cyclophosphamide, ifosfamide, etoposide, cytosine arabinoside, and carboplatin (Montillo et al. (2004) Leukemia 18:57-62; Dasgupta et al. (1996) J. Infusional Chemother. 6:12; and Wright et al. (2001) Blood 97:2278-2285).

The HSCs and/or cells derived therefrom of interest may also be subjected to further selection, purification, and/or isolation, which may include both positive and negative selection methods, to obtain a substantially pure population of cells. In one aspect, fluorescence activated cell sorting (FACS), also referred to as flow cytometry, is used to sort and analyze the different cell populations. Cells having the cellular markers specific for HSC or a desired HSC lineage cell population are tagged with an antibody, or typically a mixture of antibodies, that bind the cellular markers. Each antibody directed to a different marker is conjugated to a detectable molecule, particularly a fluorescent dye that may be distinguished from other fluorescent dyes coupled to other antibodies. A stream of tagged or “stained” cells is passed through a light source that excites the fluorochrome and the emission spectrum from the cells detected to determine the presence of a particular labeled antibody. By concurrent detection of different fluorochromes, also referred to in the art as multicolor fluorescence cell sorting, cells displaying different sets of cell markers may be identified and isolated from other cells in the population. Other FACS parameters, including, by way of example and not limitation, side scatter (SSC), forward scatter (FSC), and vital dye staining (e.g., with propidium iodide) allow selection of cells based on size and viability. FACS sorting and analysis of HSC and related lineage cells is well known in the art and described in, for example, U.S. Pat. Nos. 5,137,809; 5,750,397; 5,840,580; 6,465,249; Manz et al. (202) Proc. Natl. Acad. Sci. U.S.A. 99:11872-11877; and Akashi et al. (200) Nature 404:193-197. General guidance on fluorescence activated cell sorting is described in, for example, Shapiro (2003) Practical Flow Cytometry, 4th Ed., Wiley-Liss (2003) and Ormerod (2000) Flow Cytometry: A Practical Approach, 3rd Ed., Oxford University Press.

Another method of isolating useful cell populations involves a solid or insoluble substrate to which is bound antibodies or ligands that interact with specific cell surface markers. In immunoadsorption techniques, cells are contacted with the substrate (e.g., column of beads, flasks, magnetic particles, etc.) containing the antibodies and any unbound cells removed. Immunoadsorption techniques may be scaled up to deal directly with the large numbers of cells in a clinical harvest. Suitable substrates include, by way of example and not limitation, plastic, cellulose, dextran, polyacrylamide, agarose, and others known in the art (e.g., Pharmacia Sepharose 6 MB macrobeads). When a solid substrate comprising magnetic or paramagnetic beads is used, cells bound to the beads may be readily isolated by a magnetic separator (see, e.g., Kato and Radbruch (1993) Cytometry 14:384-92). Affinity chromatographic cell separations typically involve passing a suspension of cells over a support bearing a selective ligand immobilized to its surface. The ligand interacts with its specific target molecule on the cell and is captured on the matrix. The bound cell is released by the addition of an elution agent to the running buffer of the column and the free cell is washed through the column and harvested as a homogeneous population. As apparent to the skilled artisan, adsorption techniques are not limited to those employing specific antibodies, and may use nonspecific adsorption. For example, adsorption to silica is a simple procedure for removing phagocytes from cell preparations.

FACS and most batch wise immunoadsorption techniques may be adapted to both positive and negative selection procedures (see, e.g., U.S. Pat. No. 5,877,299). In positive selection, the desired cells are labeled with antibodies and removed away from the remaining unlabeled/unwanted cells. In negative selection, the unwanted cells are labeled and removed. Another type of negative selection that may be employed is use of antibody/complement treatment or immunotoxins to remove unwanted cells.

It is to be understood that the purification or isolation of cells also includes combinations of the methods described above. A typical combination may comprise an initial procedure that is effective in removing the bulk of unwanted cells and cellular material, for example leukapharesis. A second step may include isolation of cells expressing a marker common to one or more of the progenitor cell populations by immunoadsorption on antibodies bound to a substrate. For example, magnetic beads containing anti-CD34 antibodies are able to bind and capture HSC, CMP, and GMP cells that commonly express the CD34 antigen. An additional step providing higher resolution of different cell types, such as FACS sorting with antibodies to a set of specific cellular markers, may be used to obtain substantially pure populations of the desired cells. Another combination may involve an initial separation using magnetic beads bound with anti-CD34 antibodies followed by an additional round of purification with FACS.

Determining the differentiation potential of cells, and thus the type of stem cells or progenitor cells isolated, is typically conducted by exposing the cells to conditions that permit development into various terminally differentiated cells. These conditions generally comprise a mixture of cytokines and growth factors in a culture medium permissive for development of the myeloid or lymphoid lineage. Colony forming culture assays rely on culturing the cells in vitro via limiting dilution and assessing the types of cells that arise from their continued development. A common assay of this type is based on methylcellulose medium supplemented with cytokines (e.g., MethoCult, Stem Cell Technologies, Vancouver, Canada and Kennedy et al. (1997) Nature 386:488-493). Cytokine and growth factor formulations permissive for differentiation in the hematopoietic pathway are described in Manz et al. (2002) Proc. Natl. Acad. Sci. U.S.A. 99:11872-11877; U.S. Pat. No. 6,465,249; and Akashi et al., Nature 404:193-197). Cytokines include SCF, FLT-3 ligand, GM-CSF, IL-3, TPO, and EPO. Another in vitro assay is long-term culture initiating cell (LTC-IC) assay, which typically uses stromal cells to support hematopoiesis (see, e.g., Ploemache et al. (1989) Blood 74:2755-2763 and Sutherland et al. (1995) Proc. Natl. Acad. Sci. U.S.A. 87:3745).

Another type of assay suitable for determining the differentiation potential of isolated cells relies upon in vivo administration of cells into a host animal and assessment of the repopulation of the hematopoietic system. The recipient is immunocompromised or immunodeficient to limit rejection and permits acceptance of allogeneic or xenogeneic cell transplants. A useful animal system of this kind is the NOD/SCID (Pflumio et al. (1996) Blood 88:3731; Szilvassym et al. (2002) “Hematopoietic Stem Cell Protocol” in Methods in Molecular Medicine, Humana Press; Greiner et al. (1998) Stem Cells 16:166-177; Piacibello et al. (1999) Blood 93:3736-3749) or Rag2 deficient mouse (Shinkai et al. (1992) Cell 68:855-867). Cells originating from the infused cells are assessed by recovering cells from the bone marrow, spleen, or blood of the host animal and determining presence of cells displaying specific cellular markers (i.e., marker phenotyping), typically by FACS analysis. Detection of markers specific to the transplanted cells permits distinguishing between endogenous and transplanted cells. For example, antibodies specific to human forms of the cell markers (e.g., HLA antigens) identify human cells when they are transplanted into suitable immunodeficient mouse.

The initial populations of cells obtained by the methods above may be used directly for transduction or frozen for use at a later date. A variety of mediums and protocols for cryopreservation are known in the art. Generally, the freezing medium will comprise DMSO from about 5-10%, 10-90% serum albumin, and 50-90% culture medium. Other additives useful for preserving cells include, by way of example and not limitation, disaccharides such as trehalose (Scheinkonig et al. (2004) Bone Marrow Transplant. 34:531-536), or a plasma volume expander, such as hetastarch (i.e., hydroxyethyl starch). In some embodiments, isotonic buffer solutions, such as phosphate-buffered saline, may be used. An exemplary cryopreservative composition has cell-culture medium with 4% HSA, 7.5% dimethyl sulfoxide (DMSO), and 2% hetastarch. Other compositions and methods for cryopreservation are well known and described in the art (see, e.g., Broxmeyer et al. (2003) Proc. Natl. Acad. Sci. U.S.A. 100:645-650). Cells are preserved at a final temperature of less than about −135° C.

In some embodiments, the cell-based agent encompassed by the present invention comprises engineered immune cells (e.g., CD8⁺ T cells). As used herein, the term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. For example, antigen-reactive T cells are T cells that selectively bind to an antigen of interest and modulate immunological responses based upon the recognition of antigen. Immune cells can be found in the peripheral blood. The term “peripheral blood cell subtypes” refers to cell types normally found in the peripheral blood including, but is not limited to, eosinophils, neutrophils, T cells, monocytes, NK cells, granulocytes, and B cells. Some immune cells are “antigen presenting cells,” include professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

Immune cells mediated immune responses. The term “immune response” refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus. In one embodiment, the response is specific for a particular antigen (an “antigen-specific response”), and refers to a response by a CD4 T cell, CD8 T cell, or B cell via their antigen-specific receptor. In another embodiment, an immune response is a T cell response, such as a CD4⁺ response or a CD8+ response. Such responses by these cells can include, for example, cytotoxicity, proliferation, cytokine or chemokine production, trafficking, or phagocytosis, and can be dependent on the nature of the immune cell undergoing the response. In still another embodiment, an immune response is an effector T cell response, such as occurs when a cytotoxic CD8⁺ cell produces an antigen-specific response. The term “immune response” includes T cell-mediated and/or B cell-mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

T cells are a class of immune cell and are generally divided into two subclasses, regulatory T cells (Tregs) and conventional T cells (Tconv).

Tregs are naturally occurring CD4+CD25+FOXP3+ T lymphocytes that comprise ˜5-10% of the circulating CD4⁺ T cell population, act to dominantly suppress autoreactive lymphocytes, and control innate and adaptive immune responses (Piccirillo and Shevach (2004) Semin. Immunol. 16:81-88; Fehervari and Sakaguchi (2004) Curr. Opin. Immunol. 16:203-208; Azuma et al. (2003) Cancer Res. 63:4516-4520; Cederbom et al. (2000) Eur. J. Immunol. 30:1538-1543; Maloy et al. (2003) J. Exp. Med 197:111-119; Serra et al. (2003) Immunity 19:877-889; Thornton and Shevach (1998) J. Fxp. Afed 188:287-296; Janssens et al. (2003) J. Immunol. 171:4604-4612; Gasteiger et al. (2013) J. Exp. Med 210:1167-1178; Sitrin et al. (2013) J. Exp. Med 210:1153-1165; Schmitt and Williams (2013) Front. Immunol. 4:1-13). Natural Tregs also express low amounts of CD127, develop in the thymus, express GITR and CTLA-4. Induced Tregs are CD4⁺ T cells that acquire CD25 expression outside of the thymus in the periphery (e.g., mucosa-associated lymphoid tissue (MALT)), express low levels of CD45RB and do not natively express Foxp3 or CD25. Induced Tregs acquire Foxp3, CD25, CTLA-4, and GITR/AITR expression based on the influence of TGFbeta on CD4⁺ naïve conventional T cells in the periphery. Tregs achieve this suppression, at least in part, by inhibiting the proliferation, expansion, and effector activity of conventional T cells (Tcons). Tregs suppress effector T cells from destroying their (self-)target, either through cell-cell contact by inhibiting T cell help and activation, through release of immunosuppressive cytokines such as IL-10 or TGF-β, through production of cytotoxic molecules such as Granzyme B, through depleting IL-2 levels, or by changing nutrients in tissues. Depletion of Tregs was shown to enhance IL-2 induced anti-tumor immunity (Imai et al. (2007) Cancer Sci. 98:416-23).

By contrast, conventional T cells, also known as Tconv or Teffs, have effector functions (e.g., cytokine secretion, cytotoxic activity, anti-self-recognition, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4⁺ Teffs or CD8⁺ Teffs, such as CD4⁺ helper T lymphocytes (e.g., Th0, Th1, Tfh, or Th17) and CD8+ cytotoxic T lymphocytes.

“Naïve Tcons” are CD4⁺ T cells or CD8⁺ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers, such as CD25, CD44 or CD69, and absence of memory markers, such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses.

Unlike Tregs, “effector Tcons” are not anergic and can proliferate in response to antigen-based T cell receptor activation (Lechler et al. (2001) Philos. Trans. R. Soc. Lond Biol. Sci. 356:625-637). Effector Tcons can be CD4⁺ or CD8⁺ T cells. They recognize antigens associated with MHC class I or II molecules, respectively, generally express activation markers, such as CD25, CD44 or CD69, but generally do not express memory markers, such as CD45RO. Generally, increasing the number of Tregs, increasing Treg activity, and/or decreasing Treg cell death (e.g., apoptosis) is useful for suppressing unwanted immune reactions associated with a range of immune disorders (e.g., cGVHD). Tregs are also important in suppressing inflammation as well. In the context of ongoing inflammation, treatments can preferentially enhance Tregs without activating Tcons or other effectors that may worsen GVHD. Effective augmentation of Tregs in vivo is also directly relevant to other disorders of impaired peripheral tolerance (e.g., autoimmune diseases like SLE, T1D, MS, psoriasis, RA, IBD, vasculitis), where Treg dysfunction is increasingly implicated (Grinberg-Bleyer et al. (2010) J. Exp. Med 207:1871-1878; Buckner (2010) Nat. Rev. Immunol. 10:849-859; Humrich et al. (2010) Proc. Natl. Acad Sci. U.S.A. 107:204-209; Carbone et al. (2014) Nat. Med. 20:69-74).

“Memory Tcons” are antigen-experienced T cells (i.e., T cells that have previously been exposed to and responded to an antigen) represented by at least three distinct subpopulations of T cells. Memory Tcons can reproduce quickly and elicit a stronger immune response when re-exposed to the antigen. Memory Tcons subpopulationcs can be differentiated based on the differential expression of the chemokine receptor, CCR7, and L-selection (CD62L) (Sallusto et al. (2000) Curr. Top. Microbiol. Immunol. 251:167-171). For example, stem memory T cells (Tscm), like naïve cells, are CD45RO−, CCR7+, CD45RA+, CD62L+(L-selectin), CD27+, CD28+, and IL-7Rα+, but they also express large amounts of CD95, IL-2Rβ, CXCR3, and LFA-1, and show numerous functional attributes distinctive of memory cells (Gattinoni et al. (2011) Nat. Med 17:1290-1297). Central memory cells (Tcm) express L-selectin and the CCR7 and secrete IL-2, but not IFNγ or IL-4. Effector memory cells (Tem) do not express L-selectin or CCR7, but produce effector cytokines like IFNγ and IL-4.

“Exhausted Tcons” are T cells that have progressively lost T-cell function. “Exhaustion” or “unresponsiveness” refers to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation or cell division, entrance into the cell cycle, cytokine production, cytotoxicity, trafficking, phagocytotic activity, or any combination thereof. Normal input signals can include, but are not limited to, stimulation via a receptor (e.g., T cell receptor, B cell receptor, co-stimulatory receptor, and the like).

Exhausted immune cells can have a reduction of at least 10%, 15%, 20%, 25%, 30%, 35%, 40%, 45%, 50%, 55%, 60%, 65%, 70%, 75%, 80%, 85%, 90%, 95%, 99% or more in cytotoxic activity, cytokine production, proliferation, trafficking, phagocytotic activity, or any combination thereof, relative to a corresponding control immune cell of the same type. In one embodiment, a cell that is exhausted is a CD8⁺ T cell (e.g., an effector CD8⁺ T cell that is antigen-specific). CD8 cells normally proliferate (e.g., clonally expand) in response to T cell receptor and/or co-stimulatory receptor stimulation, as well as in response to cytokines such as IL-2. Thus, an exhausted CD8 T cell is one which does not proliferate and/or produce cytokines in response to normal input signals. It is well known that the exhaustion of effector functions can be delineated according to several stages, which eventually lead to terminal or full exhaustion and, ultimately, deletion (Yi et al. (2010) Immunol. 129:474-481; Wherry and Ahmed (2004) J. Virol. 78:5535-5545). In the first stage, functional T cells enter a “partial exhaustion r” phase characterized by the loss of a subset of effector functions, including loss of IL-2 production, reduced TNFα production, and reduced capacity for proliferation and/or ex vivo lysis ability. In the second stage, partially exhausted T cells enter a “partial exhaustion II” phase when both IL-2 and TNFα production ceases following antigenic stimulation and IFNγ production is reduced. “Full exhaustion” or “terminal exhaustion” occurs when CD8⁺ T cells lose all effector functions, including the lack of production of IL-2, TNFα, and IFNγ and loss of ex vivo lytic ability and proliferative potential, following antigenic stimulation. A fully exhausted CD8⁺ T cell is one which does not proliferate, does not lyse target cells (cytotoxicity), and/or does not produce appropriate cytokines, such as IL-2, TNFα, or IFNγ, in response to normal input signals. Such lack of effector functions can occur when the antigen load is high and/or CD4 help is low. This hierarchical loss of function is also associated with the expression of co-inhibitor immune receptors, such as PD-1, TIM-3, LAG-3, and the like (Day et al. (2006) Nature 443:350-4; Trautmann et al. (2006) Nat. Med. 12:1198-202; and Urbani et al. (2006) J. Virol. 80:1398-1403). Other molecular markers distinguish the hierarchical stages of immune cell exhaustion, such as high eomesodermin (EOMES) and low TBET expression as a marker of terminally exhausted T cells (Paley et al. (2012) Science 338:1220-1225). Additional markers of exhausted T cells, such as the reduction of Bcl-b and the increased production of BLIMP-1 (Pdrm1).

In certain embodiments, the T cells of interest can be obtained from particular sources. For example, a mammalian animal model of a condition that would be benefit from an increased immune response can be used as the source of T cells of interest. In another example, the immune systems of host subjects can be engineered or otherwise elected to be immunological compatible with transplanted cells. For example, in one embodiment, the subject may be “humanized” in order to be compatible with human cells. The term “immune-system humanized” refers to an animal, such as a mouse, comprising human HSCs and/or cells derived therefrom and human acquired and innate immune cells, survive without being rejected from the host animal, thereby allowing human hematopoiesis and both acquired and innate immunity to be reconstituted in the host animal. Acquired immune cells include T cells and B cells. Innate immune cells include macrophages, granulocytes (basophils, eosinophils, and neutrophils), DCs, NK cells and mast cells. Representative, non-limiting examples include SCID-hu, Hu-PBL-SCID, Hu-SRC-SCID, NSG (NOD-SCID IL2r-gamma(null) lack an innate immune system, B cells, T cells, and cytokine signaling), NOG (NOD-SCID IL2r-gamma(truncated)), BRG (BALB/c-Rag2 (null)IL2r-gamma(null)), and H2dRG (Stock-H2d-Rag2(null)IL2r-gamma(null)) mice (see, for example, Shultz et al. (2007) Nat. Rev. Immunol. 7:118; Pearson et al. (2008) Curr. Protocol. Immunol. 15:21; Brehm et al. (2010) Clin. Immunol. 135:84-98; McCune et al. (1988) Science 241:1632-1639, U.S. Pat. No. 7,960,175, and U.S. Pat. Publ. 2006/0161996), as well as related null mutants of immune-related genes like Rag1 (lack B and T cells), Rag2 (lack B and T cells), TCR alpha (lack T cells), perforin (cD8+ T cells lack cytotoxic function), FoxP3 (lack functional CD4⁺ T regulatory cells), IL2rg, or Prfl, as well as mutants or knockouts of PD-1, PD-L1, Tim3, and/or 2B4, allow for efficient engraftment of human immune cells in and/or provide compartment-specific models of immunocompromised animals like mice (see, for example, PCT Publ. WO2013/062134). In addition, NSG-CD34+ (NOD-SCID IL2r-gamma(null) CD34+) humanized mice are useful for studying human gene and tumor activity in animal models like mice.

Well-known immune cell characteristics can be used to purify, enrich, and/or isolate T cells of interest. “Enriched T cells” refer to a composition comprising a desired T cell population (e.g., engineered T cells encompassed by the present invention) to other cells and/or T cells in a proportion where the composition has at least a 1:2, 1:1.9, 1:1.8, 1:1.7, 1:1.6, 1:1.5, 1:1.4, 1:1.3, 1:1.2, 1:1.1, 1:1, 1:0.9, 1:0.8, 1:0.7, 1:0.6, 1:0.5, 1:0.4, 1:0.3, 1:0.2, 1:0.1, or more, or any range in between or any value in between, ratio of desired T cells to other cells. Such ratios can be achieved by purifying a composition comprising T cells with various methodologies. For example, purification of Tregs can be performed using CD8⁺ and CD19+ co-depletion in combination with positive selection for CD25+ cells. Such enriched Tregs can further be defined in terms of cell markers and/or viability. For example, an enriched Tregs cell composition can have greater than 40%, 45%, 50%, 55%, 60%, 65%, 70%, 75%, 80%, 85%, 90%, 95%, 99%, or more, or any range in between or any value in between, total cell viability. It can comprise greater than 40%, 45%, 50%, 55%, 60%, 65%, 70%, 75%, 80%, 85%, 90%, 95%, 99%, or more, or any range in between or any value in between, CD4+CD25+ cells. It can comprise greater than 40%, 45%, 50%, 55%, 60%, 65%, 70%, 75%, 80%6, 85%, 90%, 95%, 99%, or more, or any range in between or any value in between, FoxP3+ cells. Tregs can be administered in any suitable route as described herein, such as by infusion. Tregs can also be administered before, concurrently with, or after, other immunomodulatory agents. Such methodologies and metrics can be adapted to any T cell population of interest using well-known methods in the art.

Cells of interest can be genetically modified according to the present invention, wherein the genome of a cell can be engineered to inhibit the copy number, the expression level, and/or the activity of PTPN2. In one embodiment, the genetic modification is a deletion of all or a portion of PTPN2 gene locus or an enhancer genomic region for PTPN2 gene. The deletion can be a per se deletion or an effective deletion by inserting a sequence not present in PTPN2 gene locus or an enhancer genomic region for PTPN2 gene prior to genetic modification. Deletion of an enhancer genomic region will reduce transcription of the gene of interest.

Genome editing methods are well-known in the art. For example, targeted or untargeted gene knockout methods can be used to recombinantly engineer cells of hematopoietic stem cell lineage ex vivo prior to infusion into the subject. For example, the target DNA in the genome can be manipulated by deletion, insertion, and/or mutation using retroviral insertion, artificial chromosome techniques, gene insertion, random insertion with tissue specific promoters, gene targeting, transposable elements and/or any other method for introducing foreign DNA or producing modified DNA/modified nuclear DNA.

Other modification techniques include deleting DNA sequences from a genome and/or altering nuclear DNA sequences. Nuclear DNA sequences, for example, may be altered by site-directed mutagenesis using homologous recombination. Such methods generally use host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. Vector DNA can be introduced into prokaryotic or eukaryotic cells via conventional transformation or transfection techniques.

As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook et al. (supra), and other laboratory manuals. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

Similarly, the CRISPR-Cas system can be used for precise editing of genomic nucleic acids (e.g., for creating non-functional or null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

Modulation of gene expression of at least one gene of interest, as well as T cell function, can be determined according to well-known methods in the art and as exemplified in the Examples. For example, T cell activity, proliferation, apoptosis, cytokine production repertoire, cell surface marker expression, and the like can be analyzed. Moreover, phenotypic analyses of lymphocyte subsets, functional assays of immunomodulation leading to reduced immune responses, plasma cytokines, and the like can be analyzed as described further herein. In particular, methods for determining the results of the methods described herein, such as modulation of immune responses, metastasis, disease remission, disease relapse, tumor recurrence, death, autoimmunity, allergy (e.g., asthma, atopic dermatitis, allergic conjunctivitis, pollen allergy, food allergy, etc.), vaccination response, immune tolerance, immune exhaustion, immunological memory, immunological epitope responses, cytokine responses, relative representation of cells, genetic perturbations, and/or other immunologic effects are well-known in the art and as described herein. For example, determination of target nucleic acid gene expression and/or sequences of interest can be performed using variety of sequencing methods known in the art. In preferred embodiments, a particular genetic perturbation is characterized by a measure of a nucleic acid or product thereof (e.g., mRNA). Marker expression may be monitored in a variety of ways, including by detecting mRNA levels, protein levels, or protein activity, any of which may be measured using standard techniques (see, e.g., Ausubel et al., ed., Current Protocols in Molecular Biology, John Wiley & Sons, New York 1987-1999). Detection may involve quantification of the level of gene expression (e.g., genomic DNA, cDNA, mRNA, protein, or enzyme activity), or, alternatively, may be a qualitative assessment of the level of gene expression, in particular in comparison with a control level. The type of level being detected will be clear from the context. Various amplification and detection methods may also be used. For example, it is within the scope encompassed by the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR), real time PCR, NASBA, ligase chain reaction (Barany, 1991, Proc. Natl. Acad. Sci. USA, 88:189-193), self-sustained sequence replication (Guatelli et al., 1990, Proc. Natl. Acad. Sci. USA 87:1874-1878), transcriptional amplification system (Kwoh et al., 1989, Proc. Natl. Acad Sci. USA 86:1173-1177), Q-Beta Replicase (Lizardi et al., 1988, Bio/Technology 6:1197), rolling circle replication (Lizardi et al., U.S. Pat. No. 5,854,033), target-mediated amplification, self-sustained sequence replication (SSR), transcription amplification, and the like. Many techniques are known in the state of the art for determining absolute and relative levels of gene expression, commonly used techniques suitable for use in the present invention include in situ hybridization, microarray, chip array, serial analysis of gene expression (SAGE), Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In certain embodiments, nucleic acid detection can be accomplished using methods including, but not limited to, sequencing by hybridization (SBH), sequencing by ligation (SBL), quantitative incremental fluorescent nucleotide addition sequencing (QIFNAS), stepwise ligation and cleavage, fluorescence resonance energy transfer (FRET), molecular beacons, TaqMan® reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al. (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Ser. No. 12/120,541, filed May 14, 2008), allele-specific oligo ligation assays (e.g., oligoligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., on cyclic array sequencing using platforms such as Roche 454, Illumina Solexa or MiSeq or HiSeq, AB-SOLiD, Helicos, Polonator platforms and the like, can also be utilized. High-throughput sequencing methods are described in U.S. Ser. No. 61/162,913, filed Mar. 24, 2009. A variety of light-based sequencing technologies are known in the art (Landegren et al. (1998) Genome Res. 8:769-76; Kwok (2000) Pharmocogenom. 1:95-100; and Shi (2001) Clin. Chem. 47:164-172) (see, for example, U.S. Pat. Publ. Nos. 2013/0274117, 2013/0137587, and 2011/0039304).

Similarly, polypeptides and/or cells of interest can be distinguished according to many well-known methods in the art including, but not limited to, flow cytometry, fluorescence activated cell sorting (FACS), fluorescence microscopy, detectable cell barcode technology (U.S. Pat. Publ. 2011/0263457), immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., “Basic and Clinical Immunology,” Sites and Terr, eds., Appleton and Lange, Norwalk, Conn. Pp. 217-262, 1991, which is incorporated by reference). Preferred are binder-ligand immunoassay methods including reacting antibodies with an epitope or epitopes and competitively displacing a labeled polypeptide or derivative thereof.

In addition, T cell function of the engineered T cells can be assessed according to well-known methods in the art as described further in the Examples. For example, engineered T cells can be assessed for “reduced exhaustion” or “reduced unresponsiveness,” which refers to a given treatment or set of conditions that leads to increased T cell activity, responsiveness, and/or ability or receptiveness, with regards to activation. T cell activity can be measured by contacting T cells with recall antigen, anti-CD3 in the absence of costimulation, and/or ionomycin. Also, proliferation of T cells can be measured in the presence of a relevant antigen assayed, e.g. by a ³H-thymidine incorporation assay or cell number. Markers of T cell activation after exposure to the relevant antigen can also be assayed, e.g. flow cytometry analysis of cell surface markers indicative of T cell activation (e.g., CD69, CD30, CD25, and HLA-DR) and/or T cell exhaustion. In some embodiments, the assays can be in vivo assays, such as through challenging immune cells with antigen in vivo. For example, animal models expressing homogeneous populations of T cells from TCR transgenic and other transgenic mice can be transferred into hosts that constitutively express an antigen recognized by the transferred T cells, e.g., the H-Y antigen TCR transgenic; pigeon cytochrome C antigen TCR transgenic; or hemagglutinin (HA) TCR transgenic. In such models, T cells expressing the TCR specific for the antigen constitutively or inducibly expressed by the recipient mice typically undergo an immediate expansion and proliferative phase, followed by a period of unresponsiveness, which is reversed when the antigen is removed and/or antigen expression is inhibited. Accordingly, if the T cells proliferate or expand, show cytokine activity, etc. significantly more in an assay (e.g., with or without additional treatment of immunomodulatory agents) than control T cells, then T cell exhaustion is reduced. Such measurements of proliferation can occur in vivo using T cells labeled with BrDU, CFSE or another intravital dye that allows tracking of proliferation prior to transferring to a recipient animal expressing the antigen, or cytokine reporter T cells, or using ex vivo methods to analyze cellular proliferation and/or cytokine production, such as thymidine proliferation assays, ELISA, cytokine bead assays, and the like. Moreover, reduction of immune cell exhaustion can be assessed by examination of tumor infiltrating lymphocytes or T lymphocytes within lymph nodes that drain from an established tumor. Such T cells exhibit features of exhaustion through expression of cell surface molecules, such as immunoinhibitory receptors described above, for example, and decreased secretion of cytokines, such as those described above. Accordingly, if increased quantities and/or activities of T cells are observed with, for example, 1) antigen specificity for tumor associated antigens (e.g., as determined by major histocompatibility complex class I or class II tetramers which contain tumor associated peptides) and/or 2) that are capable of secreting high levels of appropriate cytokines and cytolytic effector molecules such as granzyme-B, then T cell exhaustion has been reduced.

### IV. Methods of Selecting Agents that Upregulate Immune Responses

Another aspect encompassed by the present invention relates to methods of selecting agents (e.g., antibodies, fusion proteins, peptides, or small molecules) which modulate an immune response by inhibit the copy number, the expression, and/or the activity of PTPN2. Such methods utilize screening assays, including cell based and non-cell based assays. In one embodiment, the assays provide a method for identifying agents that inhibit the phosphatase activity and/or the substrate binding activity of PTPN2.

In one embodiment, the present invention relates to assays for screening test agents which bind to, or modulate the biological activity of, at least one biomarker described herein (e.g., in the tables, figures, examples, or otherwise in the specification). In one embodiment, a method for identifying such an agent entails determining the ability of the agent to modulate, e.g. inhibit, the at least one biomarker described herein.

In one embodiment, an assay is a cell-free or cell-based assay, comprising contacting at least one biomarker described herein, with a test agent, and determining the ability of the test agent to modulate (e.g., inhibit) the enzymatic activity of the biomarker, such as by measuring direct binding of substrates or by measuring indirect parameters as described below.

For example, in a direct binding assay, biomarker protein (or their respective target polypeptides or molecules) can be coupled with a radioisotope or enzymatic label such that binding can be determined by detecting the labeled protein or molecule in a complex. For example, the targets can be labeled with ¹²⁵I, ³⁵S, ¹⁴C, or ³H, either directly or indirectly, and the radioisotope detected by direct counting of radioemmission or by scintillation counting. Alternatively, the targets can be enzymatically labeled with, for example, horseradish peroxidase, alkaline phosphatase, or luciferase, and the enzymatic label detected by determination of conversion of an appropriate substrate to product. Determining the interaction between biomarker and substrate can also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target can be accomplished in any vessel suitable for containing the reactants. Non-limiting examples of such vessels include microtiter plates, test tubes, and micro-centrifuge tubes. Immobilized forms of the antibodies described herein can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In an alternative embodiment, determining the ability of the agent to modulate the interaction between the biomarker and a substrate or a biomarker and its natural binding partner can be accomplished by determining the ability of the test agent to modulate the activity of a polypeptide or other product that functions downstream or upstream of its position within the signaling pathway (e.g., feedback loops). Such feedback loops are well-known in the art (see, for example, Chen and Guillemin (2009) Int. J. Tryptophan Res. 2:1-19).

The present invention further pertains to novel agents identified by the above-described screening assays. Accordingly, it is within the scope of this invention to further use an agent identified as described herein, such as in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

### V. Pharmaceutical Compositions

A agents that modulate (e.g., inhibit) the copy number, the expression level, and/or the activity of PTPN2, including, e.g., blocking antibodies, peptides, fusion proteins, or small molecules, can be incorporated into pharmaceutical compositions suitable for administration to a subject. Such compositions typically comprise the antibody, peptide, fusion protein or small molecule and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary active compounds can also be incorporated into the compositions.

A pharmaceutical composition encompassed by the present invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, modulatory agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms encompassed by the present invention are dictated by, and directly dependent on, the unique characteristics of the active compound, the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method encompassed by the present invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The above described modulating agents may be administered in the form of expressible nucleic acids which encode said agents. Such nucleic acids and compositions in which they are contained, are also encompassed by the present invention. For instance, the nucleic acid molecules encompassed by the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### VI. Uses and Methods of the Invention

The modulatory agents described herein can be used according to a number of methods related to the inhibition of the copy number, the expression level, and/or the activity of PTPN2, and corresponding upregulation of immune responses.

1. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect encompassed by the present invention relates to diagnostic assays for determining the amount and/or activity level of a biomarker described herein in the context of a biological sample (e.g., blood, serum, cells, or tissue) to thereby determine whether an individual afflicted with a condition that would benefit from an increased immune response is likely to respond to inhibitors of PTPN2, such as in a cancer. Such assays can be used for prognostic or predictive purpose alone, or can be coupled with a therapeutic intervention to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers described herein, such as those in the tables, figures, examples, and otherwise described in the specification.

Another aspect encompassed by the present invention pertains to monitoring the influence of agents (e.g., drugs, compounds, and small nucleic acid-based molecules) on the expression or activity of a biomarker described herein. These and other agents are described in further detail in the following sections.

The skilled artisan will also appreciated that, in certain embodiments, the methods encompassed by the present invention implement a computer program and computer system. For example, a computer program can be used to perform the algorithms described herein. A computer system can also store and manipulate data generated by the methods encompassed by the present invention which comprises a plurality of biomarker signal changes/profiles which can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from cancerous or pre-cancerous tissue. In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part encompassed by the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods encompassed by the present invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

In certain embodiments, the computer comprises a database for storage of biomarker data. Such stored profiles can be accessed and used to perform comparisons of interest at a later point in time. For example, biomarker expression profiles of a sample derived from the non-cancerous tissue of a subject and/or profiles generated from population-based distributions of informative loci of interest in relevant populations of the same species can be stored and later compared to that of a sample derived from the cancerous tissue of the subject or tissue suspected of being cancerous of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

2. Diagnostic Assays

The present invention provides, in part, methods, systems, and code for accurately classifying whether a biological sample is associated with a condition that would benefit from an increased immune response that is likely to respond to inhibitors of PTPN2. In some embodiments, the present invention is useful for classifying a sample (e.g., from a subject) as associated with or at risk for a condition that would benefit from an increased immune response responding to or not responding to such inhibitor using a statistical algorithm and/or empirical data (e.g., the amount or activity of a biomarker described herein, such as in the tables, figures, examples, and otherwise described in the specification).

An exemplary method for detecting the amount or activity of a biomarker described herein, and thus useful for classifying whether a sample is likely or unlikely to respond to inhibitors of PTPN2 involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample as, for example, a likely immunotherapy responder or progressor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method encompassed by the present invention further comprises sending the sample classification results to a clinician, e.g., an oncologist.

In another embodiment, the diagnosis of a subject is followed by administering to the individual a therapeutically effective amount of a defined treatment based upon the diagnosis.

In one embodiment, the methods further involve obtaining a control biological sample (e.g., biological sample from a subject who does not have a condition that would benefit from an increased immune response or whose condition is susceptible to inhibitors of PTPN2), a biological sample from the subject during remission, or a biological sample from the subject during treatment for developing a condition that would benefit from an increased immune response progressing despite such inhibitors.

3. Prognostic Assays

The diagnostic methods described herein can furthermore be utilized to identify subjects having or at risk of developing a condition that would benefit from an increased immune response (e.g., cancer or viral infection) that is likely or unlikely to be responsive to inhibitors of PTPN2. The assays described herein, such as the preceding diagnostic assays or the following assays, can be utilized to identify a subject having or at risk of developing a disorder associated with a misregulation of the amount or activity of at least one biomarker described herein, such as in cancer. Alternatively, the prognostic assays can be utilized to identify a subject having or at risk for developing a disorder associated with a misregulation of the at least one biomarker described herein, such as in cancer. Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with the aberrant biomarker expression or activity.

4. Prophylactic Methods

In one aspect, the present invention provides a method for preventing in a subject, a disease or condition associated with less than desirable immune response. Subjects at risk for a disease that would benefit from treatment with the claimed agents or methods can be identified, for example, by any or a combination of diagnostic or prognostic assays known in the art. Administration of a prophylactic agent can occur prior to the manifestation of symptoms associated with less than desirable immune response. The appropriate agent used for treatment (e.g. antibodies, peptides, fusion proteins or small molecules) can be determined based on clinical indications and can be identified, e.g., using screening assays described herein.

5. Therapeutic Methods

Another aspect encompassed by the present invention pertains to therapeutic methods of upregulating an immune response, e.g., by inhibiting the copy number, the expression level, and/or the activity of PTPN2. The therapeutic compositions described herein, such as the combination of inhibitors of PTPN2, can be used in a variety of in vitro and in vivo therapeutic applications using the formulations and/or combinations described herein. In one embodiment, the therapeutic agents can be used to treat cancers determined to be responsive thereto. For example, single or multiple agents that inhibit or block both such inhibitors and a immunotherapy can be used to treat cancers in subjects identified as likely responders thereto.

Modulatory methods encompassed by the present invention involve contacting a cell, such as an immune cell with an agent that inhibits or blocks the expression and/or activity of PTPN2. Exemplary agents useful in such methods are described above. Such agents can be administered in vitro or ex vivo (e.g., by contacting the cell with the agent) or, alternatively, in vivo (e.g., by administering the agent to a subject). As such, the present invention provides methods useful for treating an individual afflicted with a condition that would benefit from an increased immune response, such as a viral infection or a cancer.

Agents that upregulate immune responses can be in the form of enhancing an existing immune response or eliciting an initial immune response. Thus, enhancing an immune response using the subject compositions and methods is useful for treating cancer, but can also be useful for treating an infectious disease (e.g., bacteria, viruses, or parasites), a parasitic infection, and an immunosuppressive disease.

Exemplary infectious disorders include viral skin diseases, such as Herpes or shingles, in which case such an agent can be delivered topically to the skin. In addition, systemic viral diseases, such as influenza, the common cold, and encephalitis might be alleviated by systemic administration of such agents. In one preferred embodiment, agents that upregulate the immune response described herein are useful for modulating the arginase/iNOS balance during Trypanosoma cruzi infection in order to facilitate a protective immune response against the parasite.

Immune responses can also be enhanced in an infected patient through an ex vivo approach, for instance, by removing immune cells from the patient, contacting immune cells in vitro with an agent described herein and reintroducing the in vitro stimulated immune cells into the patient.

In certain instances, it may be desirable to further administer other agents that upregulate immune responses, for example, forms of other B7 family members that transduce signals via costimulatory receptors, in order to further augment the immune response. Such additional agents and therapies are described further below.

Agents that upregulate an immune response can be used prophylactically in vaccines against various polypeptides (e.g., polypeptides derived from pathogens). Immunity against a pathogen (e.g., a virus) can be induced by vaccinating with a viral protein along with an agent that upregulates an immune response, in an appropriate adjuvant.

In another embodiment, upregulation or enhancement of an immune response function, as described herein, is useful in the induction of tumor immunity.

In another embodiment, the immune response can be stimulated by the methods described herein, such that preexisting tolerance, clonal deletion, and/or exhaustion (e.g., T cell exhaustion) is overcome. For example, immune responses against antigens to which a subject cannot mount a significant immune response, e.g., to an autologous antigen, such as a tumor specific antigens can be induced by administering appropriate agents described herein that upregulate the immune response. In one embodiment, an autologous antigen, such as a tumor-specific antigen, can be coadministered. In another embodiment, the subject agents can be used as adjuvants to boost responses to foreign antigens in the process of active immunization.

In one embodiment, immune cells are obtained from a subject and cultured ex vivo in the presence of an agent as described herein, to expand the population of immune cells and/or to enhance immune cell activation. In a further embodiment the immune cells are then administered to a subject. Immune cells can be stimulated in vitro by, for example, providing to the immune cells a primary activation signal and a costimulatory signal, as is known in the art. Various agents can also be used to costimulate proliferation of immune cells. In one embodiment immune cells are cultured ex vivo according to the method described in PCT Application No. WO 94/29436. The costimulatory polypeptide can be soluble, attached to a cell membrane, or attached to a solid surface, such as a bead.

The therapeutic agents encompassed by the present invention can be used alone or can be administered in combination therapy with, e.g., chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, agents encompassed by the present invention can be administered with a therapeutically effective dose of chemotherapeutic agent. In another embodiment, agents encompassed by the present invention are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, agents and therapies other than immunotherapy or in combination thereof can be used with in combination with inhibitors of PTPN2 to stimulate an immune response to thereby treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E et al. (2005) Nature 434:913-917; Farmer H et al. (2005) Nature 434:917-921). The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, surgical intervention can occur to physically remove cancerous cells and/or tissues.

In still another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In yet another embodiment, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early non-small cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser. This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light can be directed to parts of the body without making a large incision. More procedures may be done on an outpatient basis. Lasers can be used in two ways to treat cancer: by shrinking or destroying a tumor with heat, or by activating a chemical-known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

The duration and/or dose of treatment with therapies may vary according to the particular therapeutic agent or combination thereof. An appropriate treatment time for a particular cancer therapeutic agent will be appreciated by the skilled artisan. The present invention contemplates the continued assessment of optimal treatment schedules for each cancer therapeutic agent, where the phenotype of the cancer of the subject as determined by the methods encompassed by the present invention is a factor in determining optimal treatment doses and schedules.

6. Upregulation of Immune Responses by Inhibiting the Copy Number, the Expression Level, and/or the Activity of PTPN2

Agents described herein can also be used to upregulate immune responses. In one embodiment, inhibiting the copy number, the expression level, and/or the activity of PTPN2, results in upregulation of an immune response. Upregulation of immune responses can be in the form of enhancing an existing immune response or eliciting an initial immune response. For instance, enhancing an immune response using the subject compositions and methods is useful in treating cancer, an infectious disease (e.g., bacteria, viruses, or parasites), a parasitic infection, asthma associated with impaired airway tolerance, a neurological disease, and an immunosuppressive disease.

Exemplary infectious disorders include viral skin diseases, such as Herpes or shingles, in which case such an agent can be delivered topically to the skin. In addition, systemic viral diseases, such as influenza, the common cold, and encephalitis might be alleviated by systemic administration of such agents. In one preferred embodiment, agents upregulate CD8⁺ T cell immune response against chronic viral infection.

Alternatively, immune responses can be enhanced in an infected patient through an ex vivo approach, for instance, by removing immune cells from the patient, contacting immune cells in vitro with an agent that inhibits the copy number, the expression level, and/or the activity of PTPN2, and reintroducing the in vitro stimulated immune cells into the patient.

In certain instances, it may be desirable to further administer other agents that upregulate immune responses, in order to further augment the immune response.

Agents that upregulate an immune response can be used prophylactically in vaccines against various polypeptides (e.g., polypeptides derived from pathogens). Immunity against a pathogen (e.g., a virus) can be induced by vaccinating with a viral protein along with an agent that upregulates an immune response, in an appropriate adjuvant.

In another embodiment, upregulation or enhancement of an immune response function, as described herein, is useful in the induction of tumor immunity.

In another embodiment, the immune response can be stimulated by the methods described herein, such that pre-existing exhaustion (e.g., T cell exhaustion) is overcome. For example, immune responses against antigens to which a subject cannot mount a significant immune response, e.g., to an autologous antigen, such as a tumor specific antigens can be induced by administering appropriate agents described herein that upregulate the immune response. In one embodiment, an autologous antigen, such as a tumor-specific antigen, can be coadministered. In another embodiment, an immune response can be stimulated against an antigen (e.g., an autologous antigen) to treat a neurological disorder. In another embodiment, the subject agents can be used as adjuvants to boost responses to foreign antigens in the process of active immunization.

In one embodiment, immune cells are obtained from a subject and cultured ex vivo in the presence of an agent as described herein, to expand the population of immune cells and/or to enhance immune cell activation. In a further embodiment the immune cells are then administered to a subject. Immune cells can be stimulated in vitro by, for example, providing to the immune cells a primary activation signal and a costimulatory signal, as is known in the art. Various agents can also be used to costimulate proliferation of immune cells. In one embodiment immune cells are cultured ex vivo according to the method described in PCT Application No. WO 94/29436. The costimulatory polypeptide can be soluble, attached to a cell membrane, or attached to a solid surface, such as a bead.

In still another embodiment, agents described herein useful for upregulating immune responses can further be linked, or operatively attached, to toxins using techniques that are known in the art, e.g., crosslinking or via recombinant DNA techniques. Such agents can result in cellular destruction of desired cells. In one embodiment, a toxin can be conjugated to an antibody, such as a bispecific antibody. Such antibodies are useful for targeting a specific cell population, e.g., using a marker found only on a certain type of cell, e.g., a cell expressing PTPN2. The preparation of immunotoxins is, in general, well-known in the art (see, e.g., U.S. Pat. No. 4,340,535, and EP 44167). Numerous types of disulfide-bond containing linkers are known which can successfully be employed to conjugate the toxin moiety with a polypeptide. In one embodiment, linkers that contain a disulfide bond that is sterically “hindered” are preferred, due to their greater stability in vivo, thus preventing release of the toxin moiety prior to binding at the site of action. A wide variety of toxins are known that may be conjugated to polypeptides or antibodies of the invention. Examples include: numerous useful plant-, fungus- or even bacteria-derived toxins, which, by way of example, include various A chain toxins, particularly ricin A chain, ribosome inactivating proteins such as saporin or gelonin, α-sarcin, aspergillin, restrictocin, ribonucleases, such as placental ribonuclease, angiogenic, diphtheria toxin, and Pseudomonas exotoxin, etc. A preferred toxin moiety for use in connection with the invention is toxin A chain which has been treated to modify or remove carbohydrate residues, deglycosylated A chain. (U.S. Pat. No. 5,776,427). Infusion of one or a combination of such cytotoxic agents, (e.g., ricin fusions) into a patient may result in the death of immune cells.

### VII. Administration of Agents

The immune modulating agents encompassed by the present invention are administered to subjects in a biologically compatible form suitable for pharmaceutical administration in vivo, to either enhance or suppress immune cell mediated immune responses. By “biologically compatible form suitable for administration in vivo” is meant a form of the protein to be administered in which any toxic effects are outweighed by the therapeutic effects of the protein. The term “subject” is intended to include living organisms in which an immune response can be elicited, e.g., mammals. Examples of subjects include humans, dogs, cats, mice, rats, and transgenic species thereof. Administration of an agent as described herein can be in any pharmacological form including a therapeutically active amount of an agent alone or in combination with a pharmaceutically acceptable carrier.

Administration of a therapeutically active amount of the therapeutic composition encompassed by the present invention is defined as an amount effective, at dosages and for periods of time necessary, to achieve the desired result. For example, a therapeutically active amount of an agent may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

The agents or the invention described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of agents, by other than parenteral administration, it may be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, nonionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

As described in detail below, the pharmaceutical compositions encompassed by the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “therapeutically-effective amount” as used herein means that amount of an agent that modulates (e.g., inhibits) biomarker expression and/or activity, or expression and/or activity of the complex, or composition comprising an agent that modulates (e.g., inhibits) biomarker expression and/or activity, or expression and/or activity of the complex, which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity, or expression and/or activity of the complex encompassed by the present invention. These salts can be prepared in situ during the final isolation and purification of the therapeutic agents, or by separately reacting a purified therapeutic agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods encompassed by the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression and/or activity, or expression and/or activity of the complex. These salts can likewise be prepared in situ during the final isolation and purification of the therapeutic agents, or by separately reacting the purified therapeutic agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods encompassed by the present invention include those suitable for oral, nasal, topical (including buccal and sublingual), rectal, vaginal, aerosol and/or parenteral administration. The formulations may conveniently be presented in unit dosage form and may be prepared by any methods well-known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a therapeutic agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a therapeutic agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well-known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more therapeutic agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) biomarker expression and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a therapeutic agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits) biomarker expression and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a therapeutic agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more therapeutic agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions encompassed by the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the therapeutic agents encompassed by the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods encompassed by the present invention so as to obtain an amount of the active ingredient, which is effective to achieve the desired therapeutic response for a particular subject, composition, and mode of administration, without being toxic to the subject.

The nucleic acid molecules encompassed by the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The agent may also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions of agents suitable for injectable use include sterile aqueous solutions (where water soluble) or dispersions and sterile powders for the extemporaneous preparation of sterile injectable solutions or dispersion. In all cases the composition will preferably be sterile and must be fluid to the extent that easy syringeability exists. It will preferably be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating an agent encompassed by the present invention (e.g., an antibody, peptide, fusion protein or small molecule) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the agent plus any additional desired ingredient from a previously sterile-filtered solution thereof.

When the agent is suitably protected, as described above, the protein can be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well-known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. “Dosage unit form”, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms encompassed by the present invention are dictated by, and directly dependent on, (a) the unique characteristics of the active compound and the particular therapeutic effect to be achieved, and (b) the limitations inherent in the art of compounding such an active compound for the treatment of sensitivity in individuals.

In one embodiment, an agent encompassed by the present invention is an antibody. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays.

As described above, in some embodiments, agents for administration are cell-based. Cell-based agents have an immunocompatibility relationship to a subject host and any such relationship is contemplated for use according to the present invention. For example, the cells, such as adoptive T cells, can be syngeneic. The term “syngeneic” can refer to the state of deriving from, originating in, or being members of the same species that are genetically identical, particularly with respect to antigens or immunological reactions. These include identical twins having matching MHC types. Thus, a “syngeneic transplant” refers to transfer of cells from a donor to a recipient who is genetically identical to the donor or is sufficiently immunologically compatible as to allow for transplantation without an undesired adverse immunogenic response (e.g., such as one that would work against interpretation of immunological screen results described herein).

A syngeneic transplant can be “autologous” if the transferred cells are obtained from and transplanted to the same subject. An “autologous transplant” refers to the harvesting and reinfusion or transplant of a subject's own cells or organs. Exclusive or supplemental use of autologous cells may eliminate or reduce many adverse effects of administration of the cells back to the host, particular graft versus host reaction.

A syngeneic transplant can be “matched allogeneic” if the transferred cells are obtained from and transplanted to different members of the same species yet have sufficiently matched major histocompatibility complex (MHC) antigens to avoid an adverse immunogenic response. Determining the degree of MHC mismatch may be accomplished according to standard tests known and used in the art. For instance, there are at least six major categories of MHC genes in humans, identified as being important in transplant biology. HLA-A, HLA-B, HLA-C encode the HLA class I proteins while HLA-DR, HLA-DQ, and HLA-DP encode the HLA class II proteins. Genes within each of these groups are highly polymorphic, as reflected in the numerous HLA alleles or variants found in the human population, and differences in these groups between individuals is associated with the strength of the immune response against transplanted cells. Standard methods for determining the degree of MHC match examine alleles within HLA-B and HLA-DR, or HLA-A, HLA-B and HLA-DR groups. Thus, tests may be made of at least 4, and even 5 or 6 MHC antigens within the two or three HLA groups, respectively. In serological MHC tests, antibodies directed against each HLA antigen type are reacted with cells from one subject (e.g., donor) to determine the presence or absence of certain MHC antigens that react with the antibodies. This is compared to the reactivity profile of the other subject (e.g., recipient). Reaction of the antibody with an MHC antigen is typically determined by incubating the antibody with cells, and then adding complement to induce cell lysis (i.e., lymphocytotoxicity testing). The reaction is examined and graded according to the amount of cells lysed in the reaction (see, for example, Mickelson and Petersdorf (1999) Hematopoietic Cell Transplantation, Thomas, E. D. et al. eds., pg 28-37, Blackwell Scientific, Malden, Mass.). Other cell-based assays include flow cytometry using labeled antibodies or enzyme linked immunoassays (ELISA). Molecular methods for determining MHC type are well-known and generally employ synthetic probes and/or primers to detect specific gene sequences that encode the HLA protein. Synthetic oligonucleotides may be used as hybridization probes to detect restriction fragment length polymorphisms associated with particular HLA types (Vaughn (2002) Method. Mol. Biol. MIC Protocol. 210:45-60). Alternatively, primers may be used for amplifying the HLA sequences (e.g., by polymerase chain reaction or ligation chain reaction), the products of which may be further examined by direct DNA sequencing, restriction fragment polymorphism analysis (RFLP), or hybridization with a series of sequence specific oligonucleotide primers (SSOP) (Petersdorf et al. (1998) Blood 92:3515-3520; Morishima et al. (2002) Blood 99:4200-4206; and Middleton and Williams (2002) Method Mol. Biol. MHC Protocol. 210:67-112).

A syngeneic transplant can be “congenic” if the transferred cells and cells of the subject differ in defined loci, such as a single locus, typically by inbreeding. The term “congenic” refers to deriving from, originating in, or being members of the same species, where the members are genetically identical except for a small genetic region, typically a single genetic locus (i.e., a single gene). A “congenic transplant” refers to transfer of cells or organs from a donor to a recipient, where the recipient is genetically identical to the donor except for a single genetic locus. For example, CD45 exists in several allelic forms and congenic mouse lines exist in which the mouse lines differ with respect to whether the CD45.1 or CD45.2 allelic versions are expressed.

By contrast, “mismatched allogeneic” refers to deriving from, originating in, or being members of the same species having non-identical major histocompatibility complex (MHC) antigens (i.e., proteins) as typically determined by standard assays used in the art, such as serological or molecular analysis of a defined number of MHC antigens, sufficient to elicit adverse immunogenic responses. A “partial mismatch” refers to partial match of the MHC antigens tested between members, typically between a donor and recipient. For instance, a “half mismatch” refers to 50% of the MHC antigens tested as showing different MHC antigen type between two members. A “full” or “complete” mismatch refers to all MHC antigens tested as being different between two members.

Similarly, in contrast, “xenogeneic” refers to deriving from, originating in, or being members of different species, e.g., human and rodent, human and swine, human and chimpanzee, etc. A “xenogeneic transplant” refers to transfer of cells or organs from a donor to a recipient where the recipient is a species different from that of the donor.

In addition, cells can be obtained from a single source or a plurality of sources (e.g., a single subject or a plurality of subjects). A plurality refers to at least two (e.g., more than one). In still another embodiment, the non-human mammal is a mouse. The animals from which cell types of interest are obtained may be adult, newborn (e.g., less than 48 hours old), immature, or in utero. Cell types of interest may be primary cancer cells, cancer stem cells, established cancer cell lines, immortalized primary cancer cells, and the like. In certain embodiments, the immune systems of host subjects can be engineered or otherwise elected to be immunological compatible with transplanted cancer cells. For example, in one embodiment, the subject may be “humanized” in order to be compatible with human cancer cells. The term “immune-system humanized” refers to an animal, such as a mouse, comprising human HSCs and/or cells derived therefrom and human acquired and innate immune cells, survive without being rejected from the host animal, thereby allowing human hematopoiesis and both acquired and innate immunity to be reconstituted in the host animal. Acquired immune cells include T cells and B cells. Innate immune cells include macrophages, granulocytes (basophils, eosinophils, neutrophils), DCs, NK cells and mast cells. Representative, non-limiting examples include SCID-hu, Hu-PBL-SCID, Hu-SRC-SCID, NSG (NOD-SCID IL2r-gamma(null) lack an innate immune system, B cells, T cells, and cytokine signaling), NOG (NOD-SCID IL2r-gamma(truncated)), BRG (BALB/c-Rag2(null)IL2r-gamma(null)), and H2dRG (Stock-H2d-Rag2(null)IL2r-gamma(null)) mice (see, for example, Shultz et al. (2007) Nat. Rev. Immunol. 7:118; Pearson et al. (2008) Curr. Protocol. Immunol. 15:21; Brehm et al. (2010) Clin. Immunol. 135:84-98; McCune et al. (1988), Science 241:1632-1639, U.S. Pat. No. 7,960,175, and U.S. Pat. Publ. 2006/0161996), as well as related null mutants of immune-related genes like Rag1 (lack B and T cells), Rag2 (lack B and T cells), TCR alpha (lack T cells), perforin (cD8+ T cells lack cytotoxic function), FoxP3 (lack functional CD4⁺ T regulatory cells), IL2rg, or Prfl, as well as mutants or knockouts of PD-1, PD-L1, Tim3, and/or 2B4, allow for efficient engraftment of human immune cells in and/or provide compartment-specific models of immunocompromised animals like mice (see, for example, PCT Publ. WO2013/062134). In addition, NSG-CD34+ (NOD-SCID IL2r-gamma(null) CD34+) humanized mice are useful for studying human gene and tumor activity in animal models like mice.

As used herein, “obtained” from a biological material source means any conventional method of harvesting or partitioning a source of biological material from a donor. For example, biological material may obtained from a solid tumor, a blood sample, such as a peripheral or cord blood sample, or harvested from another body fluid, such as bone marrow or amniotic fluid. Methods for obtaining such samples are well-known to the artisan. In the present invention, the samples may be fresh (i.e., obtained from a donor without freezing). Moreover, the samples may be further manipulated to remove extraneous or unwanted components prior to expansion. The samples may also be obtained from a preserved stock. For example, in the case of cell lines or fluids, such as peripheral or cord blood, the samples may be withdrawn from a cryogenically or otherwise preserved bank of such cell lines or fluid. Such samples may be obtained from any suitable donor.

The obtained populations of cells may be used directly or frozen for use at a later date. A variety of mediums and protocols for cryopreservation are known in the art. Generally, the freezing medium will comprise DMSO from about 5-10%, 10-90% serum albumin, and 50-90% culture medium. Other additives useful for preserving cells include, by way of example and not limitation, disaccharides such as trehalose (Scheinkonig et al. (2004) Bone Marrow Transplant. 34:531-536), or a plasma volume expander, such as hetastarch (i.e., hydroxyethyl starch). In some embodiments, isotonic buffer solutions, such as phosphate-buffered saline, may be used. An exemplary cryopreservative composition has cell-culture medium with 4% HSA, 7.5% dimethyl sulfoxide (DMSO), and 2% hetastarch. Other compositions and methods for cryopreservation are well-known and described in the art (see, e.g., Broxmeyer et al. (2003) Proc. Natl. Acad. Sci. U.S.A. 100:645-650). Cells are preserved at a final temperature of less than about −135° C.

Cells can be administered at 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, or more, or any range in between or any value in between, cells per kilogram of subject body weight. The number of cells transplanted may be adjusted based on the desired level of engraftiment in a given amount of time. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be transplanted. In some embodiment, transplantation of at least about 0.1×10⁶, 0.5×10⁶, 1.0×10⁶, 2.0×10⁶, 3.0×10⁶, 4.0×10⁶, or 5.0×10⁶ total cells relative to an average size mouse is effective.

Cells can be administered in any suitable route as described herein, such as by infusion. Cells can also be administered before, concurrently with, or after, other anti-cancer agents.

Administration can be accomplished using methods generally known in the art. Agents, including cells, may be introduced to the desired site by direct injection, or by any other means used in the art including, but are not limited to, intravascular, intracerebral, parenteral, intraperitoneal, intravenous, epidural, intraspinal, intrasternal, intra-articular, intra-synovial, intrathecal, intra-arterial, intracardiac, or intramuscular administration. For example, subjects of interest may be engrafted with the transplanted cells by various routes. Such routes include, but are not limited to, intravenous administration, subcutaneous administration, administration to a specific tissue (e.g., focal transplantation), injection into the femur bone marrow cavity, injection into the spleen, administration under the renal capsule of fetal liver, and the like. In certain embodiment, the cancer vaccine encompassed by the present invention is injected to the subject intratumorally or subcutaneously. Cells may be administered in one infusion, or through successive infusions over a defined time period sufficient to generate a desired effect. Exemplary methods for transplantation, engraftment assessment, and marker phenotyping analysis of transplanted cells are well-known in the art (see, for example, Pearson et al. (2008) Curr. Protoc. Immunol. 81:15.21.1-15.21.21; Ito et al. (2002) Blood 100:3175-3182; Traggiai et al. (2004) Science 304:104-107; Ishikawa et al. Blood (2005) 106:1565-1573; Shultz et al. (2005) J. Immunol. 174:6477-6489; and Holyoake et al. (1999) Exp. Hematol. 27:1418-1427).

Two or more cell types can be combined and administered, such as cell-based therapy and adoptive cell transfer of stem cells, cancer vaccines and cell-based therapy, and the like. For example, adoptive cell-based immunotherapies can be combined with the cell-based therapies encompassed by the present invention. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like. The ratio of cancer cells in the cancer vaccine described herein to other cell types can be 1:1, but can modulated in any amount desired (e.g., 1:1, 1.1:1, 1.2:1, 1.3:1, 1.4:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, or greater).

Engraftment of transplanted cells may be assessed by any of various methods, such as, but not limited to, tumor volume, cytokine levels, time of administration, flow cytometric analysis of cells of interest obtained from the subject at one or more time points following transplantation, and the like. For example, a time-based analysis of waiting 1, 2, 3, 4, 5, 6, 7, 8, 9, 10, 11, 12, 13, 14, 15, 16, 17, 18, 19, 20, 21, 22, 23, 24, 25, 26, 27, 28 days or can signal the time for tumor harvesting. Any such metrics are variables that can be adjusted according to well-known parameters in order to determine the effect of the variable on a response to anti-cancer immunotherapy. In addition, the transplanted cells can be co-transplanted with other agents, such as cytokines, extracellular matrices, cell culture supports, and the like.

The present invention is further illustrated by the following examples which should not be construed as limiting. The contents of all references, patents and published patent applications cited throughout this application, as well as the Figures, are incorporated herein by reference.

### VIII. Kits

The present invention also encompasses kits for detecting and/or modulating biomarkers described herein. A kit encompassed by the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

### IX. Methods for Generating Transduced Hematopoietic Stem Cells (HSCs)

1. Viral Vectors and Transduction of HSCs and/or Cells Derived Therefrom

Viral vectors are well known in the art for transducing target cells and incorporating transgenes.

a. Transgenes

By “transgene” is meant any nucleotide sequence, particularly a DNA sequence, that is integrated into one or more chromosomes of a host cell by human intervention, such as by the methods encompassed by the present invention. In one embodiment, a transgene is an “RNA coding region.” In another embodiment the transgene comprises a “gene of interest.” In other embodiments the transgene may be a nucleotide sequence, preferably a DNA sequence, that is used to mark the chromosome where it has integrated or may indicate a position where nucleic acid editing, such as by the CRSPR-CAS system, may occur. In this situation, the transgene does not have to comprise a gene that encodes a protein that may be expressed.

A “gene of interest” is a nucleic acid sequence that encodes a protein or other molecule, such as an RNA or targeting nucleic acid sequence, that is desirable for integration in a host cell. The gene of interest may be a 1, 2, 3, 4, 5, 6, 7, 8, 9, 10, or more genes of interest expressed from the same or different vectors.

Genes of interest are useful for modulating the expression and/or activity of target biomolecules either within the transduced cell or expressed for secretion outside of the transduced cell. Generally, genes of interest may be nucleic acids themselves or encode a polypeptide, a naturally-occurring binding partner of a target of interest, an antibody against a target of interest, a combination of antibodies against a target of interest and antibodies against other immune-related targets, an agonist or antagonist of a target of interest, a peptidomimetic of a target of interest, a peptidomimetic of a target of interest, a small RNA directed against or a mimic of a target of interest, and the like. Such modulators are well known in the art and include, for example, an antisense nucleic acid molecule, RNAi molecule, shRNA, mature miRNA, pre-miRNA, pri-miRNA, miRNA*, anti-miRNA, or a miRNA binding site, or a variant thereof, or other small RNA molecule such as a Piwi RNA, triplex oligonucleotide, ribozyme, coding sequence for a target of interest. Such agents modulate the expression and/or activity of target biomolecules, which includes any decrease in expression or activity of the target biomolecule of at least about 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more as compared to the expression or activity of the target biomolecule which has not been targeted by a modulating agent.

In one embodiment, the gene of interest is useful for overexpressing and/or enhancing the activity of a nucleic acid or protein of interest. For example, the gene of interest may encode a protein or other molecule the expression of which is desired in the host cell. Such protein-encoding nucleic acid sequences are not particularly limited and are selected based on the desired exogenous perturbation desired. Thus, the gene of interest includes any gene that the skilled practitioner desires to have integrated and/or expressed. For example, exogenous expression of proteins related to autoimmune, allergic, vaccination, immunotolerance, cancer immunotherapy, immune exhaustion, immunological memory, or immunological epitope responses may be used. The gene of interest encode a protein or be a nucleic acid that serves as a marker to identify cells of interest or transduced cells. The gene of interest may encode a protein that modifies a physical characteristic of the transduced cell, such as a protein that modifies size, growth, or eventual tissue composition. In another example, the gene of interest may encode a protein of commercial value that may be harvested. Generally, the gene of interest is operatively linked to other sequences that are useful for obtaining the desired expression of the gene of interest, such as transcriptional regulatory sequences like inducible promoters, as described further below.

In one embodiment, the viral vector may be engineered to express the CRISPR-Cas system for precise editing of genomic nucleic acids (e.g., for creating null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PIoS One 6:e19722; Li et al. (2011) Nucl. Acid Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47).

In another embodiment, the gene of interest is useful for inhibiting the expression and/or activity of a nucleic acid or protein of interest. For example, target biomolecule expression and/or activity, such as an RNA coding region, may be reduced or inhibited using inhibitory RNAs. An “RNA coding region” is a nucleic acid that may serve as a template for the synthesis of an RNA molecule, such as an siRNA. “RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see, for example, Coburn and Cullen (2002) J. Virol. 76:9225), thereby inhibiting expression of the target biomarker nucleic acid. In one embodiment, the RNA coding region is a DNA sequence. The ability to down-regulate a target gene has many therapeutic and research applications, including identifying the biological functions of particular genes. Moreover, such inhibition may be achieved in screening assays that take advantage of pooling techniques, whereby groups of 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more, or any number or range in between, of RNA inhibitory agents, either co-expressed from the same vector or more than one vector, are transduced into cells of interest. Suitable inhibitory RNAs include, but are not limited to siRNAs, shRNAs, miRNAs, Piwis, dicer-substrate 27-mer duplexes, single-stranded interfering RNA, and the like. In particular, the combination of RNA inhibitory technology and lentiviruses as a tool for a gene specific knock-down in animal models is well known in the art (see, for example, U.S. Pat. Publ. 2005/0251872; EP Pat. Publ. 2166107; PCT Publs. WO 2004/022722 and 2007/109131; Tiscornia et al. (2003) Proc. Natl. Acad. Sci. U.S.A. 100:1844-1848; Rubinson et al. (2003) Nat. Genet. 33:401-406; and Dann et al. (2006) Proc. Natl. Acad. Sci. U.S.A. 103:11246-11251).

siRNAs typically refer to a double-stranded interfering RNA unless otherwise noted. In various embodiments, suitable siRNA molecules include double-stranded ribonucleic acid molecules comprising two nucleotide strands, each strand having about 19 to about 28 nucleotides (i.e. about 19, 20, 21, 22, 23, 24, 25, 26, 27, or 28 nucleotides). Thus, the phrase “interfering RNA having a length of 19 to 49 nucleotides” when referring to a double-stranded interfering RNA means that the antisense and sense strands independently have a length of about 19 to about 49 nucleotides, including interfering RNA molecules where the sense and antisense strands are connected by a linker molecule.

In addition to siRNA molecules, other interfering RNA molecules and RNA-like molecules may be used. Examples of other interfering RNA molecules that may to inhibit target biomolecules include, but are not limited to, short hairpin RNAs (shRNAs), single-stranded siRNAs, microRNAs (miRNAs), piwiRNA, dicer-substrate 27-mer duplexes, and variants thereof containing one or more chemically modified nucleotides, one or more non-nucleotides, one or more deoxyribonucleotides, and/or one or more non-phosphodiester linkages. Typically, all RNA or RNA-like molecules that may interact with transcripts RISC complexes and participate in RISC-related changes in gene expression may be referred to as “interfering RNAs” or “interfering RNA molecules.”

Suitable interfering RNAs may readily be produced based on the well-known nucleotide sequences of target biomolecules. In various embodiments interfering RNAs that inhibit target biomolecules may comprise partially purified RNA, substantially pure RNA, synthetic RNA, recombinantly produced RNA, as well as altered RNA that differs from naturally-occurring RNA by the addition, deletion, substitution, and/or alteration of one or more nucleotides. Such alterations may include, for example, addition of non-nucleotide material, such as to the end(s) of the interfering RNAs or to one or more internal nucleotides of the interfering RNAs, including modifications that make the interfering RNAs resistant to nuclease digestion. Such alterations result in sequences that are generally at least about 80%, 81%, 82%, 83%, 84%, 85%, 86%, 87%, 88%, 89%, 90%, 91%, 92%, 93%, 94%, 95%, 96%, 97%, 98%, 99%, 99.5%, or more, or 100% identical to the sequence of the target biomolecule. When the gene to be down regulated is in a family of highly conserved genes, the sequence of the duplex region may be chosen with the aid of sequence comparison to target only the desired gene. On the other hand, if there is sufficient identity among a family of homologous genes within an organism, a duplex region may be designed that would down regulate a plurality of genes simultaneously.

In various embodiments one or both strands of the interfering RNAs may comprise a 3′ overhang. As used herein, a “3′ overhang” refers to at least one unpaired nucleotide extending from the 3′-end of an RNA strand. Thus in one embodiment, the interfering RNAs comprises at least one 3′ overhang of from 1 to about 6 nucleotides (which includes ribonucleotides or deoxynucleotides) in length, from 1 to about 5 nucleotides in length, from 1 to about 4 nucleotides in length, or about 2 to about 4 nucleotides in length. In an illustrative embodiment in which both strands of the interfering RNAs molecule comprise a 3′ overhang, wherein the length of the overhangs may be the same or different for each strand. In certain embodiments the 3′ overhang is present on both strands of the interfering RNAs and is one, two, or three nucleotides in length. For example, each strand of the interfering RNAs may comprise 3′ overhangs of dithymidylic acid (“TT”) or diuridylic acid (“uu”).

In order to enhance the stability of the interfering RNAs, the 3′ overhangs may be also stabilized against degradation. In one embodiment, the overhangs are stabilized by including purine nucleotides, such as adenosine or guanosine nucleotides. In certain embodiments, substitution of pyrimidine nucleotides by modified analogues, e.g., substitution of uridine nucleotides in the 3′ overhangs with 2′-deoxythymidine, is tolerated and does not affect the efficiency of RNA interference degradation. In particular, it is believed the absence of a 2′ hydroxyl in the 2′-deoxythymidine may significantly enhance the nuclease resistance of the 3′ overhang.

Interfering RNAs may be expressed from a vector described herein either as two separate, complementary RNA molecules, or as a single RNA molecule with two complementary regions. Selection of vectors suitable for expressing interfering RNAs, methods for inserting nucleic acid sequences for expressing the interfering RNAs into the vector, and methods of delivering the recombinant plasmid to the cells of interest are well known in the art (Tuschl (2002) Nat. Biotechnol. 20: 446-448; Brummelkamp et al. (2002) Science 296:550 553; Miyagishi et al. (2002) Nat. Biotechnol. 20:497-500; Paddison et al. (2002) Genes Dev. 16:948-958; Lee et al. (2002) Nat. Biotechnol. 20:500-505; and Paul et al. (2002) Nat. Biotechnol. 20:505-508).

In certain embodiments, the interfering RNAs may be delivered as a small hairpin RNA or short hairpin RNA (shRNA) (see, for example, U.S. Pat. Nos. 8,697,359 and 8,642,569). shRNA is a sequence of RNA that makes a tight hairpin turn that may be used to silence gene expression via RNA interference. In typical embodiments, shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA that is bound to it.

In certain embodiments, the sense sequence of the shRNA will be from about 19 to about 30, more nucleotides (e.g. about 19, 20, 21, 22, 23, 24, 25, 26, 27, 28, 29, or 30 nucleotides) in length, more typically from about 19 to about 22 nucleotides in length, the antisense sequence will be from about 19 to about 30, more typically from 19 to about 22 nucleotides (e.g. about 19, 20, 21 or 22 nucleotides), in length, and the loop region will be from about 3 to about 19 nucleotides (e.g., about 3, 4, 5, 6, 7, 8, 9, 10, 11, 12, 13, 14, 15, 16, 17, 18, and 19 nucleotides) in length. In some embodiments, the sense and antisense sequences are the same length, i.e. the shRNA will form a symmetrical hairpin, but this is not necessarily the case. In some cases, the sense or antisense strand may be shorter than its complementary strand, and an asymmetric hairpin is formed. Further, while in some instances the base pairing between the sense and antisense sequences is exact, this also need not be the case. Thus, some mismatch between the sequences may be tolerated, or even desired, e.g. to decrease the strength of the hydrogen bonding between the two strands. However, in one illustrative embodiment, the sense and antisense sequences are the same length, and the base pairing between the two is exact and does not contain any mismatches. The shRNA molecule may also comprise a 5′-terminal phosphate group that may be chemically modified. In addition, the loop portion of the shRNA molecule may comprise, for example, nucleotides, non-nucleotides, linker molecules, conjugate molecules, etc.

In certain embodiments, the PIWI RNA pathway is used to provide inhibition of target biomolecules. Piwi-interacting RNAs (piRNAs) were identified through association with Piwi proteins in mammalian testes (Aravin et al. (2006); Girard et al. (2006); Grivna et al. (2006); Lau et al. (2006). piRNAs and methods of making and using same to target and degrade nucleic acids are well known in the art (see, for example, U.S. Pat. Publ. 2011-0207625). These RNAs range from 26-30 nucleotides in length and are produced from discrete loci. Generally, genomic regions spanning 50-100 kB in length give rise to abundant piRNAs with profound strand asymmetry. Although the piRNAs themselves are not conserved, even between closely related species, the positions of piRNA loci in related genomes are conserved, with virtually all major piRNA-producing loci having synthetic counterparts in mice, rats and humans (Girard et al. (2006)). The loci and consequently the piRNAs themselves are relatively depleted of repeat and transposon sequences, with only 17% of human piRNAs corresponding to known repetitive elements as compared to a nearly 50% repeat content for the genome as a whole. In certain embodiments, methods are provided for inhibiting such targets in a cell, comprising administering an effective amount of a siRNA/shRNA/piwiRNA to the cell, such that target mRNA is degraded.

As described below, internal promoters may be engineered into viral vectors in order to allow for the independent expression of more than one gene of interest. If a second or additional gene of interest is included, an internal ribosomal entry site (IRES) sequence may be included (see, for example, U.S. Pat. No. 4,937,190). The IRES sequence may facilitate the expression of the reporter gene and may be used to create multigene, or polycistronic, messages. IRES elements are able to bypass the ribosome scanning model of 5′-methylated cap-dependent translation and begin translation at internal sites (Pelletier and Sonenberg, 1988). IRES elements are well known in the art and be isolated from, for example, at least two members of the picornavirus family (polio and encephalomyocarditis) have been described (Pelletier and Sonenberg, 1988), as well as from a mammalian message (Macejak and Sarnow, 1991). IRES elements may be linked to heterologous open reading frames. Multiple open reading frames may be transcribed together, each separated by an IRES, creating polycistronic messages. By virtue of the IRES element, each open reading frame is accessible to ribosomes for efficient translation. Multiple genes may be efficiently expressed using a single promoter/enhancer to transcribe a single message.

In certain embodiments encompassed by the present invention, cells transduced with the lentivectors encompassed by the present invention may be identified in vitro or in vivo by including a marker in the expression vector. Such markers would confer an identifiable change to the transduced cell permitting easy identification of cells containing the expression vector. For example, a gene of interest encoding a marker protein may be placed after the primary gene of interest that is, for example, an RNA interfering nucleic acid, to allow for identification of cells that are expressing the desired protein.

Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker. Usually the inclusion of a drug selection marker aids in the cloning and identification of transformants, for example, genetic constructs that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

Many useful reporter markers are known and include, for example, a fluorescence marker, preferably selected from green fluorescent protein (GFP), enhanced GFP (eGFP), DsRed, AsRed, HcRed, Tomatoe, Cherry, Katushka, and variants thereof (see, for example, U.S. Pat. Nos. 5,487,932 and 5,464,763). Examples of other useful reporters include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I ³⁵S or ³H.

b. Viral Vectors

In general, viral vectors are plasmid-based or virus-based, and are configured to carry the essential sequences for incorporating foreign nucleic acid, for selection and for transfer of the nucleic acid into a host cell. The viral construct is a nucleotide sequence that comprises sequences necessary for the production of recombinant retrovirus in a packaging cell. In one embodiment, the viral construct additionally comprises genetic elements that allow for the desired expression of a gene of interest in the host cell. Generation of the viral construct may be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. (1989) Molecular Cloning: A Laboratory Manual. Cold Spring Harbor Laboratory Press, N.Y.; Coffin et al. (997) Retroviruses. Cold Spring Harbor Laboratory Press, N.Y.; and “RNA Viruses: A Practical Approach” (Alan J. Cann, Ed., Oxford University Press, 2000).

Exemplary viral vectors include, for example, adenovirus vectors, adeno-associated virus vectors, retrovirus vectors, and lentivirus vectors. In some embodiments, viral vectors that integrate transgenes are used (e.g., virus other than adenoviral vectors). Exemplary types of viruses include HSV (herpes simplex virus), AAV (adeno associated virus), HIV (human immunodeficiency virus), BIV (bovine immunodeficiency virus), and MLV (murine leukemia virus). Nucleic acids may be transduced in any desired format that provides sufficiently efficient delivery levels, including in virus particles. A viral gene delivery vehicle may optionally comprise viral sequences such as a viral origin of replication or packaging signal. These viral sequences may be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al. (1983) (Cell 33:153; Cane and Mulligan (1984) Proc. Natl. Acad. Sci. U.S.A. 81:6349; Miller et al. (1990) Hum. Gene Therap. 1:5-14; U.S. Pat. Nos. 4,405,712, 4,861,719, and 4,980,289; and PCT Publs. WO 89/02,468, WO 89/05,349, and WO 90/02,806. Numerous retroviral gene delivery vehicles may be utilized in the present invention, including for example those described in EP Pat. Publ. 0415731; PCT Publs. WO 90/07936, WO 94/03622, WO 93/25698, and WO 93/25234; U.S. Pat. No. 5,219,740; PCT. Pubis. WO 93/11230 and WO 93/10218; Vile and Hart (1993) Cancer Res. 53:3860-3864: Vile and Hart (1993) Cancer Res. 53:962-967; Ram et al. (1993) Cancer Res. 53:83-88; Takamiya et al. (1992) J. Neurosci. Res. 33:493-503; Baba et al. (1993) J. Neurosurg. 79:729-735; U.S. Pat. No. 4,777,127; G.B. Patent No. 2,200,651; EP. Pat. Publ. 0345242; and PCT Publs. WO91/02805.

Other viral vector systems that may be used to deliver a polynucleotide of the invention have been derived from herpes virus, e.g., Herpes Simplex Virus (U.S. Pat. No. 5,631,236 and PCT Publ. WO 00/08191), vaccinia virus (Ridgeway (1988) “Mammalian expression vectors,” In: Rodriguez and Denhardt, eds. Vectors: A survey of molecular cloning vectors and their uses. Stoneham: Butterworth; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Exemplary viruses include an alphavirus, a poxivirus, an arena virus, a vaccinia virus, a polio virus, and the like. They offer several attractive features for various mammalian cells (see, for example, Friedmann (1989) Science 244:1275-1281; Ridgeway (1988) supra; Baichwal and Sugden (1986) supra; and Horwich et al. (1990) J. Virol. 64:642-650).

In some embodiments, lentiviral vectors are useful. Numerous lentiviruses suitable for use in the present invention are well known in the art. “Lentivirus” refers to a genus of retroviruses that are capable of infecting dividing and non-dividing cells. Lentiviruses may infect nondividing cells owing to the karyophilic properties of their preintegration complex, which allow for its active import through the nucleopore. Several examples of lentiviruses include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates.

A lentiviral genome is generally organized into a 5′ long terminal repeat (LTR), the gag gene, the pol gene, the env gene, the accessory genes (nef, vif, vpr, vpu) and a 3′ LTR. The viral LTR is divided into three regions called U3, R and U5. The U3 region contains the enhancer and promoter elements. The U5 region contains the polyadenylation signals. The R (repeat) region separates the U3 and U5 regions and transcribed sequences of the R region appear at both the 5′ and 3′ ends of the viral RNA. The 5′ and 3′ LTR's serve to promote transcription and polvadenylation of the virion RNAs. The LTR contains all other cis-acting sequences necessary for viral replication. Lentiviruses have additional genes including vif, vpr, tat, rev, vpu, nef and vpx. Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins. Engineered lentiviral vectors are also known that may transduce hematopoietic stem cells and HSC lineages (see, for example, “RNA Viruses: A Practical Approach” (Alan J. Cann, Ed., Oxford University Press, (2000); O Narayan and Clements (1989) J. Gen. Virol. 70:1617-1639; Fields et al. (1990) Fundamental Virology, Raven Press.; Miyoshi et al. (1998) J. Virol. 72:8150-8157; U.S. Pat. Nos. 5,994,136, 6,013,516, 8,551,773, and 8,361,787; Evans et al. (1999) Hum. Gene Ther. 10:1479-1489; Case et al. (1999) Proc. Natl. Acad. Sci. U.S.A. 96:2988-2993; Uchida et al. (1998) Proc. Natl. Acad. Sci. U.S.A. 95:11939-11944; Miyoshi et al. (1999) Science 283:682-686; Sutton et al. (1998) J. Virol. 72:5781-5788).

The viral virus vectors may be psedudotyped. A “pseudotyped” virus is a viral particle having an envelope protein that is from a virus other than the virus from which the RNA genome is derived. The envelope protein may be from a different virus. For example, an envelope protein is the vesicular stomatitius virus G (VSV G) protein or from measles virus. However, to eliminate the possibility of human infection, viruses may alternatively be pseudotyped with ecotropic envelope protein that limit infection to a specific species, such as mice or birds. For example, in one embodiment, a mutant ecotropic envelope protein is used, such as the ecotropic envelope protein 4.17 (see, for example, Powell et al. (2000) Nat. Biotech. 18:1279-1282).

The viral virus vectors may also be self-inactiving. For example, a “self-inactivating 3′ LTR” is a 3′ long terminal repeat (LTR) that contains a mutation, substitution or deletion that prevents the LTR sequences from driving expression of a downstream gene. A copy of the U3 region from the 3′ LTR acts as a template for the generation of both LTR's in the integrated provirus. Thus, when the 3′ LTR with an inactivating deletion or mutation integrates as the 5′ LTR of the provirus, no transcription from the 5′ LTR is possible. This eliminates competition between the viral enhancer/promoter and any internal enhancer/promoter. For example, a deletion in the U3 region of the 3′ LTR of the vector DNA, i.e., the DNA used to produce the vector RNA may be made. Thus, during reverse transcription, this deletion is transferred to the 5′ LTR of the proviral DNA. It is desirable to eliminate enough of the U3 sequence to greatly diminish or abolish altogether the transcriptional activity of the LTR, thereby greatly diminishing or abolishing the production of full-length vector RNA in transduced cells. However, it is generally desirable to retain those elements of the LTR that are involved in polyadenylation of the viral RNA, a function spread out over U3, R and U5. Accordingly, it is desirable to eliminate as many of the transcriptionally important motifs from the LTR as possible while sparing the polyadenylation determinants. The LTR may be rendered about 80%, 81%, 82%, 83%, 84%, 85%, 86%, 87%, 88%, 89%, 90%, 91%, 92%, 93%, 94%, 95% 96% 97%, 98%, to about 99% transcriptionally inactive.

Self-inactivating 3′ LTRs and other viral self-inactivating methods and reagents are well known in the art (see, for example, Zufferey et al. (1998). Virol. 72:9873-9880; Miyoshi et al. (1998) J. Virol. 72:8150-8157; and Iwakuma et al. (1999) Virol. 261:120-132).

Other elements commonly found in viral vectors and generally operably linked to genes of interest in order to enhance the expression or utility of the viral vectors are well known and described further below.

c. Enhancers, Promoters, and Inducible Forms Thereof

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter, or other regulatory element or useful element of the vector, is in a correct functional location and/or orientation in relation to a nucleic acid sequence to regulate the sequence (e.g., control transcriptional initiation and/or expression of that sequence).

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, the 5′ end of the transcription initiation site of the transcriptional reading frame is placed “downstream” of (i.e., 3′ of) the chosen promoter. The spacing between promoter elements frequently is flexible, so that promoter function is preserved when elements are inverted or moved relative to one another. In the tk promoter, the spacing between promoter elements may be increased to 50 bp apart before activity begins to decline. Depending on the promoter, it appears that individual elements may function either cooperatively or independently to activate transcription.

In addition, a specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons may be either natural or synthetic. The efficiency of expression may be enhanced by the inclusion of appropriate transcription enhancer elements.

A promoter may or may not be used in conjunction with an “enhancer,” which refers to a cis-acting regulatory sequence involved in the transcriptional activation of a nucleic acid sequence. Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Aside from this operational distinction, enhancers and promoters are very similar entities. Promoters and enhancers have the same general function of activating transcription in the cell. They are often overlapping and contiguous, often seeming to have a very similar modular organization. Taken together, these considerations suggest that enhancers and promoters are homologous entities and that the transcriptional activator proteins bound to these sequences may interact with the cellular transcriptional machinery in fundamentally the same way. For example the CMV enhancer (Karasuyama et al. (1989) J. Erp. Med. 169:13) may be used in combination with the chicken β-actin promoter (see, e.g., JP 1990005890-A1). Again, one of skill in the art will be able to select the appropriate enhancer based on the desired expression pattern.

A promoter may be one naturally associated with a nucleic acid sequence, as may be obtained by isolating the 5′ non-coding sequences located upstream of the coding segment and/or exon. Such a promoter may be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the P-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906). Furthermore, it is contemplated the control sequences that direct transcription and/or expression of sequences within non-nuclear organelles such as mitochondria, chloroplasts, and the like, may be employed as well. Control sequences comprising promoters, enhancers and other locus or transcription controlling/modulating elements are also referred to as “transcriptional cassettes”.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. (1989) supra). The promoters employed may be constitutive, tissue-specific, cell-specific, developmental stage-specific, inducible, and/or useful under the appropriate conditions to direct high level expression of the introduced DNA segment, such as is advantageous for gene therapy or for applications such as the large-scale production of recombinant proteins and/or peptides. The promoter may be heterologous or endogenous. Use of a T3, T7 or SP6 cytoplasmic expression system is another possible embodiment. Eukaryotic cells may support cytoplasmic transcription from certain bacterial promoters if the appropriate bacterial polymerase is provided, either as part of the delivery complex or as an additional genetic expression construct. To determine whether a particular promoter is useful, a selected promoter may be tested in the construct in vitro in an HSC lineage cell and, if the promoter is capable of promoting expression of the transgene at a detectable signal-to-noise ratio, it will generally be useful in accordance with the present invention. A desirable signal-to-noise ratio is one between about 10 and about 200, a more desirable signal-to-noise ratio is one 40 and about 200, and an even more desirable signal-to-noise ratio is one between about 150 and about 200. One means of testing such a promoter, described in more detail herein below, is through the use of a signal generating transgene such as a reporter, like a fluorescent protein such as the green fluorescent protein (GFP).

Non-limiting examples of promoters that may be used include the promoter for ubiquitin, CMV (U.S. Pat. No. 5,168,062 and Karasuyama et al. (1989) J. Exp. Med 169:13), β-actin (Gunning et al. (1987) Proc. Natl. Acad. Sci. U.S.A. 84:4831-4835), and pgk (U.S. Pat. Nos. 4,615,974 and 5,104,795; Adra et al. (1987) Gene 60:65-74; Singer-Sam et al. (1984) Gene 32:409-417; and Dobson et al. (1982) Nucl. Acids Res. 10:2635-2637). Alternatively, the promoter may be a tissue specific promoter. Several non-limiting examples of tissue specific promoters that may be used include lck (see, for example, Garvin et al. (1988) Mol. Cell. Biol. 8:3058-3064 and Takadera et al. (1989) Mol. Cell. Biol. 9:2173-2180), myogenin (Yee et al. (1993) Genes Dev. 7:1277-1289), and thy 1 (Gundersen et al. (1992) Gene 113:207-214). In addition, promoters may be selected to allow for inducible expression of the transgene.

For expressing short RNAs, such as interfering RNAs, RNA Polymerase III promoters are well known to one of skill in the art. For example, a wide range of RNA Polymerase III promoters are disclosed in Paule and White (2000) Nucl. Acids Res. 28:1283-1298. The definition of RNA Polymerase III promoters also include any synthetic or engineered DNA fragment that may direct RNA Polymerase III to transcribe a downstream RNA coding sequence. Suitable promoters include, but are not limited to, the U6 or HI RNA pol III promoter sequences and the cytomegalovirus promoter.

Further, viral vector promoters, such as the RNA Polymerase III (Pol III) promoter or other promoters used as part of the viral vector, may be inducible. Any suitable inducible promoter may be used with the methods encompassed by the present invention and such promoters are well known in the art (see, for example, PCT Publ. WO 2004/056964; U.S. Pat. No. 8,679,845; and U.S. Pat. Publ. 2010/0077495). Transcription-regulatory elements conferring inducibility on the promoters may be placed within the promoter region, such as between the proximal sequence element (PSE) and the transcription start site, upstream or downstream from the TATA box. Such sequences may also be placed outside the promoter, such as downstream from the end of an interfering RNA sequence. In addition, a viral vector contain 1, 2, 3, 4, 5, 6, 7, 8, 9, 10, or more of such inducibility conferring elements in order to more or less tightly regulate transcription in response to the inducing signal.

Useful inducible Pol III promoters include tetracycline responsive promoters (see, for example, Ohkawa and Taira (2000) Hum. Gene Therap. 11:577-585 and Meissner et al. (2001) Nucl. Acids Res. 29:1672-1682), operator sequences (tetO) of the E. coli tetracycline resistance operon (Czauderna et al. (2003) Nucl. Acids Res. 31:e127; Matsukura et al. (2003) Nucl. Acids Res. 31:e77; van de Wetering et al. (2003) EMBO Rep. 4:609-615; and Ohkawa et al. (2000) Hum. Gene Ther. 11:577-585). Many inducible promoters may be used as a cis-regulatory element and these commonly, but not necessarily, use an element that serves a landing pad function of providing a place to which a tethering factor (a sequence-specific DNA binding protein) may bind to the DNA and bring a diversification factor, fused to the tethering factor, into sufficient proximity of the coding region so that diversification of the coding region is capable of reversible regulation. A tethering factor is one that binds to the cis-regulatory element in a sequence-specific manner. In the embodiments in which LacO serves as a cis-regulatory element, the Lac repressor, Lacl, may serve as the tethering factor, and its binding to the cis-regulatory element, LacO, may be regulated by isopropyl-β-D-thio-galactoside (IPTG). In the absence of IPTG, Lac binds LacO and diversification is accelerated (or otherwise regulated) by the presence of the diversification factor. IPTG may be added in the event that a halt or reduction in activity of the diversification factor is desired. In embodiments in which TetO serves as the cis-regulatory element, TetR may be a suitable tethering factor, and the activity of the diversification factor may be regulated by tetracycline or doxycycline. Other transcription-regulatory elements that allow or inducible expression are well known in the art and may be inserted into the promoter region for controlled expression of genes of interest. For example, LPTG-inducible systems based on LacO and LacI repressors are well known in the art, as are inducible systems based on Cre, GalO, MTII (phorbol ester, TFA), MMTV (glucocorticoids), beta-interferon (poly(rI) or poly(rc)), adenovirus 5 E2 (E1A), collagenase (phorbol ester, TFA), and the like. For RNA Polymerase I- or Pol LI-based transcription units, well-established inducible systems such as tetracycline transactivator systems, reverse tetracycline transactivator systems, and ecdysone systems may be used.

Additional regulatory elements are also well known that may enhance expression of the gene of interest. One type of posttranscriptional regulatory sequence is an intron positioned within the expression cassette, which may serve to stimulate gene expression. Since introns placed in such a manner may expose the RNA transcript of the gene of interest to the normal cellular splicing and processing mechanisms, it may be desirable to locate intron-containing transgenes in an orientation opposite to that of the vector genomic transcript. Alternatively, a method of enhancing expression of a gene of interest is through the use of a posttranscriptional regulatory element which does not rely on splicing events, such as the posttranscriptional processing element of herpes simplex virus, the posttranscriptional regulatory element of the hepatitis B virus (HPRE) or that of the woodchuck hepatitis virus (WPRE), which contains an additional cis-acting element not found in the HPRE. The regulatory element is positioned within the vector so as to be included in the RNA transcript of the transgene, but outside of stop codon of the transgene translational unit. The use of such regulatory elements are particularly preferred in the context of modest promoters, but may be contraindicated in the case of very highly efficient promoters.

d. Other Vector Elements

Vectors encompassed by the present invention may include a multiple cloning site (MCS), which is a nucleic acid region that contains multiple restriction enzyme sites, any of which may be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression.

The vectors useful for the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements may serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. For example, Pol III terminators preferably comprise of stretches of 4 or more thymidine (“T”) residues. In a preferred embodiment, a cluster of 5 consecutive Ts is linked immediately downstream of the RNA coding region to serve as the terminator. In such a construct pol III transcription is terminated at the second or third T of the DNA template, and thus only 2 to 3 uridine (“U”) residues are added to the 3′ end of the coding sequence. In certain embodiments, the termination signal may be a lack of transcribable or translatable sequence, such as due to a sequence truncation.

In eukaryotic gene expression, a polyadenylation signal is generally added in order to effect proper polyadenylation of the transcript. The nature of the polyadenylation signal is not believed to be crucial to the successful practice of the invention, and any such sequence may be employed. Some examples include the SV40 polyadenylation signal or the bovine growth hormone polyadenylation signal, convenient and known to function well in various target cells. Polyadenylation may increase the stability of the transcript or may facilitate cytoplasmic transport.

In order to propagate a vector of the invention in a host cell, it may contain one or more origins of replication sites (often termed “ori”), which is a specific nucleic acid sequence at which replication is initiated. Alternatively an autonomously replicating sequence (ARS) may be employed if the host cell is yeast.

e. Production of Virus

Any method known in the art may be used to produce infectious retroviral particles whose genome comprises an RNA copy of the viral construct described above. Preferably, the viral construct is introduced into a packaging cell line. The packaging cell line provides the viral proteins that are required in trans for the packaging of the viral genomic RNA into viral particles. The packaging cell line may be any cell line that is capable of expressing retroviral proteins. Useful packaging cell lines include 293 (ATCC CCL X), HeLa (ATCC CCL 2), D17 (ATCC CCL 183), MDCK (ATCC CCL 34), BHK (ATCC CCL-10) and Cf2Th (ATCC CRL 1430). The packaging cell line may stably express the necessary viral proteins (see, for example, U.S. Pat. No. 6,218,181). Alternatively a packaging cell line may be transiently transfected with plasmids comprising nucleic acid that encodes the necessary viral proteins. In one embodiment a packaging cell line that stably expresses the viral proteins required for packaging the RNA genome is transfected with a plasmid comprising the viral construct described above. In another embodiment a packaging cell line that does not stably express the necessary viral proteins is co-transfected with two or more plasmids (see, for example, Yee et al. (1994)Meth. Cell. Biol. 43A:99-112). In some embodiments, the packaging cell line may not express envelope gene products. In this case, the packaging cell line will package the viral genome into particles that lack an envelope protein. As the envelope protein is responsible, in part, for the host range of the viral particles, the viruses may be pseudotyped as described above. In other embodiments, RNA interference activity of the packaging cells may be suppressed in order to improve the production of recombinant virus. This includes, without limitation, the use of cotransfection or stable transfection of constructs expressing siRNA molecules to inhibit Dicer, an RNase III family member of ribonuclease which is essential for RNA interference (Hammond et al. (2001) Nat. Rev. Genet. 2:110-119). The recombinant virus is then preferably purified from the packaging cells, titered and diluted to the desired concentration according to standard protocols well known in the art.

f. Delivery of Virus

Target cells may be transduced in any way that allows the virus to contact the target cells in which delivery of a sequence containing a gene of interest is desired according to well-known methods in the art (see, for example U.S. Pat. No. 8,552,150). In some embodiments, a suitable amount of virus is introduced into a subject directly (in vivo), for example though injection into the host's body. In some preferred embodiments, the viral particles are injected into a subject's peripheral blood stream. In other preferred embodiments, the viral particles are injected into a subject through intra-dermal injection, subcutaneous injection, intra-peritoneal cavity injection, or intra-venal injection. The virus may be delivered using a subdermal injection device, such as those disclosed in U.S. Pat. Nos. 7,241,275, 7,115,108, 7,108,679, 7,083,599, 7,083,592, 7,047,070, 6,971,999, 6,808,506, 6,780,171, 6,776,776, 6,689,118, 6,670,349, 6,569,143, 6,494,865, 5,997,501, 5,848,991, 5,328,483, 5,279,552, 4,886,499. Other injection locations also are suitable, such as directly into organs comprising target cells. For example intra-lymph node injection, intra-spleen injection, or intra-bone marrow injection may be used to deliver virus to the lymph node, the spleen and the bone marrow, respectively. Transduced cell populations of interest may then be selected.

In other embodiments encompassed by the present invention, a suitable amount of virus is introduced into target cells obtained from a subject (ex vivo), for example through incubation of the virus with target primary cells or target cells in culture. The target cells may be cells obtained from bone marrow, fetal liver, peripheral blood, amniotic fluid, cord blood, and the like. Methods to obtain cells from a subject are well known in the art as described above. The virus may be suspended in media and added to the wells of a culture plate, tube or other container. The media containing the virus may be added prior to the plating of the cells or after the cells have been plated. Preferably cells are incubated in an appropriate amount of media to provide viability and to allow for suitable concentrations of virus in the media such that infection of the host cell occurs.

In still other embodiments, target cells are provided and contacted with the virus in vitro, such as in culture plates.

The cells may be incubated with the virus for a sufficient amount of time to allow the virus to infect the cells. Preferably the cells are incubated with virus for at least 1 hour, more preferably at least 5 hours and even more preferably at least 10 hours.

In ex vivo, in vitro, and in vivo delivery embodiments, any concentration of virus that is sufficient to infect the desired target cells may be used, as may be readily determined by the skilled artisan. When the target cell is to be cultured, the concentration of the viral particles is at least 1 PFU/μl, more preferably at least 10 PFU/μl, even more preferably at least 400 PFU/μl and even more preferably at least 1×10⁴ PFU/μl. The titer of the virus may be adjusted to allow for, on average, 1, 2, 3, 4, 5, or more independent cellular transductions with independent viral constructs. In one embodiment, the viral titer is adjusted to allow for 1 or fewer such cellular transduction events in order to prevent multiple integration events.

The methods of infecting cells disclosed above do not depend upon individual-specific characteristics of the cells. As a result, they are readily extended to all mammals. In some embodiments the recombinant virus is delivered to a human or to human HSC cell lineages. In other embodiments, the recombinant virus is delivered to a mouse or to mouse HSC cell lineages. In still other embodiments, the recombinant virus is delivered to an animal other than a human or a mouse, or to cells from an animal other than a human or a mouse.

As discussed above, the recombinant virus may be pseudotyped to confer upon it a broad host range as well as target cell specificity. One of skill in the art would also be aware of appropriate internal promoters to achieve the desired expression of a polynucleotide or gene of interest in a particular animal species. Thus, one of skill in the art will be able to modify the method of infecting dendritic cells derived from any species.

The transduced cells may be analyzed, for example for integration, transcription, and/or expression of genes of interest, the number of copies of the gene integrated, and the location of the integration. Such analysis may be carried out at any time and may be carried out by any methods known in the art. Incubator animals in which a recombinant virus or virus-infected target cells are administered may be analyzed for location of infected cells, expression of the virus-delivered gene of interest, modulation of an immune response, and/or monitored for symptoms associated with a disease or disorder by any methods known in the art.

2. Transplantation and Selection of Transduced Cells in Incubator Animals

Transduced HSCs and/or cells derived therefrom may be transplanted into incubator animals such that they proliferate, develop, and/or differentiate in an in vivo environment. Transduced cell populations of interest may then be selected from the incubator animals.

“Incubator animals” are host animals in which transduced HSCs and/or cells derived therefrom may proliferate, develop, and/or differentiate in an in vivo environment. The host animals, animal ages, transplantation routes, cellular isolation methods, marker phenotyping methods, and the like are not particularly restricted and include all of the various animals from which the transduced HSCs and/or cells derived therefrom were obtained, as described above. Following transduction, the transduced HSCs and/or cells derived therefrom may be introduced or re-introduced into an incubator animal. In some embodiments, the cells may be introduced into the peripheral blood stream by, for example, intravenous infusion. The cells introduced into a subject may be cells derived from that subject, to avoid an adverse immune response. Cells also may be used that are derived from a donor subject having a similar immune background. Other cells also may be used, including those designed to avoid an adverse immunogenic response.

In one embodiment, incubator animals are autologous with respect to the transduced HSCs and/or cells derived therefrom. “Autologous” refers to deriving from or originating in the same subject or patient. An “autologous transplant” refers to the harvesting and reinfusion or transplant of a subject's own cells or organs. Exclusive or supplemental use of autologous cells may eliminate or reduce many adverse effects of administration of the cells back to the host, particular graft versus host reaction.

In another embodiment, incubator animals are allogeneic with respect to the transduced HSCs and/or cells derived therefrom. “Allogeneic” refers to deriving from, originating in, or being members of the same species, where the members are genetically related or genetically unrelated but genetically similar. An “allogeneic transplant” refers to transfer of cells or organs from a donor to a recipient, where the recipient is the same species as the donor.

In still another embodiment, incubator animals are mismatched allogeneic with respect to the transduced HSCs and/or cells derived therefrom. “Mismatched allogeneic” refers to deriving from, originating in, or being members of the same species having non-identical major histocompatibility complex (MHC) antigens (i.e., proteins) as typically determined by standard assays used in the art, such as serological or molecular analysis of a defined number of MHC antigens. A “partial mismatch” refers to partial match of the MHC antigens tested between members, typically between a donor and recipient. For instance, a “half mismatch” refers to 50% of the MHC antigens tested as showing different MHC antigen type between two members. A “full” or “complete” mismatch refers to all MHC antigens tested as being different between two members.

Determining the degree of MHC mismatch may be accomplished according to standard tests known and used in the art. For instance, there are at least six major categories of MHC genes in humans, identified as being important in transplant biology. HLA-A, HLA-B, HLA-C encode the HLA class I proteins while HLA-DR, HLA-DQ, and HLA-DP encode the HLA class II proteins. Genes within each of these groups are highly polymorphic, as reflected in the numerous HLA alleles or variants found in the human population, and differences in these groups between individuals is associated with the strength of the immune response against transplanted cells. Standard methods for determining the degree of MHC match examine alleles within HLA-B and HLA-DR, or HLA-A, HLA-B and HLA-DR groups. Thus, tests may be made of at least 4, and even 5 or 6 MHC antigens within the two or three HLA groups, respectively.

In serological MHC tests, antibodies directed against each HLA antigen type are reacted with cells from one subject (e.g., donor) to determine the presence or absence of certain MHC antigens that react with the antibodies. This is compared to the reactivity profile of the other subject (e.g., recipient). Reaction of the antibody with an MHC antigen is typically determined by incubating the antibody with cells, and then adding complement to induce cell lysis (i.e., lymphocytotoxicity testing). The reaction is examined and graded according to the amount of cells lysed in the reaction (see, for example, Mickelson and Petersdorf (1999) Hematopoietic Cell Transplantation, Thomas, E. D. et al. eds., pg 28-37, Blackwell Scientific, Malden, Mass.). Other cell-based assays include flow cytometry using labeled antibodies or enzyme linked immuno assays (ELISA).

Molecular methods for determining MHC type are well known and generally employ synthetic probes and/or primers to detect specific gene sequences that encode the HLA protein. Synthetic oligonucleotides may be used as hybridization probes to detect restriction fragment length polymorphisms associated with particular HLA types (Vaughn (2002) Method. Mol. Biol. MHC Protocol. 210:45-60). Alternatively, primers may be used for amplifying the HLA sequences (e.g., by polymerase chain reaction or ligation chain reaction), the products of which may be further examined by direct DNA sequencing, restriction fragment polymorphism analysis (RFLP), or hybridization with a series of sequence specific oligonucleotide primers (SSOP) (Petersdorf et al. (1998) Blood 92:3515-3520; Morishima et al. (2002) Blood 99:4200-4206; and Middleton and Williams (2002) Method. Mol. Biol. MHC Protocol. 210:67-112).

In yet another embodiment, incubator animals are syngeneic with respect to the transduced HSCs and/or cells derived therefrom. “Syngeneic” refers to deriving from, originating in, or being members of the same species that are genetically identical, particularly with respect to antigens or immunological reactions. These include identical twins having matching MHC types. Thus, a “syngeneic transplant” refers to transfer of cells or organs from a donor to a recipient who is genetically identical to the donor.

In another embodiment, incubator animals are xenogeneic with respect to the transduced HSCs and/or cells derived therefrom. “Xenogeneic” refers to deriving from, originating in, or being members of different species, e.g., human and rodent, human and swine, human and chimpanzee, etc. A “xenogeneic transplant” refers to transfer of cells or organs from a donor to a recipient where the recipient is a species different from that of the donor. In one embodiment, the incubator animal may be “humanized” in order to be compatible with human transduced HSCs and/or cells derived therefrom. The term “immune-system humanized” refers to an animal such as a mouse comprising human HSCs and/or cells derived therefrom and human acquired and innate immune cells, wherein the human HSCs and/or cells derived therefrom and human acquired and innate immune cells differentiated from the HSCs and/or cells derived therefrom survive without being rejected from the host animal, thereby allowing human hematopoiesis and both acquired and innate immunity to be reconstituted in the host animal. Acquired immune cells include T cells and B cells. Innate immune cells include macrophages, granulocytes (basophils, eosinophils, neutrophils), DCs, NK cells and mast cells. Representative, non-limiting examples include SCID-hu, Hu-PBL-SCID, Hu-SRC-SCID, NSG (NOD-SCID IL2r-gamma(null)), NOG (NOD-SCID IL2r-gamma(truncated)), BRG (BALB/c-Rag2(null)IL2r-gamma(null))), and H2dRG (Stock-H2d-Rag2(null)IL2r-gamma(null)) mice (see, for example, Shultz et al. (2007) Nat. Rev. Immunol. 7:118; Pearson et al. (2008) Curr. Protocol. Immunol. 15:21; Brehm et al. (2010) Clin. Immunol. 135:84-98), as well as related null mutants of immune-related genes like Rag1, Rag2, IL2rg, or Prfl, allow for efficient engraftment of human immune cells in mice (see, for example, PCT Publ. WO2013/062134).

Besides the species or immunological match between the transduced HSCs and/or cells derived therefrom and the incubator animal, the incubator animal may be distinguished from the transduced HSCs and/or cells derived therefrom in other ways. For example, the incubator animal may be congenic with respect to the transduced HSCs and/or cells derived therefrom. “Congenic” refers to deriving from, originating in, or being members of the same species, where the members are genetically identical except for a small genetic region, typically a single genetic locus (i.e., a single gene). A “congenic transplant” refers to transfer of cells or organs from a donor to a recipient, where the recipient is genetically identical to the donor except for a single genetic locus. For example, CD45 exists in several allelic forms and congenic mouse lines exist in which the mouse lines differ with respect to whether the CD45.1 or CD45.2 allelic versions are expressed.

In one embodiment, the incubator animal is immunocompromised. An “immunocompromised” animal is an animal who is incapable of developing or unlikely to develop a robust immune response due to a lack or reduction in functioning mature immune system cells, such as B cells and/or T cells. Immunocompromised subjects are more susceptible to opportunistic infections, for example viral, fungal, protozoan, or bacterial infections, prion diseases, and certain neoplasms.

In some embodiments, the immunocompromised incubator animal is “immunodeficient” in which no native host immune response may be mounted. In one embodiment, immunodeficient mice are useful. For example, such mice may have severe combined immune deficiency. The term “severe combined immune deficiency (SCID)” refers to a condition characterized by absence of T cells and lack of B cell function. Common forms of SCID include: X-linked SCID which is characterized by gamma chain gene mutations in the IL2RG gene and the lymphocyte phenotype T(−) B(+) NK(−); and autosomal recessive SCID characterized by Jak3 gene mutations and the lymphocyte phenotype T(−) B(+) NK(−), ADA gene mutations and the lymphocyte phenotype T(−) B(−) NK(−), IL-7R alpha-chain mutations and the lymphocyte phenotype T(−) B(+) NK(+), CD3 delta or epsilon mutations and the lymphocyte phenotype T(−) B(+) NK(+), RAG1/RAG2 mutations and the lymphocyte phenotype T(−) B(−) NK(+), Artemis gene mutations and the lymphocyte phenotype T(−) B(−) NK(+), CD45 gene mutations and the lymphocyte phenotype T(−) B(+) NK(+). In one embodiment, the immunodeficient mouse used in the present invention is a mouse having the severe combined immunodeficiency mutation (Prkdc^{scid}), commonly referred to as the scid mutation. The scid mutation is well-known and located on mouse chromosome 16 (see, for example, Bosma et al. (1989) Immunogenet. 29:54-56). Mice homozygous for the scid mutation are characterized by an absence of functional T cells and B cells, lymphopenia, hypoglobulinemia and a normal hematopoietic microenvironment. The scid mutation may be detected, for example, by detection of markers for the scid mutation using well-known methods.

Immunocompromised and immunodeficient incubator animals allow for ablation of the native host immune system such that the immune system may be repopulated substantially or completely from the transplanted transduced HSCs and/or cells derived therefrom. Aside from genetic manipulations, incubator animals may be rendered immunocompromised or immunodeficient using any number of well-known techniques. For example, they may be conditioned with sub-lethal irradiation or lethal irradiation with high frequency electromagnetic radiation, generally using gamma radiation, or treated with a radiomimetic drug such as busulfan or nitrogen mustard, or treated with immunotherapy to deplete immune system-mediating cell populations (see, for example, Hayakawa et al. (2009) Stem Cells 27:175-182).

Transplantation of cells into incubator animals may be accomplished using methods generally known in the art. For example, incubator animals of interest may be engrafted with transplanted transduced HSCs and/or cells derived therefrom by various routes. Such routes include, but are not limited to, intravenous administration, injection into the femur bone marrow cavity, injection into the spleen, or administration under the renal capsule of fetal liver. Cells may be administered in one infusion, or through successive infusions over a defined time period sufficient to generate a therapeutic effect. Exemplary methods for transplantation, engraftment assessment, and marker phenotyping analysis of transplanted transduced HSCs and/or cells derived therefrom are well known in the art (see, for example, Pearson et al. (2008) Curr. Protocol. Immunol. 81:15.21.1-15.21.21; Ito et al. (2002) Blood 100:3175-3182; Traggiai et al. (2004) Science 304:104-107; Ishikawa et al. Blood (2005) 106:1565-1573; Shultz et al. (2005) J. Immunol. 174:6477-6489; and Holyoake et al. (1999) Exp. Hematol. 27:1418-1427).

The number of transduced HSCs and/or cells derived therefrom transduced may be adjusted based on the desired level of engraftment. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be transplanted. Transplantation of at least about 1.0×10⁶, 2.0×10⁶, 3.0×10⁶, 4.0×10⁶, or 5.0×10⁶ per kg of incubator host is also generally effective (see, for example, Olivieri et al. (1998) Haematologica 83:329-337; Mavroudis et al. (1996) Blood Vo. 88:3223-3229; Singhal et al. (2000) Bone Marrow Transplant. 26:489-96; and Bittencourt et al. (2002) Blood 99:2726-2733).

Engraftment of transplanted transduced HSCs and/or cells derived therefrom may be assessed by any of various methods, such as, but not limited to, flow cytometric analysis of cells of interest obtained from the incubator animals at one or more time points following transplantation. For example, the number of colony forming cells, the number of granulocyte-macrophage colony forming cells, the number of burst forming unit-erythroid cells, the number of colony forming unit-granulocyte erythroid monocyte macrophage cells, that are collected or administered, may be analyzed. “Engraftment” is successful where transplanted transduced HSCs and/or cells derived therefrom and cells differentiated therefrom in the incubator animal are detected at a time when the majority of any transplanted non-HSCs and/or cells derived therefrom has degenerated. Serial transfer of cells into a secondary recipient and engraftment thereof is a further test of engraftment in the primary incubator animal. In one embodiment, the engraftment level of transplanted transduced HSCs and/or cells derived therefrom may be calculated as the percentage of transplanted transduced HSCs and/or cells derived therefrom as assessed by analysis of a phenotypic marker relative to the total numbers of cells expressing the marker, such as in a population of cells from bone marrow, peripheral blood, etc. The engraftment level is generally 70% or more, preferably 80% or more, more preferably 90% or more, particularly preferably 95% or more. The engraftment level of transplanted transduced HSCs and/or cells derived therefrom in spleen is generally 70% or more, preferably 80% or more, more preferably 85% or more, more preferably 90% or more. The engraftment level of transplanted transduced HSCs and/or cells derived therefrom in peripheral blood is generally 60% or more, preferably 70% or more, more preferably 80% or more. Engraftment may be detected by flow cytometry as 0.05% or greater transplanted cells in the blood, spleen or bone marrow at 10-12 weeks after transplantation.

After transplantation and engraftment, transduced HSC cell lineage populations and progeny thereof may be obtained, isolated, and/or purified using methods described above. At any time, engrafted cells may be analyzed by marker phenotyping, gene expression analyses, reporter activity, and the like to determine the cell state of the cells.

In one embodiment, the cells are naïve. “Naïve” cells are immune cells that have differentiated in bone marrow, successfully undergone positive and negative selection in the thymus, and are mature, but have not been activated and are not memory cells. Naïve T cells are commonly characterized by the surface expression of L-selectin (CD62L); the absence of the activation markers, CD25, CD44, or CD69; and the absence of memory CD45RO isoform. They also express functional IL-7 receptors, consisting of subunits IL-7 receptor-α, CD127, and common-γ chain, CD132. In the naive state, T cells are thought to be quiescent and non-dividing, requiring the common-gamma chain cytokines IL-7 and IL-15 for homeostatic survival mechanisms. By contrast, activated T cells express or upregulate expression of surface markers, CD25, CD44, CD62L^{low}, and CD69 and may further differentiate into memory T cells. Naïve B cells have not been exposed to antigen since they would either become a memory B cell or a plasma cell that secretes antibodies.

3. Uses of Transduced HSCs and/or Cells Derived Therefrom

The methods described herein for generating transduced HSCs and/or cells derived therefrom that are differentiated in vivo, as well as progeny thereof and compositions thereof. Such compositions have various utilities such as, but not limited to, as models of growth and differentiation of immune cells, in vivo study of immune response, and for the testing of agents (e.g., gene products and compounds) affecting hematopoietic and immune cell function. The preservation of biologically faithful immune cell development allows for the embodiments encompassed by the present invention to be useful for analyzing various autoimmune, allergic (e.g., asthma, atopic dermatitis, allergic conjunctivitis, pollen allergy, food allergy, etc.), vaccination, immunotolerance, cancer immunotherapy, immune exhaustion, immunological memory, or immunological epitope responses. Such compositions can also be used for prognostic, diagnostic, and therapeutic purposes as described herein.

Methods of analyzing such responses may use cell populations selected from the incubator animals in vitro or upon additional transplantation into an experimental animal. An “experimental animal” is an animal in which transduced cell types of interest are transplanted and exogenous perturbations are made in order to analyze the effects on or achieved through the transplanted transduced cell types. Experimental animals and transplantation methods may follow any or all of the criteria described for incubator animals above. For assays in which a gene of interest is expressed from HSC cells are inducibly expressed, transcriptional and/or translational induction may be achieved before, simultaneously with, or after, the exogenous perturbation according to well-known methods in the art described above.

a. Screening Methods

One aspect encompassed by the present invention relates to methods of selecting agents (e.g., nucleic acids, proteins, antibodies, fusion proteins, peptides, or small molecules) which modulate an immune response. Such methods utilize screening assays using cell based assays either in vitro, ex vivo, or in vivo. The term “immune response” includes T cell mediated and/or B cell mediated immune responses that are influenced by modulation of T cell costimulation. Exemplary immune responses include T cell responses, e.g., cytokine production, and cellular cytotoxicity. In addition, the term immune response includes immune responses that are indirectly effected by T cell activation, e.g., antibody production (humoral responses) and activation of cytokine responsive cells, e.g., macrophages. Immune responses encompass assays testing autoimmune, allergic, vaccination, immunotolerance, cancer immunotherapy, immune exhaustion, immunological memory, or immunological epitope responses. The test agent may be analyzed to determine whether it improves a response, condition, or symptom of interest. For example, a test agent that induces differentiation of cells, such as stem cells or terminally differentiated cells will be identified as an agent that induces differentiation of cells.

In some embodiments, the screening methods encompassed by the present invention are adapted for high-throughput analysis. For example, methods for preparing a combinatorial library of molecules that may be tested for a desired activity are well known in the art and include, for example, methods of making a phage display library of peptides, which may be constrained peptides (see, for example, U.S. Pat. Nos. 5,622,699 and 5,206,347; Scott and Smith (1992) Science 249:386-390; and Markland et al. (1991) Gene 109:13-19); a peptide library (see, for example, U.S. Pat. No. 5,264,563); a peptidomimetic library (see, for example, Blondelle et al. (1995) Trends Anal. Chem. 14:83-92; a nucleic acid library (see, for example, O'Connell et al. (1996) Proc. Natl. Acad. Sci. U.S.A. 93:5883-5887; and Tuerk and Gold (1990) Science 249:505-510; Gold et al. (1995) Ann. Rev. Biochem. 64:763-797); an oligosaccharide library (see, for example, York et al. (1996) Carb. Res. 285:99-128; Liang et al. (1996) Science 274:1520-1522; and Ding et al. (1995) Adv. Expt. Med Biol. 376:261-269); a lipoprotein library (see, for example, de Kruif et al. (1996) FFBS Lett. 399:232-236); a glycoprotein or glycolipid library (see, for example, Karaoglu et al. (1995) J. Cell Biol. 130:567-577); or a chemical library containing, for example, drugs or other pharmaceutical agents (see, for example, Gordon et al. (1994) J. Med. Chem. 37:1385-1401 and Ecker and Crooke (1995) BioTechnol. 13:351-60).

For a high throughput format, cells of interest may be introduced into wells of a multiwell plate or of a glass slide or microchip, and may be contacted with the test agent. Generally, the cells are organized in an array, particularly an addressable array, such that robotics conveniently may be used for manipulating the cells and solutions and for monitoring the cells of the invention, particularly with respect to the function being examined. An advantage of using a high-throughput format is that a number of test agents may be examined in parallel, and, if desired, control reactions also may be run under identical conditions as the test conditions. As such, the methods encompassed by the present invention provide a means to screen one, a few, or a large number of test agents in order to identify an agent that may alter a function of desired cells.

In one embodiment, the invention relates to assays for screening agents that bind to, or modulate the expression and/or activity of an immune-related biomolecule in the context of HSCs and/or cells derived therefrom expressing a gene of interest described above. In one embodiment, a method for identifying an agent to modulate an immune response entails determining the ability of the agent to modulate, e.g. enhance or inhibit, the interaction between immune-related biomolecules in the context of HSCs and/or cells derived therefrom expressing a gene of interest. Such agents include, without limitation, antibodies, proteins, fusion proteins, small molecules, and nucleic acids.

Modulation of an immune response may be determined using standard methods in the art, including, for example, (1) increased or decreased copy number (e.g., by FISH, FISH plus SKY, single-molecule sequencing, e.g., as described in the art at least at J. Biotechnol., 86:289-301, or qPCR), overexpression or underexpression of a nucleic acid (e.g., by ISH, Northern Blot, or qPCR), increased or decreased biomarker protein (e.g., by IHC) and/or biomarker metabolite, or increased or decreased activity (determined by, for example, analyzing modulation of direct protein function or downstream effects thereof; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human; (3) its absolute or relatively modulated presence or absence in clinical subset of patients such as those having defined or undefined genetic backgrounds.

For example, methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods may be used in a wide variety of formats including, but not limited to, substrate (e.g. membrane or glass) bound methods or array-based approaches. Other suitable amplification methods include, but are not limited to, ligase chain reaction (LCR) (see Wu and Wallace (1989) Genomics 4: 560, Landegren et al. (1988) Science 241:1077, and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc. Loss of heterozygosity (LOH) and major copy proportion (MCP) mapping (Wang, Z. C. et al. (2004) Cancer Res 64(1):64-71; Seymour, A. B. et al. (1994) Cancer Res 54, 2761-4; Hahn, S. A. et al. (1995) Cancer Res 55, 4670-5; Kimura, M. et al. (1996) Genes Chromosomes Cancer 17, 88-93; Li et al., (2008) MBC Bioinform. 9, 204-219) may also be used to identify regions of amplification or deletion.

Expression of immune-related biomolecules may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods. In preferred embodiments, activity of a particular gene is characterized by a measure of gene transcript (e.g. mRNA), by a measure of the quantity of translated protein, or by a measure of gene product activity. Marker expression may be monitored in a variety of ways, including by detecting mRNA levels, protein levels, or protein activity, any of which may be measured using standard techniques. Detection may involve quantification of the level of gene expression (e.g., genomic DNA, cDNA, mRNA, protein, or enzyme activity), or, alternatively, may be a qualitative assessment of the level of gene expression, in particular in comparison with a control level. The type of level being detected will be clear from the context. Various amplification and detection methods may also be used. For example, it is within the scope encompassed by the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR), real time PCR, NASBA, Q-beta amplification, target-mediated amplification, ligase chain reaction, self-sustained sequence replication (SSR), transcription amplification, and the like. Many techniques are known in the state of the art for determining absolute and relative levels of gene expression, commonly used techniques suitable for use in the present invention include in situ hybridization, microarray, chip array, serial analysis of gene expression (SAGE), Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

The activity or level of an immune-related biomolecule polypeptide may be detected and/or quantified by detecting or quantifying the expressed polypeptide. The polypeptide may be detected and quantified by any of a number of means well known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a cancer to an anti-immune checkpoint inhibitor therapy. Any method known in the art for detecting polypeptides may be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., “Basic and Clinical Immunology”, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

In one embodiment, a method for identifying an agent which promotes an immune response entails determining the ability of the candidate agent to promote or inhibit the interaction of immune-related biomolecule in the context of HSC cells expressing a gene of interest.

In another embodiment, a method for identifying an agent which inhibits an immune response entails determining the ability of the candidate agent to promote or inhibit the interaction of immune-related biomolecule in the context of HSC cells expressing a gene of interest.

The assays are cell-based assays and may comprise, for example, contacting (a) an HSC cell expressing a gene of interest, with a test agent and determining the ability of the test agent to modulate (e.g. stimulate or inhibit) the interaction between immune-related biomolecules (e.g., polypeptides) of interest. Determining the ability of the polypeptides to bind to, or interact with, each other may be accomplished, e.g., by measuring direct binding or by measuring a parameter of immune cell response.

For example, in a direct binding assay, polypeptides may be coupled with a radioisotope or enzymatic label such that binding of immune-related biomolecules may be determined by detecting the labeled protein in a complex. For example, the polypeptides may be labeled with ¹²⁵I, ³⁵S, ¹⁴C, or ³H, either directly or indirectly, and the radioisotope detected by direct counting of radioemission or by scintillation counting. Alternatively, the polypeptides may be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to modulate the interaction between immune-related biomolecules of interest without the labeling of any of the interactants. For example, a microphysiometer may be used to detect the interaction of immune-related biomolecule polypeptides without the labeling of either polypeptide (McConnell et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate may be used as an indicator of the interaction between compound and receptor.

In a preferred embodiment, determining the ability of the test agents (e.g. nucleic acids, polypeptides, antibodies, fusion proteins, peptides, or small molecules) to antagonize the interaction between a given set of immune-related biomolecules may be accomplished by determining the activity of one or more members of a set of immune-related biomolecule polypeptides. For example, the activity of polypeptides may be determined by detecting induction of a cellular second messenger (e.g., tyrosine kinase activity), detecting catalytic/enzymatic activity of an appropriate substrate, detecting the induction of a reporter gene (comprising a target-responsive regulatory element operatively linked to a nucleic acid encoding a detectable marker, e.g., chloramphenicol acetyl transferase), or detecting a cellular response regulated by the polypeptides, such as various autoimmune, allergic (e.g., asthma, atopic dermatitis, allergic conjunctivitis, pollen allergy, food allergy, etc.), vaccination, immunotolerance, cancer immunotherapy, immune exhaustion, immunological memory, or immunological epitope responses. Determining the ability of the test agent to bind to or interact with said polypeptide may be accomplished, for example, by measuring the ability of a compound to modulate immune cell costimulation or inhibition in a proliferation assay, or by interfering with the ability of said polypeptide to bind to antibodies that recognize a portion thereof.

Test agents that inhibit immune responses may be identified by their ability to inhibit immune cell proliferation, and/or effector function, or to induce anergy, clonal deletion, and/or exhaustion when added to an assay. For example, cells may be cultured in the presence of an agent that stimulates signal transduction via an activating receptor. A number of recognized readouts of cell activation may be employed to measure cell proliferation or effector function (e.g., antibody production, cytokine production, phagocytosis) in the presence of the activating agent. The ability of a test agent to block this activation may be readily determined by measuring the ability of the agent to effect a decrease in proliferation or effector function being measured, using techniques known in the art.

For example, agents of this invention may be tested for the ability to inhibit or enhance costimulation in a T cell assay, as described in Freeman et al. (2000) J. Exp. Med. 192:1027 and Latchman et al. (2001) Nat. Immumol. 2:261. HSC cells expressing a gene of interest may be CD4⁺ T cells or, alternatively, CD4⁺ T cells may be isolated from human PBMCs and stimulated with activating anti-CD3 antibody. Proliferation of T cells may be measured by ³H thymidine incorporation. An assay may be performed with or without CD28 costimulation in the assay. Similar assays may be performed with Jurkat T cells and PHA-blasts from PBMCs.

Alternatively, agents encompassed by the present invention may be tested for the ability to modulate cellular production of cytokines which are produced by or whose production is enhanced or inhibited in immune cells in response to immune response modulation. For example, HSC cells expressing a gene of interest may be suboptimally stimulated in vitro with a primary activation signal. For example, T cells may be stimulated with phorbol ester, anti-CD3 antibody or preferably antigen in association with an MHC class II molecule, and given a costimulatory signal, e.g., by a stimulatory form of B7 family antigen, for instance by a cell transfected with nucleic acid encoding a B7 polypeptide and expressing the peptide on its surface or by a soluble, stimulatory form of the peptide. Known cytokines released into the media may be identified by ELISA or by the ability of an antibody which blocks the cytokine to inhibit immune cell proliferation or proliferation of other cell types that is induced by the cytokine. For example, an IL-4 ELISA kit is available from Genzyme (Cambridge Mass.), as is an IL-7 blocking antibody. Blocking antibodies against IL-9 and IL-12 are available from Genetics Institute (Cambridge, Mass.). The effect of stimulating or blocking the interaction of immune-related biomolecules on the cytokine profile may then be determined. To identify cytokines which may play a role the induction of tolerance, an in vitro T cell costimulation assay as described above may be used. In this case, T cells would be given the primary activation signal and contacted with a selected cytokine, but would not be given the costimulatory signal. After washing the immune cells, the cells would be rechallenged with both a primary activation signal and a costimulatory signal. If the immune cells do not respond (e.g., proliferate or produce cytokines) they have become tolerized and the cytokine has not prevented the induction of tolerance. However, if the immune cells respond, induction of tolerance has been prevented by the cytokine. Those cytokines which are capable of preventing the induction of tolerance may be targeted for blockage in vivo in conjunction with reagents which block B lymphocyte antigens as a more efficient means to induce tolerance in transplant recipients or subjects with autoimmune diseases.

In one or more embodiments of the above described assay methods, it may be desirable to immobilize either polypeptides to facilitate separation of complexed from uncomplexed forms of one or both of the proteins, as well as to accommodate automation of the assay. Binding of a test compound to a polypeptide, may be accomplished in any vessel suitable for containing the reactants. Examples of such vessels include microtiter plates, test tubes, and micro-centrifuge tubes. In one embodiment, a fusion protein may be provided which adds a domain that allows one or both of the proteins to be bound to a matrix. For example, glutathione-S-transferase/immune-related polypeptide fusion proteins, or glutathione-S-transferase/target fusion proteins, may be adsorbed onto glutathione sepharose beads (Sigma Chemical, St. Louis, Mo.) or glutathione derivatized microtiter plates, which are then combined with the test compound, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes may be dissociated from the matrix, and the level of polypeptide binding or activity determined using standard techniques.

In an alternative embodiment, determining the ability of the test compound to modulate the activity of an immune-related polypeptide of interest may be accomplished as described above for cell-based assays, such as by determining the ability of the test compound to modulate the activity of a polypeptide that functions downstream of the polypeptide. For example, levels of second messengers may be determined, the activity of the interactor polypeptide on an appropriate target may be determined, or the binding of the interactor to an appropriate target may be determined as previously described.

In some embodiments, determination as to modulation of an immune-related indication of interest may be made in comparison to a control. The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels may be used in combination as controls in the methods encompassed by the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient may be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the invention are not limited to use of a specific cut-point in comparing the level of expression product in the test sample to the control.

As described above, control and experimental assays may involve the use of samples. The term “sample” used for detecting or determining the presence or level of at least one biomarker is typically whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method encompassed by the present invention further comprises obtaining the sample from the individual prior to detecting or determining the amount or expression of at least one marker in the sample. Samples are typically from a diseased tissue, such as cancer cells or tissues. The control sample may be from the same subject or from a different subject. The control sample is typically a normal, non-diseased sample. However, in some embodiments, such as for staging of disease or for evaluating the efficacy of treatment, the control sample may be from a diseased tissue. The control sample may be a combination of samples from several different subjects.

This invention further pertains to novel agents identified by the above-described screening assays. Accordingly, it is within the scope of this invention to further use an agent identified as described herein in an appropriate animal model. For example, an agent identified as described herein may be used in an animal model to determine the efficacy, toxicity, or side effects of treatment with such an agent. Alternatively, an agent identified as described herein may be used in an animal model to determine the mechanism of action of such an agent. Furthermore, this invention pertains to uses of novel agents identified by the above-described screening assays for treatments as described herein.

b. Therapeutic Methods

As described above, numerous therapeutic methods are contemplated regarding modulating Ptpn2 and other genes identified using the animal models described herein. In one aspect, the present invention provides a method for preventing and/or treating in a subject, a disease or condition associated with less than desirable immune response. The term “subject” refers to a) any healthy animal, such as a mammal or human; b) any animal, such as a mammal or human, afflicted with a immune-related disorder of interest; or c) any animal as described above from which HSC cells expressing a gene of interest is expressed are expressed. Subjects at risk for a disease that would benefit from treatment with the claimed agents or methods may be identified, for example, by any or a combination of diagnostic or prognostic assays known in the art. Administration of a prophylactic agent may occur prior to the manifestation of symptoms associated with an unwanted or less than desirable immune response. The appropriate agent used for treatment (e.g. antibodies, peptides, fusion proteins or small molecules) may be determined based on clinical indications and may be identified, e.g., using screening assays described herein.

Another aspect of the invention pertains to therapeutic methods of modulating an immune response, e.g., by modulating the interaction between immune-related biomolecules a) within an HSC expressing a gene of interest, b) between such an HSC and another cell, or c) between cells other than the HSC expressing a gene of interest. Without being bound by theory, it is believed that engineered HSC expressing a gene of interest described herein faithfully reproduce in vivo-generated, normal HSCs and/or cells derived therefrom to thereby produce more physiologically relevant responses relative to other methods of HSC cell engineering.

Modulatory methods encompassed by the present invention involve contacting a cell and/or an HSC expressing a gene of interest with an agent that modulates the interaction between immune-related biomolecules. Exemplary agents that modulate the interaction between immune-related biomolecules have been described above. For example, an agent that modulates immune-related biomolecule polypeptide activity includes a nucleic acid or a protein molecule, a naturally-occurring target molecule of the immune-related biomolecule protein, an anti-immune-related biomolecule protein antibody, immune-related biomolecule protein agonists or antagonists (e.g., antisense nucleic acid molecule, triplex oligonucleotide, and ribozymes), a peptidomimetic of an immune-related biomolecule protein agonist or antagonist, nucleic acid agonists or antagonists of immune-related biomolecule protein expression or activity, or other small molecule.

These modulatory agents may be administered in vitro or ex vivo (e.g., by contacting the cell with the agent) or, alternatively, in vivo (e.g., by administering the agent to a subject). As such, the present invention relates to methods of treating an individual afflicted with a disease or disorder that would benefit from upregulation of an immune response.

In some embodiments, agents described herein may be used to upregulate immune responses. In one embodiment, blockage of the interaction between immune-related biomolecules of interest results in upregulation of an immune response. Upregulation of immune responses may be in the form of enhancing an existing immune response or eliciting an initial immune response. For instance, enhancing an immune response using the subject compositions and methods is useful in treating cancer, an infectious disease (e.g., bacteria, viruses, or parasites), a parasitic infection, asthma associated with impaired airway tolerance, a neurological disease, and an immunosuppressive disease.

Exemplary infectious disorders include viral skin diseases, such as Herpes or shingles, in which case such an agent may be delivered topically to the skin. In addition, systemic viral diseases, such as influenza, the common cold, and encephalitis might be alleviated by systemic administration of such agents. In one preferred embodiment, agents that upregulate the immune response described herein are useful for modulating the arginase/iNOS balance during Trypanosoma cruzi infection in order to facilitate a protective immune response against the parasite.

Alternatively, immune responses may be enhanced in an infected patient through an ex vivo approach, for instance, by removing immune cells from the patient, contacting immune cells in vitro with an agent that modulate the interaction between immune-related biomolecules of interest and reintroducing the in vitro stimulated immune cells into the patient.

In certain instances, it may be desirable to further administer other agents that upregulate immune responses, for example, forms of other B7 family members that transduce signals via costimulatory receptors, in order to further augment the immune response.

Agents that upregulate an immune response may be used prophylactically in vaccines against various polypeptides (e.g., polypeptides derived from pathogens). Immunity against a pathogen (e.g., a virus) may be induced by vaccinating with a viral protein along with an agent that upregulates an immune response, in an appropriate adjuvant.

In another embodiment, upregulation or enhancement of an immune response function, as described herein, is useful in the induction of tumor immunity.

In another embodiment, the immune response may be stimulated by the methods described herein, such that preexisting tolerance, clonal deletion, and/or exhaustion (e.g., T cell exhaustion) is overcome. For example, immune responses against antigens to which a subject cannot mount a significant immune response, e.g., to an autologous antigen, such as a tumor specific antigens may be induced by administering appropriate agents described herein that upregulate the immune response. In one embodiment, an autologous antigen, such as a tumor-specific antigen, may be coadministered. In another embodiment, an immune response may be stimulated against an antigen (e.g., an autologous antigen) to treat a neurological disorder. In another embodiment, the subject agents may be used as adjuvants to boost responses to foreign antigens in the process of active immunization.

In one embodiment, immune cells are obtained from a subject and cultured ex vivo in the presence of an agent as described herein, to expand the population of immune cells and/or to enhance immune cell activation. In a further embodiment the immune cells are then administered to a subject. Immune cells may be stimulated in vitro by, for example, providing to the immune cells a primary activation signal and a costimulatory signal, as is known in the art. Various agents may also be used to costimulate proliferation of immune cells. In one embodiment immune cells are cultured ex vivo according to the method described in PCT Application No. WO 94/29436. The costimulatory polypeptide may be soluble, attached to a cell membrane, or attached to a solid surface, such as a bead.

In still another embodiment, agents described herein useful for upregulating immune responses may further be linked, or operatively attached, to toxins using techniques that are known in the art, e.g., crosslinking or via recombinant DNA techniques. Such agents may result in cellular destruction of desired cells. In one embodiment, a toxin may be conjugated to an antibody, such as a bispecific antibody. Such antibodies are useful for targeting a specific cell population, e.g., using a marker found only on a certain type of cell. The preparation of immunotoxins is, in general, well known in the art (see, e.g., U.S. Pat. No. 4,340,535, and EP 44167). Numerous types of disulfide-bond containing linkers are known which may successfully be employed to conjugate the toxin moiety with a polypeptide. In one embodiment, linkers that contain a disulfide bond that is sterically “hindered” are preferred, due to their greater stability in vivo, thus preventing release of the toxin moiety prior to binding at the site of action. A wide variety of toxins are known that may be conjugated to polypeptides or antibodies of the invention. Examples include: numerous useful plant-, fungus- or even bacteria-derived toxins, which, by way of example, include various A chain toxins, particularly ricin A chain, ribosome inactivating proteins such as saporin or gelonin, α-sarcin, aspergillin, restrictocin, ribonucleases, such as placental ribonuclease, angiogenic, diphtheria toxin, and Pseudomonas exotoxin, etc. A preferred toxin moiety for use in connection with the invention is toxin A chain which has been treated to modify or remove carbohydrate residues, deglycosylated A chain. (U.S. Pat. No. 5,776,427). Infusion of one or a combination of such cytotoxic agents, (e.g., ricin fusions) into a patient may result in the death of immune cells.

The terms “therapeutic response” or “therapeutic responsiveness” refer to a beneficial endpoint attained when exposed to a stimulus, such as an immunomodulatory response sufficient to modulate a target immune response. The terms may also refer to an improved prognosis, for example, as reflected by an increased time to cancer recurrence, which is the period to first recurrence censoring for second primary cancer as a first event or death without evidence of recurrence, or an increased overall survival, which is the period from treatment to death from any cause. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

The amount of cells needed for achieving a therapeutic effect may be determined empirically in accordance with conventional procedures for the particular purpose. Generally, for administering cells for therapeutic purposes, the cells are given at a pharmacologically effective dose. By “pharmacologically effective amount” or “pharmacologically effective dose” is an amount sufficient to produce the desired physiological effect or amount capable of achieving the desired result. Therapeutic benefit also includes halting or slowing the progression of the underlying disease or disorder, regardless of whether improvement is realized. Pharmacologically effective dose, as defined above, will also apply to therapeutic agents described herein used either alone or in combination with the cells. Effective doses of such therapeutic agents are well known in the art and may be determined by the ordinarily skilled artisan based on standard criteria, such as regulatory information, age, weight, state of health of the patient, and the nature and the severity of the indication. Suitable dosage ranges can vary according to these considerations. Moreover, the mode of administration may vary depending on such factors as well. Agents, including cells, may be introduced to the desired site by direct injection, or by any other means used in the art including, but are not limited to, intravascular, intracerebral, parenteral, intraperitoneal, intravenous, epidural, intraspinal, intrasternal, intra-articular, intra-synovial, intrathecal, intra-arterial, intracardiac, or intramuscular administration.

### EXEMPLIFICATION

The present invention is further illustrated by the following examples, which should not be construed as limiting.

### Example 1: Materials and Methods for Examples 2-12

a. Mouse Breeding and Production

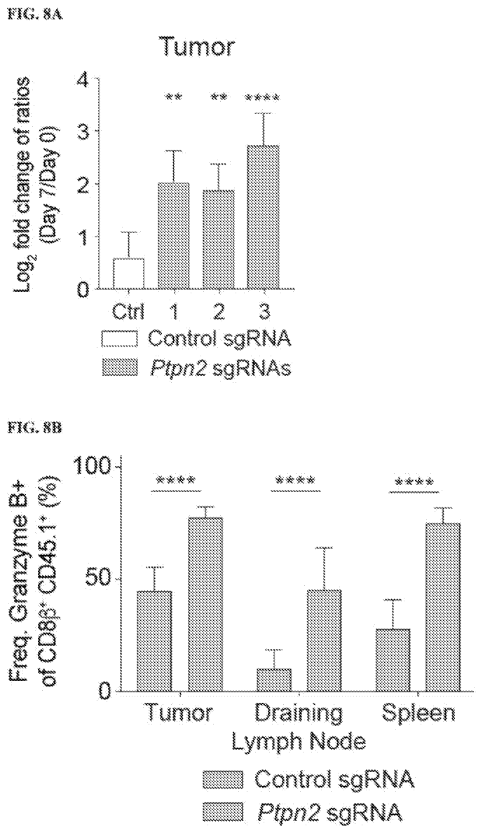

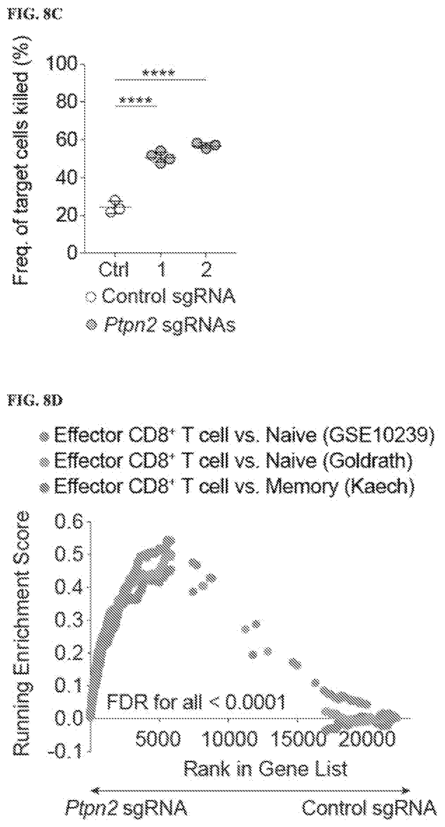

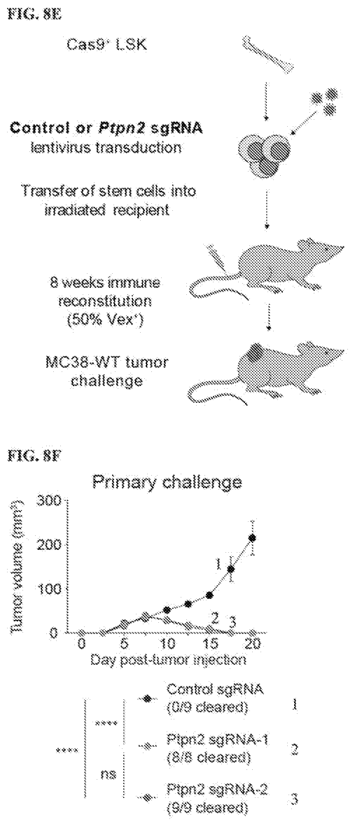

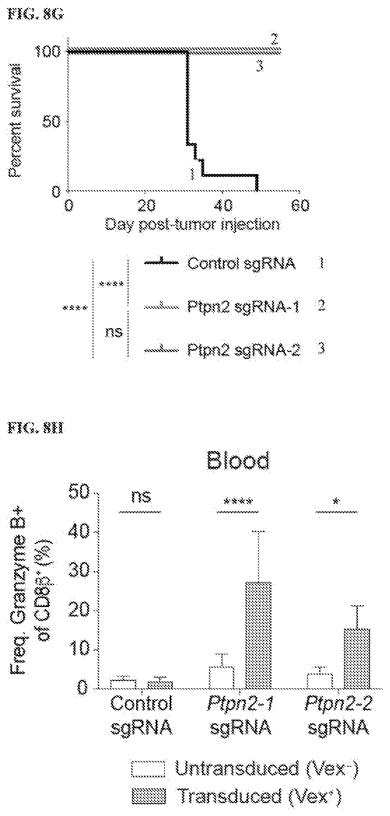

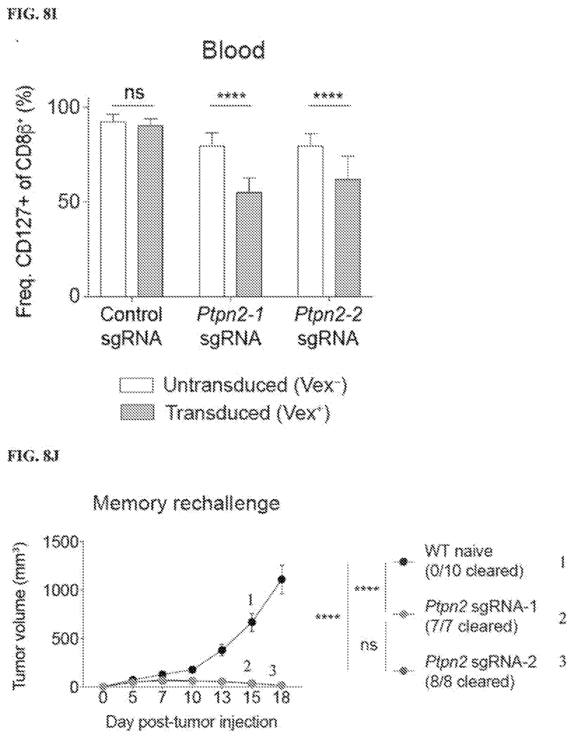

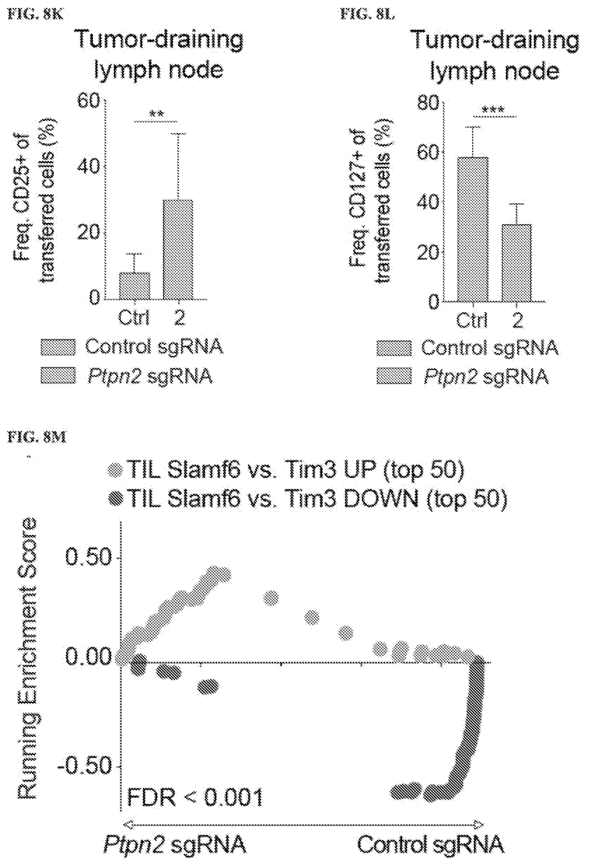

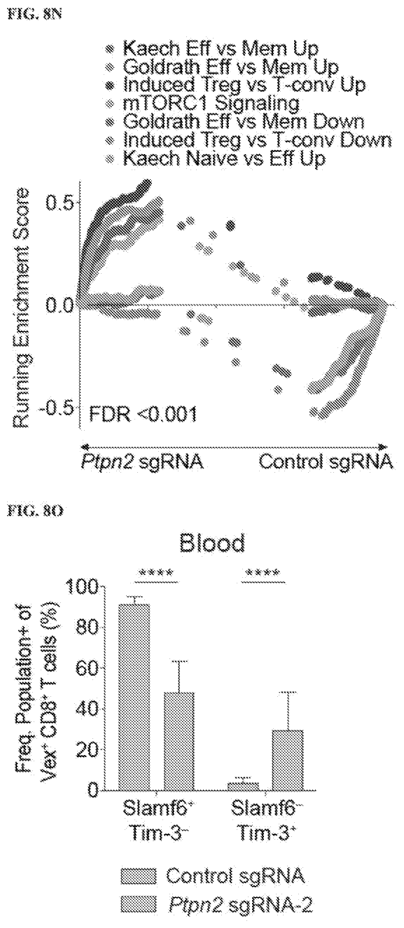

Seven to 10-week-old female or male mice were used for all experiments and 7 to 14-week-old female or male mice were used as donors for bone marrow chimera experiments. Wild-type (WT) C57BL/6 mice were purchased from The Jackson Laboratory. LoxP-STOP-LoxP Cas9 mice (B6J.129(B6N)-Gt(ROSA) 26Sortm1(CAG-cas9*,−EGFP)Fezh/J) were a generous gift from Dr. Feng Zhang, Massachusetts Institute of Technology (Platt et al. (2014) Cell 159:440-455). These mice were bred to Zp3-Cre mice (C57BL/6-Tg(Zp3-cre)1Gwh/J) to delete the loxP-STOP-LoxP in the female germline. The resulting Cas9-expressing strain was then bred to OT-1 (C57BL/6-Tg(TcraTcrb)1100Mjb/J) or P14 (Taconic B6.Cg-Tcratm1Mom Tg(TcrLCMV)327Sdz backcrossed 10 generations to Jackson C57BL/6J) TCR transgenic mice on the CD45.1 (B6.SJL-Ptprca Pepcb/BoyJ) congenic background. All strains used were backcrossed at least 10 generations to Jackson C57BL/6J. The sample size was chosen to ensure the possibility of statistical analysis and minimize the use of animals. Data exclusion was not used. Age and sex-matched animals were used for each experiment. For chimerism experiments LSK donor, LSK recipients, and CD8⁺ T cell transfer recipients were sex matched. Animals were also co-housed when possible. All attempts to reproduce the findings were successful. The LCMV Clone 13 infection, MC38 tumor, and B16 tumor experiments (FIGS. 8C, 8M, 8N, 8E-8G, 8O, 8H-8J, 15D-15G, and 17A-17F) were blinded during data collection. All experimental mice were housed in specific pathogen-free conditions and used in accordance with animal care guidelines from the Harvard Medical School Standing Committee on Animals and the National Institutes of Health.

b. Guide RNA Design and Cloning

The sgRNA oligonucleotides having sequences shown in Table 2 below were designed using the Broad CRISPR algorithm (Doench et al. (2016) Nat. Biotechnol. 34:184-191). Off-target sites were identified using the Benchling CRISPR design tool, which incorporates off-target rules from the MIT CRISPR algorithm (Hsu et al. (2013) Nat. Biotechnol. 31:827-832). sgRNAs were cloned into the sgRNA vector using a BsmBI restriction digest. This sgRNA vector was created by modifying an existing lentiviral shRNA vector (Godec et al. (2015) Proc. Natl. Acad. Sci. U.S.A. 112:512-517). Briefly, the modified vector contains the human U6 promoter (with Lac operator site) to express a sgRNA as well as the human PGK promoter to express the fluorophore, Vex. The plasmid and full sequence are available to the research community through Addgene.

TABLE 2
sgRNA and TIDE/Miseq primer sequences

| | | | |
|---|---|---|---|
| Gene | | | |
| Target | sgRNA | TIDE Primer Forward | TIDE Primer Reverse |

Pdcd1

| | | | |
|---|---|---|---|
| Pdcd1-1 | GGTACCCTGGTCATTCACTT | CCCCACCTCTAGTTGCCTGTT | GGCATTTCACCTGTAAAACCCAC |
| Pdcd1-2 | ACAGCCCAAGTGAATGACCA | CACCTCTAGTTGCCTGTTCTCCC | GGGGTGGATTTTGAGCCCCA |
| Pdcd1-3 | GACACACGGCGCAATGACAG | GTACAGGCTCCTTCCTCACAGC | TCCATCCCTTAAAGGTAAATGGGCATC |

Batf

| | | | |
|---|---|---|---|
| Batf-1 | AGAGATCAAACAGCTCACCG | ATAGACAGCAATCAGCAGTTGCC | AAGGGATCACGGGAGTAGCAT |
| Batf-2 | GTGGGTACTCACCAGGTGAA | AGGAGACCCAAGGGTGGGTA | TACATGCATGGGAGAGCGAAG |
| Batf-3 | TGTGAAGTACTTGAGCTCCT | ATAGACAGCAATCAGCAGTTGCC | AAGGGATCACGGGAGTAGCATC |

Ptpn2

| | | | |
|---|---|---|---|
| Ptpn2-1 | GAATATGAGAAAGTATCGAA | GGGCACTGAGCAGCAAACTTTAT | GTGACTAGCTTTCATCTTTGCCTCTT |
| Ptpn2-2 | CTCACTTCCATTATACCACC | CTGGAAGGCTGGCTGTAGTGTT | CTAACCTCCTCAGGCACCAGTC |
| Ptpn2-3 | ATGTGCACAGTACTGGCCAA | GCTGAAGCCAGCTTGATGTTC | CCCCCAAGAATTCTTAAGACCATC |

Ly75

| | |
|---|---|
| Ly75-1 | GTCACGAAACTCCATAATGG |
| Ly75-2 | GCTTGCTTGAGAAAACGTAA |

Ms4a1

| | |
|---|---|
| Ms4a1-1 | GTCACGAAACTCCATAATGG |
| Ms4a1-2 | GCTTGCTTGAGAAAACGTAA |

Fcgr1

| | |
|---|---|
| Fcgr1-1 | AGAGTACCATATAGCAAGGG |
| Fcgr1-2 | TGGGATGCTATAACTAGGCG |

Control

| | |
|---|---|
| Control-1 | GCGAGGTATTCGGCTCCGCG |
| Control-2 | GCTTTCACGGAGGTTCGACG |

*Sequencing Primer Bolded

c. Bone Marrow Isolation Aid Chimera Setup

Bone marrow cells were isolated and cultured as previously described in Godec et al. (2015) Proc. Natl. Acad Sci. U.S.A. 112:512-517. Femurs and tibias were isolated from donor mice, crushed, and ACK-lysed. LSK cells (lineage⁻ Sca-1⁺ Kit⁺) were enriched with a CD117 MACS isolation kit and then sorted to purity. The LSK (lineage⁻ Sca-1⁺ Kit⁺) cells were spin transduced with lentiviral constructs on retronectin-coated plates. LSK cells were then transferred intravenously into irradiated CD45.2⁺ recipients.

d. Cell Lines

MC38-OVA (gift from Natalie Collins, Dana Farber Cancer Institute), MC38 (gift from D. Vignali, University of Pittsburgh School of Medicine), B16.F10 and B16/GMCSF (both gifts from G. Dranoff, Novartis Institutes for Biomedical Research), 293× (gift from C. Kadoch, Dana Farber Cancer Institute), and MC38-GP₃₃₋₄₁ cells (Juneja et al. (2017) J. Exp. Afed 214:895-904) were cultured in DMEM supplemented with 10% FBS, 1% penicillin/streptomycin, and 20 μg/ml gentamicin. MC38-OVA cells were produced by transduction of MC38 cells with the lentiviral vector TRC-pLX305 (Broad Institute) containing ovalbumin (OVA) protein. MC38-OVA cells were selected for 2 days with 2 μg/mL puromycin prior to use to ensure expression of OVA (construct is OVA IRES Puromycin resistance). MC38-GP₃₃₋₄₁ cells were monitored for expression of GFP to ensure expression of GP₃₃₋₄₁ peptide (construct is GP₃₃₋₄₁ IRES GFP). B16/GMCSF were validated by ELISA. Parental MC38 and B16.F10 cell lines were validated by exome sequencing. BHK-21 cells (gift from E. John Wherry, University of Pennsylvania) were cultured in DMEM supplemented with 10% FBS, 1% penicillin/streptomycin, and 5% tryptose phosphate broth. Vero cells (gift from E. John Wherry, University of Pennsylvania) were cultured in EMEM supplemented with 10% FBS and 1% penicillin/streptomycin. All cell lines were confirmed mycoplasma negative.

e. In Vitro Stimulation

To analyze PD-1 expression by flow cytometry, sorted naïve CD4⁺ or CD8⁺ T cells were stimulated with 4 μg/mL αCD3/CD28 for 72 hours. Cells were then stained and analyzed by flow cytometry.

f. TIDE Assays and Next Generation CRISPR Sequencing

TIDE (Tracking of Indels by DEcomposition) assays were performed as previously described in Brinkman et al. (2014) Nucl. Acids Res. 42:e168. DNA was extracted from cells (DNeasy® kit), PCR was used to amplify the expected sgRNA target site, which was purified (PCR Purification kit), and analyzed using Sanger sequencing. For NGS sequencing, MiSeq sequencing was performed, and results were analyzed using the CRISPR algorithm at basepairtech.com to quantify indel/frameshift rates. All TIDE/MiSeq primers are listed in Table 2 described above.

g. Adoptive T Cell Transfer

Spleens were isolated from chimeric mice (>8 week post reconstitution) and naïve CD8⁺ T cells were purified using a naïve CD8⁺ MACS kit (>95% purity; Miltenyi Biotec). Cells were stained with lineage-specific antibodies (TER-119, B220, and Gr-1) and 7-Aminoactinomycin D (7-AAD) and then sorted (Lineage⁻, 7-AAD⁻, Vex⁺ cells). For LCMV studies, cells were transferred (500:500 mix) to recipient mice on day −1, and mice were infected with LCMV Clone 13 (as described below) on day 0. For tumor studies, cells were transferred (1000:1000 mix) to recipient mice on day −1, and mice were injected with MC38-OVA (as below) on day 0.

h. LCMV Production and Plaque Assay

LCMV Clone 13 virus was produced by infecting BHK-21 cells with an LCMV Clone 13 virus stock at an MOI of 0.01 and harvesting viral supernatants 48 hours later. Viral titers were determined by plating diluted viral stocks or serum/tissue samples on Vero cells with an agarose overlay. Four days later, the Vero cells were stained with neutral red dye, and then plaques were quantified 14 hours later.

i. LCMV Infection and Analysis

Mice were infected with 4×10⁶ PFU LCMV Clone 13 i.v., monitored for weight loss, and bled or sacrificed at day 8, 15, 22, or 30 post infection for flow cytometry analyses. For viral titer studies, mice were bled at days 8, 15, and 30 post-infection. Liver lymphocytes were isolated by dissociation of the liver followed by a 40%/60% Percoll® gradient. Lung lymphocytes were isolated by dissociation of the lung on a gentleMACS™ Dissociator followed by a 37° C. incubation in collagenase for 30 minutes. Lymphocytes were enriched on a 40%/60% Percoll® gradient. To deplete CD4⁺ T cells, mice were injected i.p. with 200 μg αCD4 on days −1 and 1 (relative to LCMV Clone 13 injection on day 0). To block IFNAR1, mice were injected i.p. with 1 mg αIFNAR (MAR1-5A3) or isotype (MOPC-21) on day 01 and day 1 (relative to LCMV clone 13 injection on day 0).

j. Tumor Injection

Mice were anesthetized with 2.5% 2,2,2-Tribromoethanol (Avertin) and injected in the flank subcutaneously with 2×10⁶ MC38-OVA tumor cells (competitive experiments) or 1×10⁶ MC38-WT (chimera primary challenge). For memory rechallenge experiments, chimeras were allowed to rest for 60 days post-primary tumor clearance and then were rechallenged with 5×10⁶ MC38-WT tumor cells on the opposite flank subcutaneously (as described above). For B16F10 experiments mice were challenged with 1×10⁶ B16.F10 subcutaneously (day 0), followed by injections on the opposite flank of 1×10⁶ irradiated B16/GM-CSF cells (days 1 and 4). Mice were then treated i.p. with 100 μg rat monoclonal αPD-1 (clone 29F.1A12) on days 12, 14, 16, 18, 20, 22, 24, and 26). Tumors were measured every 2-3 days once palpable using a caliper. Tumor volume was determined by the volume formula for an ellipsoid: ½×D×d² where D is the longer diameter, and d is the shorter diameter. Mice were sacrificed when tumors reached 2 cm³ or upon ulceration. To deplete CD8⁺ T cells, mice were injected i.p. with 100 μg αCD80 (or isotype) on days −3, 0, 3, 6, 9 and 200 μg αCD8α (or isotype) on days 12, 15, 18, 21, 24 (relative to MC38 tumor injection on day 0). For depletion of CD8⁺ T cells in memory mice mice were injected i.p. with a different clone and different species origin of αCD8β (or isotype) on days −3, 0, 3, 6, 9 (relative to MC38 rechallenge on day 0).

k. Tumor Infiltrating Lymphocyte Isolation

Tumors were excised and dissociated using a gentleMACS™ Dissociator. Tumors were then incubated in collagenase for 20 minutes at 37° C. Lymphocytes were enriched using a 40%/70% Percoll® gradient.

l. Monitoring of T Cell Responses in the Blood

To monitor the stability of Vex transduction, mice were bled via the tail vein. Blood was then lysed twice using ACK (ammonium-chloride-potassium; Thermo Fisher) lysis buffer, stained, and analyzed by flow cytometry. For monitoring of T cell responses to LCMV infection or tumor, mice were anesthetized with isoflurane, retro-orbitally bled, and lymphocytes were isolated by centrifugation at 400 g on a Histopaque®-1083 gradient, and stained for flow cytometry.

m. Flow Cytometry/Sorting

Flow cytometry analyses were performed on a BDTM LSR II or BD FACSymphony™ and cell sorting was performed on a BD FACSAria™ II. Antibodies and dyes were purchased from BD Biosciences (7-AAD, BrdU, Ki67); Biolegend (B220, CD11b, CD127, CD25, CD3ε, CD4, CD44, CD45.1, CD45.2, CD5, CD62L, CD8α, CD8β, c-Kit, CXCR5, Gr-1, Granzyme B, IFNγ, IFNAR1, PD-1, Sca-1, Slamf6, TCR Vα2, TCR Vβ5, TCR Vβ8, Ter-119, Tcf7, Tim-3, TNFα, TruStain fcX, Rat IgG2a κ Isotype, Rat IgG2b κ Isotype, Streptavidin BV421); BioXCell (CD3E, CD28, CD4); Thermo Fisher Scientific (Near-IR Fixable Live/Dead); and Cell Signaling Technology (pSTAT1). Additional antibodies used are included in Table 3.

n. In Vitro T Cell Differentiation Assay

Naive CD8⁺ T cells were obtained from spleens of control and Ptpn2-2 sgRNA-containing mice, enriched for naive CD8⁺ (MACS) and sorted on lineage (B220, TER-119, Gr-1)⁻, 7-AAD⁻ and Vex⁺. Naive CD8⁺ T cells were then activated on plate-bound αCD3 (5 μg/ml) with or without αCD28 (5 μg/ml), and supplemented with 200 U/mL IL-2, 1000 U/mL IFN-α, 50 ug/mL αIL-2, or 50 ug/mL αIFNAR blocking antibodies for 72 hours.

o. BrdU Incorporation and Detection

Mice were injected with 1 mg of BrdU i.p. 16 hours prior to sacrifice and analysis. Cells were processed and stained using the BD Biosciences BrdU flow kit.

TABLE 3
Reagent list

| | | |
|---|---|---|
| Reagent or Resource | Source | Identifier |

Antibodies/Stains

| | | |
|---|---|---|
| 7-AAD | BD Biosciences | 559926 |
| B220 (Clone RA3-8B2) | Biolegend | 103236 |
| B220 (Clone RA3-8B2) | Biolegend | 103208 |
| β-Actin (Polyclonal) | Abcam | ab88227 |
| CD11b (Clone M1/70 | Biolegend | 101218 |
| CD11b (Clone M1/70 | Biolegend | 101208 |
| CD11c (Clone N418) | Biolegend | 117328 |
| CD11c (Clone N418) | Biolegend | 117307 |
| CD127 (Clone A7R34) | Biolegend | 135024 |
| CD19 (Clone 805) | Biolegend | 115533 |
| CD20 (Clone SA275A11) | Biolegend | 150412 |
| CD244 (Clone Eolo244F4) | Thermo Fisher Scientific | 25-2441-80 |
| CD25 (Clone 3C7) | Biolegend | 101904 |
| CD28 (Clone 7.51) | BioXCell | BE0015-1 |
| CD3e (Clone 145-2C11) | Biolegend | 100336 |
| CD3e (Clone 145-2C11) | Biolegend | 100308 |
| CD3e (Clone 145-2C11) | BioXCell | BE0001-1 |
| CD3e (Clone 17A2) | Biolegend | 100220 |
| CD4 (Clone GK1.5) | BioXCell | BE0003-1 |
| CD4 (Clone RM4-5) | Biolegend | 100531 |
| CD4 (Clone RM4-5) | Biolegend | 100516 |
| CD4 (Clone RM4-5) | Biolegend | 100543 |
| CD44 (Clone IM7) | Biolegend | 103008 |
| CD44 (Clone IM7) | Biolegend | 103030 |
| CD44 (Clone IM7) | Biolegend | 103025 |
| CD45.1 (Clone A20) | Biolegend | 110716 |
| CD45.1 (Clone A20) | Biolegend | 110706 |
| CD45.2 (Clone 104) | Biolegend | 109824 |
| CD45.2 (Clone 104) | Biolegend | 109832 |
| CD49b (Clone PX5) | Biolegend | 108909 |
| CD5 (Clone 53-7.3) | Biolegend | 100608 |
| CD62L (Clone MEL-14) | Biolegend | 104417 |
| CD64 (Clone X54-517.1) | Biolegend | 139303 |
| CD89 (Clone H1.2F3) | Biolegend | 104513 |
| CD8α (Clone 53-6.7) | Biolegend | 100737 |
| CD8β (Clone eBioH35-17.2) | Thermo Fisher Scientific | 11-0083-82 |
| CD8β (Clone YTS1567.7) | Biolegend | 126606 |
| CD8β (Clone YTS1567.7) | Biolegend | 126610 |
| CD8β (Clone YTS1567.7) | Biolegend | 125620 |
| CXCR5 (Clone 2G8) | BD Biosciences | 551960 |
| c-Kit (Clone ACK2) | Biolegend | 136108 |
| Donkey anti-rabbit IgG (H + L) | LI-COR Biosciences | 925-32213 |
| F4/80 (clone 8MB) | Biolegend | 123116 |
| Goat anit-mouse IgG (H + L) | LI-COR Biosciences | 825-68070 |
| GP33-41 Tetramer PE | NH Tetramer Core Facility | NA |
| Gr-1 (Clone RB-808) | Biolegend | 108405 |
| Granzyme B (Clone GB11) | Biolegend | 515403 |
| KI67 (Clone B56) | BD Pharmingen | 561284 |
| Ly-108 (Clone 13G3) | BD Pharmingen | 561547 |
| Mouse IgG1, κ Isotype Ctrl (Clone MOPC-21) | Biolegend | 400112 |
| Near-IR Fixable Live/Dead | Thermo Fisher Scientific | L10119 |
| NK1.1 (Clone PK138) | Biolegend | 108732 |
| PD-1 (Clone 29F.1A12) | Biolegend | 135206 |
| PD-1 (Clone 29F.1A12) | Biolegend | 135209 |
| RatigG2a.κ Isotype Ctrl (Clone RTK2758) | Biolegend | 400508 |
| RatigG2b.κ Isotype Ctrl (Clone (RTK4530) | Biolegend | 400612 |
| Sca-1 (Clone D7) | Biolegend | 108128 |
| Streptavidin-BV421 | Biolegend | 405225 |
| TCFγ (Clone S33-966) | BD Biosciences | 564217 |
| TC-PTP (Clone 8F3) | Medimabs | MM-0019 |
| TCR Vα2 (Clone B20.1) | Biolegend | 127814 |
| TCR Vβ5 (Clone MR9-4) | BD Biosciences | 562087 |
| Ter-119 (Clone Ter-119) | Biolegend | 118208 |
| Tim-3 (Clone RMT3-23) | Biolegend | 118703 |
| Tim-3 (Clone RMT3-23) | Biolegend | 119723 |
| TruStain 1cX (Clone 93) | Biolegend | 101320 |

Bacterial and virus strains

| | | |
|---|---|---|
| LCMV Cl. 13 virus | A gift from E. John Wherry | N/A |
| Stb13 competent E. coli | Thermo Fisher Scientific | C737303 |

Chemicals, Recombinant Proteins, and Media

| | | |
|---|---|---|
| 10x Tris Buffered Saline (TBS) | Bio-Rad | 1706435 |
| 2-Mercaptoethanol (1000x) | Thermo Fisher Scientific | 21985-023 |
| 2,2,2-Triteromoethanol, 97% (Avertin) | Sigma-Aldrich | T48402 |
| ACK Lysing buffer | Lonza | 10-548E |
| BSA | Sigma-Aldrich | A2153 |
| Collagenase type 1 | Worthington | LS004194 |
| DMEM | Invitrogen | 11965-118 |
| DPBS, no calcium, no magnesium | Life Technologies | 14190-250 |
| EMEM | ATCC | 30-2003 |
| FBS | Sigma | F2442 |
| Gentamicin | Thermo Fisher Scientific | 16710072 |
| Hall Protease and Phosphatase Inhibitor | Thermo Fisher Scientific | 78440 |
| HEPES | Fisher Scientific | 15630-130 |
| Histopaqus-10B3 | Sigma-Aldrich | 10831 |
| IsoThesis (Isoflurane) | Henry Schein | 29404 |
| Medium 199 | Thermo Fisher Scientific | 31100035 |
| Neutral Red | Sigma-Aldrich | N7005 |
| NuPage 4-12% Bis-Tris Protein Gels | Thermo Fisher Scientific | NP0323BOX |
| Odyssey blocking buffer | LI-COR Biosciences | 327-50000 |
| Odyssey Nitrocellulose Membrane | LI-COR Biosciences | 928-31092 |
| Opti-MEM \| Reduced Serum Medium | Life Technologies | 31985-062 |
| Pen-Strep | Thermo Fisher Scientific | 15140122 |
| Percell | VWR | 59428-524 |
| Polybrene | Sigma-Aldrich | H8157 |
| Polyethylenimine (Mw 40,000) | Polysciences | 24765-2 |
| Puromycin dihydrochloride | Sigma-Aldrich | P7255 |
| Recombinant Human IL-2 | R&D Systems | 202-IL-060 |
| Recombinant Murine FY3-Ligand | Pepro Tech | 250-31L |
| Recombinant Mruine IFN-γ | Pepro Tech | 315-05 |
| Recombinant Murine IL-12 p70 | Pepro Tech | 210-12 |
| Recombinant Murine IL-7 | Pepro Tech | 217-17 |
| Recombinant Murine SCF | Pepro Tech | 250-03 |
| Recombinant Murine TPG | Pepro Tech | 315-14 |
| RetroNectin | Takara Bio | T100B |
| RIPA lysis buffer | Thermo Fisher Scientific | 8990 |
| RLT Buffer | QIAGEN | 79216 |
| RPMI 1640 | Invitrogen | 11875-119 |
| StemSpan SFEM | STEMCELL Technologies | 9600 |
| Sulfatrim | Patterson Veterinary Supply | 07-891-6040 |
| Trypsin-EDTA (0.08%) | Thermo Fisher Scientific | 26350-064 |
| Trytose phosphate broth solution | Sigma-Aldrich | T8169 |
| Tween 20 | Sigma-Aldrich | P1379 |
| UltraPure 0.5M EDTA, pH 8.0 | Invitrogen | 15575-020 |

Commercial Assays

| | | |
|---|---|---|
| Agencourt AMPure XP 60 mL Kit | Beckman Coulter | A63851 |
| Anti-Ter-118 Microbeads | Miltenyl | 130-049-901 |
| CD117 MicroBeads, mouse | Miltenyl | 190-093-224 |
| CD11b MicroBeads, human and mouse | Miltenyl | 130-049-801 |
| CD11c MicroBeads UltraPure.mouse | Miltenyl | 130-108-338 |
| CD8 microbeads (Ly-2) | Miltenyl | 130-049-401 |
| DNessy Blood & Tissue Kit | GIAGEN | 695004 |
| Gateway BP Clonase II enzyme mix | Life Technologies | 11789020 |
| Gateway LR Clonase II enzyme mix | Life Technologies | 11791020 |
| Mouse CD19 Micorbeads | Miltenyl | 130-052-201 |
| Naive CD8α T cell Isolation Kit | Miltenyl | 130-098-543 |
| Nextera XT DNA Sample Preparation Kit | Illumina | 131-1096 |
| Pierce BCA Protein Assay Kit | Thermo Fisher Scientific | 23225 |

Deposited Data

| | | |
|---|---|---|
| RAN-seq data | This study | GEO submission pending |

Experimental Models: Cell Lines

| | | |
|---|---|---|
| Hamster cell line: BHK-21 | A gift from E. John Wherry | N/A |
| Human cell line: HEK-293x | A gift from Cigali Kadoch | N/A |
| Mouse cell line: MC38 | A gift from Darlo Vignali | N/A |
| Mouse cell line: MC38-GP33-41-GFP | Juneja et al, JEM 2017 | N/A |
| Mouse cell line: MC38-OVA-Puro | This sturdy | N/A |
| Hamster cell line: Vero | A gift from E. John Wherry | N/A |

Experimental Models: Mouse Strains

| | | |
|---|---|---|
| 86.Cg-Toratm1Mom T0 (tcrLCMV)27 Sdz | Taconic | 4138 |
| 86.SJL-PorcsPepcb/BoyJ | The Jackson Laboratory | JAX: 002014 |
| BBJ.129(B0N)-GH(ROSA)26 Sorhm1 | A gift from Feng Zhang | N/A |
| (GAG-cas9\hat{ },EGFP.Fezg.H | | |
| C57BL/6-Tg(Tora Tcrb)1100Mjb/J | The Jackson Laboratory | JAX: 003831 |
| C57BL/6-Tg(2p3-crs)1Gwh/J | The Jackson Laboratory | JAX: 006888 |
| C678L/6J | The Jackson Labroatory | JAX: 000664 |

Oligonucleotides

| | |
|---|---|
| See Table 1 | IDT |

Plasmids

| | | |
|---|---|---|
| Gateway pDONR221 | Life Technologies | 11791020 |
| pMD2.G | A gift from Cigali Kadoch | N/A |
| MSCV-Vex | A gift from E. John Wherry | N/A |
| ρsPAX2 | A gift from Cigali Kadoch | N/A |
| TRC-pLX305 | Broad Institute | N/A |
| TRC-pLX931 | Broad Institute | N/A |

Restriction Enzymes

| | | |
|---|---|---|
| BamHi | NEW ENGLAND BioLabs | R0136S |
| BsIWi | NEW ENGLAND BioLabs | R0663S |
| BsmBi | NEW ENGLAND BioLabs | R0580S |
| EcoRi | NEW ENGLAND BioLabs | R0101S |
| PpuMi | NEW ENGLAND BioLabs | R0506S |
| PspXi | NEW ENGLAND BioLabs | R0658S |
| Psll | NEW ENGLAND BioLabs | R0140S |
| Sall | NEW ENGLAND BioLabs | R0138S |

Software and Algorithms

| | | |
|---|---|---|
| Benchling | Benchling Inc | http://benchling.com |
| Bowtie2 | Langmead et al, Nat Methods 2012 | http://bowtie-bio.sourceforge.net/bowtie2index.shtmi |
| CRISPR Design | Broad Institute | http://crispr.mit.edu |
| DESeq2 R Package | Love et al, Genome Biology 2014 | http://github.com/mikelove/DESeq2 |
| FlowJo 10.4.2 | FLOWJO | http://flowjo.com |
| GraphPad Prism 8 | GraphPad Software | http://graphpad.com |
| GSEA | Subramanian et al, PNAS 2005 | http://www.gsea-msigdb.org/gsea/index.jsp |
| HTSeq | Anders et al, Bioinformatics 2015 | https://htseq.readthedocs.iofen/release_0.1.0/ |
| ImageJ | NIH | https://imagej.nihgov/ij/ |
| NGS CRISPR Workflow | Ben Ebert (DFCI) | https://app.basepairtech.com/#workflows/ |
| TIDE | Brinkman et al, Nucleic Acid Research 2014 | https://tide.deskgen.com |
| Timmomatic | Bolger et al, Bioinformatics 2014 | https://www.usedelab.org/cms/?page=trimmomatic |

p. Western Blotting

Spleen and lymph nodes (cervical, axillary, and inguinal) were isolated from control, Pqpn2-1, or Ptpn2-2 sgRNA-containing chimeric mice. Spleen and lymph node were pooled and CD8⁺ T cells were enriched using CD8α microbeads. The spleen and lymph node samples were then sorted for CD8β⁺ Vex⁺. Whole cell lysates were generated using a mixture of Pierce RIPA buffer and protease/phosphatase inhibitor at a final concentration of 1 mg/mL. Protein concentration was measured with a BCA protein assay kit. Subsequently, 30 μg of protein was run on a NuPage® 4-12% bis-tris protein gel and then transferred to a nitrocellulose membrane. The membrane was incubated overnight in Odyssey® blocking buffer followed by staining with anti-TC-PTP (C-term) mouse monoclonal IgG antibody and anti-β actin rabbit polyclonal IgG antibody at a 1/1000 dilution for 1 hour at room temperature (Wiede et al. (2017) J. Autoimmun. 76:85-100). The membrane was washed with TBS-T and then incubated with secondary antibodies IRDye® 680RD Goat anti-mouse IgG and IRDye® 800CW Donkey anti-Rabbit IgG (H+L), at a 1/10000 dilution for 1 hour at room temperature. The membrane was washed and visualized using the Li-Cor Clx Imaging System (Li-Cor). The blot was then analyzed using ImageJ software.

q. RNA-Sequencing Analysis of T Cells

Day 7 or 8 post-tumor or virus injection respectively, transferred T cells were isolated from the tumor or spleen (LCMV) (as described above) and replicates of 500 cells were sorted into 25 μL of buffer RLT+1% beta-mercaptoethanol v/v. After flash-freezing on dry ice and storage at −80° C., lysates were converted to cDNA following capture with Agencourt® RNAClean™ beads using the SmartSeq2 protocol as previously described in Trombetta et al. (2014) Curr. Protoc. Mol. Biol. 107:1-17. The cDNA was amplified using 16 PCR enrichment cycles prior to quantification and dual-index barcoding with the Illumina Nextera® XT kit. The libraries were enriched with 12 cycles of PCR, then combined in equal volumes prior to final bead clean-up and sequencing on an Illumina NextSeq500 sequencer by 37 bp paired-end reads. After demultiplexing, low quality base-reads were trimmed with Trimmomatic software using the following parameters: LEADING: 15, TRAILING: 15, SLIDINGWINDOW: 4:15, MINLEN: 16 (Bolger et al. (2014) Bioinformatics 30:2114-2120). Trimmed reads were then aligned to the mm10 mouse genome using Bowtie 2 software. HTSeq was used to map aligned reads to genes and to generate a gene count matrix. Normalized counts and differential expression analysis were performed using the DESeq2 R software package. Gene set enrichment analysis was performed as previously described in Subramanian et al. (2005) Proc. Natl. Acad. Sci. U.S.A. 102:15545-15550.

r. Statistical Analysis

Statistical analyses were performed using GraphPad Prism 7 software or R. Data were considered statistically significant with p values<0.05 by paired Student's t test for comparing two groups, one-way ANOVA for single comparisons with groups greater than two, two-way ANOVA for repeated measures comparisons or for multiple comparisons within groups, log-rank Mantel-Cox test for survival comparisons, and the Kolmogorov-Smirnov test for GSEA. For GSEA analysis of RNA-seq data the Kolmogorov-Smirnov test was used. For analysis of single cell RNA-seq data the Wilcoxon rank sum test was used for signature enrichments and a binomial tested was used to determine proportional differences of control or Ptpn2-deleted cells in the clusters.

s. In Vitro Cytotoxicity Assay

Naive CD8⁺ T cells were obtained from spleens of control, Ptpn2-1, and Ptpn2-2 sgRNA-containing mice and enriched using a naive CD8⁺ MACS kit (Miltenyi Biotec). Samples were then sorted according to lineage markers (B220, TER-119, Gr-1)⁻, 7-AAD⁻, and Vex⁺. CD8⁺ T cells were then activated on a plate coated with 1 μg/ml αCD3/CD28 and cultured with 100 U/mL of IL-2 and 10 ng/mL IL-12 for 72 hours. One day prior to co-culture, 10,000 MC38-GP₃₃₋₄₁-GFP tumor cells were seeded in a 96-well plate with 20 ng/mL IFNγ. The next day, activated CD8⁺ T cells were plated on top of the tumor cells at a 4:1 effector to target ratio (or 0:1 as baseline control) for 21 hours. Cells were then trypsinized, stained, and analyzed by flow cytometry to determine the number of remaining live tumor cells. Killing percentage was calculated by the formula:

100%×[1−(# live tumor cells in well with T cells/# live tumor cells in well with no T cells)].

t. Restimulation and Flow Cytometry of Phosphorylated Proteins

Splenocytes were isolated from LCMV Clone 13 infected mice on day 8 p.i., ACK lysed and resuspended in MACS buffer. Cells were stained with the following surface marker antibodies: CD8β, CD45.1, CD45.2, Slamf6, Tim-3 for 30 minutes on ice. For cytokine stimulation, splenocytes were stimulated with 200 U/mL IL-2 or 1000 U/mL IFN-α for 0, 2, 5, 10, 15 and 30 min at 37° C. After stimulation, cells were pelleted at 500 g and fixed in 2% methanol free-formaldehyde, washed in MACS buffer and permeabilized with ice cold 90% methanol. Cells were then stained with pSTAT1 antibody (Cell Signaling Technology).

u. ATAC-Seq Library Preparation and Analysis

50,000 control or Ptpn2 sgRNA-containing Slamf6⁺ or Tim-3⁺ P14 T cells per replicate were sorted from spleens of day 8 LCMV Clone 13 infected mice into PBS with 10% FBS. Pelleted cells were incubated in 50 μl of reaction mix (containing 2× TD, Tn5 enzyme, 2% digitonin in nuclease-free water) as previously described (Corces et al. (2016) Nat. Genet. 48: 1193-1203). The transposase reaction was performed at 37° C. for 30 minutes with agitation at 300 RPM. DNA was then purified using a QIAgen MinElute® Reaction Cleanup kit. A post PCR cleanup was performed using Agencourt® AMPure XP beads (Beckman Coulter/Agencourt) and library quality was verified using Tapestation analysis. Samples were sequenced on an Illumina NextSeq500 sequencer using paired-end 37 bp reads.

Quality trimming and primer removal within raw fastq files were done with Trimmomatic 0.33 using the following parameters: LEADING: 15 TRAILING: 15 SLIDINGWINDOW: 4:15 MINLEN: 36. Trimmed reads were aligned to mm9 with bowtie2.2.4 using a maximum insert size of 1000. Aligned barns were sorted, duplicates marked, and reads mapping to the blacklist region removed (Buenrostro et al. (2013) Nat. Methods 10:1213-1218). Peak-calling using MACS 2.1.1 was performed on merged bam files (samtools 1.3) from biological replicates using a q-value threshold of 0.001. Consensus peaks from all biological conditions were merged to create a single peak universe. Cut sites were extracted from each biological replicate and the number of cuts within each peak region was quantified to generate a raw counts matrix. DESeq2 was used to normalize the counts matrix and perform differential accessibility analysis between all relevant comparisons. Tracks were visualized using Integrative Genomics Viewer 2.3.77 (Broad Institute).

v. Single Cell RNA-Seq Library Preparation and Analysis

Control or Ptpn2 sgRNA-containing P14 CD8⁺ T cells were sorted from spleens of day 30 LCMV Clone 13 infected, CD4-depleted mice based on the markers CD8p, CD45.1, CD45.2, Vex, and Live. Cells were counted and loaded onto the Chromium Controller (10× Genomics) for a target recovery of 5,000 single cells. Samples were processed per the manufacturer's protocol and sequenced on an Illumina NextSeq500 sequencer using a 75 bp kit with paired-end reads. The Cell Ranger analysis pipeline version 1.2 was used for sample demultiplexing, barcode processing, alignment, filtering, UMI counting, and aggregation of sequencing runs. The R Seurat package (Satija et al. (2015) Nat. Biotech. 33:495-502) was used for downstream analyses.

For each cell, two quality control metrics were calculated: (1) the total number of genes detected and (2) the proportion of UMIs contributed by mitochondrially encoded transcripts. Cells were excluded from downstream analysis if fewer than 200 genes were detected and if mitochondrially encoded transcripts constituted greater than 5% of the total library, yielding an expression matrix of 7,027 cells by 13,133 genes. Each gene expression measurement was normalized by total expression within the corresponding cell and multiplied by a scaling factor of 10,000. Mean and dispersion values were calculated for each gene across all cells; 1,829 genes (LCMV) classified as highly variable. Highly variable genes were used for principal components analysis (PCA). Principal components were determined to be significant (P<0.001) using the jackstraw method and tSNE was performed on these significant PCs (PCs 1-17) using default parameters for 1,000 iterations for visualization in two dimensions. Unsupervised clustering was performed using a shared nearest neighbor modularity optimization-based algorithm (Waltman and van Eck (2013) Eur. Phys. J. B 86:471). Single-cell signature scoring using FastProject (DeTomaso and Yosef (2016) BMC Bioinformatics 17:315). was performed with the Hallmark database from MSigDB and using signatures of the subpopulations derived from the prior analysis of exhausted CD8⁺ splenocytes from LCMV Clone 13 infected mice (Miller et al. (2019) Nat. Immunol.). Differential gene expression and signature enrichment analysis was performed using a Wilcoxon rank sum test. To determine the relative proportion of Ptpn2-deleted cells within each cluster, a binomial test was performed against the proportion of Ptpn2-deleted cells within the total dataset.

### Example 2: Candidate Genes are Efficiently Deleted in the Hematopoietic System

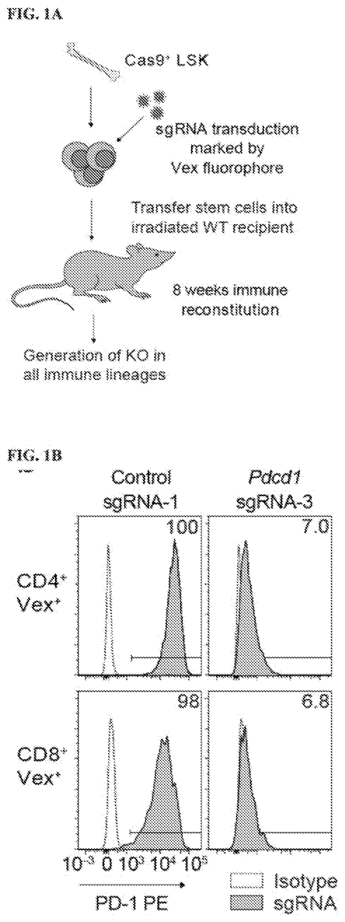

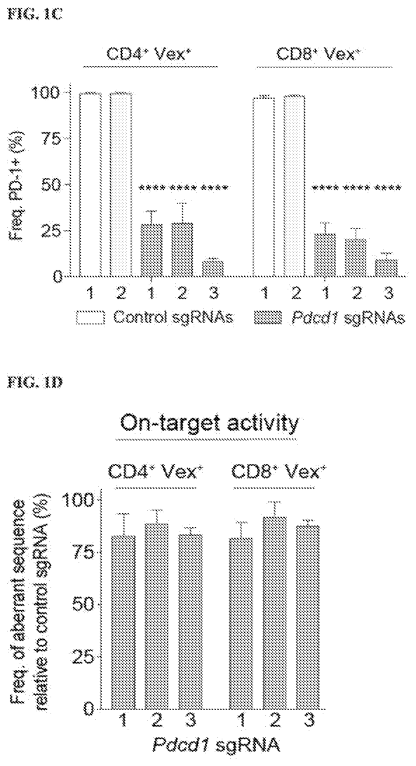

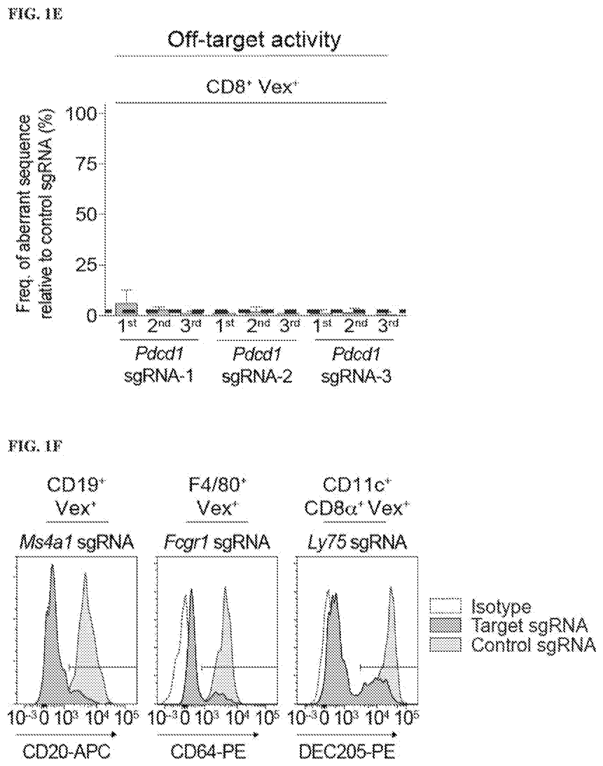

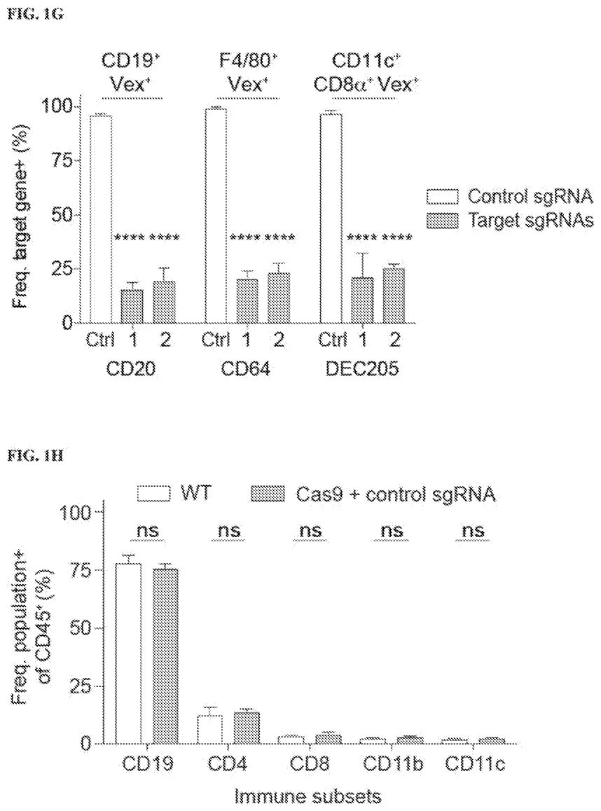

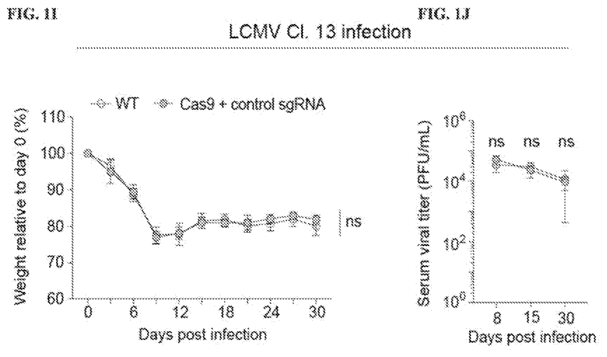

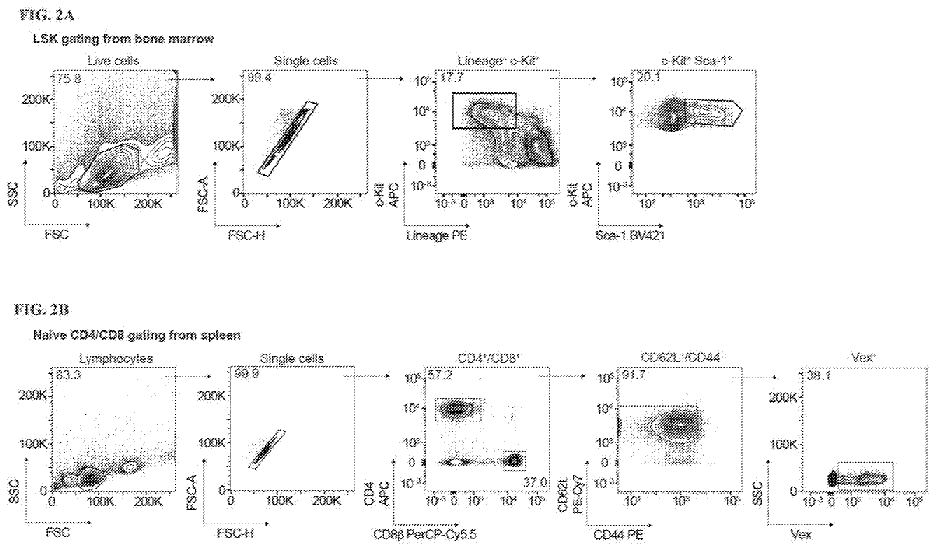

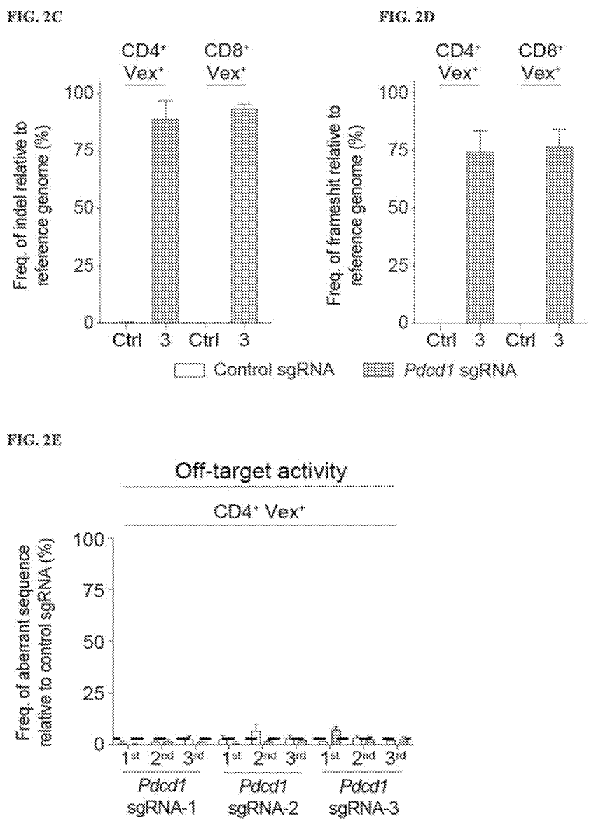

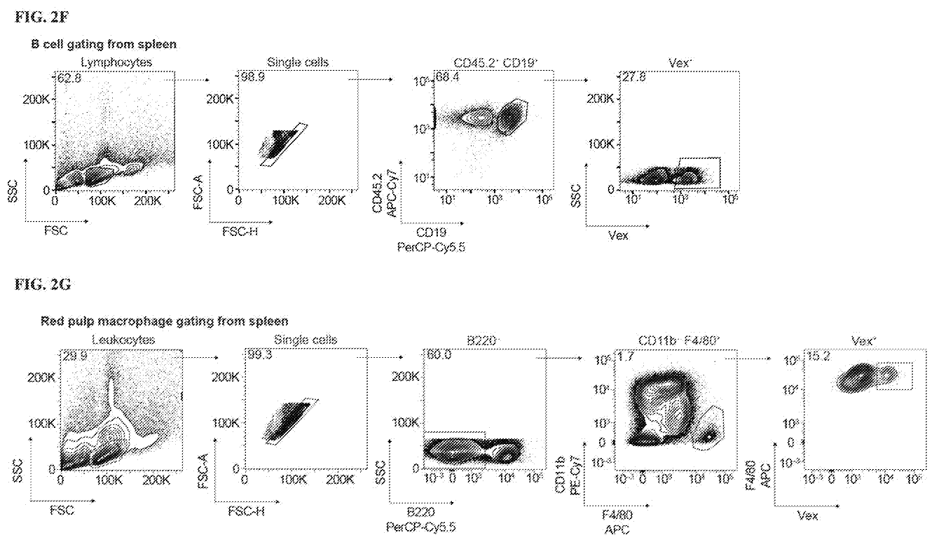

Therapies that target the function of immune cells have significant clinical efficacy, particularly in cancer, where immunotherapy with checkpoint blockade has become a mainstay of treatment. Although functional genomics has accelerated therapeutic target discovery in cancer, its use as a discovery tool in primary immune cells is limited because vector delivery to many immune cell types is inefficient and perturbs their cell state, potentially obscuring important phenotypes. To create gene deletions in hematopoietic lineages, a chimeric guide RNA delivery system was developed using bone marrow from Cas9-expressing mice (FIG. 1A) (Platt et al. (2014) Cell 159:440-455). To do this, Cas9-expressing Lineage⁻ Sca-1⁺ c-Kit⁺ (LSK) cells were isolated from donor mice (FIG. 2A) and the LSK cells were transduced with a lentiviral sgRNA expression vector containing a Vex (violet-excited GFP) fluorescent reporter, and transferred to irradiated recipients to create bone marrow chimeric mice. Following 8 weeks of immune reconstitution, immune cells that express Cas9 and the sgRNA (marked by Vex) were isolated.

To determine if the chimeric CRISPR system could delete genes in CD4⁺ and CD8⁺ T cells, chimeras carrying two non-targeting control sgRNAs or three Pdcd1 targeting sgRNAs were created. T cells from these chimeric mice were stimulated with αCD3/CD28 to induce PD-1 expression and a significant reduction of PD-1 expression in the presence of targeting sgRNAs, but not control sgRNAs, was found. On average, 80% deletion was achieved in both CD4⁺ and CD8⁺ T cells (FIGS. 1B and 1C). Analyses of naive CD4⁺ and CD8⁺ T cells from these mice prior to stimulation using the TIDE assay confirmed that these T cells had ˜80% aberrant sequences, indicating efficient CRISPR-mediated indel formation (FIGS. 1D and 2B) (Brinkman et al. (2014) Nucl. Acids Res. 42:e168). On-target effects were further analyzed using next-generation sequencing and it was found that both the indel and frameshift percentages correlated with loss of PD-1 protein expression (FIGS. 1D and 2C). Off-target effects in this system were analyzed by performing the TIDE assay for the top three predicted off-target sites for each of the three Pdcd1 sgRNAs, and minimal off-target editing above background was found in CD8⁺ T cells (FIG. 1E) and CD4⁺ T cells (FIG. 2E).

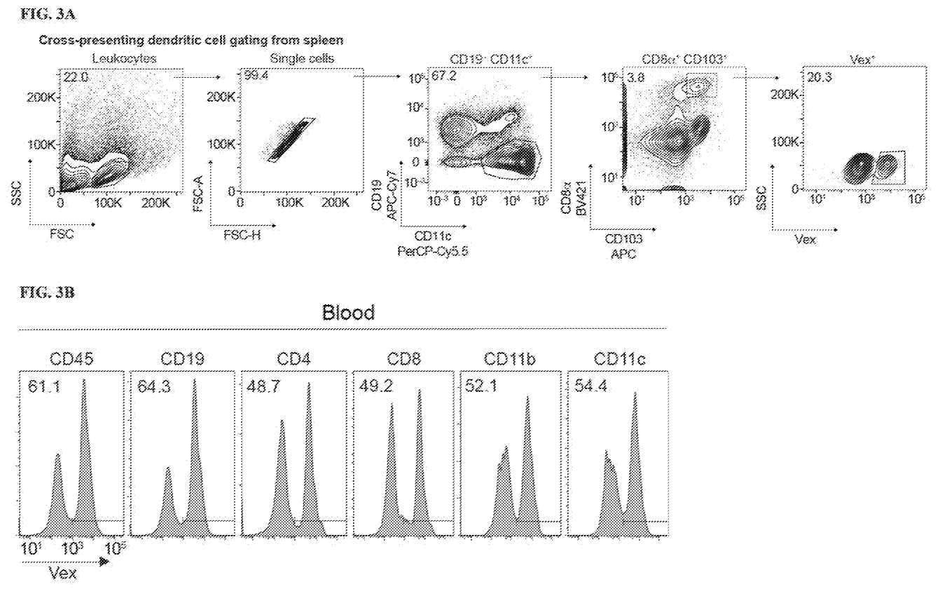

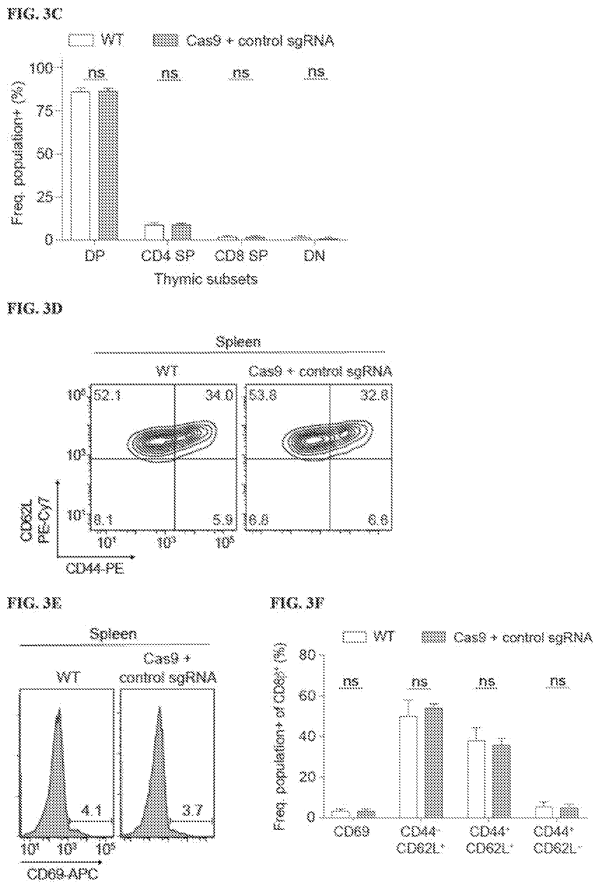

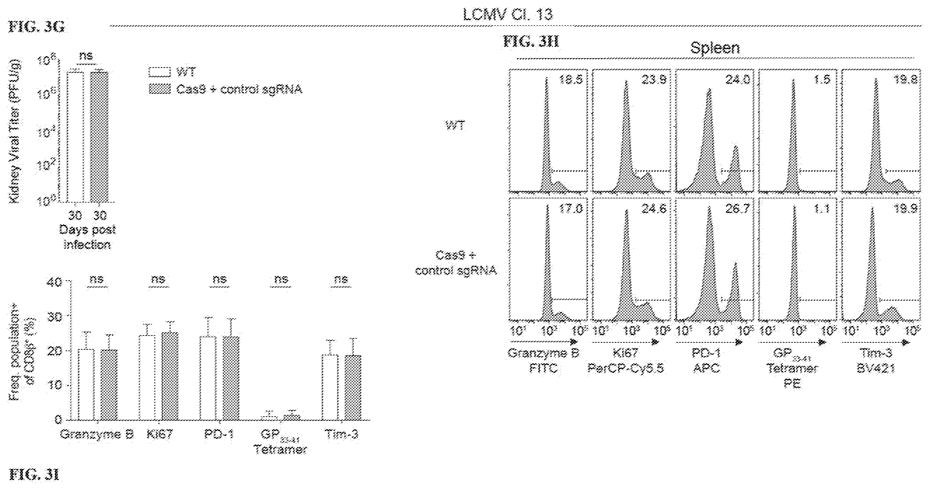

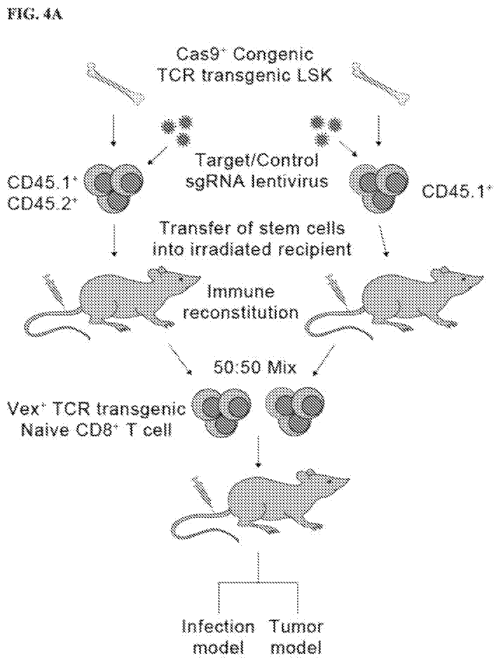

To determine whether candidate genes from other immune lineages could be deleted in vivo, sgRNAs to canonical genes expressed by B cells (Ms4a1), macrophages (Fcgr1), and dendritic cells (Ly75) were designed and chimeric mice were created using either these sgRNAs or control sgRNAs. It was found that CD20 was significantly reduced on B cells in the presence of two Ms4a1 (Cd20) sgRNAs, but not a control sgRNA, demonstrating that in vivo deletion of genes in B cells was possible (FIGS. 1F, 1G, and 2F). It was next confirmed that Fcgr1 (Cd64) could be deleted in red-pulp macrophages in the spleen by showing that CD64 expression was significantly reduced for two Fcgr1 targeting sgRNAs, but not for a control sgRNA (FIGS. 1F, 1G, and 2G). Lastly, Ly75 (Dec205) was deleted in dendritic cells in the spleen and a significant reduction in DEC205 expression with two Ly75 targeting sgRNAs, but not with a control sgRNA, was shown (FIGS. 1F, 1G, and 3A). Thus, this chimeric system can be used to delete genes of interest in innate and adaptive immune populations in vivo.

### Example 3: The Cas9-sgRNA Delivery System does not Alter Immune Development

To determine if the presence of Cas9 protein, the lentiviral sgRNA vector, or the process of transducing hematopoietic stem cells affected the development of immune cells, chimeric mice were generated using either non-transduced WT LSK cells or Cas9-expressing LSKs that were transduced with a lentiviral sgRNA vector containing a non-targeting sgRNA. The stem cells were transduced at an multiplicity of infection (MOI) such that approximately half of the immune cells expressed the fluorescent reporter, Vex, indicating the presence of the sgRNA vector (FIG. 3B). This MOI was chosen to have a sufficient quantity of transduced cells for analysis, while avoiding multiple integrations. Chimeras were analyzed after immune reconstitution, and it was found that the percentages of B cells, CD4⁺ or CD8⁺ T cells, CD11b⁺ myeloid cells, or dendritic cells in the spleen were similar in WT and Cas9+non-targeting sgRNA chimeras (FIG. 1H). To assess T cell development, thymic subsets in the chimeric mice were analyzed and no differences in the double-negative (DN), double-positive (DP), or CD4/CD8 single-positive (SP) populations were found in WT and Cas9+non-targeting sgRNA chimeras (FIG. 3C). The distribution of the DN subsets (DN1-4) in the WT and Cas9+sgRNA chimeras were further analyzed using CD25 and CD44 as markers, and no differences between the chimeric mice were found. Lastly, the naive status of CD8⁺ T cells from these chimeric mice were examined and no differences in CD44, CD62L, and CD69 percentages were found (FIGS. 3D-3F). These results confirm that the chimeric system does not alter immune cell proportions or T cell development at steady state.

### Example 4: The Cas9-sgRNA Delivery System does not Alter the Response to LCMV Viral Infection

To determine if the chimeric system altered the response of the immune system to a pathogen, chimeric mice (WT and Cas9+non-targeting sgRNA as above) were challenged with LCMV Clone 13 virus and T cell responses were examined. WT and Cas9+sgRNA chimeric mice had similar weight loss kinetics (FIG. 1I), indicating that the sgRNA delivery system did not alter the susceptibility of the mice to LCMV Clone 13. Serial viral titers in the blood and viral titers in the kidney at day 30 were comparable between WT and Cas9+sgRNA chimeras, indicating a similar response to the viral infection (FIGS. 1J and 3G). The phenotype of the CD8⁺ T cells at day 30 post-viral infection was also compared by flow cytometry and no differences were found in Granzyme B expression, T cell proliferation (Ki67) (FIGS. 3H and 3I), or expression of the co-inhibitory receptors, PD-1 and Tim-3 (FIGS. 3H and 3I). No difference was found in the percentage of GP₃₃₋₄₁ tetramer-specific cells at this time point between the two groups of chimeras (FIGS. 3H and 31). These findings demonstrate that the chimeric system does not affect CD8⁺ T cell responses or viral clearance kinetics following LCMV Clone 13 viral infection.

### Example 5: T Cell-Intrinsic Functions can be Evaluated During Chronic LCMV Infection and in Tumors

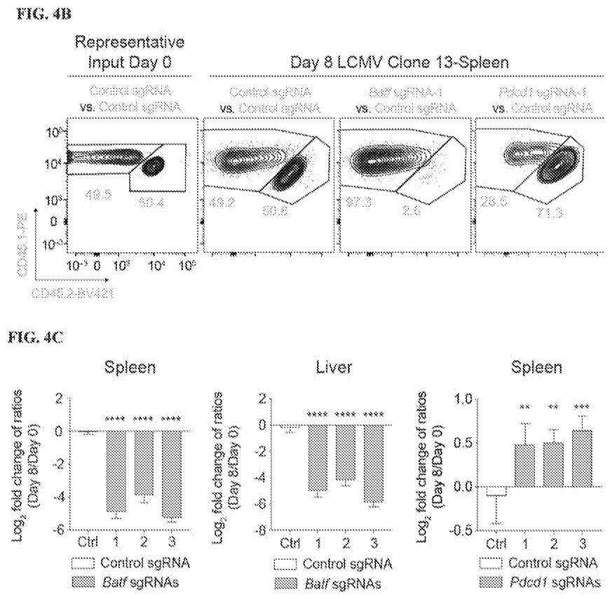

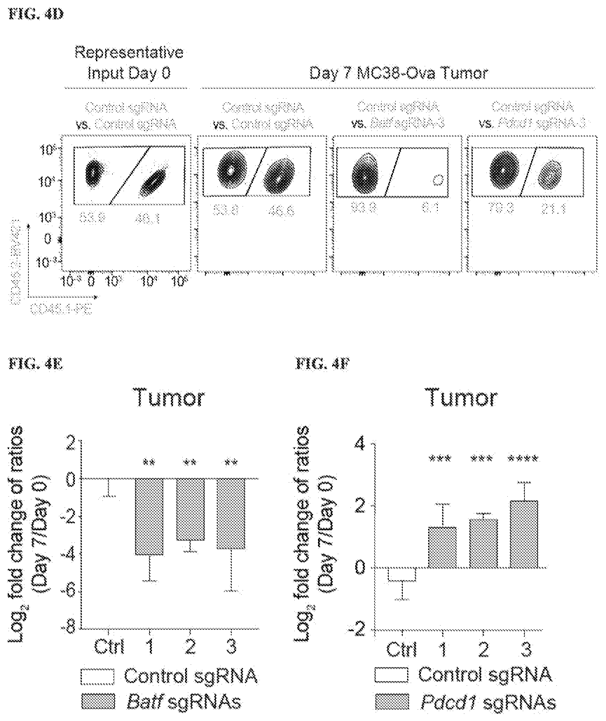

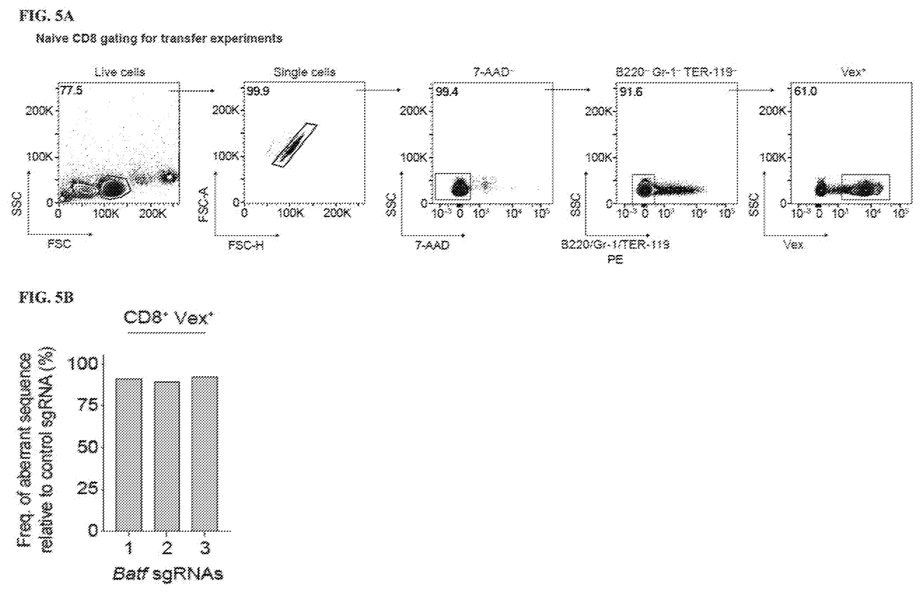

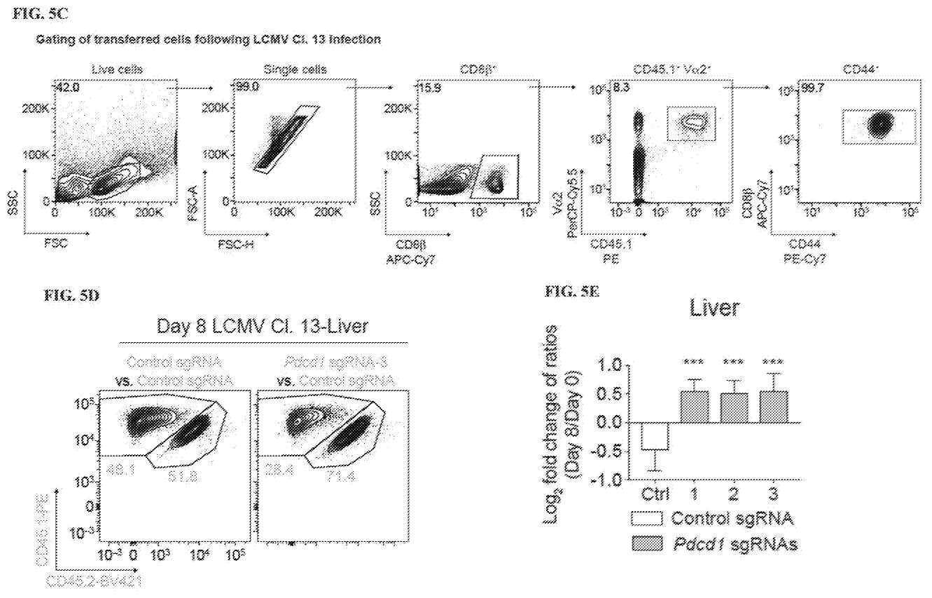

To assess whether the Cas9-sgRNA delivery system can be used to identify intrinsic regulators of T cell function in CD8⁺ T cells, two models were used: LCMV Clone 13 viral infection, as a model of T cell exhaustion, and MC38-OVA tumors as a model of tumor immunity. T cells responding to this infection encounter multiple inhibitory mechanisms, many of which are also conserved in the tumor microenvironment (TME) (Singer et al. (2017) Cell 171:1221-1223; Wherry et al. (2011) Nat. Immunol. 12:492-499; Penaloza-MacMaster et al. (2014) J. Exp. Med. 211:1905-1918; Baitsch et al. (2011) J. Clin. Invest. 121:2350-2360). The MC38-OVA tumor model was used to directly assess antigen-specific T cell suppressive mechanisms in the TME. To analyze antigen-specific T cells in vivo, Cas9-expressing donor mice with the TCR transgenic T cell receptors P14 (specific to the LCMV CD8 epitope GP₃₃₋₄₁) or OT-1 (specific to the ovalbumin CD8 epitope OVA₂₅₇₋₂₆₄) were used. Equal numbers (1:1 ratio) of congenically-marked antigen-specific gene-deleted naive CD8⁺ T cells and control cells were transferred to an unmanipulated host responding to viral infection/tumor (FIGS. 4A and 5A) to compare the phenotype and function of the gene-deleted and control T cells in the same microenvironment.

The effect of deleting Batf, an essential transcription factor for effector T cell differentiation during LCMV Clone 13 viral infection, was first evaluated (Singer et al. (2017) Cell 171:1221-1223; Odorizzi et al. (2015) J. Exp. Med. 212:1125-1137). The indel percentage of the Batf sgRNA-containing cells pre-transfer (input) was on average 90% as analyzed by the TIDE assay (FIG. 5B). Recipient mice were infected with LCMV Clone 13 and the ratio of control sgRNA-containing P14 T cells to control sgRNA or to Batf sgRNA-containing P14 T cells (FIGS. 4B, 4C, and 5C) in the spleen was analyzed on day 8 post-infection. The ratios of P14 T cells with control sgRNA vs. control sgRNA remained unchanged compared to input (FIG. 4B). In contrast, P14 TCR transgenic T cells containing the Batf sgRNA were significantly depleted for three different Batf sgRNAs, which recapitulates both germline knockout and shRNA knockdown phenotypes of BATF in CD8⁺ T cells during LCMV Cl.13 and LCMV Armstrong infection, respectively (Godec et al. (2015) Proc. Natl. Acad. Sci. U.S.A. 112:512-517; Kurachi et al. (2014) Nat. Immunol. 15:373-383). The same T cell populations from the liver of recipient mice showed a similar depletion of the Batf sgRNA-containing T cells, indicating a similar effect in multiple organs (FIGS. 4B and 4C).

Next, deletion of the negative regulator, PD-1, during LCMV Clone 13 infection was assessed. A competitive assay was performed using Pdcd1 sgRNA-containing P14 T cells and a significant expansion of T cells carrying each of three different Pdcd1 sgRNAs in the spleen and liver was shown (FIGS. 4B, 4C, 5D, and 5E), which is consistent with the germline knockout phenotype of PD-1 in CD8⁺ T cells (Odorizzi et al. (2015) J. Ep. Med. 212:1125-1137). Thus, the chimeric system can be used to evaluate the cell-intrinsic role of genes that regulate T cell differentiation and function in the LCMV Clone 13 viral infection model.

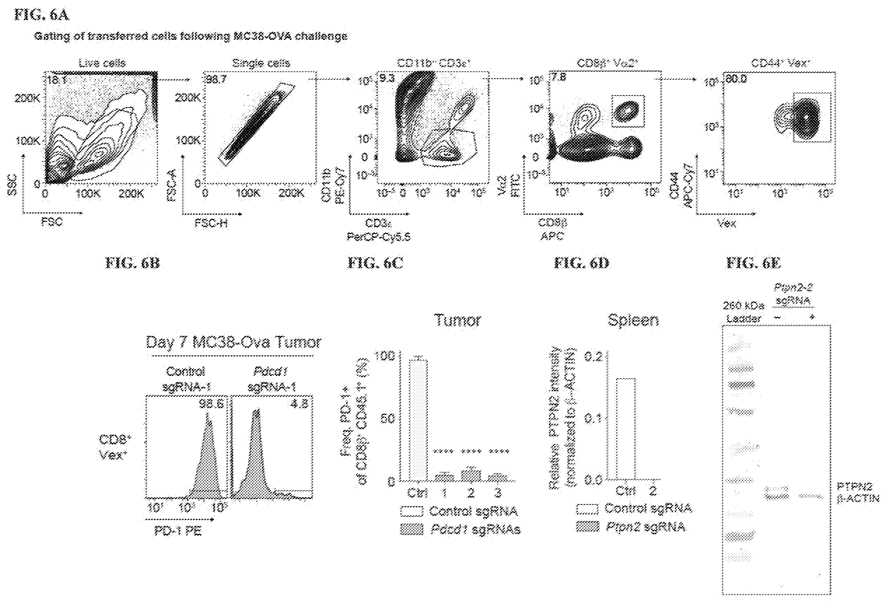

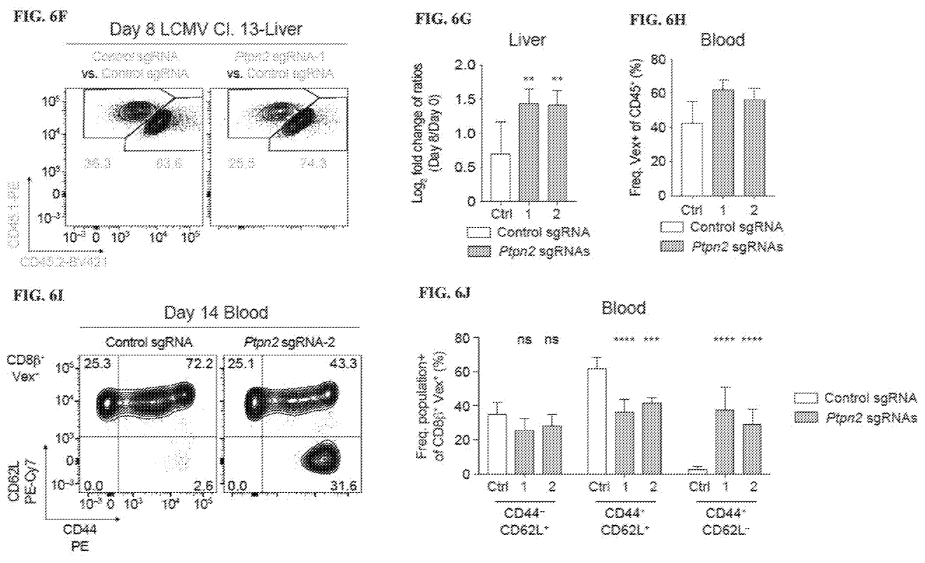

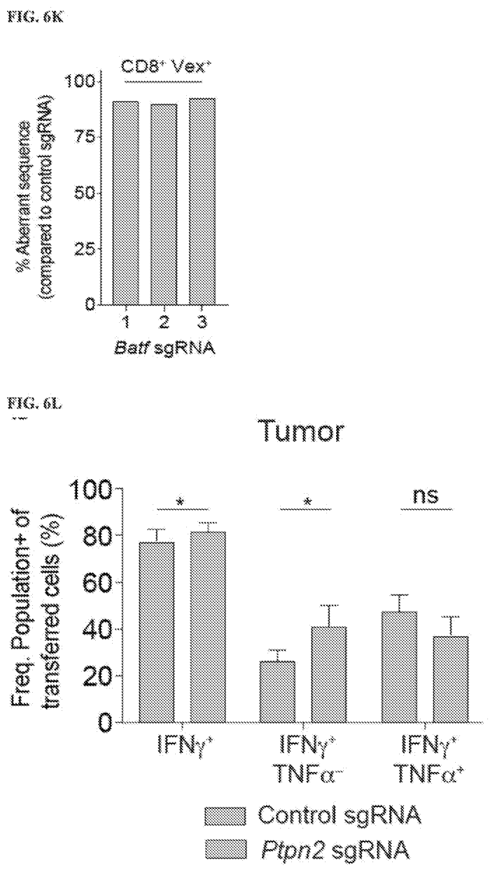

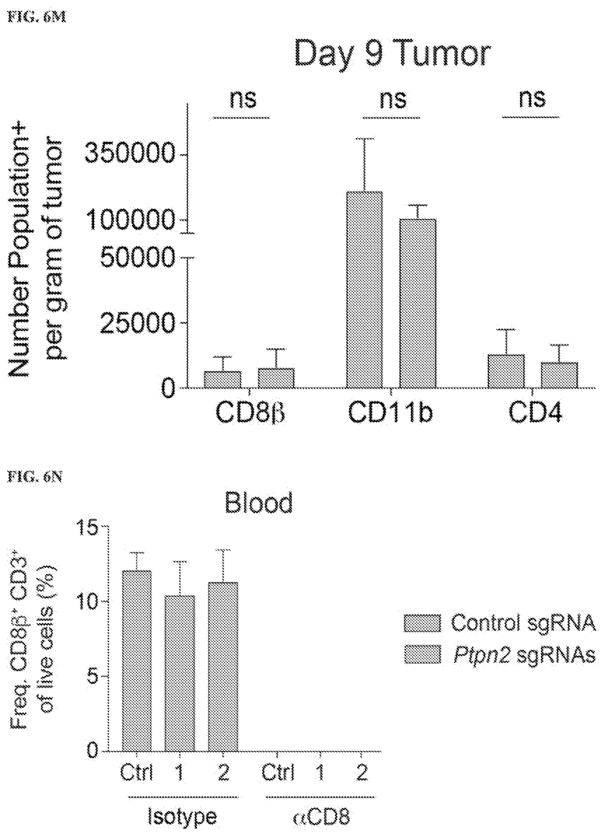

To determine if cell-intrinsic function of genetically perturbed CD8⁺ T cells in the TME could also be examined, OT-1 transgenic naive CD8⁺ T cells were transferred into recipient mice and MC38-OVA tumor cells were implanted. sgRNAs targeting Batf (to test a gene deletion that reduced T cell expansion) and Pdcd1 (to interrogate a gene deletion that promoted T cell expansion) were again used. The ratios of control sgRNA to control sgRNA-containing OT-1 T cells in the tumor did not change significantly at day 7 post-tumor implantation, compared to input (FIGS. 4D, 4E, and 6A). In contrast, OT-1 T cells with the three Batf sgRNAs had a competitive disadvantage compared to control cells (FIGS. 4D and 4E), whereas Pdcd1 sgRNA-containing OT-1 T cells were significantly enriched in the tumor (FIGS. 4D and 4F). PD-1 expression on the transferred Pdcd1 sgRNA-containing OT-1 T cells were evaluated at the end of the competitive assay and, as expected, it was found that these T cells still showed a significant reduction in PD-1 expression (FIGS. 6B and 6C). These results indicate that the chimera system can be used to perturb genes that cause gain or loss of function in multiple disease models.

### Example 6: Loss of Ptpn2 Enhances CD8⁺ T Cell Responses to LCMV Clone 13 Viral Infection

It was next determined whether this system could be used to uncover new negative regulators of T cell responses to viral infection and tumors. Ptpn2 was focused on as a candidate gene due to its role in attenuating T cell responses to maintain tolerance and prevent autoimmunity, and because of the association of PTPN2 polymorphisms with risk in human autoimmune diseases (Wiede et al. (2011) J. Clin. Invest. 121:4758-4774; Wiede et al. (2014) J. Autoimmun. 53:105-114; Todd et al. (2007) Nat. Genet. 39:857-864; Okuno et al. (2018) Diabet. Med 35:376-380). It have been previously shown that P1pn2 also negatively regulates interferon signaling in tumor cells and deletion of Ptpn2 in tumor cells leads to attenuated tumor growth (Manguso et al. (2017) Nature 547:413-418). Thus, there is significant interest in Ptpn2 as a cancer immunotherapy target, yet its role in regulating CD8⁺ T cell responses to viral infection and tumors has not been examined (Wiede et al. (2017) Immunol. Cell Biol. 95:859-861; Spalinger et al. (2018) Cell Rep. 22:1835-1848).

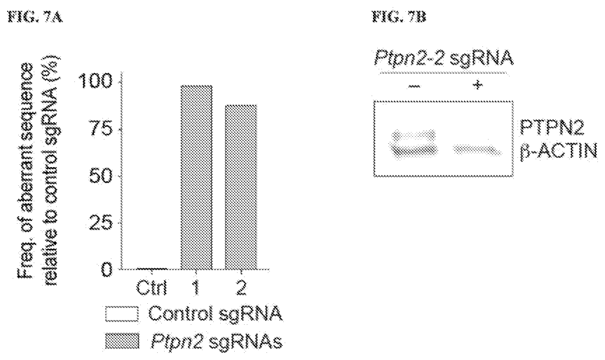

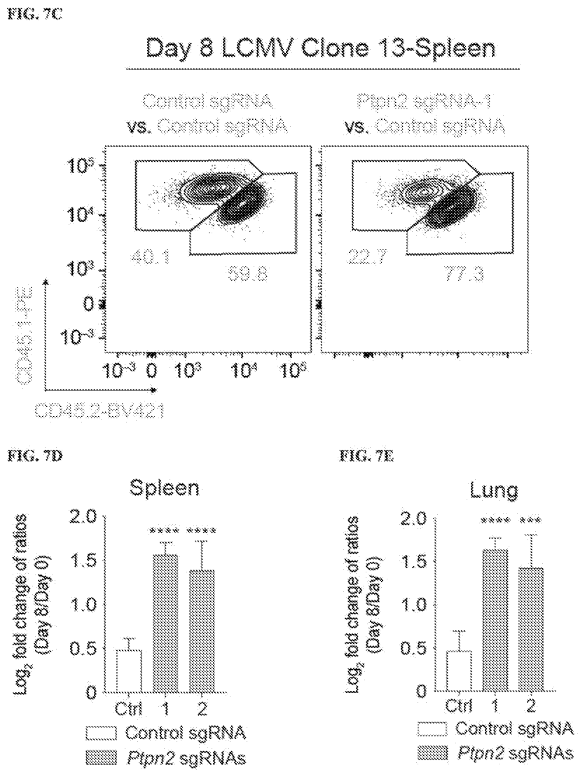

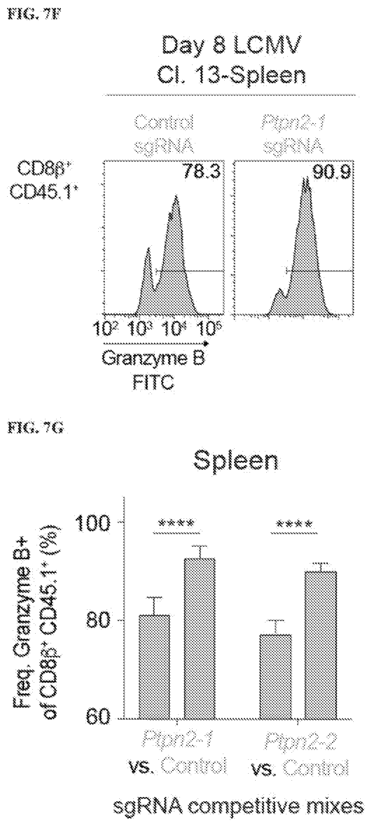

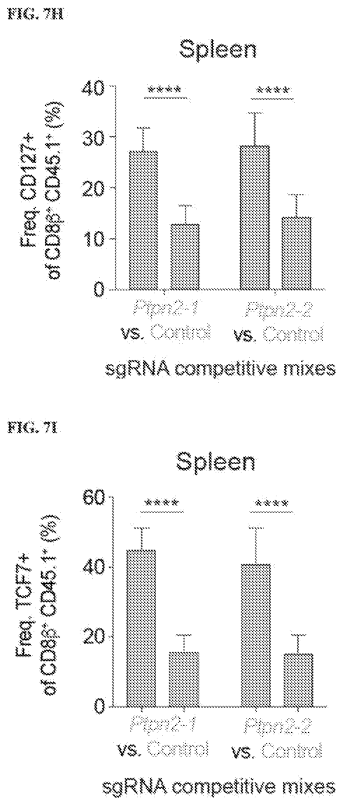

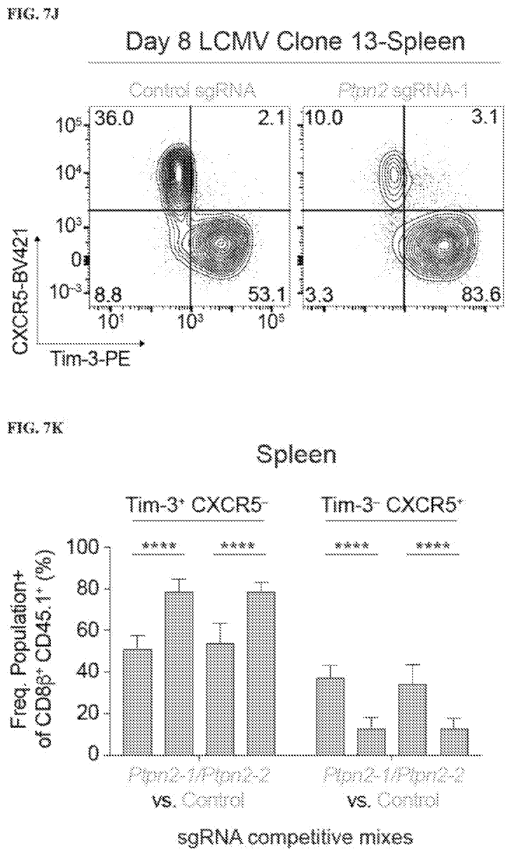

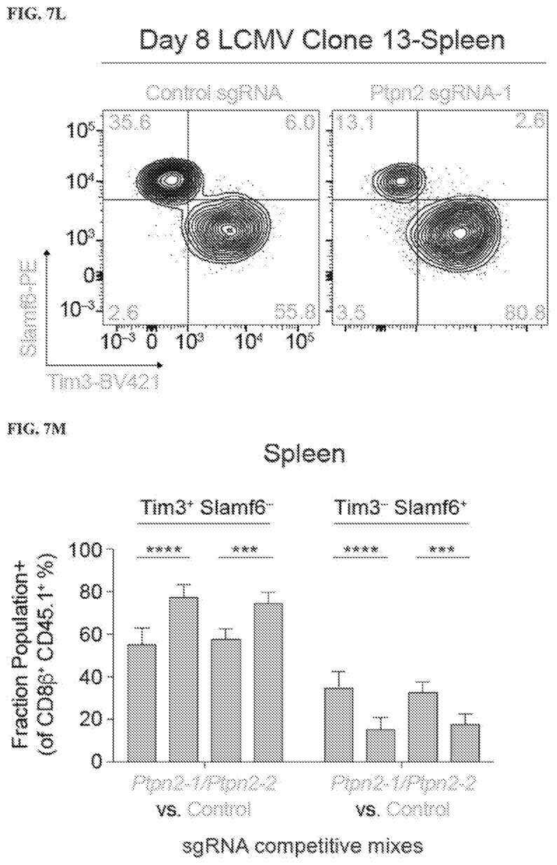

To evaluate the role of Ptpn2 in regulating anti-viral CD8⁺ T cell responses, a 1:1 competitive assay was performed with control sgRNA or Ptpn2 sgRNA-containing P14 CD8⁺ T cells during LCMV Clone 13 viral infection. Efficient deletion (˜80%) of Ptpn2 was first confirmed using the TIDE assay (FIG. 7A) and by Western blot (FIGS. 6D, 6E, and 7B). Using two sgRNAs, it was found that Ptpn2 sgRNA-containing P14 CD8⁺ T cells significantly outcompeted control sgRNA-containing cells in the spleen, lung, and liver of infected recipient animals (FIGS. 6F, 6G, and 7C-7E). Ptpn2-deleted P14 CD8⁺ T cells showed increased Granzyme B expression (FIGS. 7F and 7G) and a corresponding decrease in CD127 expression (FIG. 7H) and TCF7 expression (FIG. 7I). Given these changes, it was next determined if there was skewing of the recently described terminally and stem-like exhausted CD8⁺ T cell populations (Im et al. (2016) Nature 537:417-421; He et al. (2016) Nature 537:412-428). Tim-3 was used to mark the terminally exhausted subset and CXCR5 to mark the stem-like subset. Pqpn2 deletion skewed the CD8⁺ T cell response towards the terminally exhausted Tim-3⁺ CXCR5⁻ population (FIGS. 7J and 7K). These data indicate that loss of Ptpn2 promotes the formation of the terminally exhausted population during LCMV Clone 13 viral infection.

### Example 7: Loss of Ptpn2 Promotes the Early Expansion of CD8⁺ T Cells During LCMV Clone 13 Infection

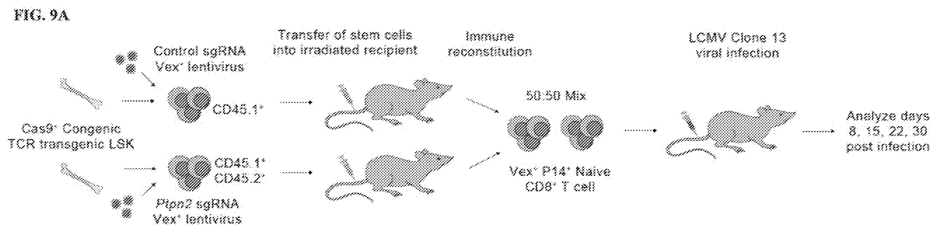

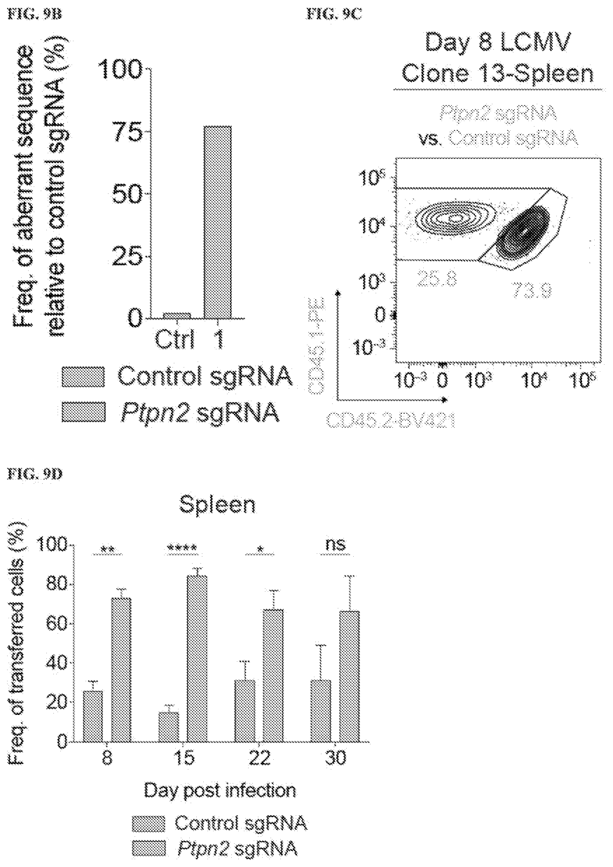

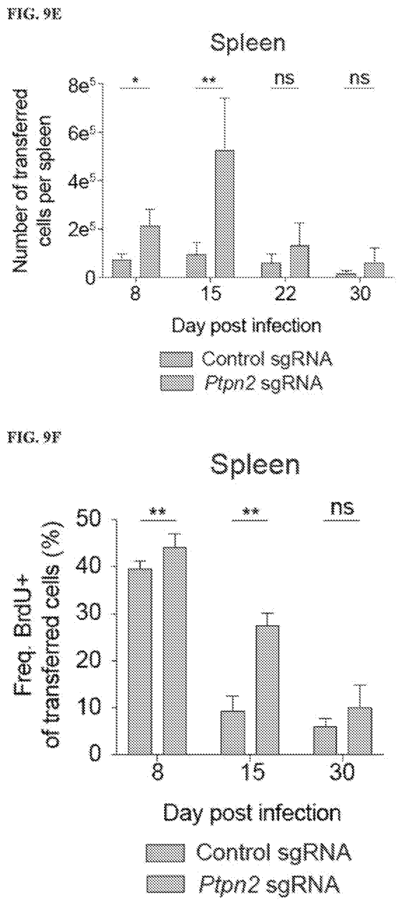

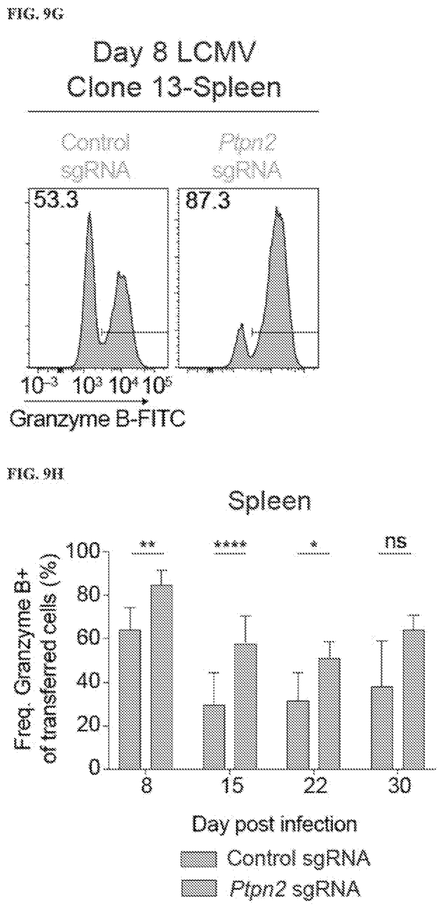

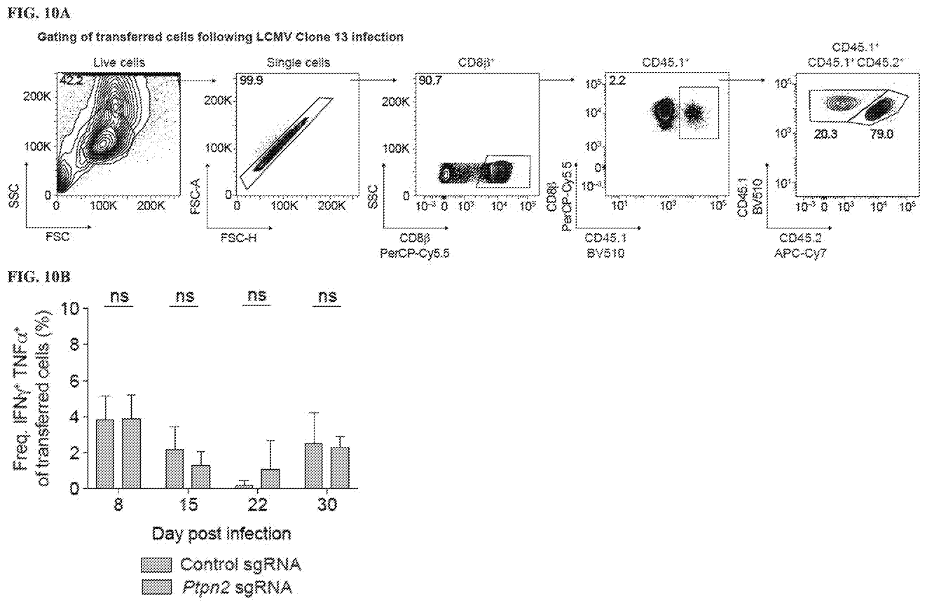

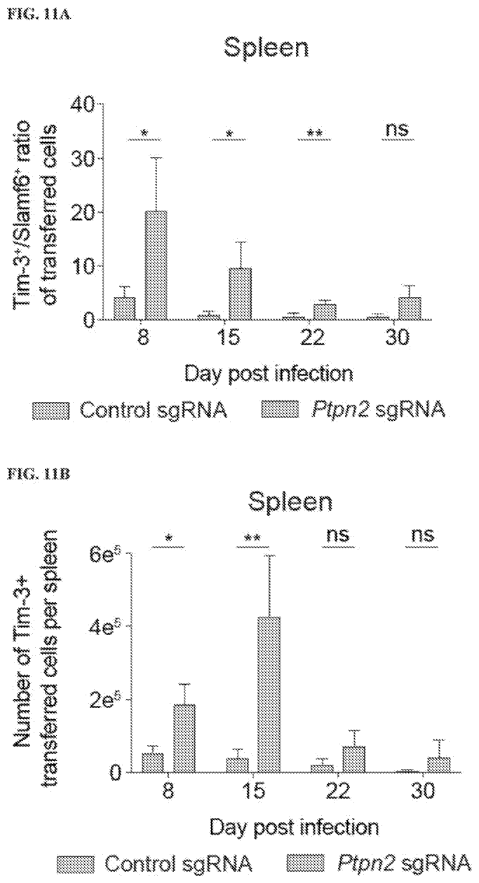

A pooled in vivo loss-of-function screen was recently conducted, and Ptpn2 was identified as a candidate regulator of CD8T cell responses. To examine the role of Ptpn2 in LCMV Clone 13 viral infection, bone marrow chimeras were created using the CHIME method (FIG. 9A) to delete Ptpn2 in hematopoietic cells from P14 TCR transgenic mice. Efficient deletion (˜80%) of Ptpn2 was first confirmed using the TIDE assay (Brinkman et al. (2014) Nucl. Acids Res. 42:e168) (FIG. 9B). To evaluate cell intrinsic functions of Ptpn2 in CD8⁺ T cells, a 1:1 ratio of P14 TCR transgenic Ptpn2 sgRNA-containing and control sgRNA-containing CD8⁺ T cells were co-transferred to wild-type recipient mice and these mice were subsequently infected with LCMV Clone 13 and responses were analyzed at multiple time points (FIGS. 9C-E and FIG. 10A). Ptpn2-deleted cells were significantly increased in percentage and number compared with control cells at days 8 and 15 post infection, but not day 30 post infection. BrdU incorporation was increased significantly in Ptpn2 sgRNA-containing cells at days 8 and 15 post infection (FIG. 9F). Ptpn2 deletion did not affect polyfunctional cytokine production as the percentage of IFNγ⁺ TNFα⁺ cells after peptide restimulation in vitro was unchanged (FIG. 10B). However, Ptpn2 deletion increased the percentage of Granzyme B⁺ cells at days 8, 15, and 22 post infection (FIGS. 9G and 9H). Thus, Ptpn2 deletion provides CD8⁺ T cells with a transient advantage early during LCMV Clone 13 infection but does not prevent contraction of these CD8⁺ T cells at later time points.

### Example 8: Deletion of Ptpn2 Enhances Formation of the Tim-3⁺ Subpopulation During LCMV Clone 13 Infection

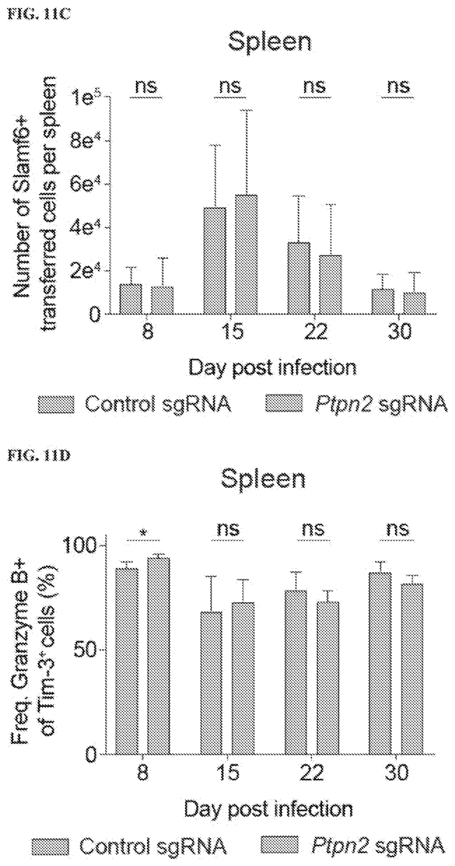

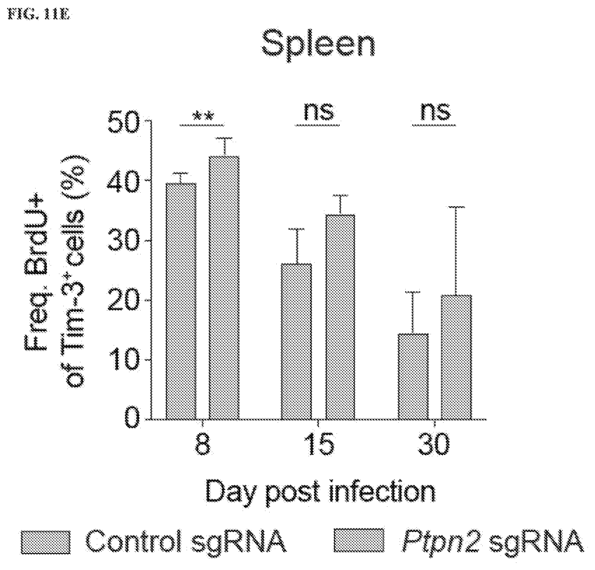

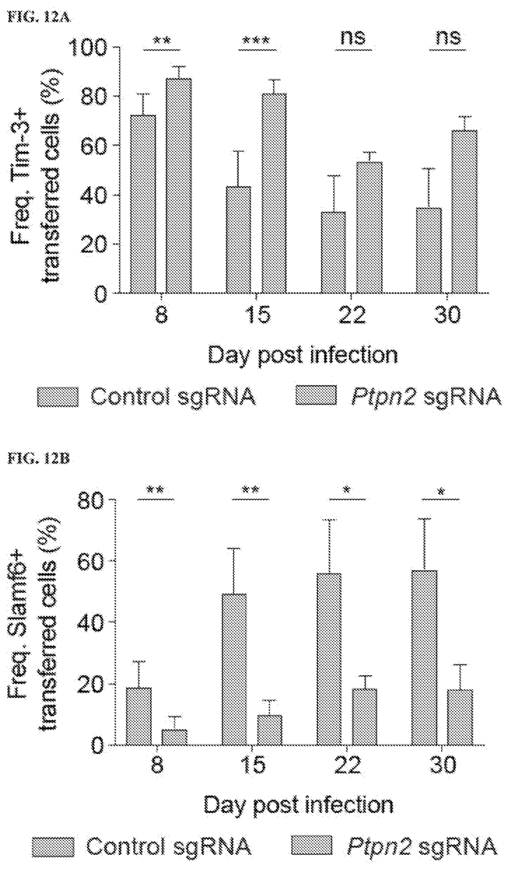

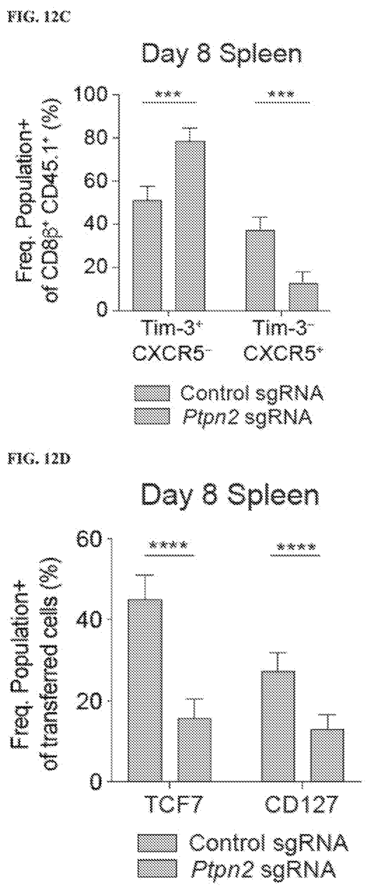

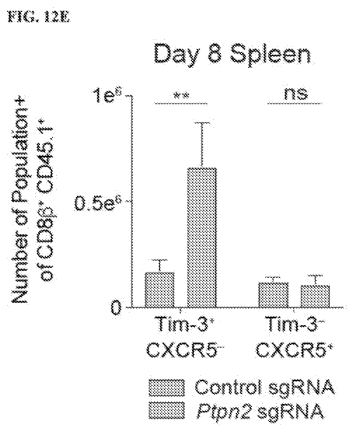

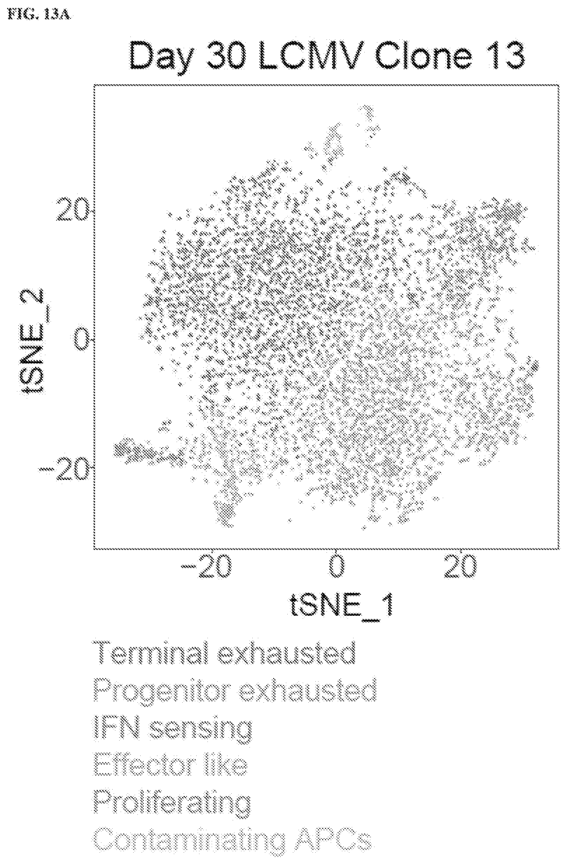

The changes in Granzyme B expression prompted the examination of the impact of Ptpn2 deletion on the generation of Slamf6⁺ progenitor and Tim-3⁺ terminally exhausted subpopulations. It was found that Ptpn2 deletion increased the ratio of Tim-3⁺ to Slamf6⁺ cells at days 8, 15, and 22 post infection (FIGS. 7L, 11A, 12A, and 12B). Analysis of the populations using Tim-3 and a distinct progenitor exhausted marker (CXCR5) gave identical results: Ptpn2 deletion resulted in an increase in the percentage of Tim-3⁺ cells and a decrease in the percentage of CXCR5⁺ cells compared with control cells (FIGS. 7J and 12C). Moreover, following Ptpn2 deletion, a decrease in expression of two additional markers of progenitor exhausted cells, CD127 and TCF7 was observed (FIG. 12D). Furthermore, this increase in the Tim-3⁺ to Slamf6⁺ ratio was driven by a specific increase in the number of Tim-3⁺ cells following Ptpn2 deletion (FIG. 11B). There was no difference in the number of Slamf6⁺ cells following Ptpn2 deletion (FIG. 11C), nor in the number of CXCR5⁺ cells (FIG. 12E). To determine if the functional changes in Granzyme B expression and BrdU incorporation were due to the increased Tim-3⁺ to Slamf6⁺ ratio or an intrinsic change in the Tim-3⁺ population, Tim-3⁺ control or P1pn2 sgRNA-containing cells were compared and found only a minimal difference at day 8 in Granzyme B expression and BrdU incorporation (FIGS. 11D and 11E). These findings demonstrate that deletion of Ptpn2 leads to a specific increase in the generation of the Tim-3⁺ subpopulation, while preserving the number of Slamf6⁺ cells, and that this altered ratio is responsible for the increase in Granzyme B expression and BrdU incorporation.

### Example 9: Ptpn2 Deletion Promotes Effector-Skewed Slamf6⁺ and Tim-3⁺ Subpopulations During LCMV Infection

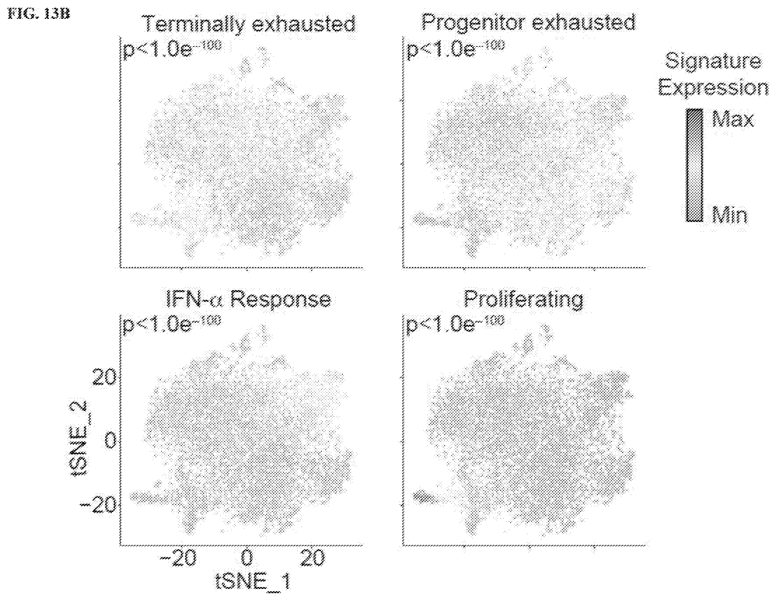

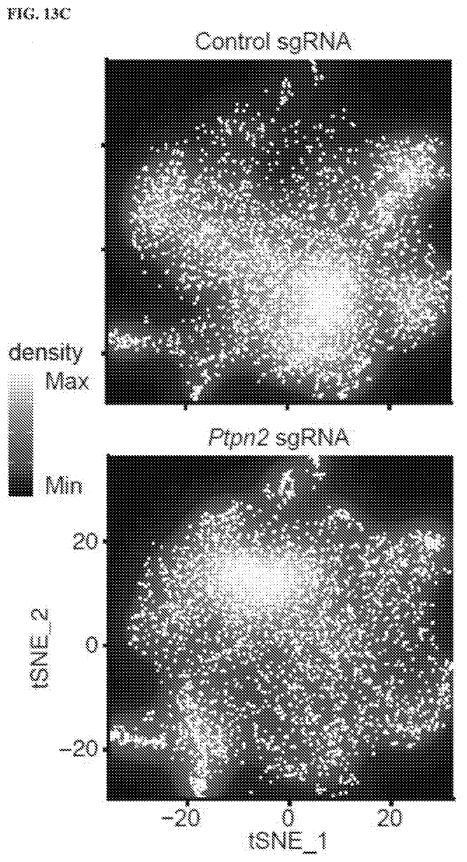

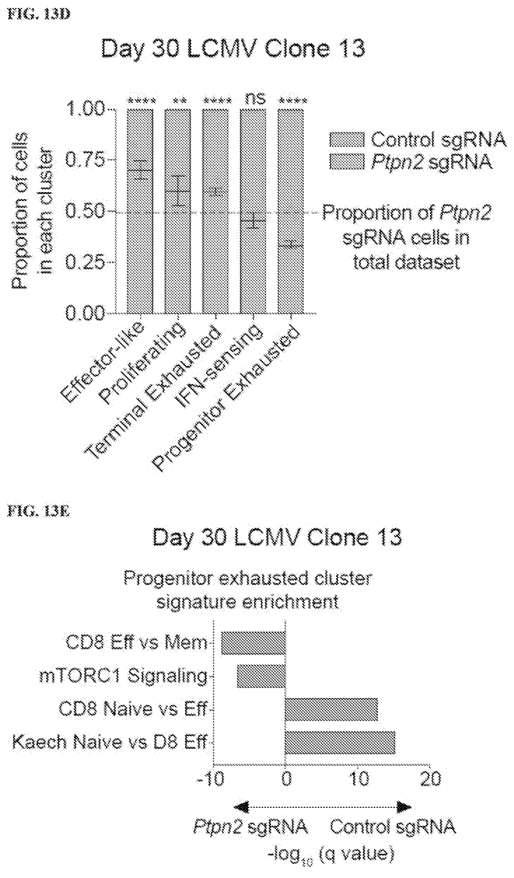

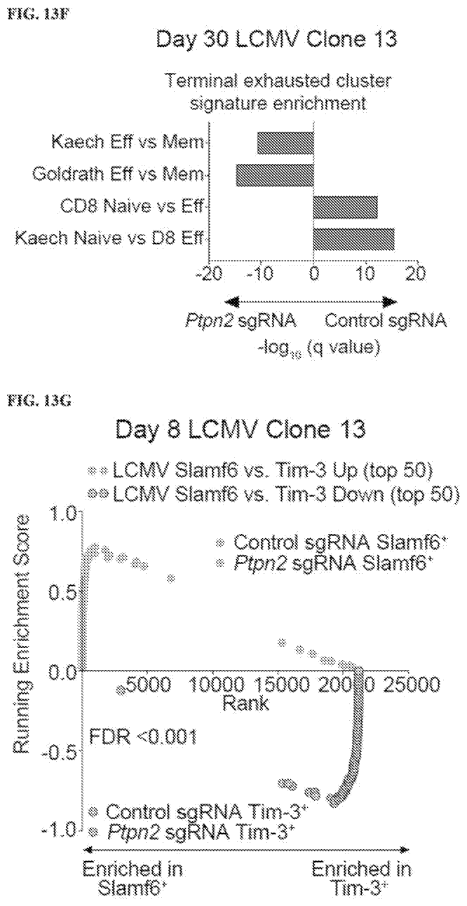

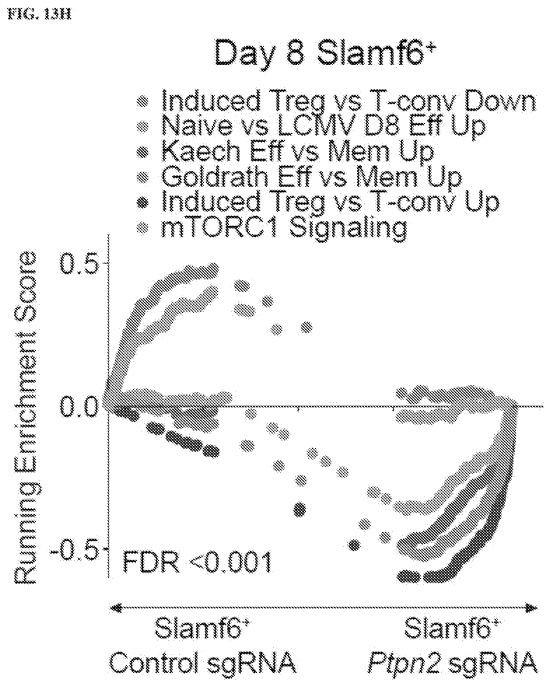

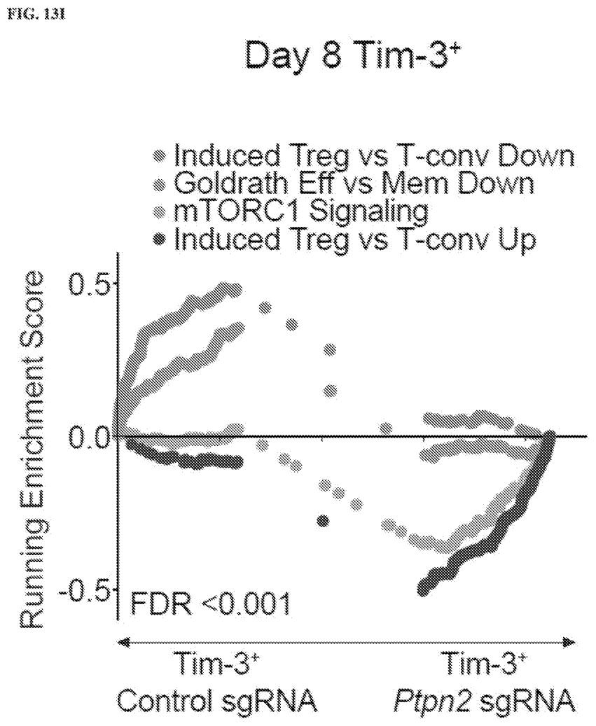

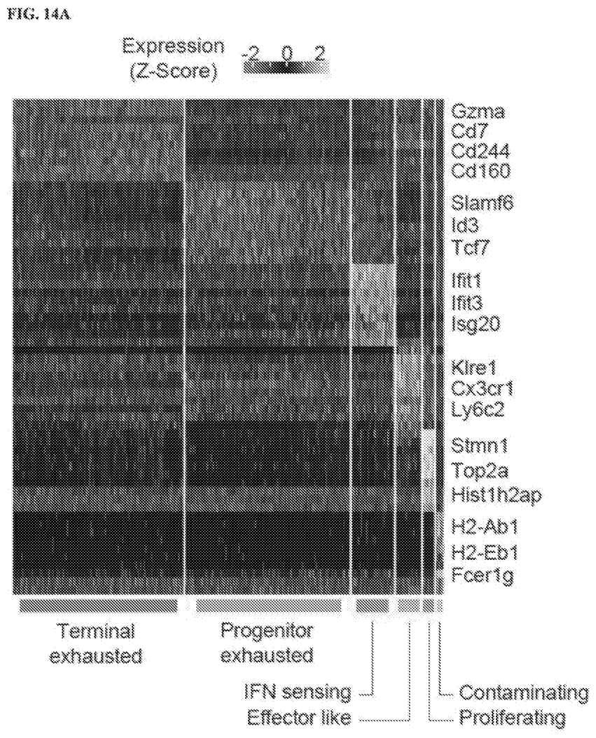

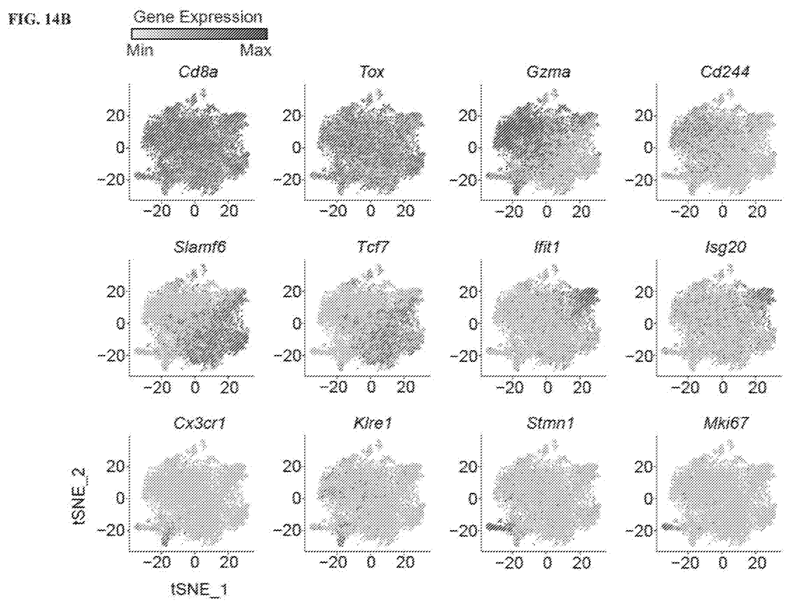

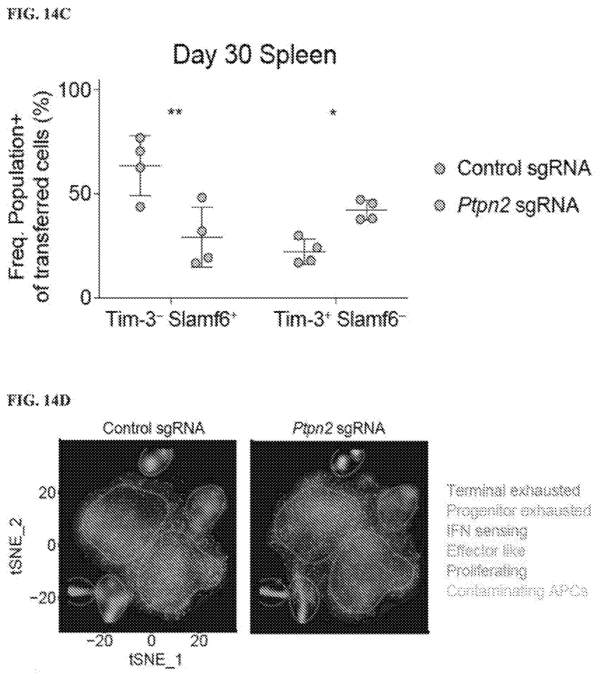

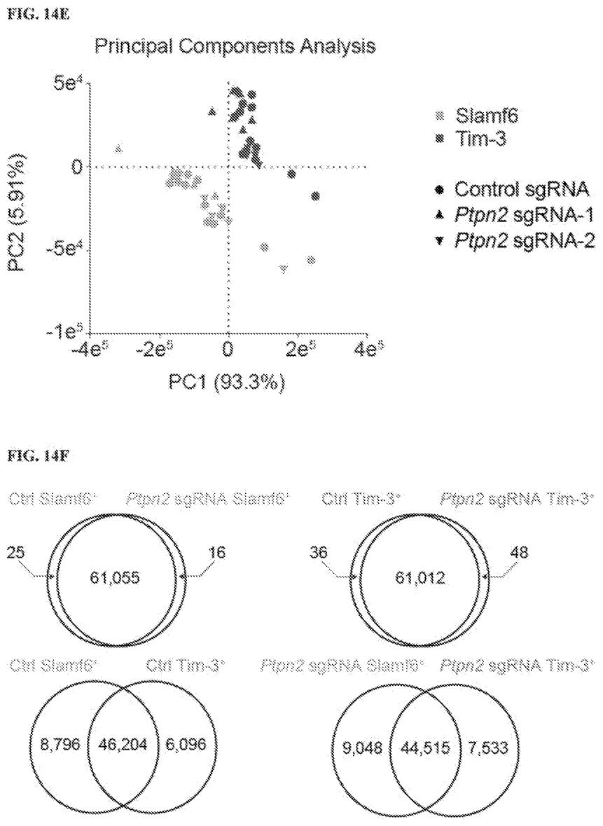

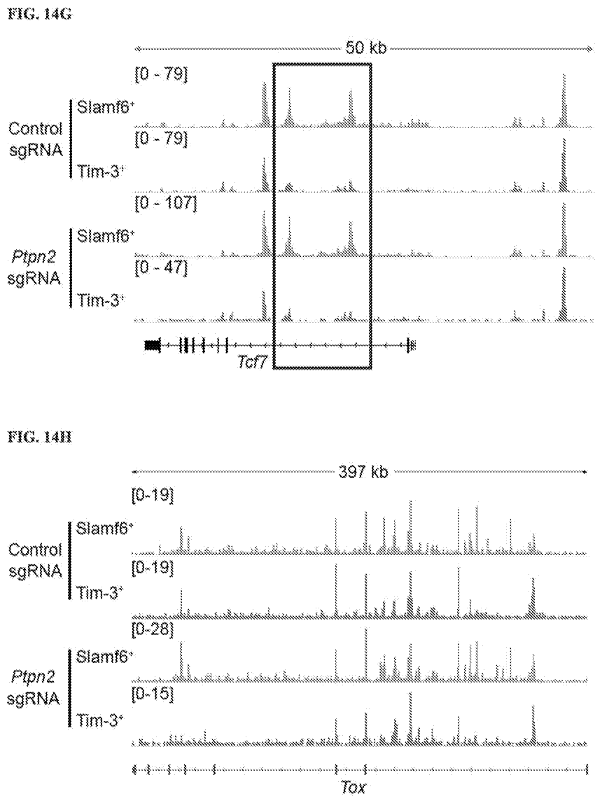

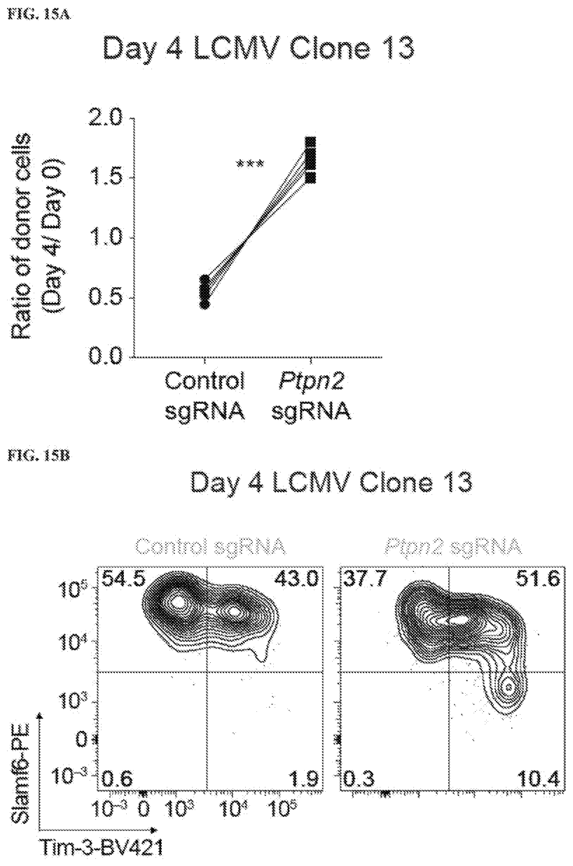

How Ptpn2 influenced cell fates as they differentiate into exhausted cells was next investigated by performing single cell RNA-seq on control and Ptpn2-deleted cells day 30 post infection, as the canonical features of exhaustion are present during this time point (Wherry et al. (2003). J. Virol. 77:4911-4927; Sen et al. (2016) Science 354:1165-1169). Unsupervised clustering of the cells revealed 6 subpopulations, which were identified by marker gene expression and previously-defined signature enrichment (FIGS. 13A, 13B and 14A). The previously described terminally exhausted, progenitor exhausted, proliferating, and effector-like populations (Miller et al. (2019) Nat. Immunol.), marked by characteristic expression of genes such as Gzma and Cd244 (terminally exhausted), Slamf6 and Tcf, (progenitor exhausted), Cx3cr1 and Klre1 (effector-like), and Stmn1 and Mki67 (proliferating) (FIG. 14B) were recapitulated. In addition, a novel subpopulation that was driven by IFN-sensing genes (i.e., Ifit1 and Isg20) and enriched for the Hallmark IFN-α signature was identified (FIGS. 13B and 14B). Of note, this IFN sensing cluster contained both progenitor exhausted and terminally exhausted cells, indicating that these were not a novel differentiation state but instead represented cells that were actively sensing IFN-α in their local microenvironment. Further analysis of the distribution of the control or Ptpn2-deleted cells across the clusters revealed a significant skewing of the control cells into the progenitor exhausted cluster and the Ppn2-deleted cells into the effector-like, proliferating, and terminally exhausted clusters (FIGS. 13C and 13D), consistent with the flow cytometry data (FIG. 14C). In addition to the significant population changes, it was noticed that within a subpopulation the Ptpn2-deleted cells or control CD8⁺ T cells tended to cluster together (FIG. 14D). By performing differential expression analysis within the progenitor and terminally exhausted clusters, it was noticed that the Ptpn2-deleted cells had increased expression of Gzma, Cd160, Stat1, Cd7, Ccl4, and Ccl5 in the terminally exhausted cluster. Similarly, in the progenitor exhausted cluster Ptpn2-deleted cells had increased expression of Gzma, Gzmk, Cd160, Stat1, Cd7, Ccl4, Ccl5, Pdcd1, Lag3, and Id2. Signature analysis revealed enrichment of effector-related gene signatures, such as mTORC1 signaling and effector vs memory profiles, in the Ptpn2-deleted cells in progenitor and terminally exhausted clusters (FIGS. 13E and 13F). Thus, at day 30 post LCMV infection, Ptpn2-deleted progenitor and terminally exhausted cells have increased transcription of effector-related genes.

Given the late changes in progenitor and terminally exhausted cells, it was asked whether Ptpn2 deletion impacted the effector profiles of Slamf6⁺ and Tim-3⁺ subpopulations at an early time point post LCMV infection. RNA-seq on co-transferred Ptpn2-deleted or control CD8⁺ T cells was performed eight days post-LCMV Clone 13 infection (Table 4A-4D).

TABLE 4A
GSEA Full Report for RNA-seq profiling of Ptpn2 sgRNA vs. control cells 8
days post LCMV Clone 13 viral infection (Tim control versus Ptpn2 up)

| | GS<br> follow | GS | | | | | | | RANK | |
|---|---|---|---|---|---|---|---|---|---|---|
| | link | DE- | | | | NOM | FDR | FWER | AT | LEADING |
| NAME | to MSigDB | TAILS | SIZE | ES | NES | p-val | q-val | p-val | MAX | EDGE |
| LCMVSLAMP6_ | LCMVSLAMP6_ | Details | 389 | 0.28666005 | 6.568165 | 0 | 0 | 0 | 5754 | tags = 55%, |
| V_LCMVTIM3_ | V_LCMVTIM3_ | . . . | | | | | | | | list = 27%, |
| DOWN_ADJP001 | DOWN_ADJP001 | | | | | | | | | signal = |
| | | | | | | | | | | 74% |
| TILSLAMF6_V_ | TILSLAMF6_V_ | Details | 50 | 0.5652034 | 4.5770946 | 0 | 0 | 0 | 5008 | tags = 80%, |
| TILTIM3_UP_50 | TILTIM3_UP_50 | . . . | | | | | | | | list = 24%, |
| | | | | | | | | | | signal = |
| | | | | | | | | | | 104% |
| LCMVSLAMF6_ | LCMVSLAMF6_ | Details | 50 | 0.44591218 | 3.6686087 | 0 | 0 | 0 | 4987 | tags = 68%, |
| V_LCMVTIM3_ | V_LCMVTIM3_ | . . . | | | | | | | | list = 24%, |
| UP_50 | UP_50 | | | | | | | | | signal = |
| | | | | | | | | | | 89% |
| HALLMARK_ | HALLMARK_ | Details | 91 | 0.29150483 | 3.3152997 | 0 | 0 | 0 | 4798 | tags = 52%, |
| INTERFERON_ | INTERFERON_ | . . . | | | | | | | | list = 23%, |
| ALPHA_ | ALPHA_ | | | | | | | | | signal = |
| RESPONSE | RESPONSE | | | | | | | | | 66% |
| HALLMARK_ | HALLMARK_ | Details | 190 | 0.18950525 | 2.985017 | 0 | 0 | 0 | 5840 | tags = 46%, |
| INTERFERON_ | INTERFERON_ | . . . | | | | | | | | list = 28%, |
| GAMMA_ | GAMMA_ | | | | | | | | | signal = |
| RESPONSE | RESPONSE | | | | | | | | | 63% |
| LCMVSLAMF6_ | LCMVSLAMF6_ | Details | 49 | 0.34334487 | 2.800357 | 0 | 0 | 0 | 5720 | tags = 61%, |
| V_LCMVTIM3_ | V_LCMVTIM3_ | . . . | | | | | | | | list = 27%, |
| DOWN_50 | DOWN_50 | | | | | | | | | signal = |
| | | | | | | | | | | 84% |
| HALLMARK_ | HALLMARK_ | Details | 194 | 0.1738449 | 2.7651145 | 0 | 0 | 0 | 5858 | tags = 45%, |
| TNFA_ | TNFA_ | . . . | | | | | | | | list = 28%, |
| SIGNALING_ | SIGNALING_ | | | | | | | | | signal = |
| VIA_NFKB | VIA_NFKB | | | | | | | | | 61% |
| HALLMARK_ | HALLMARK_ | Details | 186 | 0.17286915 | 2.7478368 | 0 | 0 | 0 | 5602 | tags = 44%, |
| HEME_ | HEME_ | . . . | | | | | | | | list = 26%, |
| METABOLISM | METABOLISM | | | | | | | | | signal = |
| | | | | | | | | | | 59% |
| HALLMARK_ | HALLMARK_ | Details | 159 | 0.15301442 | 2.2737944 | 0 | 0.00239546 | 0.003 | 5316 | tags = 40%, |
| APOPTOSIS | APOPTOSIS | . . . | | | | | | | | list = 25%, |
| | | | | | | | | | | signal = |
| | | | | | | | | | | 53% |
| HALLMARK_ | HALLMARK_ | Details | 193 | 0.12119718 | 1.9573029 | 0.00203252 | 0.01438997 | 0.182 | 1738 | tags = 20%, |
| INFLAMMATORY_ | INFLAMMATORY_ | . . . | | | | | | | | list = 8%, |
| RESPONSE | RESPONSE | | | | | | | | | signal = |
| | | | | | | | | | | 22% |
| HALLMARK_ | HALLMARK_ | Details | 185 | 0.12246529 | 1.9308792 | 0.01367188 | 0.01511828 | 0.205 | 5909 | tags = 40%, |
| COMPLEMENT | COMPLEMENT | . . . | | | | | | | | list = 28%. |
| | | | | | | | | | | signal = |
| | | | | | | | | | | 55% |
| HALLMARK_ | HALLMARK_ | Details | 140 | 0.1340673 | 1.880768 | 0.00810672 | 0.01774398 | 0.262 | 5659 | tags = 40%, |
| UV_ | UV_ | . . . | | | | | | | | list = 27%, |
| RESPONSE_DN | RESPONSE_DN | | | | | | | | | signal = |
| | | | | | | | | | | 54% |
| HALLMARK_ | HALLMARK_ | Details | 53 | 0.20704785 | 1.7775687 | 0.02012073 | 0.02973003 | 0.425 | 5624 | tags = 47%, |
| TGF_BETA_ | TGF_BETA_ | . . . | | | | | | | | list = 27%, |
| SIGNALING | SIGNALING | | | | | | | | | signal = |
| | | | | | | | | | | 64% |
| HALLMARK_ | HALLMARK_ | Details | 98 | 0.14712435 | 1.7209449 | 0.03340292 | 0.03598003 | 0.52 | 4685 | tags = 37%, |
| ANDROGEN_ | ANDROGEN_ | . . . | | | | | | | | list = 22%, |
| RESPONSE | RESPONSE | | | | | | | | | signal = |
| | | | | | | | | | | 47% |
| HALLMARK_ | HALLMARK_ | Details | 193 | 0.10121644 | 1.6534734 | 0.04233871 | 0.04724313 | 0.643 | 5895 | tags = 38%, |
| KRAS_ | KRAS_ | . . . | | | | | | | | list = 28%, |
| SIGNALING_UP | SIGNALING_UP | | | | | | | | | signal = |
| | | | | | | | | | | 52% |
| HALLMARK_ | HALLMARK_ | Details | 47 | 0.20330477 | 1.6259607 | 0.03333334 | 0.04985178 | 0.68 | 5174 | tags = 45%, |
| REACTIVE_ | REACTIVE_ | . . . | | | | | | | | list = 24%, |
| OXIGEN_ | OXIGEN_ | | | | | | | | | signal = |
| SPECIES_ | SPECIES_ | | | | | | | | | 59% |
| PATHWAY | PATHWAY | | | | | | | | | |
| HALLMARK_ | HALLMARK_ | Details | 43 | 0.20434797 | 1.5811809 | 0.05068226 | 0.05752607 | 0.758 | 13926 | tags = 86%, |
| APICAL_ | APICAL_ | . . . | | | | | | | | list = 66%, |
| SURFACE | SURFACE | | | | | | | | | signal = |
| | | | | | | | | | | 250% |
| HALLMARK_ | HALLMARK_ | Details | 198 | 0.08957239 | 1.4680673 | 0.06876228 | 0.08967117 | 0.894 | 8616 | tags = 49%, |
| APICAL_ | APICAL_ | . . . | | | | | | | | list = 41%, |
| JUNCTION | JUNCTION | | | | | | | | | signal = |
| | | | | | | | | | | 83% |
| HALLMARK_ | HALLMARK_ | Details | 34 | 0.16897428 | 1.1861672 | 0.23929961 | 0.2533092 | 1 | 12642 | tags = 76%, |
| PANCREAS_ | PANCREAS_ | . . . | | | | | | | | list = 60%, |
| BETA_CELLS | BETA_CELLS | | | | | | | | | signal = |
| | | | | | | | | | | 189% |
| HALLMARK_ | HALLMARK_ | Details | 32 | 0.15937337 | 1.0576003 | 0.37204725 | 0.36709058 | 1 | 5904 | tags = 44%, |
| NOTCH_ | NOTCH_ | . . . | | | | | | | | list = 28%, |
| SIGNALING | SIGNALING | | | | | | | | | signal = |
| | | | | | | | | | | 61% |

TABLE 4B
GSEA Full Report for RNA-seq profiling of Ptpn2 sgRNA vs. control cells 8
days post LCMV Clone 13 viral infection (Tim control versus Ptpn2 down)

| | GS<br> follow to | GS | | | | NOM |
|---|---|---|---|---|---|---|
| NAME | link MSigDB | DETAILS | SIZE | ES | NES | p-val |
| HALLMARK_E2F_TRAGETS | HALLMARK_E2F_TRAGETS | Details . . . | 199 | −0.4467453 | −7.4234233 | 0 |
| HALLMARK_MYC_TRAGETS_V1 | HALLMARK_MYC_TRAGETS_V1 | Details . . . | 197 | −0.4342686 | −7.0910573 | 0 |
| LCMVSLAMF6_V_LCMVTIM3_ | LCMVSLAMF6_V_LCMVTIM3_ | Details . . . | 472 | −0.2380247 | −5.911042 | 0 |
| UP_ADJP001 | UP_ADJP001 | | | | | |
| HALLMARK_MTORC1_ | HALLMARK_MTORC1_ | Details . . . | 199 | −0.3684572 | −5.90008 | 0 |
| SIGNALING | SIGNALING | | | | | |
| HALLMARK_G2M_CHECKPOINT | HALLMARK_G2M_CHECKPOINT | Details . . . | 197 | −0.3583644 | −5.8054795 | 0 |
| HALLMARK_MYC_TARGETS_V2 | HALLMARK_MYC_TARGETS_V2 | Details . . . | 58 | −0.5177565 | −4.656676 | 0 |
| HALLMARK_DNA_REPAIR | HALLMARK_DNA_REPAIR | Details . . . | 142 | −0.3158149 | −4.3821316 | 0 |
| HALLMARK_OXIDATIVE_ | HALLMARK_OXIDATIVE_ | Details . . . | 195 | −0.2561592 | −4.1848326 | 0 |
| PHOSPHORYLATION | PHOSPHORYLATION | | | | | |
| HALLMARK_MITOTIC_SPINDLE | HALLMARK_MITOTIC_SPINDLE | Details . . . | 197 | −0.2428027 | −3.9649718 | 0 |
| HALLMARK_UNFOLDED_ | HALLMARK_UNFOLDED_ | Details . . . | 109 | −0.3127456 | −3.7568793 | 0 |
| PROTEIN_RESPONSE | PROTEIN_RESPONSE | | | | | |
| HALLMARK_IL2_STAT5_ | HALLMARK_IL2_STAT5_ | Details . . . | 197 | −0.2229416 | −3.5924618 | 0 |
| SIGNALING | SIGNALING | | | | | |
| HALLMARK_GLYCOLYSIS | HALLMARK_GLYCOLYSIS | Details . . . | 197 | −0.2142586 | −3.3927002 | 0 |
| HALLMARK_P53_PATHWAY | HALLMARK_P53_PATHWAY | Details . . . | 197 | −0.1895346 | −3.08352 | 0 |
| HALLMARK_ADIPOGENESIS | HALLMARK_ADIPOGENESIS | Details . . . | 196 | −0.1846776 | −2.9925427 | 0 |
| HALLMARK_XENOBIOTIC_ | HALLMARK_XENOBIOTIC_ | Details . . . | 192 | −0.175952 | −2.8495858 | 0 |
| METABOLISM | METABOLISM | | | | | |
| HALLMARK_PI3K_AKT_ | HALLMARK_PI3K_AKT_ | Details . . . | 103 | −0.2430873 | −2.8269393 | 0 |
| SIGNALING | SIGNALING | | | | | |
| HALLMARK_KRAS_ | HALLMARK_KRAS_ | Details . . . | 189 | −0.1640801 | −2.6040006 | 0 |
| SIGNALING_DN | SIGNALING_DN | | | | | |
| AHALLMARK_ALLOGRAFT_ | AHALLMARK_ALLOGRAFT_ | Details . . . | 192 | −0.159661 | −2.543514 | 0 |
| REJECTION | REJECTION | | | | | |
| HALLMARK_FATTY_ACID_ | HALLMARK_FATTY_ACID_ | Details . . . | 156 | −0.1758843 | −2.519029 | 0 |
| METABOLISM | METABOLISM | | | | | |
| HALLMARK_UV_RESPONSE_UP | HALLMARK_UV_RESPONSE_UP | Details . . . | 150 | −0.1742287 | −2.5124493 | 0 |
| HALLMARK_EPITHELIAL_ | HALLMARK_EPITHELIAL_ | Details . . . | 191 | −0.1585803 | −2.4973826 | 0 |
| MESENCHYMAL_TRANSITION | MESENCHYMAL_TRANSITION | | | | | |
| HALLMARK_PROTEIN_ | HALLMARK_PROTEIN_ | Details . . . | 95 | −0.2133321 | −2.4251304 | 0 |
| SECRETION | SECRETION | | | | | |
| HALLMARK_CHOLESTEROL_ | HALLMARK_CHOLESTEROL_ | Details . . . | 73 | −0.2023749 | −2.035934 | 0.00392157 |
| HOMEOSTASIS | HOMEOSTASIS | | | | | |
| HALLMARK_ESTROGEN_ | HALLMARK_ESTROGEN_ | Details . . . | 194 | −0.1231046 | −1.9593012 | 0.0018797 |
| RESPONSE_LATE | RESPONSE_LATE | | | | | |
| HALLMARK_ESTROGEN_ | HALLMARK_ESTROGEN_ | Details . . . | 195 | −0.1163779 | −1.841975 | 0.01402806 |
| RESPONSE_EARLY | RESPONSE_EARLY | | | | | |
| HALLMARK_WNT_BETA_ | HALLMARK_WNT_BETA_ | Details . . . | 41 | −0.2258079 | −1.7303207 | 0.02840909 |
| CATENIN_SIGNALING | CATENIN_SIGNALING | | | | | |
| HALLMARK_HYPOXIA | HALLMARK_HYPOXIA | Details . . . | 194 | −0.109338 | −1.7254679 | 0.02291667 |
| HALLMARK_PEROXISOME | HALLMARK_PEROXISOME | Details . . . | 101 | −0.1448123 | −1.7131867 | 0.02191235 |
| TILSLAMF6_V_TILTIM3_ | TILSLAMF6_V_TILTIM3_ | Details . . . | 48 | −0.2071108 | −1.691352 | 0.02674897 |
| DOWN_50 | DOWN_50 | | | | | |
| HALLMARK_MYOGENESIS | HALLMARK_MYOGENESIS | Details . . . | 196 | −0.0992372 | −1.6038872 | 0.05285412 |
| HALLMARK_ANGIOGENESIS | HALLMARK_ANGIOGENESIS | | 34 | −0.2041423 | −1.4054457 | 0.09108911 |
| HALLMARK_IL6_JAK_ | HALLMARK_IL6_JAK_ | | 85 | −0.1249788 | −1.3499168 | 0.13184585 |
| STAT3_SIGNALING | STAT3_SIGNALING | | | | | |
| HALLMARK_BILE_ACID_ | HALLMARK_BILE_ACID_ | | 111 | −0.1000328 | −1.2382027 | 0.188 |
| METABOLISM | METABOLISM | | | | | |
| HALLMARK_COAGULATION | HALLMARK_COAGULATION | | 134 | −0.08452 | −1.1284028 | 0.29959515 |
| HALLMARK_HEDGEHOG_ | HALLMARK_HEDGEHOG_ | | 33 | −0.1297794 | −0.8961801 | 0.5714286 |
| SIGNALING | SIGNALING | | | | | |
| HALLMARK_ | HALLMARK_ | | 128 | −0.0492375 | −0.6440043 | 0.90569746 |
| SPERMATOGENESIS | SPERMATOGENESIS | | | | | |

GSEA Full Report for RNA-seq profiling of Ptpn2 sgRNA vs. control cells 8
days post LCMV Clone 13 viral infection (Tim control versus Ptpn2 down)

| | GS<br> follow | FDR | FWER | RANK AT | |
|---|---|---|---|---|---|
| NAME | link to MSigDB | q-val | p-val | MAX | LEADING EDGE |
| HALLMARK_E2F_TRAGETS | HALLMARK_E2F_TRAGETS | 0 | 0 | 6283 | tags = 74%, list = 30%, |
| | | | | | signal = 104% |
| HALLMARK_MYC_TRAGETS_V1 | HALLMARK_MYC_TRAGETS_V1 | 0 | 0 | 6165 | tags = 72%, list = 29%, |
| | | | | | signal = 101% |
| LCMVSLAMF6_V_LCMVTIM3_ | LCMVSLAMF6_V_LCMVTIM3_ | 0 | 0 | 6165 | tags = 52%, list = 29%, |
| UP_ADJP001 | UP_ADJP001 | | | | signal = 72% |
| HALLMARK_MTORC1_ | HALLMARK_MTORC1_ | 0 | 0 | 5050 | tags = 60%, list = 24%, |
| SIGNALING | SIGNALING | | | | signal = 78% |
| HALLMARK_G2M_CHECKPOINT | HALLMARK_G2M_CHECKPOINT | 0 | 0 | 6145 | tags = 64%, list = 29%, |
| | | | | | signal = 90% |
| HALLMARK_MYC_TARGETS_V2 | HALLMARK_MYC_TARGETS_V2 | 0 | 0 | 4409 | tags = 72%, list = 21%, |
| | | | | | signal = 91% |
| HALLMARK_DNA_REPAIR | HALLMARK_DNA_REPAIR | 0 | 0 | 5895 | tags = 59%, list = 28%, |
| | | | | | signal = 81% |
| HALLMARK_OXIDATIVE_ | HALLMARK_OXIDATIVE_ | 0 | 0 | 6257 | tags = 55%, list = 29%, |
| PHOSPHORYLATION | PHOSPHORYLATION | | | | signal = 77% |
| HALLMARK_MITOTIC_SPINDLE | HALLMARK_MITOTIC_SPINDLE | 0 | 0 | 6097 | tags = 53%, list = 29%, |
| | | | | | signal = 73% |
| HALLMARK_UNFOLDED_ | HALLMARK_UNFOLDED_ | 0 | 0 | 6440 | tags = 61%, list = 30%, |
| PROTEIN_RESPONSE | PROTEIN_RESPONSE | | | | signal = 88% |
| HALLMARK_IL2_STAT5_ | HALLMARK_IL2_STAT5_ | 0 | 0 | 5976 | tags = 50%, list = 28%, |
| SIGNALING | SIGNALING | | | | signal = 69% |
| HALLMARK_GLYCOLYSIS | HALLMARK_GLYCOLYSIS | 0 | 0 | 4974 | tags = 45%, list = 23%, |
| | | | | | signal = 58% |
| HALLMARK_P53_PATHWAY | HALLMARK_P53_PATHWAY | 1.07E−04 | 0.001 | 6032 | tags = 47%, list = 28%, |
| | | | | | signal = 65% |
| HALLMARK_ADIPOGENESIS | HALLMARK_ADIPOGENESIS | 9.89E−05 | 0.001 | 4778 | tags = 41%, list = 23%, |
| | | | | | signal = 52% |
| HALLMARK_XENOBIOTIC_ | HALLMARK_XENOBIOTIC_ | 9.23E−05 | 0.001 | 5583 | tags = 44%, list = 26%, |
| METABOLISM | METABOLISM | | | | signal = 59% |
| HALLMARK_PI3K_AKT_ | HALLMARK_PI3K_AKT_ | 8.65E−05 | 0.001 | 5167 | tags = 49%, list = 24%, |
| SIGNALING | SIGNALING | | | | signal = 64% |
| HALLMARK_KRAS_ | HALLMARK_KRAS_ | 1.63E−04 | 0.002 | 14285 | tags = 84%, list = 67%, |
| SIGNALING_DN | SIGNALING_DN | | | | signal = 254% |
| AHALLMARK_ALLOGRAFT_ | AHALLMARK_ALLOGRAFT_ | 1.54E−04 | 0.002 | 5594 | tags = 42%, list = 26%, |
| REJECTION | REJECTION | | | | signal = 57% |
| HALLMARK_FATTY_ACID_ | HALLMARK_FATTY_ACID_ | 2.92E−04 | 0.004 | 6224 | tags = 47%, list = 29%, |
| METABOLISM | METABOLISM | | | | signal = 66% |
| HALLMARK_UV_RESPONSE_UP | HALLMARK_UV_RESPONSE_UP | 2.78E−04 | 0.004 | 4675 | tags = 39%, list = 22%, |
| | | | | | signal = 50% |
| HALLMARK_EPITHELIAL_ | HALLMARK_EPITHELIAL_ | 2.65E−04 | 0.004 | 13771 | tags = 81%, list = 65%, |
| MESENCHYMAL_TRANSITION | MESENCHYMAL_TRANSITION | | | | signal = 228% |
| HALLMARK_PROTEIN_ | HALLMARK_PROTEIN_ | 3.58E−04 | 0.006 | 6214 | tags = 51%, list = 29%, |
| SECRETION | SECRETION | | | | signal = 71% |
| HALLMARK_CHOLESTEROL_ | HALLMARK_CHOLESTEROL_ | 0.0057904 | 0.089 | 6184 | tags = 49%, list = 29%, |
| HOMEOSTASIS | HOMEOSTASIS | | | | signal = 69% |
| HALLMARK_ESTROGEN_ | HALLMARK_ESTROGEN_ | 0.00937488 | 0.156 | 8348 | tags = 52%, list = 39%, |
| RESPONSE_LATE | RESPONSE_LATE | | | | signal = 84% |
| HALLMARK_ESTROGEN_ | HALLMARK_ESTROGEN_ | 0.01800284 | 0.298 | 8651 | tags = 52%, list = 41%, |
| RESPONSE_EARLY | RESPONSE_EARLY | | | | signal = 88% |
| HALLMARK_WNT_BETA_ | HALLMARK_WNT_BETA_ | 0.03315882 | 0.495 | 5568 | tags = 49%, list = 26%, |
| CATENIN_SIGNALING | CATENIN_SIGNALING | | | | signal = 66% |
| HALLMARK_HYPOXIA | HALLMARK_HYPOXIA | 0.03272482 | 0.505 | 5357 | tags = 36%, list = 25%, |
| | | | | | signal = 48% |
| HALLMARK_PEROXISOME | HALLMARK_PEROXISOME | 0.03343299 | 0.522 | 5975 | tags = 43%, list = 28%, |
| | | | | | signal = 59% |
| TILSLAMF6_V_TILTIM3_ | TILSLAMF6_V_TILTIM3_ | 0.0352966 | 0.555 | 6224 | tags = 50%, list = 29%, |
| DOWN_50 | DOWN_50 | | | | signal = 71% |
| HALLMARK_MYOGENESIS | HALLMARK_MYOGENESIS | 0.05232287 | 0.711 | 14258 | tags = 77%, list = 67%, |
| | | | | | signal = 233% |
| HALLMARK_ANGIOGENESIS | HALLMARK_ANGIOGENESIS | 0.12324566 | 0.956 | 13147 | tags = 82%, list = 62%, |
| | | | | | signal = 216% |
| HALLMARK_IL6_JAK_ | HALLMARK_IL6_JAK_ | 0.14814843 | 0.983 | 3350 | tags = 28%, list = 16%, |
| STAT3_SIGNALING | STAT3_SIGNALING | | | | signal = 33% |
| HALLMARK_BILE_ACID_ | HALLMARK_BILE_ACID_ | 0.21890168 | 0.998 | 4770 | tags = 32%, list = 22%, |
| METABOLISM | METABOLISM | | | | signal = 42% |
| HALLMARK_COAGULATION | HALLMARK_COAGULATION | 0.31322685 | 1 | 14050 | tags = 75%, list = 66%, |
| | | | | | signal = 220% |
| HALLMARK_HEDGEHOG_ | HALLMARK_HEDGEHOG_ | 0.59253967 | 1 | 8823 | tags = 55%, list = 42%, |
| SIGNALING | SIGNALING | | | | signal = 93% |
| HALLMARK_ | HALLMARK_ | 0.90680474 | 1 | 1283 | tags = 11%, list = 6%, |
| SPERMATOGENESIS | SPERMATOGENESIS | | | | signal = 12% |

TABLE 4C
GSEA Full Report for RNA-seq profiling of Ptpn2 sgRNA vs. control cells 8
days post LCMV Clone 13 viral infection (Slam control versus Ptpn2 up)

| | GS<br> follow | GS | | | | NOM |
|---|---|---|---|---|---|---|
| NAME | link to MSigDB | DETAILS | SIZE | ES | NES | p-val |
| LCMVSLAMP6_V_LCMVTIM3_ | LCMVSLAMP6_V_LCMVTIM3_ | Details . . . | 389 | 0.30891868 | 6.818469 | 0 |
| DOWN_ADJP001 | DOWN_ADJP001 | | | | | |
| LCMVSLAMP6_V_LCMVTIM3_ | LCMVSLAMP6_V_LCMVTIM3_ | Details . . . | 472 | 0.23962681 | 5.9024863 | 0 |
| UP_ADJP001 | UP_ADJP001 | | | | | |
| TILSLAMF6_V_TILTIM3_UP_50 | TILSLAMF6_V_TILTIM3_UP_50 | Details . . . | 50 | 0.5468695 | 4.560668 | 0 |
| HALLMARK_INTERFERON_ | HALLMARK_INTERFERON_ | Details . . . | 91 | 0.36888522 | 4.108726 | 0 |
| ALPHA_RESPONSE | ALPHA_RESPONSE | | | | | |
| HALLMARK_INTERFERON_ | HALLMARK_INTERFERON_ | Details . . . | 190 | 0.2563929 | 4.0704646 | 0 |
| GAMMA_RESPONSE | GAMMA_RESPONSE | | | | | |
| LCMVSLAMF6_V_ | LCMVSLAMF6_V_ | Details . . . | 50 | 0.4711572 | 3.9583337 | 0 |
| LCMVTIM3_UP_50 | LCMVTIM3_UP_50 | | | | | |
| HALLMARK_IL2_STAT5_ | HALLMARK_IL2_STAT5_ | Details . . . | 197 | 0.22718972 | 3.7981393 | 0 |
| SIGNALING | SIGNALING | | | | | |
| HALLMARK_ALLOGRAFT_ | HALLMARK_ALLOGRAFT_ | Details . . . | 192 | 0.18989493 | 3.0600293 | 0 |
| REJECTION | REJECTION | | | | | |
| HALLMARK_APOPTOSIS | HALLMARK_APOPTOSIS | Details . . . | 159 | 0.20460683 | 2.9857357 | 0 |
| HALLMARK_HEME_ | HALLMARK_HEME_ | Details . . . | 186 | 0.160511 | 2.610648 | 0 |
| METABOLISM | METABOLISM | | | | | |
| LCMVSLAMF6_V_ | LCMVSLAMF6_V_ | Details . . . | 49 | 0.30880028 | 2.6065738 | 0 |
| LCMVTIM3_DOWN_50 | LCMVTIM3_DOWN_50 | | | | | |
| HALLMARK_TNFA_ | HALLMARK_TNFA_ | Details . . . | 194 | 0.15980926 | 2.5745876 | 0 |
| SIGNALING_VIA_NFKB | SIGNALING_VIA_NFKB | | | | | |
| HALLMARK_ANDROGEN_ | HALLMARK_ANDROGEN_ | Details . . . | 98 | 0.20676808 | 2.3687918 | 0 |
| RESPONSE | RESPONSE | | | | | |
| HALLMARK_INFLAMMATORY_ | HALLMARK_INFLAMMATORY_ | Details . . . | 193 | 0.13153768 | 2.1808236 | 0 |
| RESPONSE | RESPONSE | | | | | |
| HALLMARK_UV_RESPONSE_DN | HALLMARK_UV_RESPONSE_DN | Details . . . | 140 | 0.15468915 | 2.1310802 | 0.0039604 |
| TILSLAMF6_V_TILTIM3_ | TILSLAMF6_V_TILTIM3_ | Details . . . | 48 | 0.25584102 | 2.1274705 | 0 |
| DOWN_50 | DOWN_50 | | | | | |
| HALLMARK_PROTEIN_ | HALLMARK_PROTEIN_ | Details . . . | 95 | 0.17995377 | 2.0289116 | 0.00193798 |
| SECRETION | SECRETION | | | | | |
| HALLMARK_COMPLEMENT | HALLMARK_COMPLEMENT | Details . . . | 185 | 0.11230509 | 1.7744362 | 0.02249489 |
| HALLMARK_KRAS_ | HALLMARK_KRAS_ | Details . . . | 193 | 0.10935846 | 1.7561868 | 0.02579365 |
| SIGNALING_UP | SIGNALING_UP | | | | | |
| HALLMARK_IL6_JAK_STAT3_ | HALLMARK_IL6_JAK_STAT3_ | Details . . . | 85 | 0.16374648 | 1.7495087 | 0.02647658 |
| SIGNALING | SIGNALING | | | | | |
| HALLMARK_NOTCH_ | HALLMARK_NOTCH_ | Details . . . | 32 | 0.24194296 | 1.6187168 | 0.05870445 |
| SIGNALING | SIGNALING | | | | | |
| HALLMARK_APICAL_ | HALLMARK_APICAL_ | Details . . . | 198 | 0.08807562 | 1.4394321 | 0.10021322 |
| JUNCTION | JUNCTION | | | | | |
| HALLMARK_BILE_ACID_ | HALLMARK_BILE_ACID_ | Details . . . | 111 | 0.10655326 | 1.3517694 | 0.12809917 |
| METABOLISM | METABOLISM | | | | | |
| HALLMARK_TGF_BETA_ | HALLMARK_TGF_BETA_ | Details . . . | 53 | 0.15928149 | 1.3461119 | 0.12447257 |
| SIGNALING | SIGNALING | | | | | |
| HALLMARK_WNT_BETA_ | HALLMARK_WNT_BETA_ | Details . . . | 41 | 0.15175818 | 1.1448803 | 0.27756655 |
| CATENIN_SIGNALING | CATENIN_SIGNALING | | | | | |
| HALLMARK_APICAL_ | HALLMARK_APICAL_ | Details . . . | 43 | 0.12747745 | 0.9748822 | 0.46332046 |
| SURFACE | SURFACE | | | | | |

GSEA Full Report for RNA-seq profiling of Ptpn2 sgRNA vs. control cells 8
days post LCMV Clone 13 viral infection (Slam control versus Ptpn2 up)

| | GS<br> follow | FDR | FWER | RANK AT | |
|---|---|---|---|---|---|
| NAME | link to MSigDB | q-val | p-val | MAX | LEADING EDGE |
| LCMVSLAMP6_V_LCMVTIM3_ | LCMVSLAMP6_V_LCMVTIM3_ | 0 | 0 | 6163 | tags = 59%, list = 29%, |
| DOWN_ADJP001 | DOWN_ADJP001 | | | | signal = 82% |
| LCMVSLAMP6_V_LCMVTIM3_ | LCMVSLAMP6_V_LCMVTIM3_ | 0 | 0 | 5950 | tags = 51%, list = 28%, |
| UP_ADJP001 | UP_ADJP001 | | | | signal = 70% |
| TILSLAMF6_V_TILTIM3_UP_50 | TILSLAMF6_V_TILTIM3_UP_50 | 0 | 0 | 5396 | tags = 80%, list = 25%, |
| | | | | | signal = 107% |
| HALLMARK_INTERFERON_ | HALLMARK_INTERFERON_ | 0 | 0 | 6194 | tags = 66%, list = 29%, |
| ALPHA_RESPONSE | ALPHA_RESPONSE | | | | signal = 93% |
| HALLMARK_INTERFERON_ | HALLMARK_INTERFERON_ | 0 | 0 | 5327 | tags = 51%, list = 25%, |
| GAMMA_RESPONSE | GAMMA_RESPONSE | | | | signal = 67% |
| LCMVSLAMF6_V_ | LCMVSLAMF6_V_ | 0 | 0 | 4877 | tags = 70%, list = 23%, |
| LCMVTIM3_UP_50 | LCMVTIM3_UP_50 | | | | signal = 91% |
| HALLMARK_IL2_STAT5_ | HALLMARK_IL2_STAT5_ | 0 | 0 | 5454 | tags = 48%, list = 26%, |
| SIGNALING | SIGNALING | | | | signal = 64% |
| HALLMARK_ALLOGRAFT_ | HALLMARK_ALLOGRAFT_ | 0 | 0 | 4625 | tags = 41%, list = 22%, |
| REJECTION | REJECTION | | | | signal = 51% |
| HALLMARK_APOPTOSIS | HALLMARK_APOPTOSIS | 0 | 0 | 6231 | tags = 50%, list = 29%, |
| | | | | | signal = 70% |
| HALLMARK_HEME_ | HALLMARK_HEME_ | 0 | 0 | 6204 | tags = 45%, list = 29%, |
| METABOLISM | METABOLISM | | | | signal = 63% |
| LCMVSLAMF6_V_ | LCMVSLAMF6_V_ | 0 | 0 | 1689 | tags = 39%, list = 8%, |
| LCMVTIM3_DOWN_50 | LCMVTIM3_DOWN_50 | | | | signal = 42% |
| HALLMARK_TNFA_ | HALLMARK_TNFA_ | 6.77E−05 | 0.001 | 2982 | tags = 30%, list = 14%, |
| SIGNALING_VIA_NFKB | SIGNALING_VIA_NFKB | | | | signal = 34% |
| HALLMARK_ANDROGEN_ | HALLMARK_ANDROGEN_ | 7.16E−04 | 0.01 | 4075 | tags = 40%, list = 19%, |
| RESPONSE | RESPONSE | | | | signal = 49% |
| HALLMARK_INFLAMMATORY_ | HALLMARK_INFLAMMATORY_ | 0.00288978 | 0.043 | 4818 | tags = 36%, list = 23%, |
| RESPONSE | RESPONSE | | | | signal = 46% |
| HALLMARK_UV_RESPONSE_DN | HALLMARK_UV_RESPONSE_DN | 0.00374346 | 0.058 | 5679 | tags = 42%, list = 27%, |
| | | | | | signal = 57% |
| TILSLAMF6_V_TILTIM3_ | TILSLAMF6_V_TILTIM3_ | 0.0035095 | 0.058 | 2539 | tags = 38%, list = 12%, |
| DOWN_50 | DOWN_50 | | | | signal = 43% |
| HALLMARK_PROTEIN_ | HALLMARK_PROTEIN_ | 0.00607681 | 0.104 | 6247 | tags = 47%, list = 29%, |
| SECRETION | SECRETION | | | | signal = 67% |
| HALLMARK_COMPLEMENT | HALLMARK_COMPLEMENT | 0.02750925 | 0.422 | 4976 | tags = 35%, list = 23%, |
| | | | | | signal = 45% |
| HALLMARK_KRAS_ | HALLMARK_KRAS_ | 0.02918336 | 0.466 | 4295 | tags = 31%, list = 20%, |
| SIGNALING_UP | SIGNALING_UP | | | | signal = 39% |
| HALLMARK_IL6_JAK_STAT3_ | HALLMARK_IL6_JAK_STAT3_ | 0.02904014 | 0.479 | 4775 | tags = 39%, list = 23%, |
| SIGNALING | SIGNALING | | | | signal = 50% |
| HALLMARK_NOTCH_ | HALLMARK_NOTCH_ | 0.05075361 | 0.707 | 4818 | tags = 47%, list = 23%, |
| SIGNALING | SIGNALING | | | | signal = 618% |
| HALLMARK_APICAL_ | HALLMARK_APICAL_ | 0.10837197 | 0.921 | 9076 | tags = 52%, list = 43%, |
| JUNCTION | JUNCTION | | | | signal = 89% |
| HALLMARK_BILE_ACID_ | HALLMARK_BILE_ACID_ | 0.14868538 | 0.972 | 9217 | tags = 54%, list = 43%, |
| METABOLISM | METABOLISM | | | | signal = 95% |
| HALLMARK_TGF_BETA_ | HALLMARK_TGF_BETA_ | 0.14551048 | 0.975 | 5434 | tags = 42%, list = 26%, |
| SIGNALING | SIGNALING | | | | signal = 84% |
| HALLMARK_WNT_BETA_ | HALLMARK_WNT_BETA_ | 0.28941077 | 1 | 6099 | tags = 44%, list = 29%, |
| CATENIN_SIGNALING | CATENIN_SIGNALING | | | | signal = 61% |
| HALLMARK_APICAL_ | HALLMARK_APICAL_ | 0.4655409 | 1 | 16540 | tags = 91%, list = 78%, |
| SURFACE | SURFACE | | | | signal = 411% |

TABLE 4D
GSEA Full Report for RNA-seq profiling of Ptpn2 sgRNA vs. control cells 8
days post LCMV Clone 13 viral infection (Slam control versus Ptpn2 down)

| | GS<br> follow | GS | | | | NOM |
|---|---|---|---|---|---|---|
| NAME | link to MSigDB | DETAILS | SIZE | ES | NES | p-val |
| HALLMARK_E2F_TRAGETS | HALLMARK_E2F_TRAGETS | Details . . . | 199 | −0.4752412 | −7.849448 | 0 |
| HALLMARK_OXIDATIVE_ | HALLMARK_OXIDATIVE_ | Details . . . | 195 | −0.4819503 | −7.692486 | 0 |
| PHOSPHORYLATION | PHOSPHORYLATION | | | | | |
| HALLMARK_MYC_ | HALLMARK_MYC_ | Details . . . | 197 | −0.4080904 | −6.619076 | 0 |
| TARGETS_V1 | TARGETS_V1 | | | | | |
| HALLMARK_G2M_ | HALLMARK_G2M_ | Details . . . | 197 | −0.3901002 | −6.3553305 | 0 |
| CHECKPOINT | CHECKPOINT | | | | | |
| HALLMARK_MTORC1_ | HALLMARK_MTORC1_ | Details . . . | 199 | −0.3697892 | −6.0370483 | 0 |
| SIGNALING | SIGNALING | | | | | |
| HALLMARK_MYC_ | HALLMARK_MYC_ | Details . . . | 58 | −0.5524634 | −4.9435306 | 0 |
| TARGETS_V2 | TARGETS_V2 | | | | | |
| HALLMARK_DNA_REPAIR | HALLMARK_DNA_REPAIR | Details . . . | 142 | −0.3463675 | −4.799507 | 0 |
| HALLMARK_ADIPOGENESIS | HALLMARK_ADIPOGENESIS | Details . . . | 196 | −0.287461 | −4.7243457 | 0 |
| HALLMARK_GLYCOLYSIS | HALLMARK_GLYCOLYSIS | Details . . . | 197 | −0.2437657 | −4.050974 | 0 |
| HALLMARK_P53_PATHWAY | HALLMARK_P53_PATHWAY | Details . . . | 197 | −0.2074885 | −3.4086208 | 0 |
| HALLMARK_MITOTIC_ | HALLMARK_MITOTIC_ | Details . . . | 197 | −0.210294 | −3.4025595 | 0 |
| SPINDLE | SPINDLE | | | | | |
| HALLMARK_UNFOLDED_ | HALLMARK_UNFOLDED_ | Details . . . | 109 | −0.2457554 | −3.0096867 | 0 |
| PROTEIN_RESPONSE | PROTEIN_RESPONSE | | | | | |
| HALLMARK_ESTROGEN_ | HALLMARK_ESTROGEN_ | Details . . . | 194 | −0.1890173 | −2.990862 | 0 |
| RESPONSE_LATE | RESPONSE_LATE | | | | | |
| HALLMARK_REACTIVE_ | HALLMARK_REACTIVE_ | Details . . . | 47 | −0.3773133 | −2.9862819 | 0 |
| OXIGEN_SPECIES_PATHWAY | OXIGEN_SPECIES_PATHWAY | | | | | |
| HALLMARK_FATTY_ | HALLMARK_FATTY_ | Details . . . | 156 | −0.2019098 | −2.927889 | 0 |
| ACID_METABOLISM | ACID_METABOLISM | | | | | |
| HALLMARK_KRAS_ | HALLMARK_KRAS_ | Details . . . | 189 | −0.1762453 | −2.8209713 | 0 |
| SIGNALING_DN | SIGNALING_DN | | | | | |
| HALLMARK_UV_ | HALLMARK_UV_ | Details . . . | 150 | −0.188409 | −2.70185 | 0 |
| RESPONSE_UP | RESPONSE_UP | | | | | |
| HALLMARK_EPITHELIAL_ | HALLMARK_EPITHELIAL_ | Details . . . | 191 | −0.1630102 | −2.6696599 | 0 |
| MESENCHYMAL_TRANSITION | MESENCHYMAL_TRANSITION | | | | | |
| HALLMARK_PI3K_AKT_ | HALLMARK_PI3K_AKT_ | Details . . . | 103 | −0.2133914 | −2.4827614 | 0 |
| MTOR_SIGNALING | MTOR_SIGNALING | | | | | |
| HALLMARK_PEROXISOME | HALLMARK_PEROXISOME | Details . . . | 101 | −0.1957828 | −2.2604945 | 0.00199601 |
| HALLMARK_XENOBIOTIC_ | HALLMARK_XENOBIOTIC_ | Details . . . | 192 | −0.1298211 | −2.0772073 | 0.002 |
| METABOLISM | METABOLISM | | | | | |
| HALLMARK_HYPOXIA | HALLMARK_HYPOXIA | Details . . . | 194 | −0.1280484 | −2.064944 | 0.0021645 |
| HALLMARK_ESTROGEN_ | HALLMARK_ESTROGEN_ | Details . . . | 195 | −0.1247451 | −1.9710221 | 0.0019685 |
| RESPONSE_EARLY | RESPONSE_EARLY | | | | | |
| HALLMARK_MYOGENESIS | HALLMARK_MYOGENESIS | Details . . . | 196 | −0.11174 | −1.7870141 | 0.02074689 |
| HALLMARK_CHOLESTEROL_ | HALLMARK_CHOLESTEROL_ | Details . . . | 73 | −0.1635801 | −1.6008555 | 0.04633205 |
| HOMEOSTASIS | HOMEOSTASIS | | | | | |
| HALLMARK_ANGIOGENESIS | HALLMARK_ANGIOGENESIS | Details . . . | 34 | −0.2245871 | −1.5452391 | 0.0662768 |
| HALLMARK_ | HALLMARK_ | Details . . . | 128 | −0.1211232 | −1.5448202 | 0.06309751 |
| SPERMATOGENESIS | SPERMATOGENESIS | | | | | |
| HALLMARK_PANCREAS_ | HALLMARK_PANCREAS_ | Details . . . | 34 | −0.2164186 | −1.4971824 | 0.06538462 |
| BETA_CELLS | BETA_CELLS | | | | | |
| HALLMARK_COAGULATION | HALLMARK_COAGULATION | Details . . . | 134 | −0.0950848 | −1.2773895 | 0.18348624 |
| HALLMARK_HEDGEHOG_ | HALLMARK_HEDGEHOG_ | Details . . . | 33 | −0.0974075 | −0.6699423 | 0.8858921 |
| SIGNALING | SIGNALING | | | | | |

GSEA Full Report for RNA-seq profiling of Ptpn2 sgRNA vs. control cells 8
days post LCMV Clone 13 viral infection (Slam control versus Ptpn2 down)

| | GS<br> follow | FDR | FWER | RANK AT | |
|---|---|---|---|---|---|
| NAME | link to MSigDB | q-val | p-val | MAX | LEADING EDGE |
| HALLMARK_E2F_TRAGETS | HALLMARK_E2F_TRAGETS | 0 | 0 | 4938 | tags = 70%, list = 23%, |
| | | | | | signal = 91% |
| HALLMARK_OXIDATIVE_ | HALLMARK_OXIDATIVE_ | 0 | 0 | 5210 | tags = 72%, list = 25%, |
| PHOSPHORYLATION | PHOSPHORYLATION | | | | signal = 95% |
| HALLMARK_MYC_ | HALLMARK_MYC_ | 0 | 0 | 5423 | tags = 66%, list = 26%, |
| TARGETS_V1 | TARGETS_V1 | | | | signal = 88% |
| HALLMARK_G2M_ | HALLMARK_G2M_ | 0 | 0 | 5155 | tags = 63%, list = 24%, |
| CHECKPOINT | CHECKPOINT | | | | signal = 82% |
| HALLMARK_MTORC1_ | HALLMARK_MTORC1_ | 0 | 0 | 5555 | tags = 63%, list = 26%, |
| SIGNALING | SIGNALING | | | | signal = 84% |
| HALLMARK_MYC_ | HALLMARK_MYC_ | 0 | 0 | 4772 | tags = 78%, list = 22%, |
| TARGETS_V2 | TARGETS_V2 | | | | signal = 100% |
| HALLMARK_DNA_REPAIR | HALLMARK_DNA_REPAIR | 0 | 0 | 5550 | tags = 61%, list = 26%, |
| | | | | | signal = 81% |
| HALLMARK_ADIPOGENESIS | HALLMARK_ADIPOGENESIS | 0 | 0 | 5540 | tags = 55%, list = 26%, |
| | | | | | signal = 73% |
| HALLMARK_GLYCOLYSIS | HALLMARK_GLYCOLYSIS | 0 | 0 | 5323 | tags = 49%, list = 25%, |
| | | | | | signal = 65% |
| HALLMARK_P53_PATHWAY | HALLMARK_P53_PATHWAY | 0 | 0 | 5547 | tags = 47%, list = 26%, |
| | | | | | signal = 63% |
| HALLMARK_MITOTIC_ | HALLMARK_MITOTIC_ | 0 | 0 | 5165 | tags = 45%, list = 24%, |
| SPINDLE | SPINDLE | | | | signal = 59% |
| HALLMARK_UNFOLDED_ | HALLMARK_UNFOLDED_ | 0 | 0 | 5713 | tags = 51%, list = 27%, |
| PROTEIN_RESPONSE | PROTEIN_RESPONSE | | | | signal = 70% |
| HALLMARK_ESTROGEN_ | HALLMARK_ESTROGEN_ | 0 | 0 | 7728 | tags = 55%, list = 36%, |
| RESPONSE_LATE | RESPONSE_LATE | | | | signal = 86% |
| HALLMARK_REACTIVE_ | HALLMARK_REACTIVE_ | 0 | 0 | 5555 | tags = 64%, list = 26%, |
| OXIGEN_SPECIES_PATHWAY | OXIGEN_SPECIES_PATHWAY | | | | signal = 86% |
| HALLMARK_FATTY_ | HALLMARK_FATTY_ | 0 | 0 | 5540 | tags = 46%, list = 26%, |
| ACID_METABOLISM | ACID_METABOLISM | | | | signal = 62% |
| HALLMARK_KRAS_ | HALLMARK_KRAS_ | 0 | 0 | 13917 | tags = 83%, list = 66%, |
| SIGNALING_DN | SIGNALING_DN | | | | signal = 239% |
| HALLMARK_UV_ | HALLMARK_UV_ | 6.54E−05 | 0.001 | 4942 | tags = 42%, list = 23%, |
| RESPONSE_UP | RESPONSE_UP | | | | signal = 54% |
| HALLMARK_EPITHELIAL_ | HALLMARK_EPITHELIAL_ | 1.37E−04 | 0.002 | 13900 | tags = 82%, list = 66%, |
| MESENCHYMAL_TRANSITION | MESENCHYMAL_TRANSITION | | | | signal = 235% |
| HALLMARK_PI3K_AKT_ | HALLMARK_PI3K_AKT_ | 5.44E−04 | 0.009 | 4970 | tags = 45%, list = 23%, |
| MTOR_SIGNALING | MTOR_SIGNALING | | | | signal = 58% |
| HALLMARK_PEROXISOME | HALLMARK_PEROXISOME | 0.0016094 | 0.03 | 5529 | tags = 46%, list = 26%, |
| | | | | | signal = 61% |
| HALLMARK_XENOBIOTIC_ | HALLMARK_XENOBIOTIC_ | 0.0051546 | 0.096 | 4564 | tags = 34%, list = 22%, |
| METABOLISM | METABOLISM | | | | signal = 43% |
| HALLMARK_HYPOXIA | HALLMARK_HYPOXIA | 0.00523199 | 0.099 | 4417 | tags = 34%, list = 21%, |
| | | | | | signal = 42% |
| HALLMARK_ESTROGEN_ | HALLMARK_ESTROGEN_ | 0.00810837 | 0.162 | 8040 | tags = 50%, list = 38%, |
| RESPONSE_EARLY | RESPONSE_EARLY | | | | signal = 80% |
| HALLMARK_MYOGENESIS | HALLMARK_MYOGENESIS | 0.02139234 | 0.391 | 13887 | tags = 77%, list = 65%, |
| | | | | | signal = 220% |
| HALLMARK_CHOLESTEROL_ | HALLMARK_CHOLESTEROL_ | 0.05022282 | 0.709 | 4970 | tags = 40%, list = 23%, |
| HOMEOSTASIS | HOMEOSTASIS | | | | signal = 52% |
| HALLMARK_ANGIOGENESIS | HALLMARK_ANGIOGENESIS | 0.0622654 | 0.791 | 12714 | tags = 82%, list = 60%, |
| | | | | | signal = 205% |
| HALLMARK_ | HALLMARK_ | 0.06011631 | 0.792 | 3579 | tags = 29%, list = 17%, |
| SPERMATOGENESIS | SPERMATOGENESIS | | | | signal = 35% |
| HALLMARK_PANCREAS_ | HALLMARK_PANCREAS_ | 0.07421137 | 0.868 | 12887 | tags = 82%, list = 61%, |
| BETA_CELLS | BETA_CELLS | | | | signal = 209% |
| HALLMARK_COAGULATION | HALLMARK_COAGULATION | 0.17645004 | 0.992 | 13669 | tags = 74%, list = 64%, |
| | | | | | signal = 206% |
| HALLMARK_HEDGEHOG_ | HALLMARK_HEDGEHOG_ | 0.8826265 | 1 | 8223 | tags = 48%, list = 39%, |
| SIGNALING | SIGNALING | | | | signal = 79% |

Principal component projections of these cells revealed that the Slamf6⁺ and Tim-3⁺ subpopulations clustered together regardless of Ptpn2 deletion (FIG. 14E). Moreover, GSEA analysis revealed that both control and Ptpn2-deleted Slamf6+ cells were significantly enriched for the LCMV Slamf6 vs. Tim-3 UP signature (Miller et al. (2019) Nat. Immunol.) (FIG. 13G). Likewise, the control and Ptpn2-deleted Tim-3+ cells were significantly enriched for the LCMV Slamf6 vs. Tim-3 DOWN signature (Miller et al. (2019) Nat. Immunol.) (FIG. 13G). However, GSEA analysis of the Slamf6⁺ control or Ptpn2-deleted cells revealed an enrichment for effector-related gene signatures, including several of which that were enriched at the day 30 time point (FIG. 13H). In addition, Ptpn2-deleted Tim-3⁺ cells were also enriched for effector-related gene signatures compared with control cells (FIG. 13I). These findings indicate that while Ptpn2 deletion does not fundamentally change the Slamf6⁺ and Tim-3⁺ subpopulations it does induce robust increases in effector-related genes both early and late post LCMV infection.

Consistent with this, using ATAC-seq (Corces et al. (2016)Nat. Genet. 48: 1193-1203) it was demonstrated that Pqpn2 deletion had almost no effect on the epigenetic state of either the Slamf6⁺ or Tim-3⁺ populations (<0.2% of open chromatin regions differentially expressed) eight days post LCMV infection (FIG. 14F). Both control and Ptpn2-deleted CD8⁺ T cells still showed characteristic differences between the Slamf6⁺ and Tim-3⁺ populations (FIG. 14G). In addition, both control and Ptpn2-deleted cells showed epigenetic marks at the Tox (FIG. 14H) locus characteristic of T cell exhaustion (Sen et al. (2016) Science 354:1165-1169). Thus, Ptpn2 deletion did not change the epigenetic states of the Tim-3⁺ and Slamf6⁺ subpopulations.

### Example 10: Ppn2 Deletion Increases Tim-3⁺ Cell Differentiation Through Enhanced IFN-α Signaling

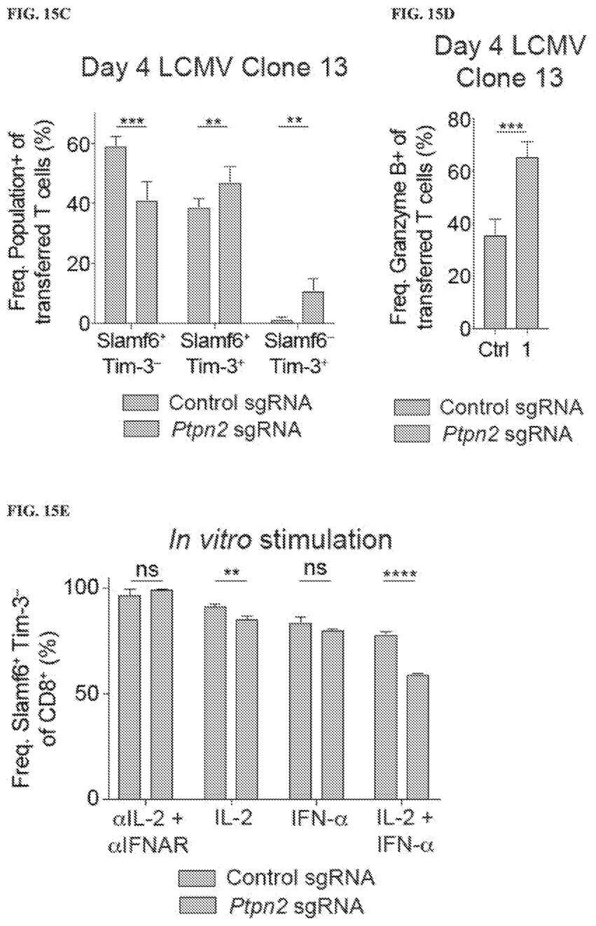

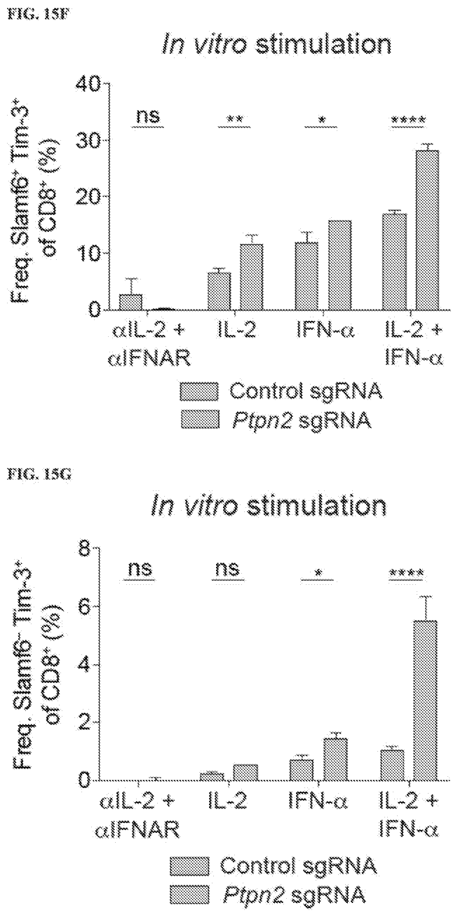

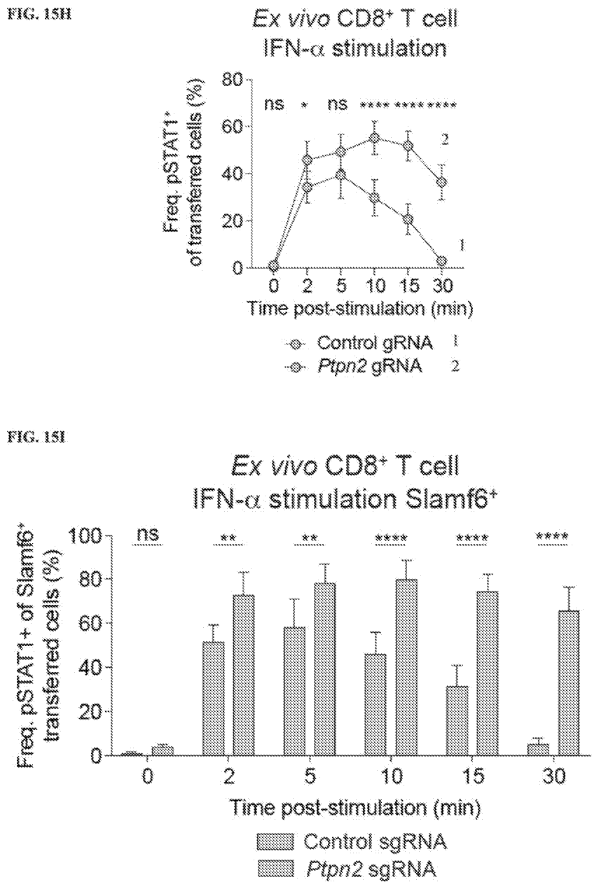

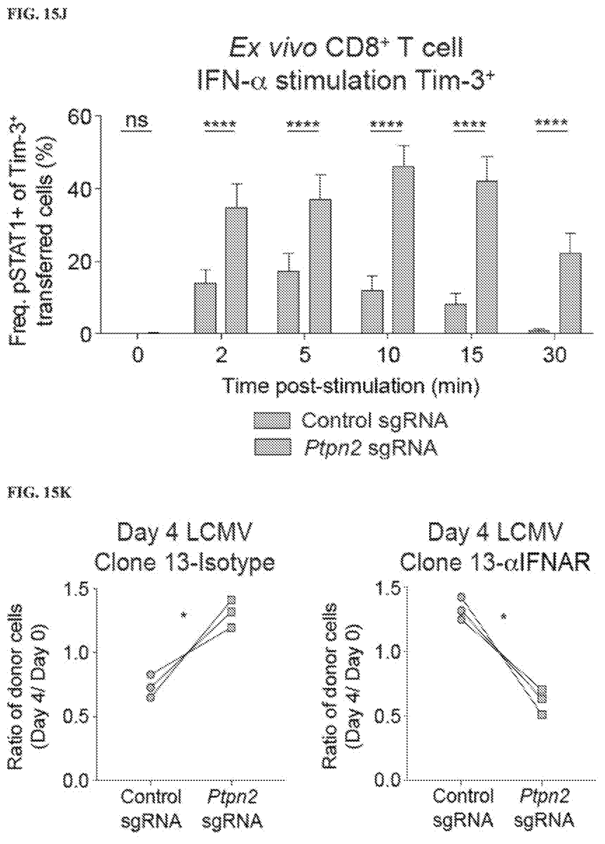

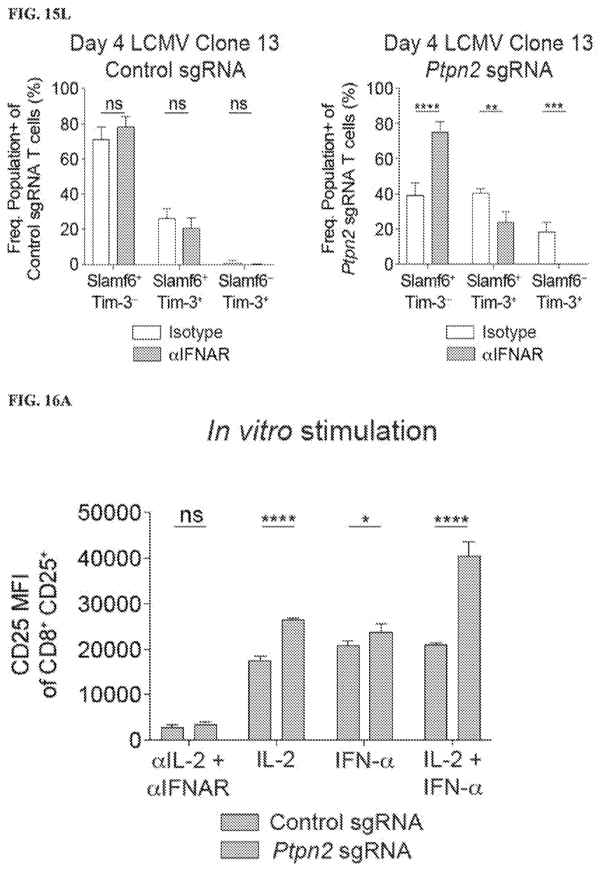

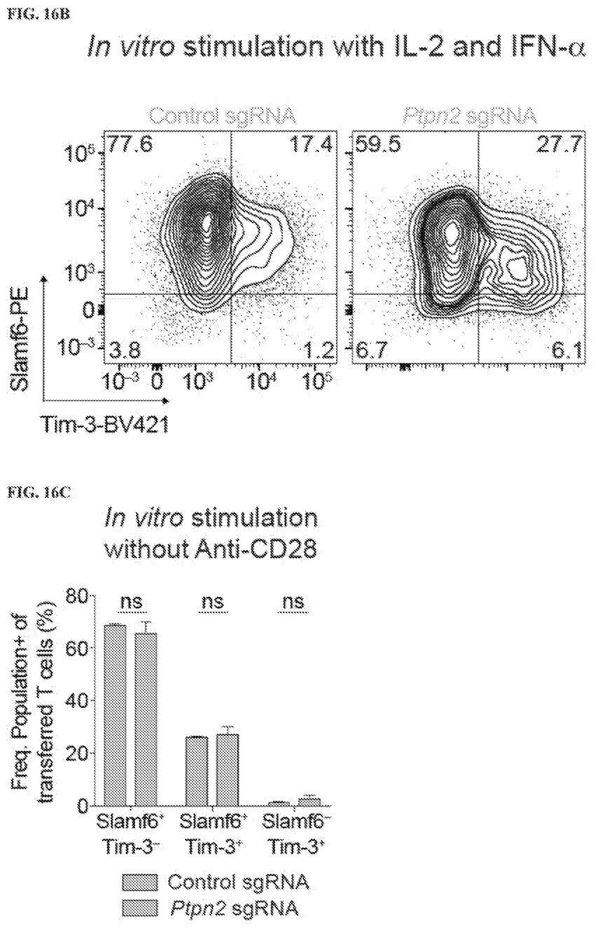

The increase in Tim-3⁺ cells at day 8 post LCMV infection coupled with the enhanced effector-related gene signatures, led to the question whether Ptpn2 was impacting the differentiation of these subpopulations. To examine this control and Ptpn2-deleted CD8⁺ T cells were co-transferred into recipient mice, the recipients were infected with LCMV Clone 13, and competitive frequencies were analyzed at day 4 post infection. Indeed, Ptpn2-deleted CD8⁺ T cells had a significant advantage over control cells at this time point (FIG. 15A). Furthermore, the Ptpn2-deleted cells, but not control cells, had already begun to differentiate into Tim-3⁺ cells (FIG. 15B). Ptpn2 deletion led to a decrease in the percentage of Slamf6⁺ Tim-3⁻ cells and an increase in the percentage of Slamf6⁺ Tim-3⁺ cells and Slamf6⁻ Tim-3⁺ cells (FIG. 15C), as well as an increase in Granzyme B expression in the Ptpn2-deleted cells (FIG. 15D).

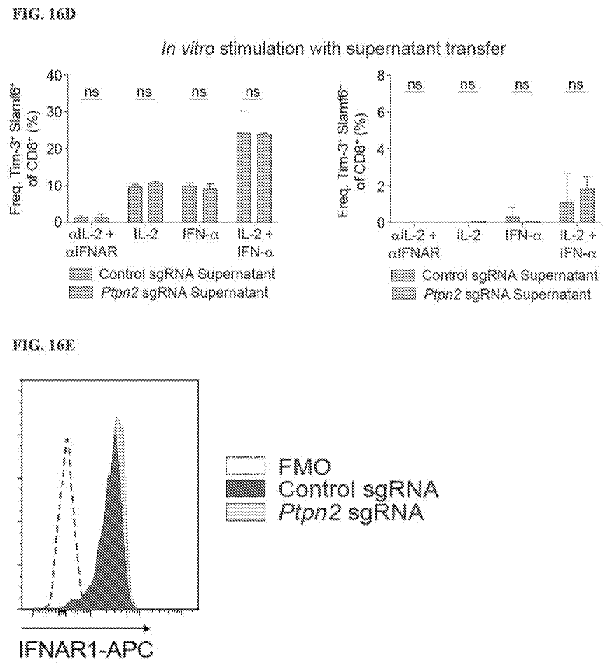

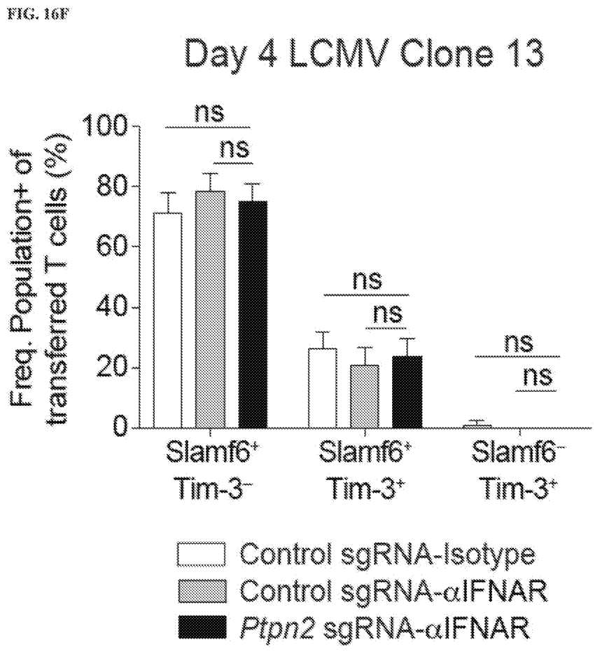

To determine the factors driving these changes an in vitro stimulation assay was used. The roles of IL-2 and IFN-α were tested, since these cytokines have important functions during T cell responses to viral infection (Cousens, Orange, and Biron (1995) J. Immunol. 155:5690-5699: Wilson et al. (2013) Science 340:202-207; Teijaro et al. (2013) Science 340:207-211). In addition, these cytokine signaling cascades lead to phosphorylation of STAT5 and STAT1, both known targets of Ptpn2 (Hoeve et al. (2002) Mol. Cell. Biol. 22:5662-5668; Simoncic et al. (2002) Curr. Biol. 12:446-453. Control or Ptpn2-deleted naive CD8⁺ T cells were stimulated with αCD3/CD28 and IL-2, IFN-α, both IL-2 and IFN-α, or blocking antibodies to abolish IL-2 and IFN-α signaling. Stimulation with IL-2, IFN-α, and the combination of IL-2 and IFN-α increased the expression level of CD25 on Ptpn2-deleted cells compared with control cells (FIG. 16A), indicating increased activation of these cells. Moreover, IL-2 plus IFN-α decreased the percentage of Tim-3-Slamf6⁺ cells and increased the percentage of Tim-3⁺ Slamf6⁺ and Tim-3⁺ Slamf6⁻ cells in the Ptpn2-deleted cells, compared with control cells or Ptpn2-deleted cells cultured with blocking antibodies to IL-2 and type 1 interferon receptor (FIGS. 15E-15G and 16B). In addition, CD28 stimulation was required for the formation of the Tim-3⁺ Slamf6⁻ subset in the presence of IL-2 and IFN-α (FIG. 16C).

It was also investigated whether a soluble factor produced by the Ptpn2-deleted cells contributed to the changes in Tim-3⁺ subset differentiation. Conditioned supernatant was isolated following stimulation of control or Ptpn2-deleted CD8⁺ T cells with αCD3/CD28 cultured with IL-2 and IFN-α and plated the supernatant on WT cells stimulated with αCD3/CD28, IL-2 and IFN-α. Conditioned supernatant did not increase the percentage of Tim-3⁺ Slamf6⁺ or Tim-3⁺ Slamf6⁻ cells, indicating that a soluble factor produced by the Ptpn2-deleted cells is unlikely to be responsible for the changes in Tim-3⁺ differentiation (FIG. 16D). Thus, IL-2, IFN-α, and CD28 are required for the enhanced generation of Tim-3⁺ cells in Ptpn2-deleted CD8⁺ T cells in the in vitro stimulation assay.

Given the requirement for IFN-α in the in vitro stimulation assay and the known role for type 1 IFN signaling in the regulation of Tim-3⁺ and Slamf6⁺ subpopulations (Wu et al. (2016) Sci. Immunol. 1:eaai8593), it was investigated whether Ptpn2-deleted CD8⁺ T cells had differential phosphorylation of STAT1 (pSTAT1) following LCMV Clone 13 viral infection. Ex vivo stimulation with IFN-α revealed that Ptpn2-deleted cells had an increased percentage and duration of expression of pSTAT1 (FIG. 15H). This increase in pSTAT1 was observed in both Slamf6⁺ and Tim-3⁺ subsets (FIGS. 15I and 15J). Control and Ptpn2-deleted CD8⁺ T cells expressed IFNAR1 at comparable levels indicating that the increased pSTAT1 was not due to a difference in receptor expression (FIG. 16E).

It was next asked whether type 1 interferon signaling was required for the expansion of the Tim-3⁺ subpopulation following Ptpn2 deletion by blocking the type 1 interferon receptor in vivo. Type 1 interferon signaling was required for the early expansion seen in Ptpn2-deficient cells (FIG. 15K), as blockade of the type 1 interferon receptor (IFNAR1) led to a competitive disadvantage for Ptpn2-deficient CD8⁺ T cells. IFNAR1 blockade led to a significant decrease in the Tim-3⁺ cells in the Ptpn2-deleted CD8⁺ T cells (FIG. 15L) and restored the percentages of Slamf6⁻ Tim-3⁻, Slamf6⁺ Tim-3⁺, and Slamf6⁻ Tim-3⁺ to that of control cells (FIG. 16F). In contrast, IFNAR1 blockade did not affect the percentages of Slamf6⁺ and Tim-3⁺ in the control CD8⁺ T cells at day 4 post LCMV Clone 13 infection (FIG. 15L). These findings indicate that enhanced type 1 interferon signaling drives the early competitive advantage and Tim-3⁺ differentiation observed in the Ptpn2-deficient cells.

### Example 11: Loss of Ptpn2 Enhances CD8⁺ T Cell Response to MC38 Tumors

Given the importance of the exhausted subpopulations in tumors (Wu et al. (2016) Sci. Immunol. 1:eaai8593; Philip et al. (2017) Nature 545:452-456; Brummelman et al. (2018). Exp. Med. 215:2520-2535; Sade-Feldman et al. (2018) Cell 175:998-1013; Thommen et al. (2018) Nat. Med. 24:994-1004; Miller et al. (2019) Nat. Immunol.; Siddiqui et al. (2019) Immunity 50:195-211; Kurtulus et al. (2019) Immunity 50:181-194), it was next asked if Ptpn2 also regulates the balance and functions of CD8⁺ T subpopulations in responses to tumors. To interrogate this, a 1:1 ratio of OT-1 TCR transgenic Ptpn2 sgRNA-containing and control sgRNA-containing CD8⁺ T cells were co-transferred to wild-type recipient mice and these mice were subsequently injected with MC38-OVA tumors. Consistent with chronic LCMV infection, Ptpn2 sgRNA-containing OT-1 CD8⁺ T cells significantly outcompeted control sgRNA-containing CD8⁺ T cells at day 7 in the tumor (FIGS. 8A and 6A), while there were no significant differences between control sgRNAs. Ptpn2 deletion also led to an increase in CD25 and a decrease in CD127 expression in transferred CD8⁺ T cells found in the tumor-draining lymph node (FIGS. 8K and 8L), indicating increased activation of these cells. In addition, Ptpn2-deleted cells had a slight increase in IFNγ production following peptide restimulation in vitro (FIG. 6L). It was next examined whether intrinsic changes arose due to Ptpn2 deletion by examining Granzyme B expression on mixed populations (as described above) of Ptpn2 and control sgRNA-containing OT-1 CD8⁺ T cells in the tumor, draining lymph node, and spleen. Deletion of Ptpn2 also increased the percentages of Granzyme B expressing OT-1 CD8⁺ T cells in the tumor, draining lymph node, and spleen, compared with control sgRNA-containing OT-1 CD8⁺ T cells (FIG. 8B). Consistent with these findings, Ptpn2-deleted CD8⁺ T cells showed increased killing of target cells in an in vitro cytotoxicity assay compared with control CD8⁺ T cells (FIG. 8C). Furthermore, transcriptional profiling of OT-1 CD8⁺ T cells containing control or Ptpn2 sgRNAs from the 1:1 competitive assays revealed that Ptpn2-deleted CD8⁺ T cells were significantly enriched for gene signatures characteristic of activated effector cells (FIG. 8D). Transcriptional profiling of control or Ptpn2-deleted OT-1 CD8⁺ T cells at day 7 from the same tumor microenvironment revealed that Ptpn2-deleted CD8 T cells were significantly enriched for the TIL Tim-3⁺ signature, whereas control cells were enriched for the TIL Slamf6⁺ signature (FIG. 8M and Table 5) (Miller et al. (2019) Nat. Immunol.).

In addition, GSEA analysis revealed that Ptpn2-deleted cells were significantly enriched for mTORC1 signaling and several effector-related signatures that were also enriched in the Ptpn2-deleted cells in the LCMV model (FIG. 8N). Thus, consistent with the LCMV Clone 13 model, Ptpn2-deficient cells outcompete control cells, have elevated Granzyme B expression, and possess a Tim-3⁺ effector-skewed transcriptional profile.

Ptpn2 is ubiquitously expressed in the hematopoietic compartment and has roles in myeloid, T, and B cell development and function (Doody et al. (2009) Immunol. Rev. 230:38-50). Thus, therapeutic targeting of PTPN2 could potentially affect multiple immune subtypes. To model this, it was next investigated whether Ptpn2 deletion in all hematopoietic cells would attenuate MC38 tumor growth. Bone marrow chimeras were created using a control sgRNA or one of two Ptpn2-targeting sgRNAs (FIG. 8E). On average, ˜50% of cells in the hematopoeitic compartment carried the sgRNA, as measured by the percentage of Vex⁺ cells in reconstituted bone marrow chimeras (FIG. 6H). Deletion of Ptpn2 using two different sgRNAs led to complete MC38 tumor clearance in all Ptpn2-deleted chimeric mice, whereas there was progressive tumor growth in control chimeras (FIGS. 8F and 8G). Moreover, analysis of peripheral blood in these mice prior to tumor clearance revealed a significant decrease in the Slamf6⁺ Tim-3⁻ subpopulation and a significant increase in the Slamf6⁻ Tim-3⁺ subpopulation in the Ptpn2-deleted chimeras compared with control chimeras (FIG. 8O). These findings demonstrate that Ptpn2 deletion not only increases CD8⁺ T cell infiltration into MC38 tumors in a cell intrinsic fashion, but also results in complete clearance of MC38 tumors when deleted from the entire hematopoietic compartment.

### Example 12: Loss of Ptpn2 Increases CD8⁺ T Cell Cytotoxicity and Improves Checkpoint Blockade Responses

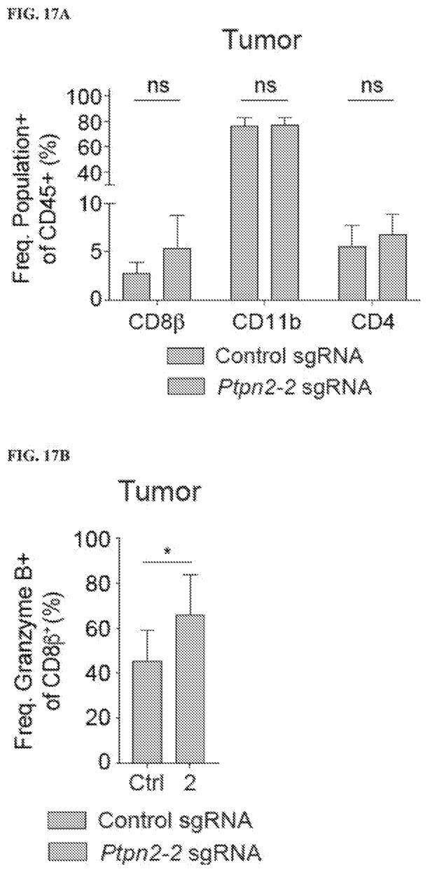

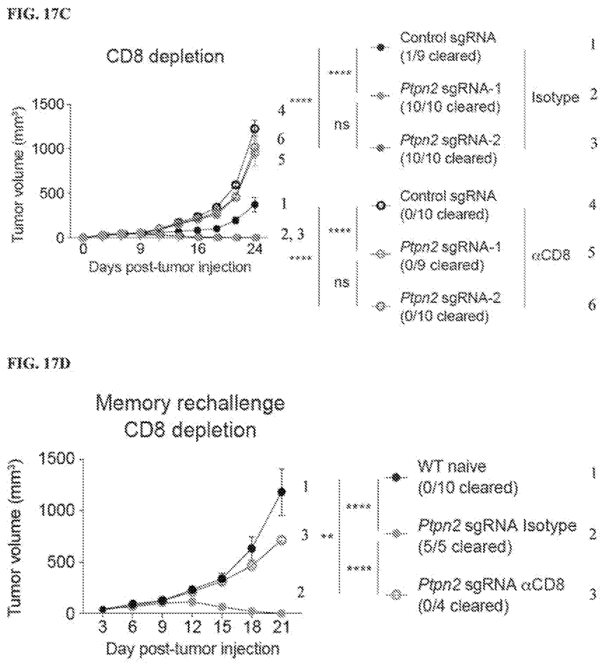

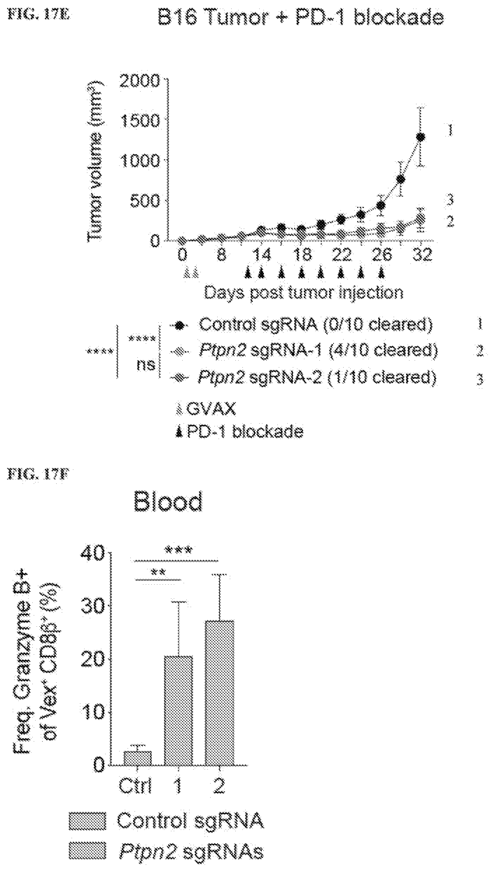

To determine the mechanism behind the potent of clearance of MC38 tumors by Ptpn2-deleted chimeras the immune infiltrate in MC38 tumors prior to clearance was examined. There were no differences in the frequencies (FIG. 17A) or absolute numbers of CD4⁺ T cell, CD8⁺ T cell, and myeloid cells in the MC38 tumors prior to clearance (FIG. 6M). However, the percentage of Granzyme B⁺ CD8⁺ T cells was increased in tumors of Ptpn2-deficient chimeras compared with control chimeras (FIG. 17B). Furthermore, peripheral blood CD8⁺ T cells of Ptpn2-deleted chimeras had significantly more Granzyme B+ cells, fewer CD127+ cells, and increased CD44⁺ CD62L⁻ effector cells (FIGS. 8H, 8I, 6I and 6J). This is consistent with Ptpn2-deficient CD8T cell responses in the RIP-mOVA model of diabetes (Wiede et al. (2014) J. Autoimmun. 53:105-114). Thus in both peripheral blood and tumors, CD8⁺ T cells were more activated when the entire hematopoietic system lacked Ptpn2. To prove that tumor clearance was due to CD8⁺ T cells in this model, CD8⁺ T cells were depleted in Ptpn2 sgRNA chimeric mice, which prevented clearance of MC38 tumors, indicating CD8⁺ T cells are required for clearance (FIG. 17C and FIG. 6N). Furthermore, Ptpn2 chimeric mice that completely eliminated primary tumors could clear a larger secondary challenge of MC38 tumor cells following a 60-day rest period post primary tumor clearance, in contrast to progressive tumor growth in naive WT mice (FIG. 8J), demonstrating that they developed functional memory. Depletion of CD8⁺ T cells also prevented clearance of secondary tumors, indicating CD8⁺ T cells are also required for secondary clearance (FIG. 17D).

It was next determined whether Ptpn2 deficiency in the immune system could improve PD-1 checkpoint blockade responses to a more immune-refractory model, B16 melanoma. Treatment of B16-challenged Ptpn2-deficient chimeras with PD-1 checkpoint blockade resulted in attenuated tumor growth compared with control chimeras (FIG. 17E). In addition, 25% of Ptpn2 chimeric mice completely cleared their tumors, in contrast to tumor growth in all control chimeric mice. This enhanced response to B16 melanoma was accompanied by an increase in Granzyme B⁺ CD8⁺ T cells in peripheral blood (FIG. 17F). These findings demonstrate that Ptpn2 deficiency in the immune system increases the cytotoxic CD8⁺ T cell response in the tumor and ultimately leads to a CD8⁺ T cell dependent clearance of MC38 tumors and improved PD-1 checkpoint blockade responses to B16 tumors.

The discovery of new regulators of immune cell function using functional genomics has been limited by the difficulty of genetically perturbing immune cells without extensive ex vivo manipulation (Godec et al. (2015) Proc. Natl. Acad. Sci. U.S.A. 112:512-517). As described herein, a system was engineered to solve this problem through delivery of gene-targeting sgRNAs to Cas9-expressing hematopoietic progenitor cells, such as with subsequent creation of gene-edited bone marrow chimeras. This system enables rapid deletion of candidate genes in both innate (macrophages and dendritic cell) and adaptive (B cells, CD4⁺, and CD8⁺ T cell) immune populations without perturbing their cell state. As a proof of concept, this system was used to discover the cell-intrinsic inhibitory effect of the phosphatase Ptpn2 on CD8⁺ T cell responses to LCMV Clone 13 viral infection and MC38 tumors. In addition, deletion of Ptpn2 in the entire hematopoietic compartment using the chimera system resulted in complete clearance of MC38 tumors accompanied by an enhanced peripheral cytotoxic effector CD8⁺ T cell response. These findings have important implications for in vivo screening of candidate immunologic targets, advance the understanding of Ptpn2's role in regulating CD8⁺ T cell responses to viral infection, and establish Ppn2 as a cancer immunotherapy target for activating the immune system.

First, it was demonstrated that the chimeric sgRNA delivery system described herein enables the rapid deletion of candidate genes in all major immune lineages in vivo. In vivo analysis of gene function in immune populations through ES cell-targeted generation of knockout mice is a lengthy process (Jaenisch et al. (1988) Science 240:1468-1474; Koller et al. (1992) Annu. Rev. Immunol. 10:705-730), while activation or cytokine stimulation of T cells to enable transduction results in altered effector T cell differentiation (Zhou et al. (2014) Nature 506:52-57; Godec et al. (2015) Proc. Natl. Acad. Sci. U.S.A. 112:512-517). The system described herein significantly improves on available approaches by allowing deletion of genes in immune cell lineages in eight weeks while maintaining normal immune development and function. Thus, it enables rapidly analyzing genes in immune cells in both physiological or disease contexts for the discovery of therapeutic targets. Further adaptation of this system is believed to enable high throughput screening using pooled genetic screens (Zhou et al. (2014) Nature 506:52-57; Milner et al. (2017) Nature 552:253-257; Manguso et al. (2017) Nature 547:413-418). In addition, the system described herein expands the classes of immune cell lineages that can be screened, allowing efficient editing of macrophages and dendritic cells in vivo, which are two cell populations that currently require the production of a knockout mouse for in vivo studies. Macrophages, in particular, have garnered attention as a source of cancer immunotherapy targets and the system described herein enables discovery of targets that alter macrophage function (Ruffell et al. (2015) Cancer Cell 27:462-472). Moreover, the system described herein expands the scope of phenotypes that can be evaluated by maintaining normal differentiation and homeostasis of the immune system. For example, the system described herein makes it possible to target genes important for the earliest stages of CD8⁺ T cell activation and differentiation in the lymph node, which has implications for developing new vaccine strategies.

Second, it has been demonstrated herein that Ptpn2 negatively regulates CD8⁺ T cell responses during LCMV Clone 13 viral infection. Deletion of Ptpn2 provides a competitive advantage to T cells responding to viral infection. In addition, Ptpn2-deleted T cells express more Granzyme B and less CD127, a signature of an increased effector profile (Wherry et al. (2007) Immunity 27:670-684). Recent work has established two subpopulations that occur in response to chronic viral infection, which are a terminally exhausted population which is more cytotoxic (Tim-3⁺ CXCR5⁻) and a stem-like exhausted population which is capable of self-renewal (Tim-3⁻ CXCR5⁺) (Im et al. (2016) Nature 537:417-421; He et al. (2016) Nature 537:412-428). The regulation of the differentiation and maintenance of these populations is currently unclear. It has been demonstrated herein that Ptpn2 deletion causes significant skewing toward the terminally exhausted (Tim-3⁺ Granzyme B⁺ population), and simultaneously decreases the stem-like population (CXCR5⁺ TCF7⁺). These findings indicate that Ptpn2 regulates the differentiation and/or maintenance of the stem-like and terminally exhausted subpopulations. Understanding how Ptpn2 deletion alters these two subpopulations is believed to provide insights into mechanisms controlling the generation, function, and plasticity of these exhausted CD8⁺ T cell subpopulations. Moreover, since Ptpn2 has a multitude of phosphorylation targets including the TCR, 1L-2, IL-7, and IFN signaling (Wiede et al. (2011) J. Clin. Invest. 121:4758-4774; Wherry et al. (2007) Immunity 27:670-684), Ptpn2 deletion is believed to help determine the relevant phosphorylation targets that control the differentiation and/or maintenance of the terminally exhausted and stem-like subpopulations.

The mechanisms that govern the generation and balance of the terminally exhausted and progenitor exhausted subpopulations in chronic infection and cancer remain unknown. Here it was demonstrated that deletion of Ptpn2 in CD8⁺ T cells enhances anti-tumor immunity by increasing the formation of the Tim-3⁺ subset. Intriguingly, it was found that at early and late time points that Ptpn2 deletion promotes a signature of effector cells in both the progenitor and terminally exhausted subsets. Deletion of Ptpn2 also increases phosphorylation of STAT1, which accelerates Tim-3⁺ cell differentiation at an early time point. Furthermore, deletion of Ptpn2 in the immune system leads to complete clearance of immunogenic MC38 tumors and improves PD-1 checkpoint blockade responses to less immunogenic B16 tumors. These findings have important implications for the understanding of the relative importance and regulation of the Tim-3⁺ and Slamf6⁺ subpopulations during immune responses, as well as for Ptpn2 as a cancer immunotherapy target.

The present work implicates Ptpn2 as a new regulator of the balance between the Tim-3⁺ and Slamf6⁺ subpopulations. Ptpn2 has a multitude of phosphorylation targets within the TCR, IL-2, IL-7, and IFN signaling cascades (Kleppe et al. (2010) Nat. Genet. 42:530-535; Wiede et al. (2011) J. Clin. Invest. 121:4758-4774). Here it was shown that Ptpn2 deletion increases phosphorylation of STAT1 after ex vivo stimulation of CD8⁺ T cells responding to LCMV Clone 13 viral infection, and results in increased IFN-α, which is required for the early competitive advantage seen for Ptpn2-deleted CD8⁺ T cells during LCMV Clone 13 viral infection. It was further demonstrated that the enhanced early differentiation of Slamf6⁺ Tim-3⁺ and Slamf6⁻ Tim-3⁺ cells observed in Ptpn2-deleted CD8⁺ T cells is also dependent on IFN-I signaling. These findings are consistent with IFN-I signaling attenuating the TCF1-Bcl6 axis during LCMV viral infection, resulting in an increase in the percentage of Tim-3⁺ cells (Wu et al. (2016) Sci. Immunol. 1:eaai8593) and highlight a crucial role for IFN-I signaling early in the differentiation of terminally exhausted cells. Overall, these findings help to further elucidate the molecular mechanisms controlling CD8⁺ T cell fate decisions into progenitor or terminally exhausted subpopulations in response to LCMV viral infection.

Currently, it is believed that an increase in the progenitor exhausted subpopulation promotes the efficacy of PD-1 blockade in chronic infection and cancer (Im et al. (2016) Nature 537:417-421; He et al. (2016) Nature 537:412-428; Sade-Feldman et al. (2018) Cell 175:998-1013; Miller et al. (2019) Nat. Immunol.). The data herein demonstrate that increasing the Tim-3⁺ subpopulation also can promote anti-tumor immunity. The Tim-3⁺ subpopulation is the primary cytotoxic population (Paley et al. (2012) Science 338:1220-1225), and thus also plays an important role in immune responses. It is likely that the relative number of progenitor exhausted cells is an absolute bottleneck on the long term potential of an effective immune response. An early skewing toward the terminally exhausted population would endow the immune response with greater cytotoxic capacity at the cost of longevity because the terminally exhausted cells eventually die and are not be able to be regenerated without a progenitor pool (Hashimoto et al. (2018) Annu. Rev. Med 69:301-318). The present work represents a new scenario where Ptpn2 deletion causes an early increase in the Tim-3⁺ subpopulation without changing the number of Slamf6⁺ CD8⁺ T cells. During LCMV Clone 13 infection, an early expansion of Tim-3⁺ cells followed by a sharp contraction down to baseline levels during the late stage of infection was shown. Furthermore, in combination with PD-1 blockade, deletion of Ptpn2 results in enhanced anti-tumor effects in the B16 melanoma model without affecting longevity. These data demonstrate that an early increase in the number of cytotoxic Tim-3⁺ cells in the tumor can enhance anti-tumor immunity. These findings indicate that both the progenitor and exhausted subpopulations can promote anti-tumor immunity, and their relative roles may change over time.

Finally, the results described herein support the development of Ptpn2 inhibitors for cancer immunotherapy and the deletion of PTPN2 in CAR T cell-based therapies. Ptpn2 is a key mediator of T cell tolerance and prevention of autoimmunity (Todd et al. (2007) Nat. Genet. 39:857-864); Wiede et al. (2011) J. Clin. Invest. 121:4758-4774; Wiede et al. (2014) J. Autoimmun. 53:105-114; Okuno et al. (2018) Diabet. Med. 35:376-380). The results described herein demonstrate that Pqpn2 has a cell-intrinsic role in CD8⁺ T cells in tumors, limiting their accumulation and expression of Granzyme B. Transcriptional profiling of Ptpn2-deleted and Pqpn2-expressing CD8⁺ T cells in tumors reveals that deletion of Ptpn2 in CD8⁺ T cells results in increased terminally exhausted and effector signatures, consistent with the increased expansion of CD8⁺ T cells during chronic LCMV infection and Ptpn2 deficient CD8⁺ T cell responses in the RIP-mOVA model of diabetes (Wiede et al. (2014) J. Autoimmun. 53:105-114). Furthermore, deletion of Pqpn2 in the whole hematopoietic compartment leads to clearance of MC38 tumors, accompanied by a significantly elevated systemic cytotoxic CD8⁺ T cell response, which could be beneficial for enhancing immunity to disseminated metastatic disease (Marabelle et al. (2013) J. Clin. Invest. 123:4980; Zamarin et al. (2014) Sci. Transl. Med 6:226ra32). These data resemble the kinetics and penetrance of MC38 tumor clearance in Pdcd1 germline knockout mice (Woo et al. (2012) Cancer Res. 72:917-927). Ptpn2-deleted mice that cleared MC38 primary tumors were also able to clear a higher dose rechallenge, indicating that Ptpn2 deletion in CD8⁺ T cells does not impair CD8⁺ T cell memory formation. Thus, the results described herein establish that Ptpn2 deletion improves anti-tumor immunity by acting on CD8⁺ T cells and with possible contributions of responses of other immune cell types. Ptpn2 deletion in the immune system also improves PD-1 checkpoint blockade responses to B16 tumors indicating its potential use as a combination therapy with PD-1 blockade. Ptpn2 is a particularly attractive cancer immunotherapy target given its established tumor-intrinsic role in restraining anti-tumor immunity (Manguso et al. (2017) Nature 547:413-418). Inhibition of Ptpn2 in a tumor-bearing host would enhance anti-tumor immunity in two ways. Ptpn2 inhibition would enhance IFNγ signaling within tumor cells thereby increasing MHC-I expression (Manguso et al. (2017) Nature 547:413-418), which would promote TCR driven differentiation of exhausted cells into the Tim-3 population (Miller et al. (2019) Nat. Immunol.). Ptpn2 deletion in CD8T cells would increase IFN-I signaling and enhance formation of the cytotoxic Tim-3⁺ population. Thus, inhibition of Ptpn2 in a tumor-bearing host negatively affects tumor cells by enhancing interferon signaling and positively affects immune responses to the tumor.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments encompassed by the present invention described herein. Such equivalents are intended to be encompassed by the following claims.

What is claimed:

1. A method of treating a subject having a condition that would benefit from an increased immune response, comprising administering to the subject a therapeutically effective amount of an agent that decreases the copy number, the expression level, and/or the activity of tyrosine-protein phosphatase non-receptor type 2 (Ptpn2) or a fragment thereof.

2. The method of claim 1, wherein the agent selectively decreases the phosphatase activity and/or the substrate binding activity of Ptpn2.

3. The method of claim 1 or 2, wherein the agent is a small molecule inhibitor, CRISPR single-guide RNA (sgRNA), RNA interfering agent, antisense oligonucleotide, peptide or peptidomimetic inhibitor, aptamer, or intrabody.

4. The method of claim 3, wherein the RNA interfering agent is a small interfering RNA (siRNA), CRISPR RNA (crRNA), a small hairpin RNA (shRNA), a microRNA (miRNA), or a piwi-interacting RNA (piRNA).

5. The method of claim 3, wherein the agent is a CRISPR single-guide RNA (sgRNA).

6. The method of claim 5, wherein the sgRNA comprises a nucleic acid sequence selected from the group consisting of nucleic acid sequence listed in Table 2.

7. The method of claim 3, wherein the agent comprises an intrabody, or an antigen binding fragment thereof, which specifically binds to Ptpn2 and/or a substrate of Ptpn2.

8. The method of claim 7, wherein the intrabody, or antigen binding fragment thereof, is murine, chimeric, humanized, composite, or human.

9. The method of claim 7 or 8, wherein the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabodies fragments.

10. The method of any one of claims 1-9, wherein the agent decreases the copy number, the expression level, and/or the activity of Ptpn2 or a fragment thereof in hematopoietic stem cells (HSCs) and/or cells derived therefrom.

11. The method of any one of claims 1-10, wherein the agent targets HSCs and/or cells derived therefrom, optionally wherein the cells are chimeric antigen receptor (CAR)-T cells.

12. The method of claim 1 or 2, wherein the agent is cell-based.

13. The method of claim 12, wherein the agent comprises engineered HSCs and/or cells derived therefrom which have a decreased copy number, expression level, and/or activity of Ptpn2 or a fragment thereof.

14. The method of claim 13, wherein the engineered HSCs and/or cells derived therefrom are administered focally or systemically.

15. The method of claim 13 or 14, wherein the systemic administration is intravenous, intramuscular, intraperitoneal, or intra-articular.

16. The method of any one of claims 13-15, wherein the engineered HSCs and/or cells derived therefrom administered to the subject are autologous, syngeneic, allogeneic, or xenogeneic to the subject.

17. The method of any one of claims 13-16, wherein the engineered HSCs and/or cells derived therefrom maintain at least 5% decreased copy number, expression level, and/or activity of Ptpn2 or a fragment thereof after administration to the subject.

18. The method of any one of claims 10-17, wherein HSCs and/or cells derived therefrom give rise to T cells which maintain a decreased copy number, expression level, and/or activity of Ptpn2 or a fragment thereof, optionally wherein the T cells are CD4⁺ T cells, CD8+ T cells, and/or CAR-T cells.

19. The method of any one of claims 10-18, wherein the HSCs and/or cells derived therefrom are CD4⁺ T cells, CD8+ T cells, and/or CAR-T cells.

20. The method of any one of claims 1-19, wherein the agent increases CD4⁺ T cell responses and/or CD8+ T cell responses.

21. The method of any one of claims 1-20, wherein the agent increases expression of genes specific to CD4⁺ T cells and/or CD8+ T cells.

22. The method of any one of claims 1-21, wherein the condition is a cancer.

23. The method of claim 22, wherein the cancer is selected from the group consisting of a solid tumor, a hematologic cancer, bladder cancer, brain cancer, breast cancer, colon cancer, gastric cancer, glioma, head cancer, leukemia, liver cancer, lung cancer, lymphoma, myeloma, neck cancer, ovarian cancer, melanoma, pancreatic cancer, renal cancer, salivary cancer, stomach cancer, thymic epithelial cancer, and thyroid cancer.

24. The method of any one of claims 1-23, wherein the agent reduces the number of proliferating cells in the cancer and/or reduces the volume or size of a tumor comprising the cancer cells.

25. The method of any one of claims 1-24, wherein the agent increases the number of CD4⁺ T cells and/or CD8+ T cells in a tumor comprising the cancer cells.

26. The method of any one of claims 1-25, wherein the agent increases activity of CD4⁺ T cells and/or CD8+ T cells.

27. The method of any one of claims 1-26, wherein the agent increases the percentage of CD4⁺ T cells and/or CD8+ T cells in tumor, spleen, draining lymph node, and/or blood, optionally wherein the CD8+ T cells are Granzye B+.

28. The method of any one of claims 1-27, wherein the agent leads to an increase in CD25 and a decrease in CD127 expression in CD8+ T cells in the tumor-draining lymph node.

29. The method of any one of claims 1-28, wherein the agent increases TIL Tim3+ signature, mTORC1 signaling, and/or effector-related signatures in CD8+ T cells in the tumor.

30. The method of any one of claims 1-29, wherein the agent increases the percentage of CD4⁺ T cells, Slamf6−Tim3+ CD8+ T cells, Granzyme B+ CD8+ T cells, and/or CD44+CD62L− CD8+ T cells in blood.

31. The method of any one of claims 1-30, wherein the agent decreases the percentage of CD4⁺ T cells, Slamf6+Tim3− CD8+ T cells, and/or CD127+ CD8+ T cells in blood.

32. The method of any one of claims 1-31, further comprising administering to the subject at least one additional cancer therapy or regimen, optionally wherein the at least one additional cancer therapy or regimen is administered before, after, or concurrently with the agent and/or the immunotherapy.

33. The method of claim 32, wherein the cancer therapy is not an immunotherapy.

34. The method of any one of claims 1-21, wherein the condition is an infection.

35. The method of claim 34, wherein the infection is a viral infection, bacterial infection, protozoan infection, or helminth infection.

36. The method of claim 34 or 35, wherein the viral infection is a chronic viral infection.

37. The method of any one of claims 34-36, wherein the viral infection is LCMV Clone 13 viral infection.

38. The method of any one of claims 1-21 and 34-37, wherein the agent increases the number of CD4⁺ T cells and/or CD8+ T cells in spleen, lung, and/or liver.

39. The method of any one of claims 1-21 and 34-38, wherein the agent increases CD4⁺ T cells and/or CD8+ T cells, optionally wherein the CD8+ T cells are Granzyme B+.

40. The method of any one of claims 1-21 and 34-39, wherein the agent increases the ratio of Tim-3+ to Slamf6+ cells.

41. The method of any one of claims 1-21 and 34-40, wherein the agent increases the percentage of Tim-3+ cells and/or decreases the percentage of CXCR5+ cells.

42. The method of any one of claims 1-21 and 34-41, wherein the agent increases the number of Tim-3+ cells.

43. The method of any one of claims 1-21 and 34-42, wherein the agent decreases CD127 expression and/or TCF7 expression in CD8+ T cells.

44. The method of any one of claims 1-21 and 34-43, wherein the agent promotes the formation of terminally exhausted CD4⁺ T cells and/or terminally exhausted CD8+ T cells.

45. The method of claim 44, wherein the terminally exhausted T cells express Gzma, Cd7, Cd244, and/or Cd160.

46. The method of claim 44, wherein the terminally exhausted T cells comprise Tim3+CXCR5−, Tim3+Slamf6−, and/or Tim3+Granzyme B+ T cells.

47. The method of any one of claims 1-21 and 34-46, wherein the agent decreases the formation of stem-like exhausted CD4⁺ T cells and/or stem-like exhausted CD8+ T cells.

48. The method of claim 47, wherein the stem-like exhausted T cells comprise Tim3− CXCR5+, Tim3−Slamf6+, and/or CXCR5+ TCF7+ T cells.

49. The method of any one of claims 1-21 and 34-48, wherein the agent decreases the formation of progenitor exhausted CD8+ T cells.

50. The method of claim 49, wherein the progenitor exhausted CD8+ T cells express Slamf6, Id3, and/or Tcf7.

51. The method of any one of claims 1-21 and 34-50, wherein the agent increases expression of Gzma, Cd160, Stat1, Cd7, Ccl4, and Ccl5 in the terminally exhausted CD8+ T cells.

52. The method of any one of claims 1-21 and 34-51, wherein the agent increases expression of Gzma, Gzmk, Cd160, Stat1, Cd7, Ccl4, Ccl5, Pdcd1, Lag3, and/or Id2 in the progenitor exhausted CD8+ T cells.

53. The method of any one of claims 1-21 and 34-52, wherein the agent increases expression of effector-related genes or gene signatures in the terminally exhausted CD8+ T cells and/or the progenior exhausted CD8+ T cells.

54. The method of any one of claims 1-21 and 34-53, wherein the effector-related gene signature is selected from the group consisting of mTORC1 signaling and effector versus memory profiles.

55. The method of any one of claims 1-21 and 34-54, wherein the agent increases expression of the effector-related genes both early and late post LCMV infection.

56. The method of any one of claims 1-21 and 34-55, wherein the agent increases Tim3+ CD8+ T cell differentiation.

57. The method of any one of claims 1-21 and 34-56, wherein the agent increases Tim3+ CD8+ T cell differentiation through enhanced IFN-α signaling.

58. A method for monitoring the progression of a condition that would benefit from an increased immune response in a subject, wherein the subject is administered a therapeutically effective amount of an agent that inhibits the copy number, amount, and/or activity of Ptpn2, the method comprising:
a) detecting in a subject sample at a first point in time the copy number, amount, and/or activity of Ptpn2 in HSCs and/or cells derived therefrom;
b) repeating step a) at a subsequent point in time; and
c) comparing the amount or activity of Ptpn2 detected in steps a) and b) to monitor the progression of the cancer in the subject.

59. A method of assessing the efficacy of an agent that inhibits the copy number, amount, and/or activity of Ptpn2 for treating a condition that would benefit from an increased immune response in a subject, comprising:
a) detecting in a subject sample at a first point in time the copy number, amount, and/or or activity of Ptpn2 in HSCs and/or cells derived therefrom;
b) repeating step a) during at least one subsequent point in time after administration of the agent; and
c) comparing the copy number, amount, and/or activity detected in steps a) and b), wherein the absence of, or a significant decrease in, the copy number, amount, and/or activity of, Ptpn2, in the subsequent sample as compared to the copy number, amount, and/or activity in the sample at the first point in time, indicates that the agent treats the condition in the subject.

60. The method of claim 58 or 59, wherein the first and/or at least one subsequent sample is selected from the group consisting of ex vivo and in vivo samples.

61. The method of any one of claims 58-60, wherein the first and/or at least one subsequent sample is a portion of a single sample or pooled samples obtained from the subject.

62. The method of any one of claims 58-61, wherein the condition is a cancer or infection.

63. The method of claim 62, wherein the cancer is selected from the group consisting of a solid tumor, a hematologic cancer, bladder cancer, brain cancer, breast cancer, colon cancer, gastric cancer, glioma, head cancer, leukemia, liver cancer, lung cancer, lymphoma, myeloma, neck cancer, ovarian cancer, melanoma, pancreatic cancer, renal cancer, salivary cancer, stomach cancer, thymic epithelial cancer, and thyroid cancer.

64. The method of any one of claims 58-63, wherein between the first point in time and the subsequent point in time, the subject has undergone treatment, completed treatment, and/or is in remission for the cancer.

65. The method of claim 64, wherein the cancer treatment is selected from the group consisting of immunotherapy, targeted therapy, chemotherapy, radiation therapy, hormonal therapy, an anti-cancer vaccine, an anti-cancer virus, and a checkpoint inhibitor.

66. The method of any one of claims 58-65, wherein the sample comprises cells, serum, peritumoral tissue, and/or intratumoral tissue obtained from the subject.

67. The method of any one of claims 1-66, wherein the agent is administered in a pharmaceutically acceptable formulation.

68. The method of any one of claims 1-67, wherein Ptpn2 comprises a nucleic acid sequence having at least 95% identity to a nucleic acid sequence listed in Table 1 and/or encodes an amino acid sequence having at least 95% identity to an amino acid sequence listed in Table 1.

69. The method of any one of claims 1-68, wherein Ptpn2 is human, mouse, chimeric, or a fusion.

70. The method of any one of claims 1-69, wherein the subject is an animal model of the condition that would benefit from an increased immune response.

71. The method of claim 70, wherein the animal model is a mouse model.

72. The method of any one of claims 1-71, wherein the subject is a mammal.

73. The method of claim 72, wherein the mammal is a mouse or a human.

74. The method of claim 73, wherein the mammal is a human.

75. A method of generating transduced hematopoietic stem cells (HSCs) and/or cells derived therefrom that are differentiated in vivo, comprising transducing the cells with at least one viral vector, wherein each viral vector integrates an exogenous nucleic acid into the genome of the cell.

76. The method of claim 75, further comprising obtaining HSCs and/or cells derived therefrom prior to transducing the cells.

77. The method of claim 75 or 76, further comprising transplanting the transduced cells to an immunocompromised incubator animal, wherein the transplanted transduced cells reconstitute the immunocompromised incubator animal immune system.

78. The method of any one of claims 75-77, further comprising selecting populations of reconstituted immune cells of interest from the incubator animal.

79. The method of any one of claims 75-78, wherein the cells are transduced with a single viral vector.

80. The method of any one of claims 75-79, wherein the viral vector is a lentiviral vector.

81. The method of any one of claims 75-80, wherein the viral vector inducibly expresses an RNA encoded by the exogenous nucleic acid.

82. The method of any one of claims 75-81, wherein the inducible expression is regulated using lactose operon operator (LacO) and lactose operon repressor (LacI) sequences.

83. The method of any one of claims 75-82, wherein the exogenous nucleic acid is selected from the group consisting of mRNA, antisense RNA, shRNA, siRNA, microRNA, PiwiRNA, and combinations thereof.

84. The method of claim 83, wherein the exogenous nucleic acid is an shRNA.

85. The method of claim 83, wherein the exogenous nucleic acid comprises a) an engineered, non-naturally occurring Clustered Regularly Interspaced Short Palindromic Repeats (CRISPR) guide RNA that hybridizes with a target nucleic acid sequence of interest and/or b) a nucleotide sequence encoding a Type-II Cas9 protein, optionally wherein the cells are transgenic for Cas9.

86. The method of any one of claims 75-85, wherein the viral vector further comprises a nucleic acid encoding a reporter.

87. The method of claim 75-86, wherein the reporter is a fluorescent protein.

88. The method of any one of claims 75-87, wherein the incubator animal is immunocompromised using lethal irradiation or chemotherapy.

89. The method of any one of claims 75-88, wherein the incubator animal is immunodeficient.

90. The method of any one of claims 75-89, wherein the immunocompromised incubator animal and the animal from which the HSCs and/or cells derived therefrom were obtained are congenic.

91. The method of any one of claims 75-90, wherein transplantation of the transduced cells to the immunocompromised incubator animal is autologous, syngeneic, allogeneic, or xenogeneic.

92. The method of any one of claims 75-91, wherein the reconstituted immune cells of interest are selected from the group consisting of terminally differentiated cells, post-mitotic cells, and/or unactivated cells.

93. The method of claim 92, wherein the reconstituted immune cells of interest have not been exogenously stimulated to divide.

94. The method of claim 91 or 92, wherein the reconstituted immune cells of interest are T cells, dendritic cells, macrophages, or B cells.

95. The method of any one of claims 75-94, wherein the reconstituted immune cells of interest are isolated.

96. The method of any one of claims 75-95, further comprising culturing the selected cells in vitro and monitoring the selected cells in response to exogenous perturbation.

97. The method of any one of claims 75-95, further comprising transplanting the transduced HSCs and/or cells derived therefrom into an experimental animal for differentiation in vivo.

98. The method of claim 97, further comprising monitoring the transplanted cells in response to exogenous perturbation.

99. The method of any one of claims 96-98, wherein the exogenous perturbation is the application of an assay for testing autoimmune, allergic, vaccination, immunotolerance, cancer immunotherapy, immune exhaustion, immunological memory, immunological epitope, stem cell, hematopoietic stem cell, viral infection, or immune disease responses.

100. Transduced HSCs and/or cells derived therefrom that are differentiated in vivo produced according to any one of methods 75-99.

101. Non-human animals comprising transduced HSCs and/or cells derived therefrom that are produced according to any one of methods 75-99.

102. The method, transduced cells, or non-human animals of any one of claims 1-101, wherein the HSCs and/or cells derived therefrom are murine or human.

103. The method, transduced cells, or non-human animals of any one of claims 1-102, wherein the HSCs and/or cells derived therefrom are selected from the group consisting of hematopoietic stem cells (HSC), common myeloid progenitor cells (CMP), common lymphoid progenitor cells (CLP), committed lymphoid progenitor cells, granulocyte/macrophage progenitor cells (GMP), megakaryocyte/erythroid progenitor cells (MEP), granulocyte progenitor cells, macrophage progenitor cells, erythroid progenitor cells, megakaryocyte progenitor cells (MKP), NK cell progenitor cells (NKP), B cell progenitor cells (BCP), and T cell progenitor cells (TCP).

104. The method, transduced cells, or non-human animals of any one of claims 1-103, wherein the HSCs and/or cells derived therefrom comprise or are T cells.

105. The method, transduced cells, or non-human animals of claim 104, wherein the T cells are CD4⁺ T cells and/or CD8+ T cells.

106. The method, transduced cells, or non-human animals of claim 104 or 105, wherein the T cells are CAR-T cells.

107. The method, transduced cells, or non-human animals of any one of claims 1-106, wherein the HSCs and/or cells derived therefrom are not terminally differentiated or post-mitotic.

108. The method, transduced cells, or non-human animals of any one of claims 1-107, wherein the HSCs and/or cells derived therefrom are not thymocytes or are not derived from the thymus.

109. The method, transduced cells, or non-human animals of any one of claims 1-108, wherein the HSCs and/or cells derived are obtained from a biological source selected from the group consisting of bone marrow, umbilical cord blood, amniotic fluid, peripheral blood, and fetal liver.


SEQUENCE LISTING (50 sequences)

SEQ ID NO: 1 - DNA, 3418 bp, Homo sapiens
gctcgggcgccgagtctgcgcgctgacgtccgacgctccaggtactttccccacggccgacagggcttggcgtgggggcggggcgcggcgcgcagcgcgcatgcgccgcagcgccagcgctctccccggatcgtgcggggcctgagcctctccgccggcgcaggctctgctcgcgccagctcgctcccgcagccatgcccaccaccatcgagcgggagttcgaagagttggatactcagcgtcgctggcagccgctgtacttggaaattcgaaatgagtcccatgactatcctcatagagtggccaagtttccagaaaacagaaatcgaaacagatacagagatgtaagcccatatgatcacagtcgtgttaaactgcaaaatgctgagaatgattatattaatgccagtttagttgacatagaagaggcacaaaggagttacatcttaacacagggtccacttcctaacacatgctgccatttctggcttatggtttggcagcagaagaccaaagcagttgtcatgctgaaccgcattgtggagaaagaatcggttaaatgtgcacagtactggccaacagatgaccaagagatgctgtttaaagaaacaggattcagtgtgaagctcttgtcagaagatgtgaagtcgtattatacagtacatctactacaattagaaaatatcaatagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttgatggaaaaaggagatgatattaacataaaacaagtgttactgaacatgagaaaataccgaatgggtcttattcagaccccagatcaactgagattctcatacatggctataatagaaggagcaaaatgtataaagggagattctagtatacagaaacgatggaaagaactttctaaggaagacttatctcctgcctttgatcattcaccaaacaaaataatgactgaaaaatacaatgggaacagaataggtctagaagaagaaaaactgacaggtgaccgatgtacaggactttcctctaaaatgcaagatacaatggaggagaacagtgagagtgctctacggaaacgtattcgagaggacagaaaggccaccacagctcagaaggtgcagcagatgaaacagaggctaaatgagaatgaacgaaaaagaaaaaggtggttatattggcaacctattctcactaagatggggtttatgtcagtcattttggttggcgcttttgttggctggacactgttttttcagcaaaatgccctataaacaattaattttgcccagcaagcttctgcactagtaactgacagtgctacattaatcataggggtttgtctgcagcaaacgcctcatatcccaaaaacggtgcagtagaatagacatcaaccagataagtgatatttacagtcacaagcccaacatctcaggactcttgactgcaggttcctctgaaccccaaactgtaaatggctgtctaaaataaagacattcatgtttgttaaaaactggtaaattttgcaactgtattcatacatgtcaaacacagtatttcacctgaccaacattgagatatcctttatcacaggatttgtttttggaggctatctggattttaacctgcacttgatataagcaataaatattgtggttttatctacgttattggaaagaaaatgacatttaaataatgtgtgtaatgtataatgtactattgacatgggcatcaacacttttattcttaagcatttcagggtaaatatattttataagtatctatttaatcttttgtagttaactgtactttttaagagctcaatttgaaaaatctgttactaaaaaaataaattgtatgtcgattgaattgtactggatacattttccatttttctaaagagaagtttgatatgagcagttagaagttggaataagcaatttctactatatattgcatttcttttatgttttacagttttccccattttaaaaagaaaagcaaacaaagaaacaaaagtttttcctaaaaatatctttgaaggaaaattctccttactgggatagtcaggtaaacagttggtcaagactttgtaaagaaattggtttctgtaaatcccattattgatatgtttatttttcatgaaaatttcaatgtagttggggtagattatgatttaggaagcaaaagtaagaagcagcattttatgattcataatttcagtttactagactgaagttttgaagtaaacacttttcagtttctttctacttcaataaatagtatgattatatgcaaaccttacattgtcattttaacttaatgaatattttttaaagcaaactgtttaatgaatttaactgctcatttgaatgctagctttcctcagatttcaacattccattcagtgtttaatttgtcttacttaaacttgaaattgttgttacaaatttaattgctaggaggcatggatagcatacattattatggatagcataccttatttcagtggttttcaaactatgctcattggatgtccaggtgggtcaagaggttactttcaaccacagcatctctgccttgtctctttatatgccacataagatttctgcataaggcttaagtattttaaagggggcagttatcatttaaaaacagtttggtcgggcgcggtggctcatgcctgtaatcccagcactttgggaggctgaagtgggcagatcacctgaggtcaggagttcaagaccagcctggccaacgtggtgaaacaccatctctactaaaaatgcaaaaattagctgggcatggtggagggcacctgtaatctcagctactcaggaggctgaggtaggagaattgcttgaacccaggagatggaggttgcagtgagctgagatcacgtcactgcactccagccagggcgacagagcgagactccatctcaaaagaaacaaacaaaaaaaacagtttgggccgggtgtggtggctcacgcttgtaatcccagcacttcggaaggccaaggcgggcggatcacgaggtcaagagatggagactgtcctggccaacatggtgaaatcccttctttactaaaaatacaaaaattatctgggcgtggtggtgcatgcctgtagtcccagctccttgggaggctaaggcaggagaatcacttgaacccgggaggcagaggttgcagtgagccgagattgcaccactgcactccagcctggcaacagagcaagacttcgtctc

SEQ ID NO: 2 - protein, 415 aa, Homo sapiens
MPTTIEREFEELDTQRRWQPLYLEIRNESHDYPHRVAKFPENRNRNRYRDVSPYDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYNGNRIGLEEEKLTGDRCTGLSSKMQDTMEENSESALRKRIREDRKATTAQKVQQMKQRLNENERKRKRWLYWQPILTKMGFMSVILVGAFVGWTLFFQQNAL

SEQ ID NO: 3 - DNA, 1721 bp, Homo sapiens
gctcgggcgccgagtctgcgcgctgacgtccgacgctccaggtactttccccacggccgacagggcttggcgtgggggcggggcgcggcgcgcagcgcgcatgcgccgcagcgccagcgctctccccggatcgtgcggggcctgagcctctccgccggcgcaggctctgctcgcgccagctcgctcccgcagccatgcccaccaccatcgagcgggagttcgaagagttggatactcagcgtcgctggcagccgctgtacttggaaattcgaaatgagtcccatgactatcctcatagagtggccaagtttccagaaaacagaaatcgaaacagatacagagatgtaagcccatatgatcacagtcgtgttaaactgcaaaatgctgagaatgattatattaatgccagtttagttgacatagaagaggcacaaaggagttacatcttaacacagggtccacttcctaacacatgctgccatttctggcttatggtttggcagcagaagaccaaagcagttgtcatgctgaaccgcattgtggagaaagaatcggttaaatgtgcacagtactggccaacagatgaccaagagatgctgtttaaagaaacaggattcagtgtgaagctcttgtcagaagatgtgaagtcgtattatacagtacatctactacaattagaaaatatcaatagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttgatggaaaaaggagatgatattaacataaaacaagtgttactgaacatgagaaaataccgaatgggtcttattcagaccccagatcaactgagattctcatacatggctataatagaaggagcaaaatgtataaagggagattctagtatacagaaacgatggaaagaactttctaaggaagacttatctcctgcctttgatcattcaccaaacaaaataatgactgaaaaatacaatgggaacagaataggtctagaagaagaaaaactgacaggtgaccgatgtacaggactttcctctaaaatgcaagatacaatggaggagaacagtgagagtgctctacggaaacgtattcgagaggacagaaaggccaccacagctcagaaggtgcagcagatgaaacagaggctaaatgagaatgaacgaaaaagaaaaaggccaagattgacagacacctaatattcatgacttgagaatattctgcagctataaattttgaaccattgatgtgcaaagcaagacctgaagcccactccggaaactaaagtgaggctcgctaaccctctagattgcctcacagttgtttgtttacaaagtaaactttacatccaggggatgaagagcacccaccagcagaagactttgcagaacctttaattggatgtgttaagtgtttttaatgagtgtatgaaatgtagaaagatgtacaagaaataaattaggggagattactttgtattgtactgccattcctactgtatttttatactttttggcagcattaaatatttttgttaaatagtcaaaaaaaaaaaaaaaaaa

SEQ ID NO: 4 - protein, 387 aa, Homo sapiens
MPTTIEREFEELDTQRRWQPLYLEIRNESHDYPHRVAKFPENRNRNRYRDVSPYDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYNGNRIGLEEEKLTGDRCTGLSSKMQDTMEENSESALRKRIREDRKATTAQKVQQMKQRLNENERKRKRPRLTDT

SEQ ID NO: 5 - DNA, 1619 bp, Homo sapiens
gctcgggcgccgagtctgcgcgctgacgtccgacgctccaggtactttccccacggccgacagggcttggcgtgggggcggggcgcggcgcgcagcgcgcatgcgccgcagcgccagcgctctccccggatcgtgcggggcctgagcctctccgccggcgcaggctctgctcgcgccagctcgctcccgcagccatgcccaccaccatcgagcgggagttcgaagagttggatactcagcgtcgctggcagccgctgtacttggaaattcgaaatgagtcccatgactatcctcatagagtggccaagtttccagaaaacagaaatcgaaacagatacagagatgtaagcccatatgatcacagtcgtgttaaactgcaaaatgctgagaatgattatattaatgccagtttagttgacatagaagaggcacaaaggagttacatcttaacacagggtccacttcctaacacatgctgccatttctggcttatggtttggcagcagaagaccaaagcagttgtcatgctgaaccgcattgtggagaaagaatcggttaaatgtgcacagtactggccaacagatgaccaagagatgctgtttaaagaaacaggattcagtgtgaagctcttgtcagaagatgtgaagtcgtattatacagtacatctactacaattagaaaatatcaatagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttgatggaaaaaggagatgatattaacataaaacaagtgttactgaacatgagaaaataccgaatgggtcttattcagaccccagatcaactgagattctcatacatggctataatagaaggagcaaaatgtataaagggagattctagtatacagaaacgatggaaagaactttctaaggaagacttatctcctgcctttgatcattcaccaaacaaaataatgactgaaaaatacaatgggaacagaataggtctagaagaagaaaaactgacaggtgaccgatgtacaggactttcctctaaaatgcaagatacaatggaggagaacagtgagaggccaagattgacagacacctaatattcatgacttgagaatattctgcagctataaattttgaaccattgatgtgcaaagcaagacctgaagcccactccggaaactaaagtgaggctcgctaaccctctagattgcctcacagttgtttgtttacaaagtaaactttacatccaggggatgaagagcacccaccagcagaagactttgcagaacctttaattggatgtgttaagtgtttttaatgagtgtatgaaatgtagaaagatgtacaagaaataaattaggggagattactttgtattgtactgccattcctactgtatttttatactttttggcagcattaaatatttttgttaaatagtcaaaaaaaaaaaaaaaaaa

SEQ ID NO: 6 - protein, 353 aa, Homo sapiens
MPTTIEREFEELDTQRRWQPLYLEIRNESHDYPHRVAKFPENRNRNRYRDVSPYDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYNGNRIGLEEEKLTGDRCTGLSSKMQDTMEENSERPRLTDT

SEQ ID NO: 7 - DNA, 1790 bp, Homo sapiens
gctcgggcgccgagtctgcgcgctgacgtccgacgctccaggtactttccccacggccgacagggcttggcgtgggggcggggcgcggcgcgcagcgcgcatgcgccgcagcgccagcgctctccccggatcgtgcggggcctgagcctctccgccggcgcaggctctgctcgcgccagctcgctcccgcagccatgcccaccaccatcgagcgggagttcgaagagttggatactcagcgtcgctggcagccgctgtacttggaaattcgaaatgagtcccatgactatcctcatagagtggccaagtttccagaaaacagaaatcgaaacagatacagagatgtaagcccatatgatcacagtcgtgttaaactgcaaaatgctgagaatgattatattaatgccagtttagttgacatagaagaggcacaaaggagttacatcttaacacagggtccacttcctaacacatgctgccatttctggcttatggtttggcagcagaagaccaaagcagttgtcatgctgaaccgcattgtggagaaagaatcggttaaatgtgcacagtactggccaacagatgaccaagagatgctgtttaaagaaacaggattcagtgtgaagctcttgtcagaagatgtgaagtcgtattatacagtacatctactacaattagaaaatatcaattatattgagaacttgtggatcacactgtatttgaaattattaatgctggatgttaaaaggtcactaaaaagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttgatggaaaaaggagatgatattaacataaaacaagtgttactgaacatgagaaaataccgaatgggtcttattcagaccccagatcaactgagattctcatacatggctataatagaaggagcaaaatgtataaagggagattctagtatacagaaacgatggaaagaactttctaaggaagacttatctcctgcctttgatcattcaccaaacaaaataatgactgaaaaatacaatgggaacagaataggtctagaagaagaaaaactgacaggtgaccgatgtacaggactttcctctaaaatgcaagatacaatggaggagaacagtgagagtgctctacggaaacgtattcgagaggacagaaaggccaccacagctcagaaggtgcagcagatgaaacagaggctaaatgagaatgaacgaaaaagaaaaaggccaagattgacagacacctaatattcatgacttgagaatattctgcagctataaattttgaaccattgatgtgcaaagcaagacctgaagcccactccggaaactaaagtgaggctcgctaaccctctagattgcctcacagttgtttgtttacaaagtaaactttacatccaggggatgaagagcacccaccagcagaagactttgcagaacctttaattggatgtgttaagtgtttttaatgagtgtatgaaatgtagaaagatgtacaagaaataaattaggggagattactttgtattgtactgccattcctactgtatttttatactttttggcagcattaaatatttttgttaaatagtcaaaaaaaaaaaaaaaaaa

SEQ ID NO: 8 - protein, 410 aa, Homo sapiens
MPTTIEREFEELDTQRRWQPLYLEIRNESHDYPHRVAKFPENRNRNRYRDVSPYDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINYIENLWITLYLKLLMLDVKRSLKSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYNGNRIGLEEEKLTGDRCTGLSSKMQDTMEENSESALRKRIREDRKATTAQKVQQMKQRLNENERKRKRPRLTDT

SEQ ID NO: 9 - DNA, 3313 bp, Homo sapiens
tattcaatgcagggaacagaccagttcatcatggaggcattccatcagagcgtctagttagagaagatatgtcatggactgcatcggcacagaagtggggtttatgtgagagaggagttggaagtcacacctgagtggagagcaacgtgaaaaggtgatgtcagcaagaatttaggatgtatggaaaggatggtaaaggcaccaactggatggatcagggagacatggaatgcagaatgcaggaaatagatgatcacagtcgtgttaaactgcaaaatgctgagaatgattatattaatgccagtttagttgacatagaagaggcacaaaggagttacatcttaacacagggtccacttcctaacacatgctgccatttctggcttatggtttggcagcagaagaccaaagcagttgtcatgctgaaccgcattgtggagaaagaatcggttaaatgtgcacagtactggccaacagatgaccaagagatgctgtttaaagaaacaggattcagtgtgaagctcttgtcagaagatgtgaagtcgtattatacagtacatctactacaattagaaaatatcaatagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttgatggaaaaaggagatgatattaacataaaacaagtgttactgaacatgagaaaataccgaatgggtcttattcagaccccagatcaactgagattctcatacatggctataatagaaggagcaaaatgtataaagggagattctagtatacagaaacgatggaaagaactttctaaggaagacttatctcctgcctttgatcattcaccaaacaaaataatgactgaaaaatacaatgggaacagaataggtctagaagaagaaaaactgacaggtgaccgatgtacaggactttcctctaaaatgcaagatacaatggaggagaacagtgagagtgctctacggaaacgtattcgagaggacagaaaggccaccacagctcagaaggtgcagcagatgaaacagaggctaaatgagaatgaacgaaaaagaaaaaggtggttatattggcaacctattctcactaagatggggtttatgtcagtcattttggttggcgcttttgttggctggacactgttttttcagcaaaatgccctataaacaattaattttgcccagcaagcttctgcactagtaactgacagtgctacattaatcataggggtttgtctgcagcaaacgcctcatatcccaaaaacggtgcagtagaatagacatcaaccagataagtgatatttacagtcacaagcccaacatctcaggactcttgactgcaggttcctctgaaccccaaactgtaaatggctgtctaaaataaagacattcatgtttgttaaaaactggtaaattttgcaactgtattcatacatgtcaaacacagtatttcacctgaccaacattgagatatcctttatcacaggatttgtttttggaggctatctggattttaacctgcacttgatataagcaataaatattgtggttttatctacgttattggaaagaaaatgacatttaaataatgtgtgtaatgtataatgtactattgacatgggcatcaacacttttattcttaagcatttcagggtaaatatattttataagtatctatttaatcttttgtagttaactgtactttttaagagctcaatttgaaaaatctgttactaaaaaaataaattgtatgtcgattgaattgtactggatacattttccatttttctaaagagaagtttgatatgagcagttagaagttggaataagcaatttctactatatattgcatttcttttatgttttacagttttccccattttaaaaagaaaagcaaacaaagaaacaaaagtttttcctaaaaatatctttgaaggaaaattctccttactgggatagtcaggtaaacagttggtcaagactttgtaaagaaattggtttctgtaaatcccattattgatatgtttatttttcatgaaaatttcaatgtagttggggtagattatgatttaggaagcaaaagtaagaagcagcattttatgattcataatttcagtttactagactgaagttttgaagtaaacacttttcagtttctttctacttcaataaatagtatgattatatgcaaaccttacattgtcattttaacttaatgaatattttttaaagcaaactgtttaatgaatttaactgctcatttgaatgctagctttcctcagatttcaacattccattcagtgtttaatttgtcttacttaaacttgaaattgttgttacaaatttaattgctaggaggcatggatagcatacattattatggatagcataccttatttcagtggttttcaaactatgctcattggatgtccaggtgggtcaagaggttactttcaaccacagcatctctgccttgtctctttatatgccacataagatttctgcataaggcttaagtattttaaagggggcagttatcatttaaaaacagtttggtcgggcgcggtggctcatgcctgtaatcccagcactttgggaggctgaagtgggcagatcacctgaggtcaggagttcaagaccagcctggccaacgtggtgaaacaccatctctactaaaaatgcaaaaattagctgggcatggtggagggcacctgtaatctcagctactcaggaggctgaggtaggagaattgcttgaacccaggagatggaggttgcagtgagctgagatcacgtcactgcactccagccagggcgacagagcgagactccatctcaaaagaaacaaacaaaaaaaacagtttgggccgggtgtggtggctcacgcttgtaatcccagcacttcggaaggccaaggcgggcggatcacgaggtcaagagatggagactgtcctggccaacatggtgaaatcccttctttactaaaaatacaaaaattatctgggcgtggtggtgcatgcctgtagtcccagctccttgggaggctaaggcaggagaatcacttgaacccgggaggcagaggttgcagtgagccgagattgcaccactgcactccagcctggcaacagagcaagacttcgtctc

SEQ ID NO: 10 - protein, 386 aa, Homo sapiens
MYGKDGKGTNWMDQGDMECRMQEIDDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYNGNRIGLEEEKLTGDRCTGLSSKMQDTMEENSESALRKRIREDRKATTAQKVQQMKQRLNENERKRKRWLYWQPILTKMGFMSVILVGAFVGWTLFFQQNAL

SEQ ID NO: 11 - DNA, 8423 bp, Mus musculus
ggcggggcggggcgcggagcgcgcatgcgccacagtgccagcgctctccccggatagagcggggcccgagcctgtccgctgtggtagttccgctcgcgctgccccgccgccatgtcggcaaccatcgagcgggagttcgaggaactggatgctcagtgtcgctggcagccgttatacttggaaattcgaaatgaatcccatgactatcctcatagagtggccaagtttccagaaaacagaaaccgaaacagatacagagatgtaagcccatatgatcacagtcgtgttaaactgcaaagtactgaaaatgattatattaatgccagcttagttgacatagaagaggcacaaagaagttacatcttaacacagggcccacttccgaacacatgctgccatttctggctcatggtgtggcagcaaaagaccaaagcagttgtcatgctaaaccgaactgtagaaaaagaatcggttaaatgtgcacagtactggccaacggatgacagagaaatggtgtttaaggaaacgggattcagtgtgaagctcttatctgaagatgtaaaatcatattatacagtacatctactacagttagaaaatatcaatactggtgaaaccagaaccatatctcacttccattataccacctggccagattttggggttccagagtcaccagcttcatttctaaacttcttgtttaaagttagagaatctggttgtttgacccctgaccatggacctgcagtgatccattgcagtgcgggcatcgggcgctctggcaccttctctcttgtagatacctgtcttgttctgatggaaaaaggagaggatgttaatgtgaaacaattattactgaatatgagaaagtatcgaatgggacttattcagacaccggaccaactcagattctcctacatggccataatagaaggagcaaagtacacaaaaggagattcaaatatacagaaacggtggaaagaactttctaaagaagatttatctcctatttgtgatcattcacagaacagagtgatggttgagaagtacaatgggaagagaataggttcagaagatgaaaagttaacagggcttccttctaaggtgcaggatactgtggaggagagcagtgagagcattctacggaaacgtattcgagaggatagaaaggctacgacggctcagaaggtgcagcagatgaaacagaggctaaatgaaactgaacgaaaaagaaaaaggtggttatattggcaacctattctcactaagatggggtttgtgtcagtcattttggttggcgctttggttggctggacactgctttttcactaaatgttctataaattaatagttttacccagcacctttctgcactagtagctgaccgtggtgcattaatctcaagggtttgttagcaatgcctcatacccagaaacactgcgctagagtagacatcagccagataagggatattacagtcacaagcccagcatctcaggactcatcactgcaggttcctctgagacccagactgtcaatggctcacaataaagacaagcatgcttgttggatactgttacttcttacagctgcgttcacaccagtgtattgagaaatcctttatcccaaggattggcttttggaggccttctggattttaacctgcacttgatataagcaataaacattgtggtttttttctacattattaatggaaagaaaatatcctttaaacaatgtatgtaatatgtaatgtactgttgaaatgggcattacaactttatataaccattttagggtaaatatattttataagtacctatttaatcttacttttgtagttaaatgtactttttaaaggttcaatctgaaagtctgttatcatagaaaaataaattgtatgttgactcagttgtatactgaatacattttccctttcctaagcagacgtttgatagaggcagttgaaactataagcaagctaagactactacacattcttatttcctttctatttatgctttatcttattttaaaaagaaaaacaaaaattttctaaacatgtcattgaaggaaattgtttttttctgcgatagttaagaagtgacagttggtcaaaatatagttgaaaacaaacaaaaacttggtttctgcaggatgtggtagcacacacagtgctcaggaagctaaaacaagaggctcaatggtttgaagccagccaaaactacatagcaaggtcctatctttaaagataagagaaaaatagaggtggtggaggagagatcagacaacaccaagaataagaaatcgattcttagccatatttaatggacaaacctgtcatctcagcttttgggagatagaggcagaaggctcacaagttcaaggccagcttcaactacatagctagccccagagtttggggccagtcaggactgcaagaaacactgtctcagaaactgaagtggtttaaaaacattttgatttctgtaaagtaaagcccatgcatgactacactgttaattttttgtgaaaatgtaaatgtaattacccagacgggataaattatggttagtaagttaaaggaaccagtgttttatacttttgatttcagttcactagactgaaattttgaagtaaaaaaaaatttaatttctttacaagttcaataaatagtacaatggtgtacaaacttacattgtcccttacctttgtaatgagtatttttaaagcataaccactaattgggttttggtggtttcaaaccctgcttggtggaaaggttccaaaccattaggacagcattgctgcttcatctcttttatatatcacgtaaaagtgcgtggtaaatcttaattagtttaaatgagacagttaatttcttaatgcagtttgaaccccataggtgtagttagaaattgtgaatggccttgaaaagcatctcacaaagcgtatgatgtatgtgtgtgtcctgactcagcatagctgtcctaaggctttgaaatggagagcaggtaagaaggatgtttcctcttgtctgtttaatctctgtttaagcggaggccttagaattagatggctatgggttttgagctttctaacacttactggtttgtttttccaaaatgtagtatgttatcctactagaccttattaaaacttacagtccaagccaataaggtggcgtacacctttaatctcaacactaagaacaccaagacagacagatccctgtgagttcaaggctagtctggtccacataataagttcccaggcagccagaaatagacattgagatcctgtcttgaaagaaagcaaaccaactgaagatagcctgagcttaaacaacttcccacaagaaaaactgataaggctgagaccagtccttccttggacgatatgctttctagagatagcattgagcaccactctttctgcctcttggtgtgtattttatgtttgtgaggattcctttggcatacggaaccctcagtgctcctccccggagcccgtctttctcccctgaacacatctttaaggatgagttttaacaggagaacctttaagtcacactgtcatgttgcttactaaaggtacatggcctgtggtgacagtgtcactggcatcatcctgagcctgtatgagatgtgctgtgctgatgagagaagggtgctgggcagagaagggatactagcagtttctgatgggttcacggctttaaacacagtgtgcgtcagtctcggtagcagcttattttaactaatttaggaataatagtttgtcttggatcaaattctgttttttgtttgtttgtttgttttttgtttttggtgtttggtttttttttaatttggggaaaaaataggctttttaaaggggattattgtttactggaaagaatcctcacttcctgtttcctcccaccttgctgtaatgtcagtggtcacaagattcaccaggtactgtgttatctcagcctcctgatttctatccatgctcaaacctaaagtgtaaaagtacacattcctttttaaaaatacgcatatgcatcatttctacgttcagcagaatctacacatttgtcaagttttccacagttctcagttctttttatccattccgttatgtgtcacctcatgtatcaaacagtgaacataaaaagatatgaagacctgtattaattagtttttgtccaaacagctgtgctctgaagctgcgtcagaggaaaggtcctaatttctgagctcagcttccatgcactcggctcggccctttgtcttaaagtaaagctagtgctgtgagtttagaactgtggcccacgtttcaagttatgacacagaacagccctctctggttgtcatttcatttccttgtttgcttttagcaccagtcccagggtgctggctcccattttctgccaggcacagaaaggctacagctgactgctttaaaaatagctctgcgtagattctgcagagaagctggaacctaatggtagtaaaagtacttttttttggccattgtatacaatctacttaacaagtttacatttctgtcaagacattgcagactgaagatctacattgccttaatttgttacttactgatacaaatctttatttgtagttgttgttttggataggtttgtatattcttttttttttttttttttttttttgtatgtgtgttgagatagtaccttgccattgcccaagcctggccttaaactcagctcaaacgactttcctacctcagcctgttgagtaactaacaccacaggtacacactgtgcacacagctttcaagtataaatcttaaagagattattttaaaactgtagataagatttcaggcccttagtcaagcgtggtgcataccttctctgagtagggccatctctgggtcctggtgagtagtgtctatgtctgtgggaaggaagggctgctcggggccttcatctggctgagctcgattcatctgttcatagcatgggacaaaataccaacagaaatgtccattctatttacatgccaacacctaacaaagtctcttatttttaaaactcctttatatggctttgccatagaatcttgtatatacttttttttttttttcaaaatagaaatgattttttttctcattaaatttgtcatcttattacttgaaacgtgggcctttgttattggcagtggcttgctcccgaggaggcctgttctgtccaccctgtcccagaacgcactcatttgagtcagatgccacagttcttcctcacactggtctttggtttataccatgcagcaccatacctagagtcacagctgtctctaattgtcccctgaatatggaatgagagactcaggctgtgccctcattcactgctgctctgcactggagcctgtccccaatcagagaacttgcctcgtggccagcagtcttccttcctgggtcctgagcagcttcaagccttctgcattagtgctttctcttagccgtggctgttgggaagaagacccactgttctccacaggttgggttgttttttttttttttcctggctgtccttgtcccagcacagtgccatcagccattgtgagcagtgcttaaagtggaaagctacaccagcctaagaggctttgtgtaagctgacgtttaggatttaaagagcctggaccatctgagttctgactctgaagctctgcttggttgtaaagttccagttgattctgagcagtgaggtgtgaggccactgtcaccggtagggtctgcttggatgccgcctgctttacttggatctgttttgttggggactgctgcaaggagaattgcatgggaattttcttctttttctttacagagacttataagcatcgagttattctttgtagtcactcattaggcatagttttttttttttaagacccatgatgctgttgctattccccccccccttttttttttggttttttgagacagggtttctctgtataactctggctgtcctggaactcactttgtagacctcaaactcagaaatccccctgcctttgcctcctgagtgctgggattaaaggcatgcgccaccacggcccggctgctgttgatattttaaatgactattttaaaaagtcgttcagtgtggaaagttgaggagaggaagcctaggtaagttcctttaaagcatgcttggctcacctcggttagtcctgatcaatctcagtcggatgctaatgtaaatgtcgtgtggcaaaacaacttttaatgcagtctgactttccctctaacacgggcaaggaagaagacaccagcatttgcctctgcagcacagaggcagcccccaggatacccacgtagctcattgcttggtttgctcgcccattttacttttgccttattaaaaataaaatggtgaagatccattcaagtgaatataatagaattatctcaaaagccatttatcttaatagtcttacaaataaagtcatttcttagaagctattccattgatttcctcttattttgctacccctaaacactatttgaaaagaagtaatgagtttcaaaaaccacagcgtgtctgttaaatggcaaatttattattcttggtaaatgtgtatttaacaaacactaggaaaggatatctcgtgtgtatgtgagagagaaagagagagtgcttcacaacactttaaataatgccagccatattttcagataagaaacccagtggaggtgtgactcacgccttattttccagcctgtgcagatagagctgagatgcagactccaggctgtggtttcagtccctccaaggctcaggctcattgtgctactccactgtgtatttacttaaaccagatgtttaagcggggaaatagtagacaccccactagtggaggggtggaatcccttttacaatgcttcactgactatggcggaccagaacgtttctgtgccaaagccccacttcattcctttctgttctgttccacattctgccagagtcagaaccagccgtttggtcccaggtcctgcgacccattgctatctaaagagtatggttccctaatgagaacactgcagagaatcactgttgggaaatcaaacaagactttgtagaccaccacaggggcttggtagatctgcctgcctatggagaaagaagccagtagacaggaagaagcttcattctcatggttggggaggagcctaagtggtggagatctagtgtattgcctgtttatacagtgataaagtcaagtattttcatgggtagagagcgagggtggaggaagggaggggctgcgatcggtgcaaaaatggaaatacctttaatctcccaaaagctttgaccactggcaaacaattgaaatatcagcaaagactactgctcttaatggtcacaccctcttgtttaaatggcgtccccctcccaagcattaaattgcgctgaactatcacagttttacttagttctagtagttataatcattagcattctccttcaggagaaaatctaaatgctggaaatctaattcagagataacaagccaactttatgtgcaaactttatatttaaactgtttctagcagtgttacagtgattgtccaaactggattagacttttgcgttgaaatcaaagtatgggtaagtctagcacatgtaataaaaccttgctgtttcttgtggctacatttttttttttaacttgtctgtctcttagcctaccatgtagaggtcatttcttgagttaagatgggatggcctaaaagattcagtgtgtagttactgaagaagtaagtcccggcgcctcagagcagtctgtctcacagccccgcttccatttggaaacctgccattctggaaggaagcacttggtgttcttggaatgttcatgttggaatgatttttgttgttgttgttgttgttgactttttagttcagtcttagttcttttgtgtttgtatctatctatgtacatctgtgtgtgtggtggccatggattgaatagatgacttcttattttatgttttaggccaagattgacagacacctaaatgttcatgacttgagactattctgcagctataaaatttgaacctttgatgtgcaaagcaagacctgaagcccactccggaaactaaagtgaggcttgctaaccctgtagattgcctcacaagttgtctgtttacaaagtaagctttccatccaggggatgaagaacgccaccagcagaagacttgcaaaccctttaatttgatgtattgttttttaacatgtgtatgaaatgtagaaagatgtaaaggaaataaattaggagcgactactttgtattgtactgccattcctaatgtatttttatactttttggcagcattaaatatttttattaaatagactatgttggttaaaaaaaaaaaaaaaaaaa

SEQ ID NO: 12 - protein, 406 aa, Mus musculus
MSATIEREFEELDAQCRWQPLYLEIRNESHDYPHRVAKFPENRNRNRYRDVSPYDHSRVKLQSTENDYINASLVDIEEAQRSYILTQGPLPNTCCHFWLMVWQQKTKAVVMLNRTVEKESVKCAQYWPTDDREMVFKETGFSVKLLSEDVKSYYTVHLLQLENINTGETRTISHFHYTTWPDFGVPESPASFLNFLFKVRESGCLTPDHGPAVIHCSAGIGRSGTFSLVDTCLVLMEKGEDVNVKQLLLNMRKYRMGLIQTPDQLRFSYMAIIEGAKYTKGDSNIQKRWKELSKEDLSPICDHSQNRVMVEKYNGKRIGSEDEKLTGLPSKVQDTVEESSESILRKRIREDRKATTAQKVQQMKQRLNETERKRKRWLYWQPILTKMGFVSVILVGALVGWTLLFH

SEQ ID NO: 13 - DNA, 1631 bp, Mus musculus
ggcggggcggggcgcggagcgcgcatgcgccacagtgccagcgctctccccggatagagcggggcccgagcctgtccgctgtggtagttccgctcgcgctgccccgccgccatgtcggcaaccatcgagcgggagttcgaggaactggatgctcagtgtcgctggcagccgttatacttggaaattcgaaatgaatcccatgactatcctcatagagtggccaagtttccagaaaacagaaaccgaaacagatacagagatgtaagcccatatgatcacagtcgtgttaaactgcaaagtactgaaaatgattatattaatgccagcttagttgacatagaagaggcacaaagaagttacatcttaacacagggcccacttccgaacacatgctgccatttctggctcatggtgtggcagcaaaagaccaaagcagttgtcatgctaaaccgaactgtagaaaaagaatcggttaaatgtgcacagtactggccaacggatgacagagaaatggtgtttaaggaaacgggattcagtgtgaagctcttatctgaagatgtaaaatcatattatacagtacatctactacagttagaaaatatcaatactggtgaaaccagaaccatatctcacttccattataccacctggccagattttggggttccagagtcaccagcttcatttctaaacttcttgtttaaagttagagaatctggttgtttgacccctgaccatggacctgcagtgatccattgcagtgcgggcatcgggcgctctggcaccttctctcttgtagatacctgtcttgttctgatggaaaaaggagaggatgttaatgtgaaacaattattactgaatatgagaaagtatcgaatgggacttattcagacaccggaccaactcagattctcctacatggccataatagaaggagcaaagtacacaaaaggagattcaaatatacagaaacggtggaaagaactttctaaagaagatttatctcctatttgtgatcattcacagaacagagtgatggttgagaagtacaatgggaagagaataggttcagaagatgaaaagttaacagggcttccttctaaggtgcaggatactgtggaggagagcagtgagagcattctacggaaacgtattcgagaggatagaaaggctacgacggctcagaaggtgcagcagatgaaacagaggctaaatgaaactgaacgaaaaagaaaaaggccaagattgacagacacctaaatgttcatgacttgagactattctgcagctataaaatttgaacctttgatgtgcaaagcaagacctgaagcccactccggaaactaaagtgaggcttgctaaccctgtagattgcctcacaagttgtctgtttacaaagtaagctttccatccaggggatgaagaacgccaccagcagaagacttgcaaaccctttaatttgatgtattgttttttaacatgtgtatgaaatgtagaaagatgtaaaggaaataaattaggagcgactactttgtattgtactgccattcctaatgtatttttatactttttggcagcattaaatatttttattaaatagactatgttggttaaaaaaaaaaaaaaaaaaa

SEQ ID NO: 14 - protein, 382 aa, Mus musculus
MSATIEREFEELDAQCRWQPLYLEIRNESHDYPHRVAKFPENRNRNRYRDVSPYDHSRVKLQSTENDYINASLVDIEEAQRSYILTQGPLPNTCCHFWLMVWQQKTKAVVMLNRTVEKESVKCAQYWPTDDREMVFKETGFSVKLLSEDVKSYYTVHLLQLENINTGETRTISHFHYTTWPDFGVPESPASFLNFLFKVRESGCLTPDHGPAVIHCSAGIGRSGTFSLVDTCLVLMEKGEDVNVKQLLLNMRKYRMGLIQTPDQLRFSYMAIIEGAKYTKGDSNIQKRWKELSKEDLSPICDHSQNRVMVEKYNGKRIGSEDEKLTGLPSKVQDTVEESSESILRKRIREDRKATTAQKVQQMKQRLNETERKRKRPRLTDT

SEQ ID NO: 15 - DNA, 20 bp, Artificial Sequence
  /note=“Description of Artificial Sequence Synthetic sgRNA sequence”
ggtaccctggtcattcactt

SEQ ID NO: 16 - DNA, 20 bp, Artificial Sequence
  /note=“Description of Artificial Sequence Synthetic sgRNA sequence”
acagcccaagtgaatgacca

SEQ ID NO: 17 - DNA, 20 bp, Artificial Sequence
  /note=“Description of Artificial Sequence Synthetic sgRNA sequence”
gacacacggcgcaatgacag

SEQ ID NO: 18 - DNA, 20 bp, Artificial Sequence
  /note=“Description of Artificial Sequence Synthetic sgRNA sequence”
agagatcaaacagctcaccg

SEQ ID NO: 19 - DNA, 20 bp, Artificial Sequence
  /note=“Description of Artificial Sequence Synthetic sgRNA sequence”
gtgggtactcaccaggtgaa

SEQ ID NO: 20 - DNA, 20 bp, Artificial Sequence
  /note=“Description of Artificial Sequence Synthetic sgRNA sequence”
tgtgaagtacttgagctcct

SEQ ID NO: 21 - DNA, 20 bp, Artificial Sequence
  /note=“Description of Artificial Sequence Synthetic sgRNA sequence”
gaatatgagaaagtatcgaa

SEQ ID NO: 22 - DNA, 20 bp, Artificial Sequence
  /note=“Description of Artificial Sequence Synthetic sgRNA sequence”
ctcacttccattataccacc

SEQ ID NO: 23 - DNA, 20 bp, Artificial Sequence
  /note=“Description of Artificial Sequence Synthetic sgRNA sequence”
atgtgcacagtactggccaa

SEQ ID NO: 24 - DNA, 20 bp, Artificial Sequence
  /note=“Description of Artificial Sequence Synthetic sgRNA sequence”
gtcacgaaactccataatgg

SEQ ID NO: 25 - DNA, 20 bp, Artificial Sequence
  /note=“Description of Artificial Sequence Synthetic sgRNA sequence”
gcttgcttgagaaaacgtaa

SEQ ID NO: 26 - DNA, 20 bp, Artificial Sequence
  /note=“Description of Artificial Sequence Synthetic sgRNA sequence”
gtcacgaaactccataatgg

SEQ ID NO: 27 - DNA, 20 bp, Artificial Sequence
  /note=“Description of Artificial Sequence Synthetic sgRNA sequence”
gcttgcttgagaaaacgtaa

SEQ ID NO: 28 - DNA, 20 bp, Artificial Sequence
  /note=“Description of Artificial Sequence Synthetic sgRNA sequence”
agagtaccatatagcaaggg

SEQ ID NO: 29 - DNA, 20 bp, Artificial Sequence
  /note=“Description of Artificial Sequence Synthetic sgRNA sequence”
tgggatgctataactaggcg

SEQ ID NO: 30 - DNA, 20 bp, Artificial Sequence
  /note=“Description of Artificial Sequence Synthetic sgRNA sequence”
gcgaggtattcggctccgcg

SEQ ID NO: 31 - DNA, 20 bp, Artificial Sequence
  /note=“Description of Artificial Sequence Synthetic sgRNA sequence”
gctttcacggaggttcgacg

SEQ ID NO: 32 - DNA, 21 bp, Artificial Sequence
  /note=“Description of Artificial Sequence Synthetic primer sequence”
ccccacctctagttgcctgtt

SEQ ID NO: 33 - DNA, 23 bp, Artificial Sequence
  /note=“Description of Artificial Sequence Synthetic primer sequence”
ggcatttcacctgtaaaacccac

SEQ ID NO: 34 - DNA, 23 bp, Artificial Sequence
  /note=“Description of Artificial Sequence Synthetic primer sequence”
cacctctagttgcctgttctccc

SEQ ID NO: 35 - DNA, 20 bp, Artificial Sequence
  /note=“Description of Artificial Sequence Synthetic primer sequence”
ggggtggattttgagcccca

SEQ ID NO: 36 - DNA, 22 bp, Artificial Sequence
  /note=“Description of Artificial Sequence Synthetic primer sequence”
gtacaggctccttcctcacagc

SEQ ID NO: 37 - DNA, 27 bp, Artificial Sequence
  /note=“Description of Artificial Sequence Synthetic primer sequence”
tccatcccttaaaggtaaatgggcatc

SEQ ID NO: 38 - DNA, 23 bp, Artificial Sequence
  /note=“Description of Artificial Sequence Synthetic primer sequence”
atagacagcaatcagcagttgcc

SEQ ID NO: 39 - DNA, 21 bp, Artificial Sequence
  /note=“Description of Artificial Sequence Synthetic primer sequence”
aagggatcacgggagtagcat

SEQ ID NO: 40 - DNA, 20 bp, Artificial Sequence
  /note=“Description of Artificial Sequence Synthetic primer sequence”
aggagacccaagggtgggta

SEQ ID NO: 41 - DNA, 21 bp, Artificial Sequence
  /note=“Description of Artificial Sequence Synthetic primer sequence”
tacatgcatgggagagcgaag

SEQ ID NO: 42 - DNA, 23 bp, Artificial Sequence
  /note=“Description of Artificial Sequence Synthetic primer sequence”
atagacagcaatcagcagttgcc

SEQ ID NO: 43 - DNA, 22 bp, Artificial Sequence
  /note=“Description of Artificial Sequence Synthetic primer sequence”
aagggatcacgggagtagcatc

SEQ ID NO: 44 - DNA, 23 bp, Artificial Sequence
  /note=“Description of Artificial Sequence Synthetic primer sequence”
gggcactgagcagcaaactttat

SEQ ID NO: 45 - DNA, 26 bp, Artificial Sequence
  /note=“Description of Artificial Sequence Synthetic primer sequence”
gtgactagctttcatctttgcctctt

SEQ ID NO: 46 - DNA, 22 bp, Artificial Sequence
  /note=“Description of Artificial Sequence Synthetic primer sequence”
ctggaaggctggctgtagtgtt

SEQ ID NO: 47 - DNA, 22 bp, Artificial Sequence
  /note=“Description of Artificial Sequence Synthetic primer sequence”
ctaacctcctcaggcaccagtc

SEQ ID NO: 48 - DNA, 21 bp, Artificial Sequence
  /note=“Description of Artificial Sequence Synthetic primer sequence”
gctgaagccagcttgatgttc

SEQ ID NO: 49 - DNA, 24 bp, Artificial Sequence
  /note=“Description of Artificial Sequence Synthetic primer sequence”
cccccaagaattcttaagaccatc

SEQ ID NO: 50 - protein, 11 aa, Artificial Sequence
  /note=“Description of Artificial Sequence C(X5)R PTP signature motif sequence”
  misc_feature (1)..(1): Xaa can be I or V
  misc_feature (4)..(4): Xaa can be any naturally occurring amino acid
  misc_feature (7)..(8): Xaa can be any naturally occurring amino acid
  misc_feature (10)..(10): Xaa can be S or T
XHCXAGXXRXG